(19) United States
(12) Patent Application Publication
(10) Pub. No.: US 2012/0022040 A1
(43) Pub. Date: Jan. 26, 2012

(54) ANTIMICROBIALS

(76) Inventors: Mitchell W. Mutz, La Jolla, CA (US); Kenneth J. Barr, Boston, MA (US); Gergely Makara, Budapest (HU); Ronald Alan Aungst, JR., Clifton Park, NY (US)

(21) Appl. No.: 13/131,846

(22) PCT Filed: Dec. 1, 2009
(86) PCT No.: PCT/US2009/006343

§ 371 Date: Sep. 16, 2011

Related U.S. Application Data
(60) Provisional application No. 61/200,694, filed on Dec. 1, 2008.
(60) Provisional application No. 61/171,279, filed on Apr. 21, 2009.

(51) Int. Cl. A61K 31/454; A61K 31/4545; A61P 31/00; C07D 477/20

(52) U.S. Cl. 514/210.13; 540/350

(57) ABSTRACT

Compounds useful as antimicrobials are provided, as are methods of use and preparation of such compounds and compositions containing such compounds. In one embodiment, the compounds are derivatives having a carbapenem core, and are useful for treating a microorganism infection.

### CROSS REFERENCE TO RELATED APPLICATIONS

This application claims priority under 35 U.S.C. §119(e) to provisional U.S. application Ser. No. 61/200,694, filed Dec. 1, 2008, and to provisional U.S. application Ser. No. 61/171,279, filed Apr. 21, 2009. The entire contents of the aforementioned applications are herein incorporated by reference.

### TECHNICAL FIELD

The present disclosure relates to compounds having a carbapenem core and useful as antimicrobials, as well as methods of use and methods of manufacture of such compounds. The disclosure finds utility, for example, in the field of pharmacology.

### BACKGROUND

Carbapenem derivatives share a core moiety having the structure

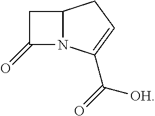

Carbapenem derivatives are commonly used as antimicrobial agents. Some known carbapenem-based antimicrobials include: meropenem, ertapenem, doripenem, panipenem, and biapenem.

In addition to the problematic development of microorganisms resistant to known antimicrobials, some carbapenem derivatives are difficult to prepare, are expensive to obtain, have a poor pharmacokinetic profile (which may be reflected in a shorter than desirable half-life), have a poor intracellular distribution for infections (where such a distribution would be desirable), and/or have significant adverse side effects; all of these drawbacks may result in lower patient compliance and/or less effective treatment. Accordingly, there continues to be a need for the development of new antimicrobials.

### SUMMARY OF THE DISCLOSURE

The present disclosure provides compounds that address one or more of the abovementioned drawbacks. In particular, the present disclosure provides carbapenem-based compounds useful as antimicrobials.

In some embodiments, the disclosure provides compounds having the structure of formula (I)

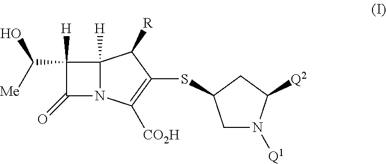

wherein: R is selected from H and lower alkyl; one of Q¹ and Q² is -L¹-U, and the other is selected from H, hydrocarbyl, heteroatom-containing hydrocarbyl, substituted hydrocarbyl, heteroatom-containing substituted hydrocarbyl, and functional groups; L¹ is a linking moiety selected from hydrocarbylene, heteroatom-containing hydrocarbylene, substituted hydrocarbylene, heteroatom-containing substituted hydrocarbylene and functional groups; and U is a group selected from Units A, B, C, and E:

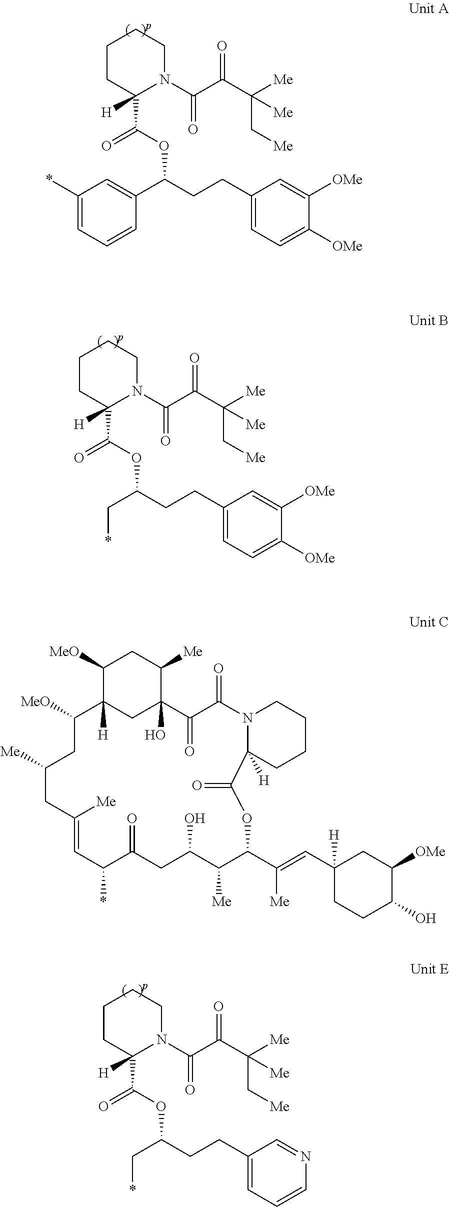

wherein p represents an integer from 0 to 2 and the stars represent the point of connection to the remainder of the compound, or pharmaceutically acceptable salts, prodrugs, or metabolites thereof.

In some embodiments, the disclosure provides a compound having the structure of formula (II)

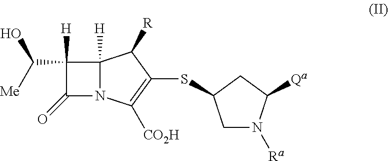

wherein: R is selected from H and lower alkyl; R^{a} is selected from H, hydrocarbyl, heteroatom containing hydrocarbyl, substituted hydrocarbyl, and substituted heteroatom-containing hydrocarbyl; Q^{a} is selected from —(CH₂)ₘ₁—X^{a}—R^{b}, and —X^{a}—NH—Ar^{a}; m1 is selected from 0 and 1; X^{a} is selected from a bond and —C(═O)—; Ar^{a} is aryl or heteroaryl substituted with one or more R^{b} groups; and R^{b} is selected from H, hydrocarbyl, and functional groups, as well as pharmaceutically acceptable salts, prodrugs, and metabolites thereof.

In still further embodiments, the disclosure provides a pharmaceutical formulation comprising a compound selected from those having the structure of formula (I) or formula (II) and a pharmaceutically acceptable carrier.

In still further embodiments, the disclosure provides a method for treating a patient with an antimicrobial compound comprising administering an effective amount of a compound selected from those having the structure of formula (I) or formula (II).

### BRIEF DESCRIPTION OF THE DRAWINGS

FIG. 1 is a graph showing percent total compound in plasma or blood cells for meropenem and Compound (2) after administration to mice.

FIG. 2 is a graph showing the plasma concentration vs. time for meropenem and Compound (2) administered to mice.

FIG. 3 provides a graph showing a comparison of the concentration of meropenem or Compound (2) in various tissue after administration.

### DETAILED DESCRIPTION OF THE INVENTION

Unless otherwise indicated, the disclosure is not limited to specific procedures, starting materials, or the like, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a reactant” includes not only a single reactant but also a combination or mixture of two or more different reactant, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term “alkyl” as used herein refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moieity). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C₁-C₆ alkoxy” or “lower alkoxy” herein may, for example, may contain 1 to 3 carbon atoms, and as a further example, such substituents may contain 1 or 2 carbon atoms (i.e., methoxy and ethoxy).

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

The term “alkylene” as used herein refers to a di-radical alkyl group. Unless otherwise indicated, such groups include saturated hydrocarbon chains containing from 1 to 24 carbon atoms, which may be substituted or unsubstituted, may contain one or more alicyclic groups, and may be heteroatom-containing. “Lower alkylene” refers to alkylene linkages containing from 1 to 6 carbon atoms. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

Similarly, the terms “alkenylene,” “alkynylene,” “arylene,” “aralkylene,” and “alkarylene” as used herein refer to di-radical alkenyl, alkynyl, aryl, aralkyl, and alkaryl groups, respectively.

The term “amino” is used herein to refer to the group —NZ¹Z² wherein Z¹ and Z² are hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation: functional groups such as halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-substituted C₁-C₂₄ alkylcarbamoyl (—(CO)—NH(C₁-C₂₄ alkyl)), di-substituted alkylcarbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N⁺≡C), cyanato (—O—C≡N), isocyanato (—O—N⁺≡C), isothiocyanato (—S—C≡N), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₅-C₂₀ arylamido (—NH—(CO)-aryl), imino (—CR═NH where R=hydrogen, C₁-C₂₄ alkyl, C₅-C₂₀ aryl, C₆-C₂₀ alkaryl, C₆-C₂₀ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O)₂), phosphinato (—P(O)(O)), phospho (—PO₂), and phosphino (—PH₂), mono- and di-(C₁-C₂₄ alkyl)-substituted phosphino, mono- and di-(C₅-C₂₀ aryl)-substituted phosphino; and the hydrocarbyl moieties C₁-C₂₄ alkyl (including C₁-C₁₈ alkyl, further including C₁-C₁₂ alkyl, and further including C₁-C₆ alkyl), C₂-C₂₄ alkenyl (including C₂-C₁₈ alkenyl, further including C₂-C₁₂ alkenyl, and further including C₂-C₆ alkenyl), C₂-C₂₄ alkynyl (including C₂-C₁₈ alkynyl, further including C₂-C₁₂ alkynyl, and further including C₂-C₆ alkynyl), C₅-C₃₀ aryl (including C₅-C₂₀ aryl, and further including C₅-C₁₂ aryl), and C₆-C₃₀ aralkyl (including C₆-C₂₀ aralkyl, and further including C₆-C₁₂ aralkyl). In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

By “linking” or “linker” as in “linking group,” “linker moiety,” etc., is meant a bivalent radical moiety. Examples of such linking groups include alkylene, alkenylene, alkynylene, arylene, alkarylene, aralkylene, and linking moieties containing functional groups including, without limitation: amido (—NH—CO—), ureylene (—NH—CO—NH—), imide (—CO—NH—CO—), epoxy (—O—), epithio (—S—), epidioxy (—O—O—), carbonyldioxy (—O—CO—O—), alkyldioxy (—O—(CH₂)ₙ—O—), epoxyimino (—O—NH—), epimino (—NH—), carbonyl (—CO—), etc.

When the term “substituted” appears prior to a list of possible substituted groups, it is intended that the term apply to every member of that group. For example, the phrase “substituted alkyl and aryl” is to be interpreted as “substituted alkyl and substituted aryl.”

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

Unless otherwise indicated, the terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, the terms include prophylactic use of active agents. “Preventing” a disorder or unwanted physiological event in a patient refers specifically to the prevention of the occurrence of symptoms and/or their underlying cause, wherein the patient may or may not exhibit heightened susceptibility to the disorder or event.

By the term “effective amount” of a therapeutic agent is meant a nontoxic but sufficient amount of a beneficial agent to provide a desirable effect.

As used herein, and unless specifically stated otherwise, an “effective amount” of a beneficial refers to an amount covering both therapeutically effective amounts and prophylactically effective amounts.

As used herein, a “therapeutically effective amount” of an active agent refers to an amount that is effective to achieve a desirable therapeutic result, and a “prophylactically effective amount” of an active agent refers to an amount that is effective to prevent or lessen the severity of an unwanted physiological condition.

By a “pharmaceutically acceptable” component is meant a component that is not biologically or otherwise undesirable, i.e., the component may be incorporated into a pharmaceutical formulation of the disclosure and administered to a patient as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “pharmacologically active” (or simply “active”), as in a “pharmacologically active” derivative or analog, refers to a derivative or analog (e.g., a salt, ester, amide, conjugate, metabolite, isomer, fragment, etc.) having the same type of pharmacological activity as the parent compound and approximately equivalent in degree.

The term “controlled release” refers to a formulation, dosage form, or region thereof from which release of a beneficial agent is not immediate, i.e., with a “controlled release” dosage form, administration does not result in immediate release of the beneficial agent in an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release and delayed release formulations.

The term “sustained release” (synonymous with “extended release”) is used in its conventional sense to refer to a formulation, dosage form, or region thereof that provides for gradual release of a beneficial agent over an extended period of time, and that preferably, although not necessarily, results in substantially constant blood levels of the agent over an extended time period.

The term “naturally occurring” refers to a compound or composition that occurs in nature, regardless of whether the compound or composition has been isolated from a natural source or chemically synthesized.

As used herein, the term “antimicrobial” refers to chemotherapeutic agents with activity against microorganisms such as bacteria, fungi, and/or viruses, and typically includes “antibiotic” chemotherapeutic agents (i.e., anti-infectives derived from bacterial sources) as well as fully synthetic agents.

In some embodiments, the disclosure provides compounds having the structure of formula (I)

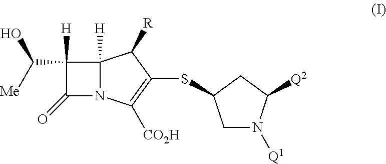

wherein:

R is selected from H and lower alkyl;

one of Q¹ and Q² is -L¹-U, and the other is selected from H, hydrocarbyl, heteroatom-containing hydrocarbyl, substituted hydrocarbyl, heteroatom-containing substituted hydrocarbyl and functional groups;

L¹ is a linking moiety selected from hydrocarbylene, heteroatom-containing hydrocarbylene, substituted hydrocarbylene, and heteroatom-containing substituted hydrocarbylene;

U is a group selected from Units A, B, C, and E:

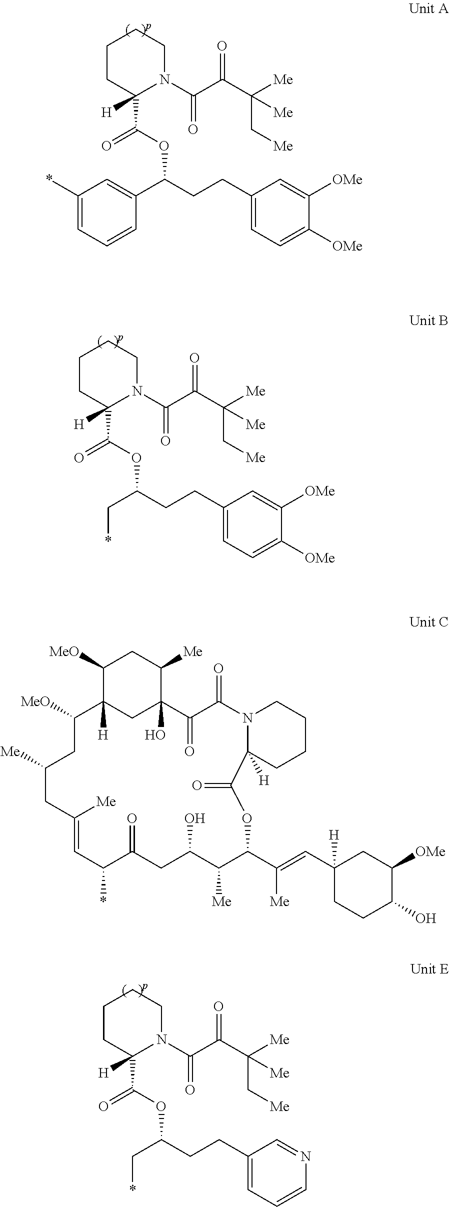

wherein p represents an integer from 0 to 2 and the stars represent the point of connection to L¹.

For example, in some embodiments, Q¹ is -L¹-U, and Q² is selected from H, —(CH₂)ₙ₂—X³—R¹, and —X³—NH—Ar¹. In such embodiments, n2 is an integer in the range of 0 to 5.

Furthermore, X³ is selected from a bond and —C(═O)—.

Furthermore, Ar¹ is aryl or heteroaryl substituted with one or more R¹ groups. For example, Ar¹ is selected from —C₆H₅ and —C₆H₅₋ₘR¹ₘ wherein m is an integer from 1 to 5.

Furthermore, R¹ is selected from H, hydrocarbyl, heteroatom-containing hydrocarbyl, substituted hydrocarbyl, heteroatom-containing substituted hydrocarbyl, and functional groups. For example, each R¹ group is a hydrocarbyl moiety independently selected from C₁-C₂₄ alkyl, C₂-C₂₄ alkenyl, C₂-C₂₄ alkynyl, C₅-C₃₀ aryl, and C₆-C₃₀ aralkyl or is a functional group independently selected from halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-substituted C₁-C₂₄ alkylcarbamoyl, di-substituted alkylcarbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₅-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, and phosphino, mono- and di-(C₁-C₂₄ alkyl)-substituted phosphino, and mono- and di-(C₅-C₂₀ aryl)-substituted phosphino.

Also for example, in some embodiments, Q² is -L¹-U, and Q¹ is selected from H and lower alkyl.

In the foregoing embodiments, L¹ is selected from alkylenes, alkenylenes, arylenes, alkarylenes, and aralkylenes, any of which may contain one or more heteroatoms and one or more substituents. In some embodiments, L¹ has the formula —Y-L-, wherein L is a linker selected from alkylenes, alkenylenes, amides, ureas, sulfoxides, sulfonamides, ethers, amines, carbonyls, and combinations thereof. Examples of suitable L linkers are provided below.

Furthermore, Y is a linker selected from a bond, —C(═O)—, —C(═NH)—, —CH(OH)—(CH₂)ₙ₃—NR⁴—, and —(CH₂)ₙ₃—NH—(SO₂)ₙ₄—, wherein n3 is an integer in the range of 1 to 3, and n4 is 0 or 1. Furthermore, R⁴ is selected from H and lower alkyl.

In some embodiments, Q² is -L¹-U, and Q¹ is selected from H, hydrocarbyl, substituted hydrocarbyl, heteroatom-containing hydrocarbyl, substituted heteroatom-containing hydrocarbyl, and functional groups. For example, L¹ is —C(X¹)(X²)-Q³-, wherein X¹ and X² are independently selected from H and OH, or may be taken together to form ═O.

Furthermore, Q³ is selected from —(CH₂)ₙ₅—N(R⁵)(Q⁴) and -L-, or Q³ has a structure selected from

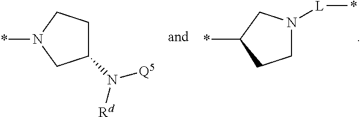

Furthermore, n5 is in the range of 0 to 5.

Furthermore, R⁵ is selected from H and lower alkyl.

Furthermore, Q⁴ is selected from -L-, —SO₂-L-, and aryl substituted with —C(═O)-L-.

Furthermore, Q⁵ is selected from -L- and βC(═O)—CH₂—NH-Q⁶.

Furthermore, Q⁶ is selected from -L-, —C(═NH)-L, and —C(═O)-L.

In some embodiments, Q¹ is -L¹-U, and Q² is selected from H and —C(X¹)(X²)-Q³-, wherein X¹ and X² are as defined above, and Q³ is —(CH₂)ₙ₅—N(R⁵)(Q⁴), or has the structure

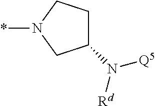

Furthermore, n5 is in the range of 0 to 5.

Furthermore, R⁵ is selected from H and lower alkyl.

Furthermore, Q⁴ is selected from lower alkyl, —SO₂NH₂, and aryl substituted with —COOH

Furthermore, Q⁵ is —C(═O)—CH₂—NH—C(═O)—NH₂ or —C(═O)—CH₂—NH—C(═NH)—NH₂.

In any of the foregoing embodiments, L is selected from

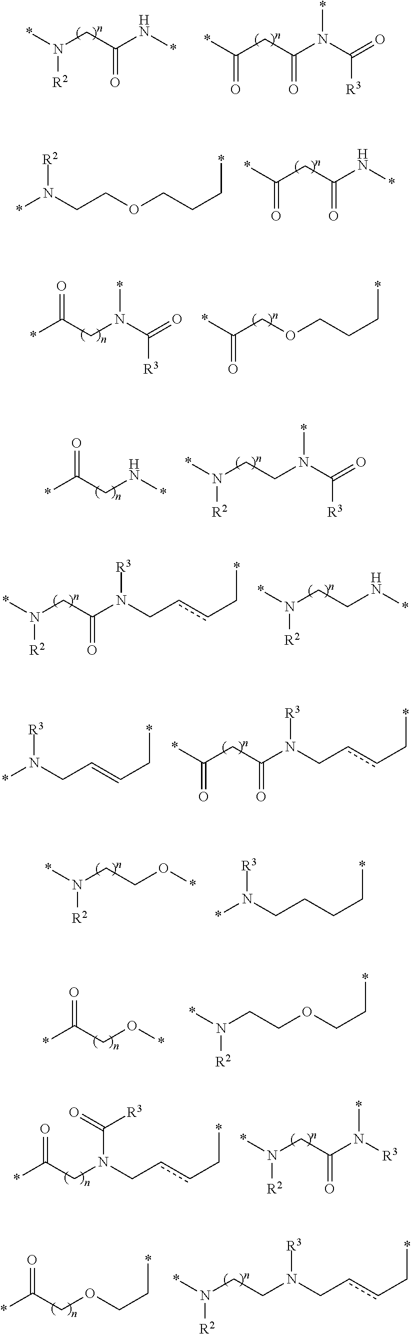

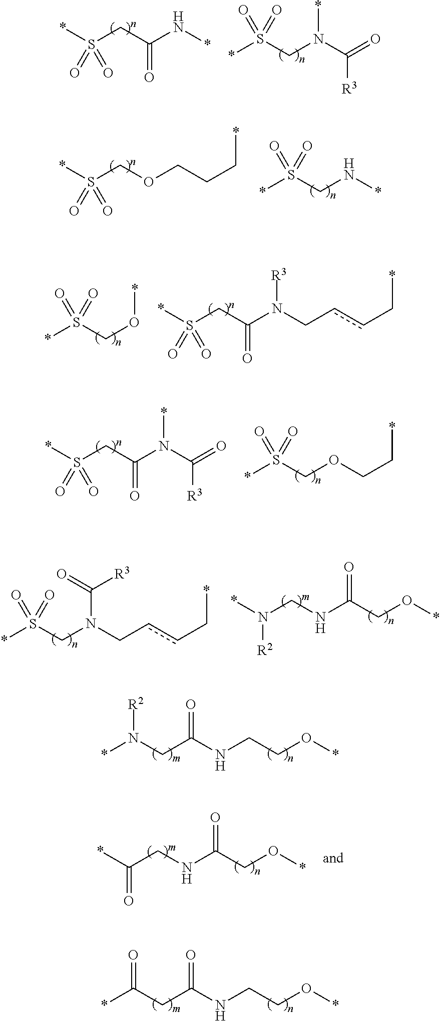

wherein:

R2 and R3 are independently selected from H, hydrocarbyl, and functional groups;

the stars represent attachment points to the remainder of the compound (e.g., to Y and to U); and

m and n are independently selected from 0, 1, and 2.

In some embodiments, there are provided compounds having the structure of formula (II)

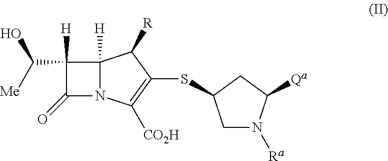

In formula (II), the variables are defined as follows.

R is as defined for Formula (I).

Furthermore, R^{a} is selected from H, hydrocarbyl, heteroatom containing hydrocarbyl, substituted hydrocarbyl, and substituted heteroatom-containing hydrocarbyl. For example, R^{a} is aralkyl which may be substitute or unsubstituted, and which may contain one or more heteroatoms.

Furthermore, Q^{a} is selected from —(CH₂)ₘ₁—X^{a}—R^{b}, and —X^{a}—NH—Ar^{a}, wherein:

m1 is selected from 0 and 1;

X^{a} is selected from a bond and —C(═O)—;

Ar^{a} is aryl or heteroaryl substituted with one or more R^{b} groups; and

R^{b} is selected from H, hydrocarbyl, and functional groups.

In some embodiments, the disclosure provides compounds having three components: a core; an additional unit; and a linker. The three components are typically linked via covalent bonds.

The core component is selected from Cores 1a, 1b, 2, 3a, 3b, 3c, 4a, 4b, 5a, 5b, 6a, 6b, 7a, 7b, and 7c:

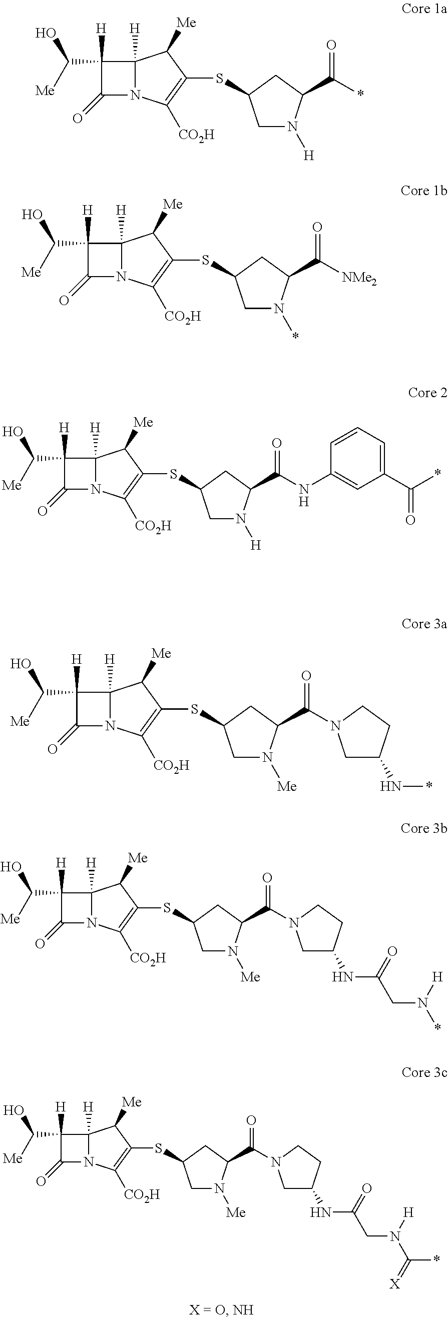

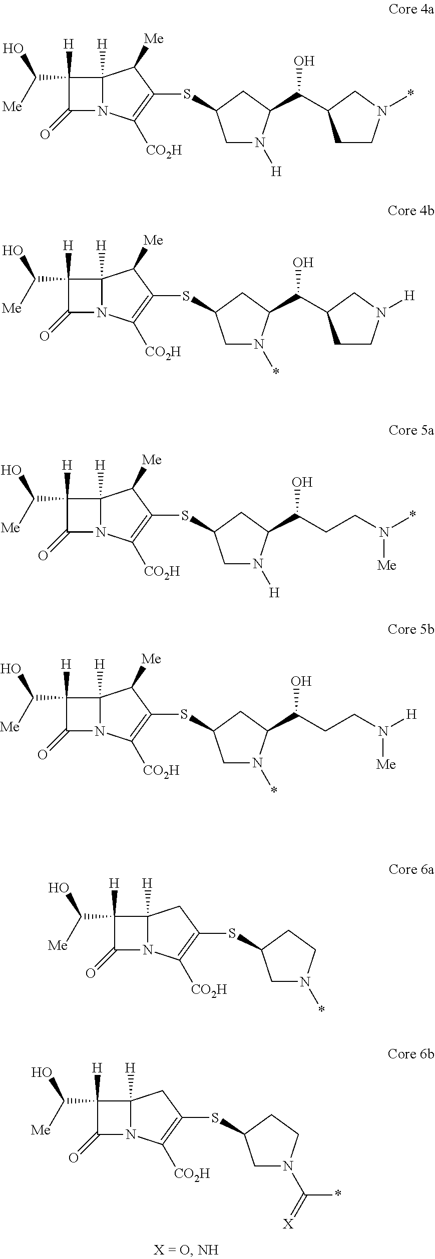

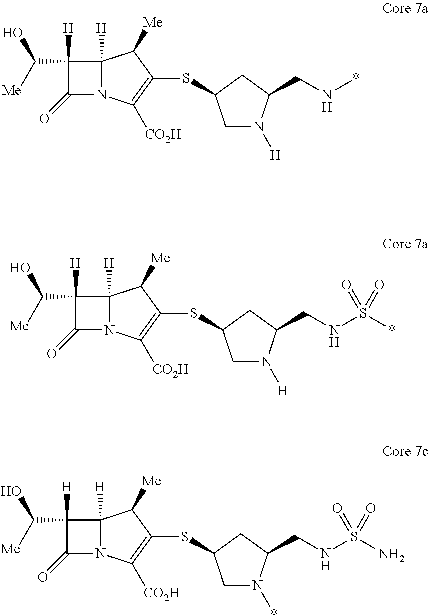

wherein the star represents the point of connection to the remainder of the compound. In preferred embodiments, the star represents the point of attachment to the linker component of the compounds. Throughout this document, such points of attachment may be alternatively and interchangeably represented by a star or by a wavy line.

The additional unit component is selected from Units A, B, C, and E:

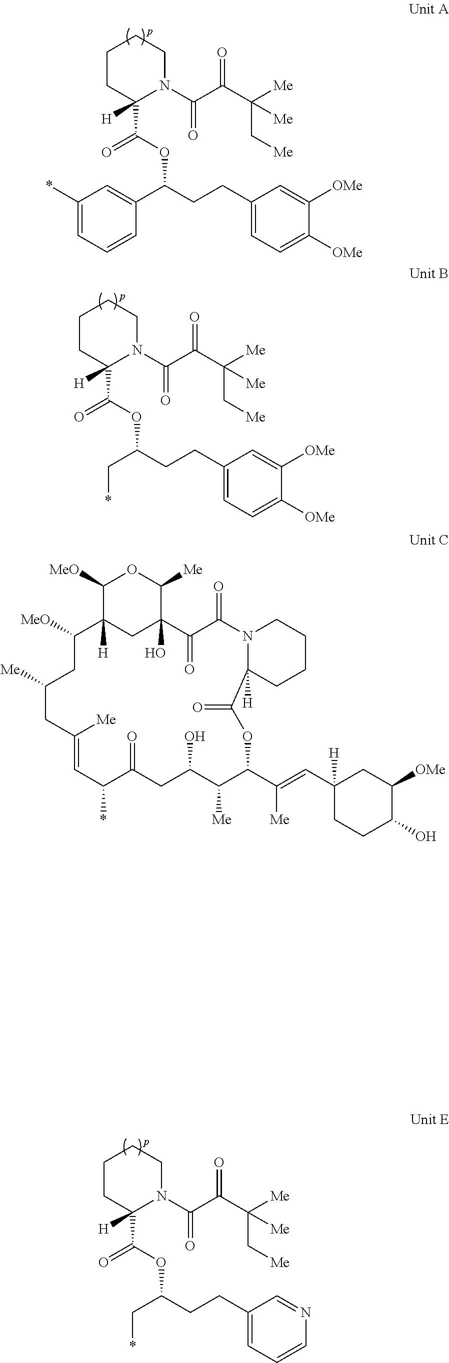

wherein p represents an integer from 0 to 2 and the stars represent the point of connection to the remainder of the compound. In preferred embodiments, the star represents the point of attachment to the linker component of the compounds, and is alternatively represented herein by a wavy line.

The linker component is a linking moiety that links the core and the additional unit components. Preferred linking moieties include alkylene linkers, alkenylenes, amides, ureas, sulfoxides, sulfonamides, amide/urea combinations, amide/amide combinations, ethers, sulfoxide/ether combinations, amide/ether combinations, amines, carbonyls, amine/ether combinations, amide/amine combinations, carbonyl/amide combinations, and other combinations as appropriate. Such linkers may include unsaturated or saturated segments, may contain one or more heteroatoms, and may be further substituted with one or more substituents where appropriate. Example substituents include hydrocarbyl, substituted hydrocarbyl, heteroatom-containing hydrocarbyl, heteroatom-containing substituted hydrocarbyl, and functional group substituents. Examples of linking moieties include the structures shown below.

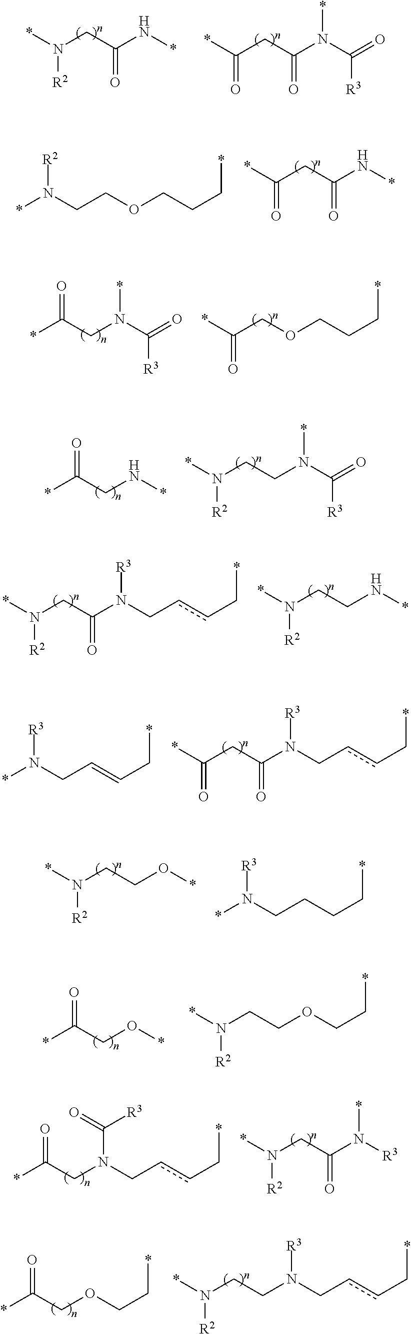

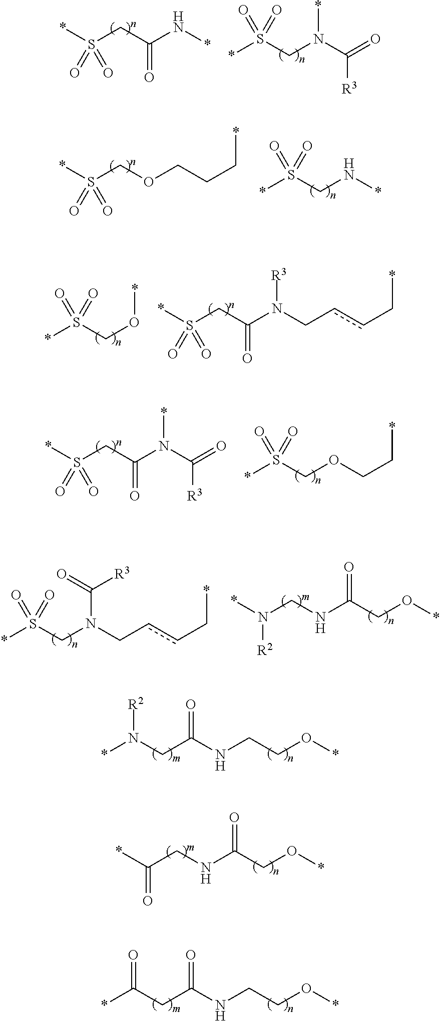

wherein R², and R³ are selected from H, hydrocarbyl, and functional groups, and the stars represent attachment points to the remainder of the compound. In preferred embodiments, one star from each linker represents the point of attachment to the core, and the other star represents the point of attachment to the additional unit U. The stars are alternatively represented herein by wavy lines. In the linker compounds disclosed herein, “m” and “n” represent integers that are independently selected. These integers may, for example, have the value 0, 1, 2, etc.

For example, the compounds may have Core 1a linked to Unit A:

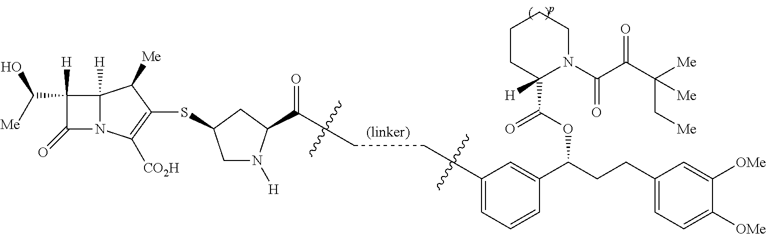

with linkers selected from

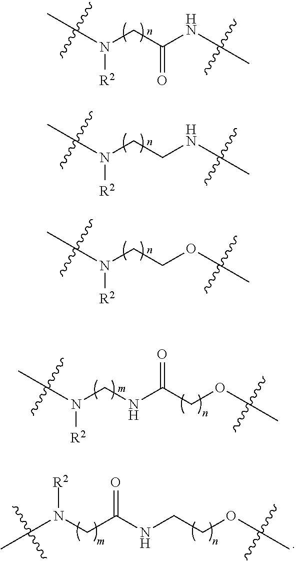

For example, the compounds may have Core 1a linked to Unit B:

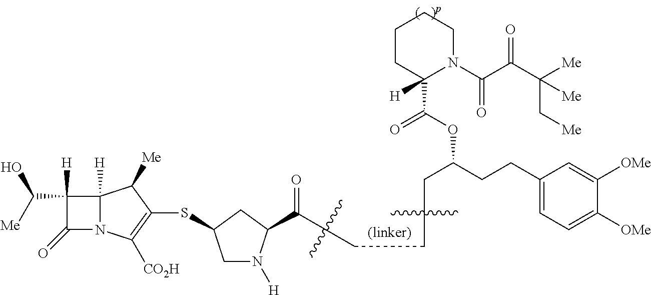

with linkers selected from

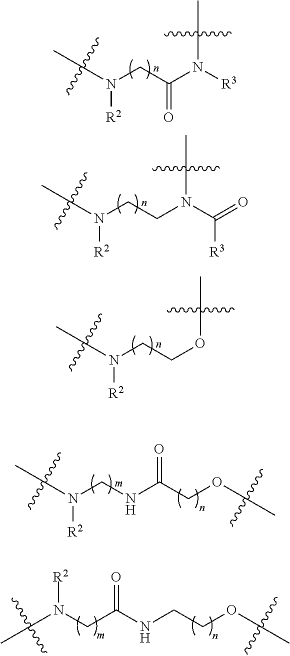

For example, the compounds may have Core 1a linked to Unit E:

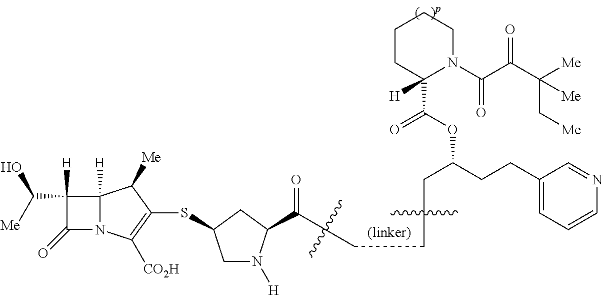

with linkers selected from

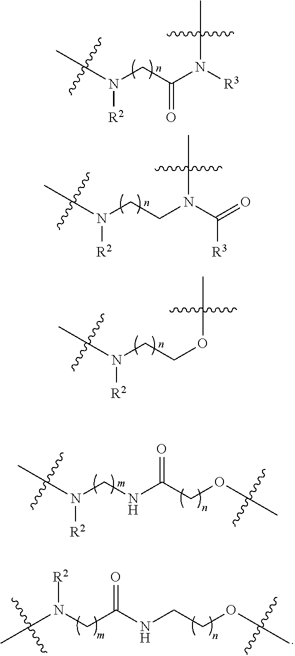

For example, the compounds may have Core 1a linked to Unit C:

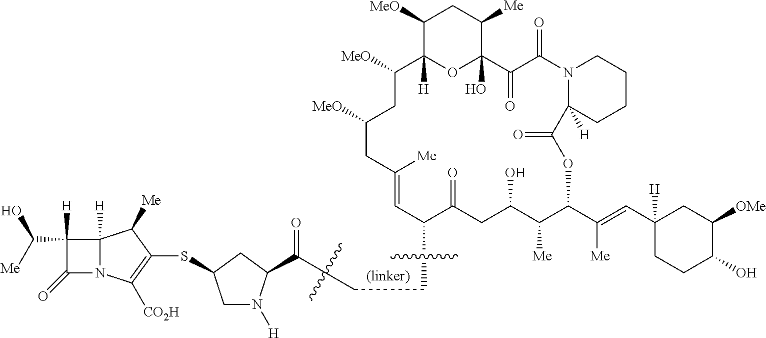

with linkers selected from

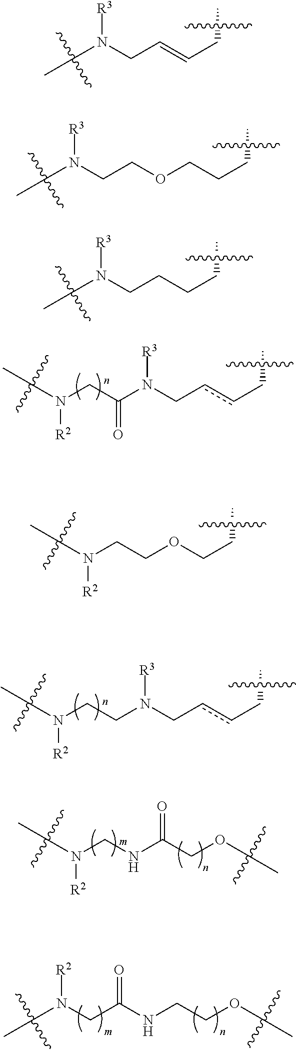

For example, the compounds may have Core 1b linked to Unit A:

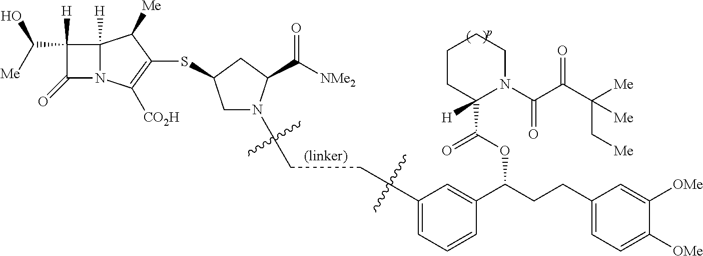

with linkers selected from

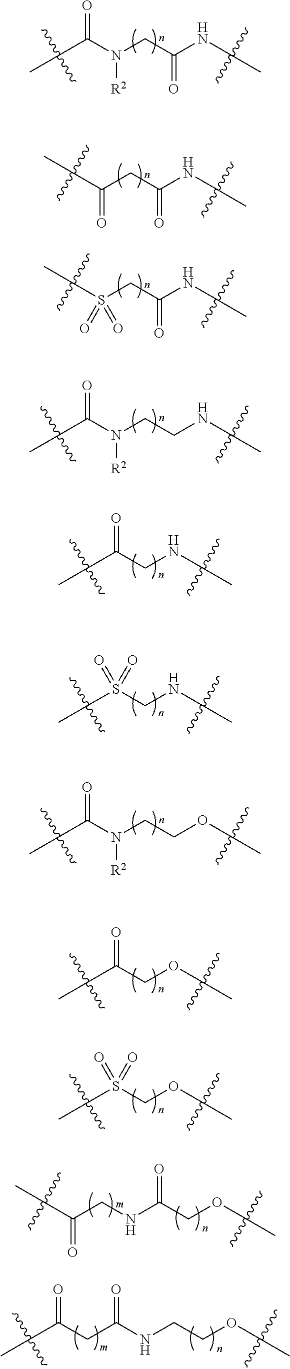

For example, the compounds may have Core 1b linked to Unit B:

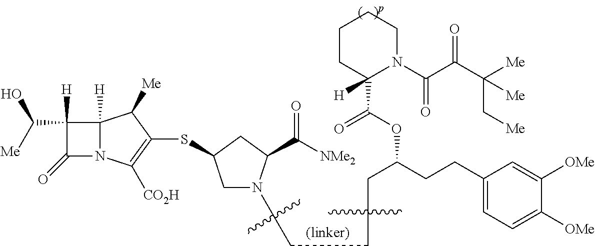

with linkers selected from

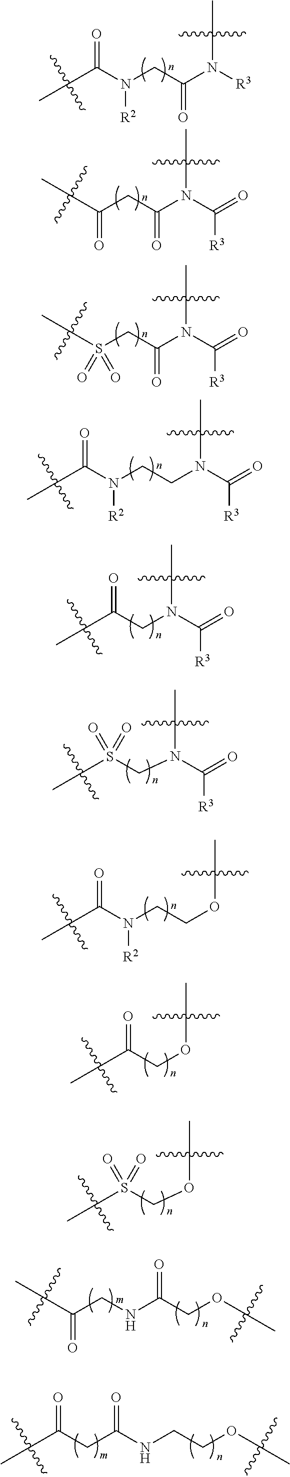

For example, the compounds may have Core 1b linked to Unit E:

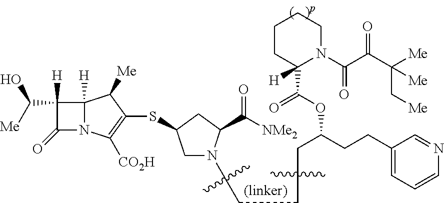

with linkers selected from

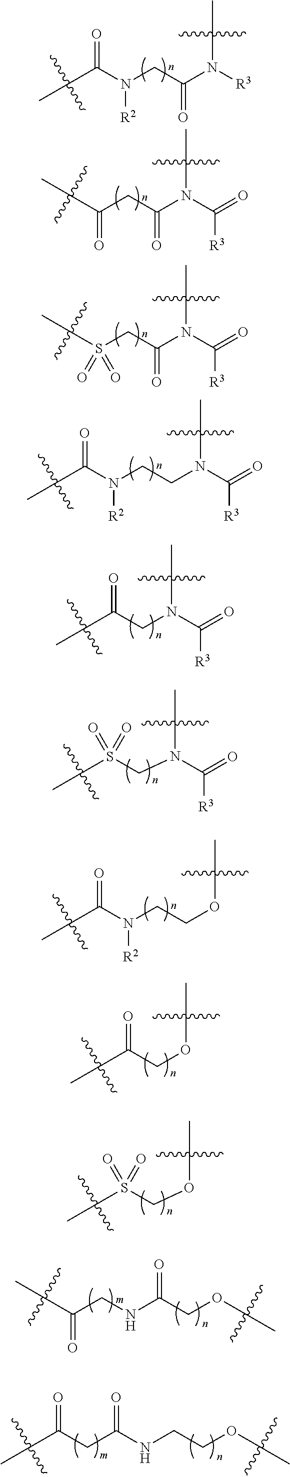

For example, the compounds may have Core 1b linked to Unit C:

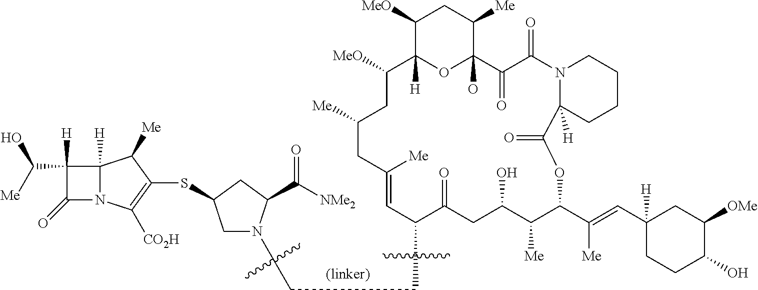

with linkers selected from

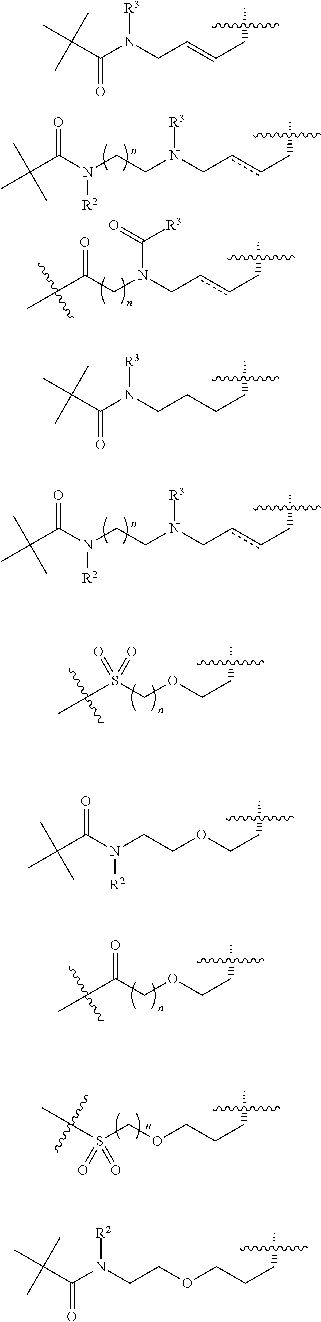

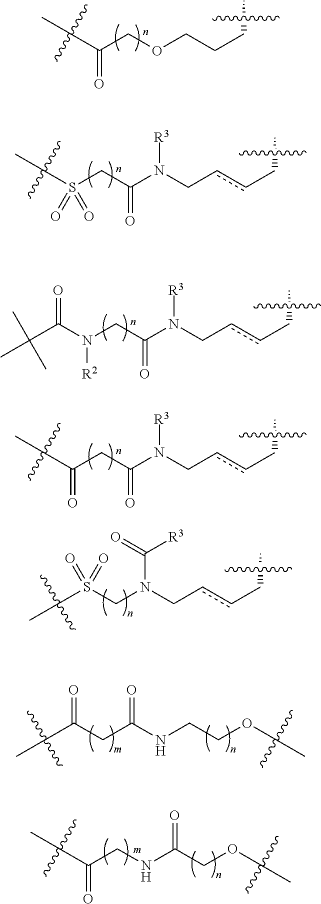

For example, the compounds may have Core 2 linked to Unit A:

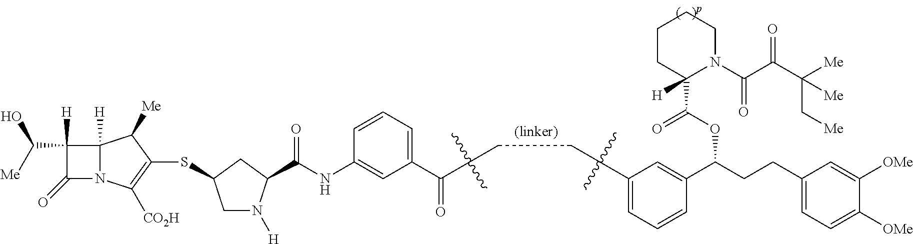

with linkers selected from

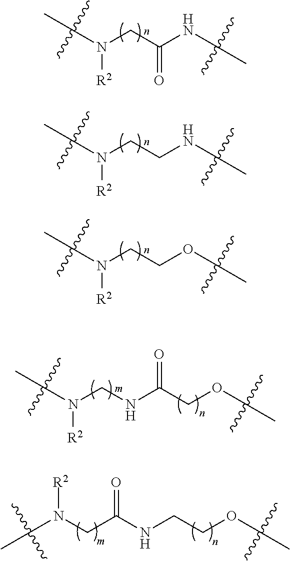

For example, the compounds may have Core 2 linked to Unit B:

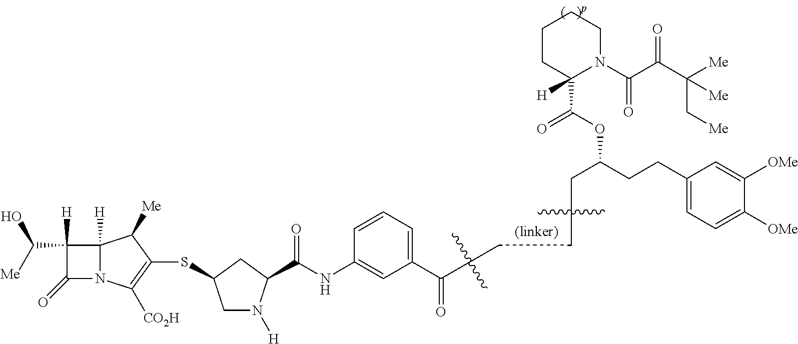

with linkers selected from

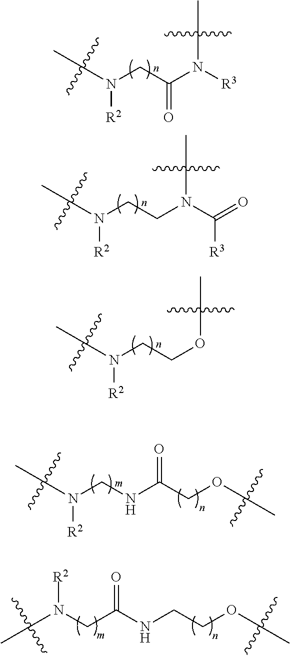

For example, the compounds may have Core 2 linked to Unit E:

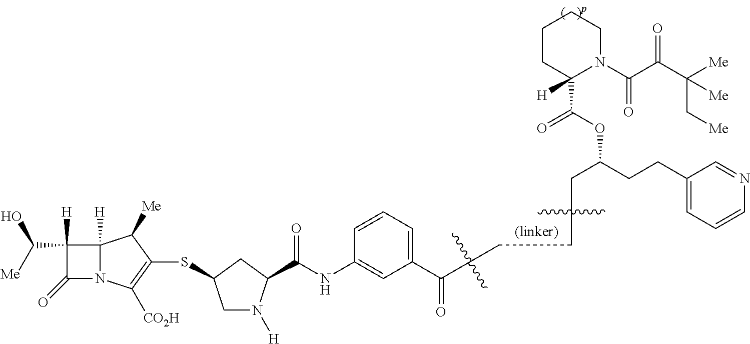

with linkers selected from

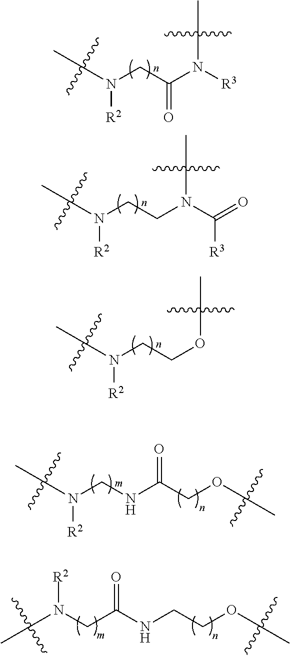

For example, the compounds may have Core 2 linked to Unit C:

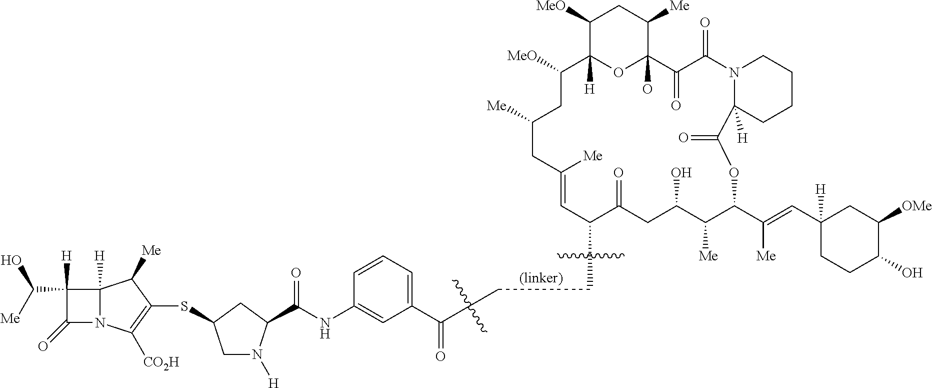

with linkers selected from

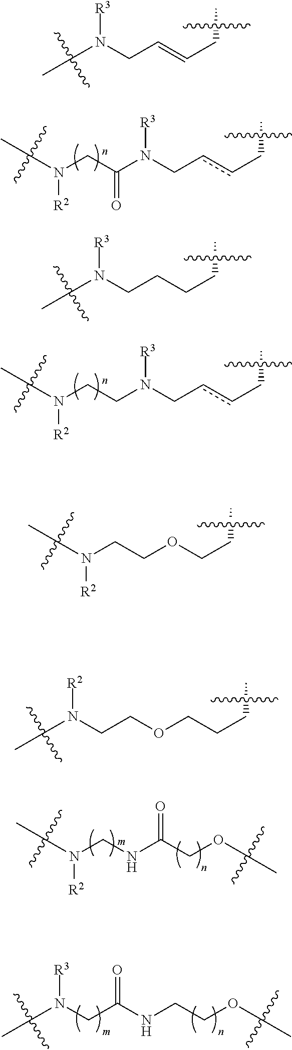

For example, the compounds may have Core 3a linked to Unit A:

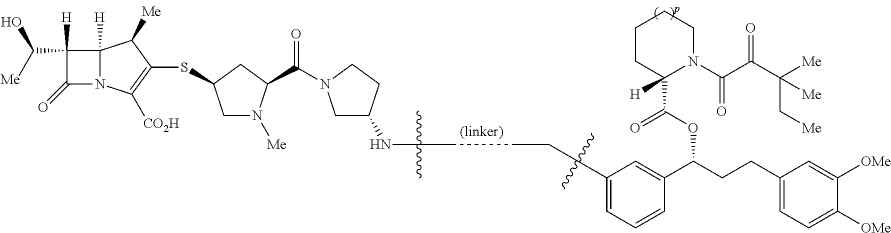

with linkers selected from

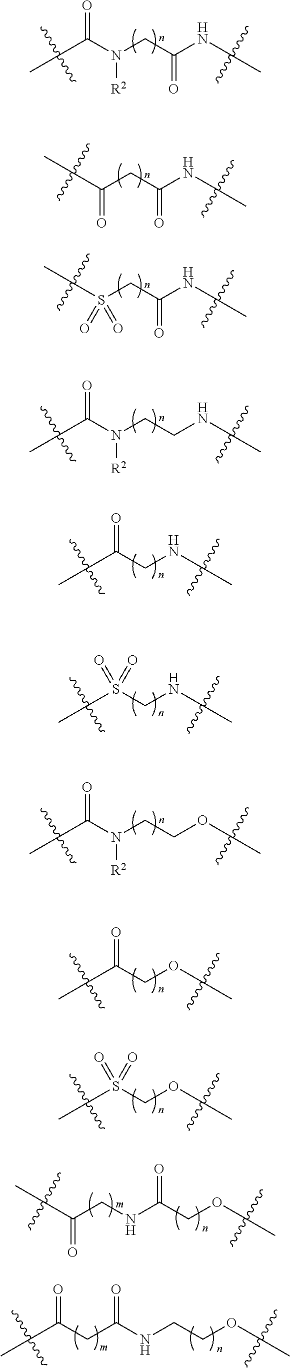

For example, the compounds may have Core 3a linked to Unit B:

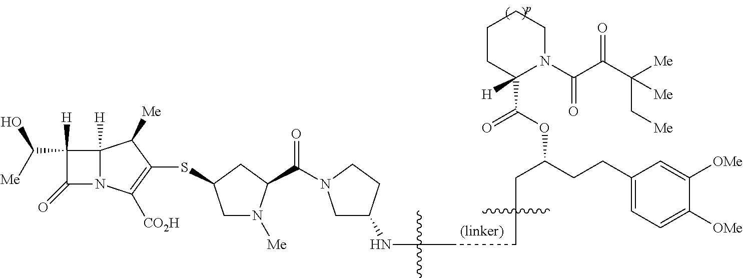

with linkers selected from

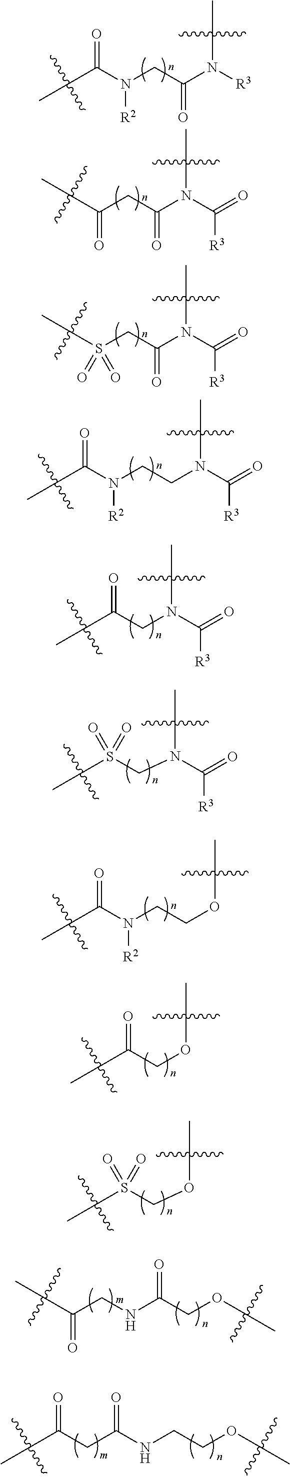

For example, the compounds may have Core 3a linked to Unit E:

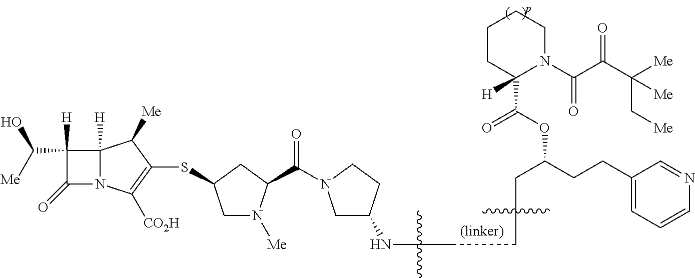

with linkers selected from

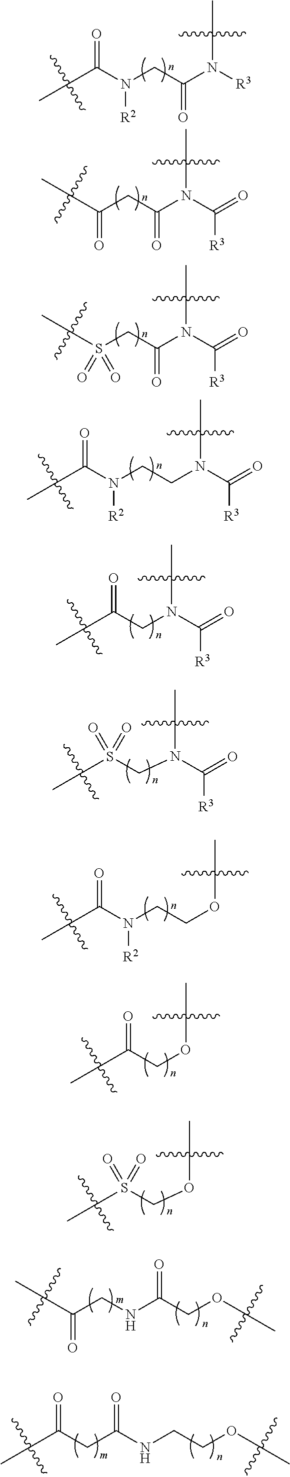

For example, the compounds may have Core 3a linked to Unit C:

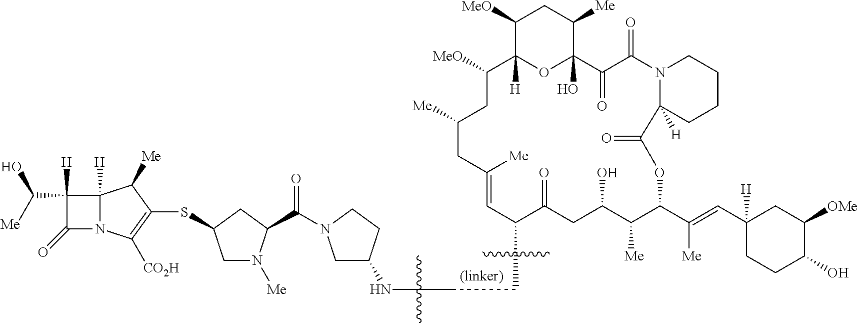

with linkers selected from

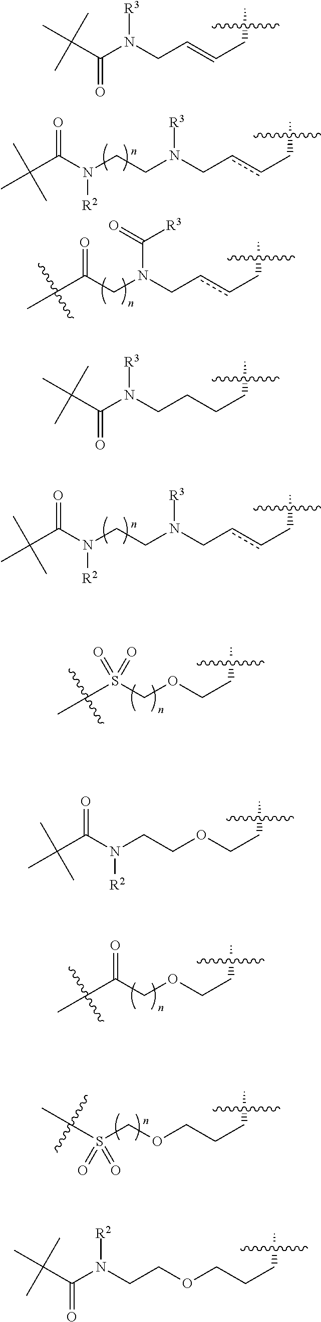

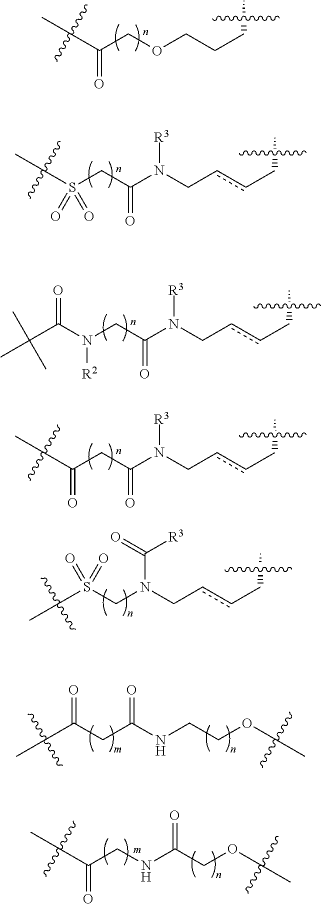

For example, the compounds may have Core 3b linked to Unit A:

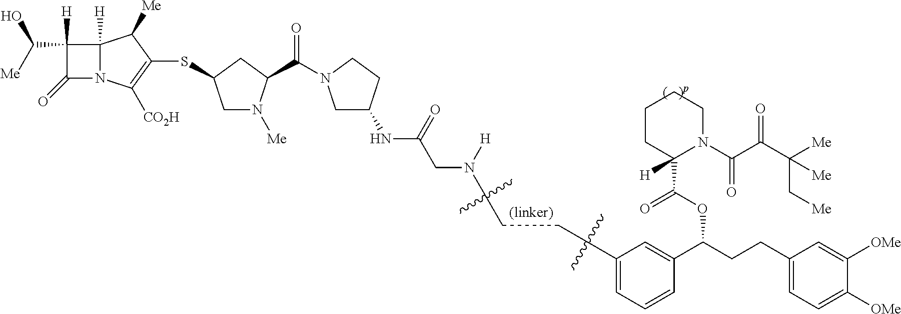

with linkers selected from

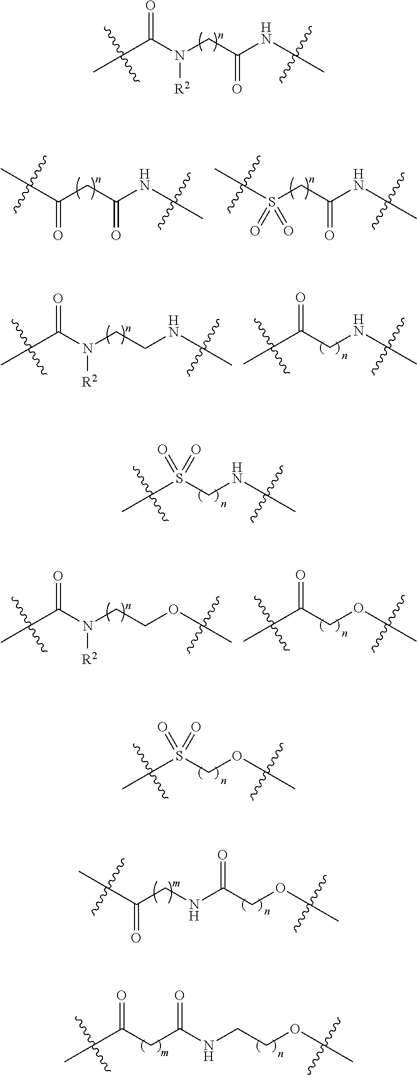

For example, the compounds may have Core 3b linked to Unit B:

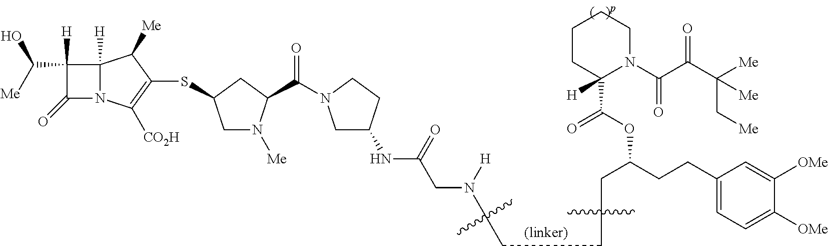

with linkers selected from

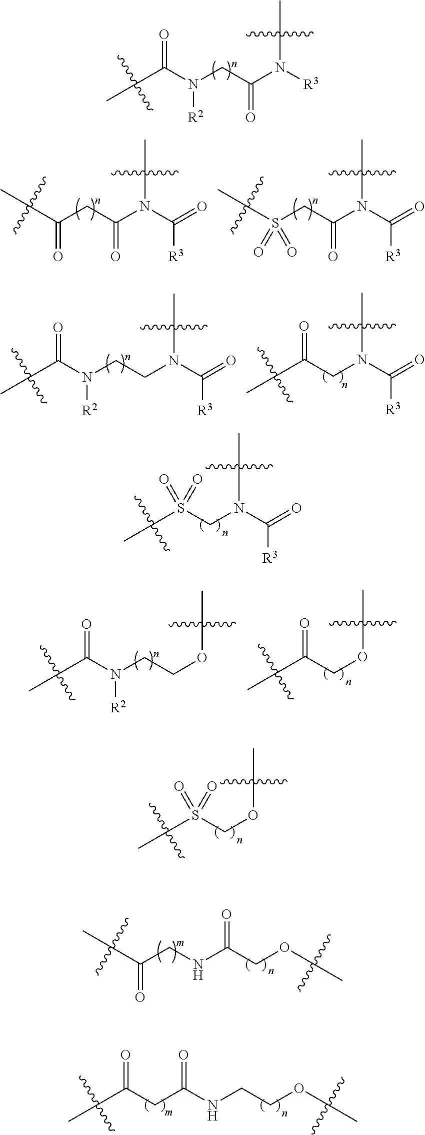

For example, the compounds may have Core 3b linked to Unit E:

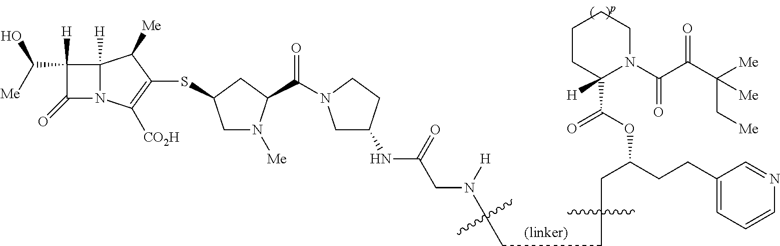

with linkers selected from

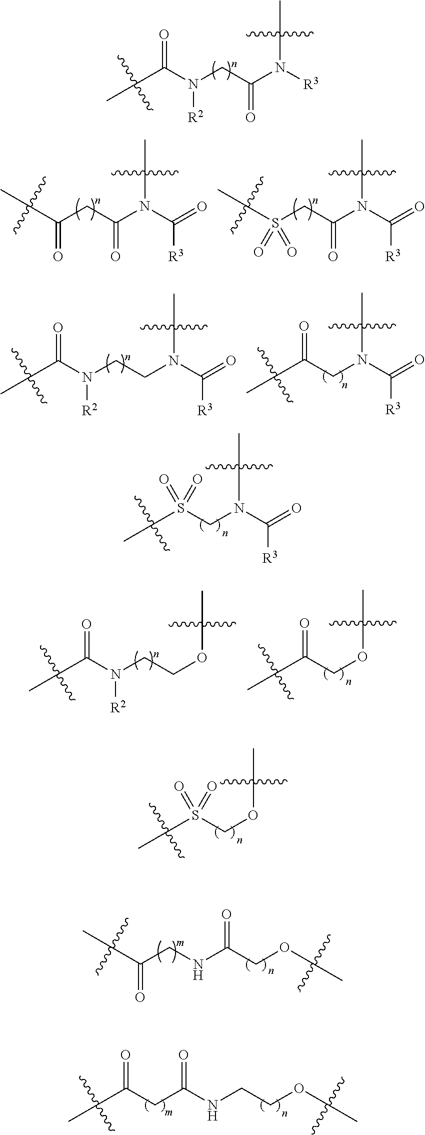

For example, the compounds may have Core 3b linked to Unit C:

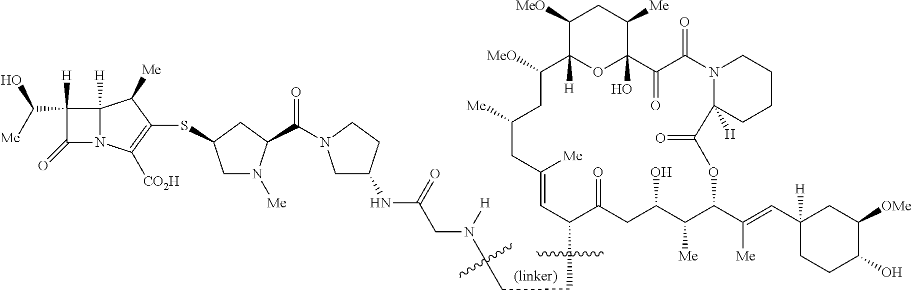

with linkers selected from

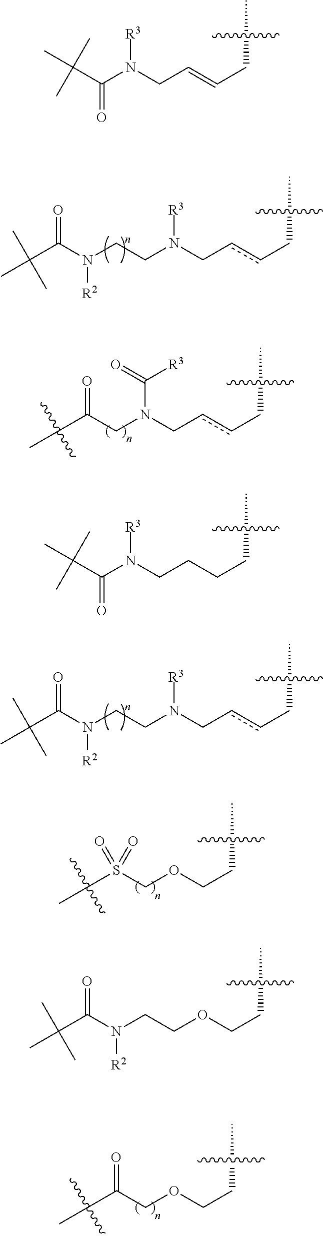

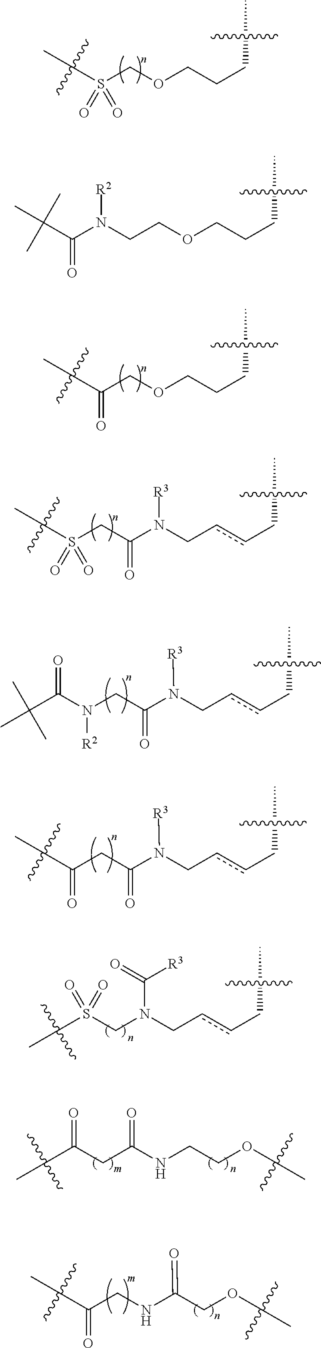

For example, the compounds may have Core 3c linked to Unit A:

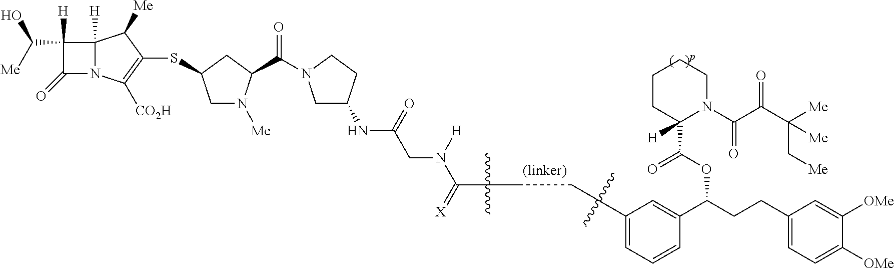

with linkers selected from

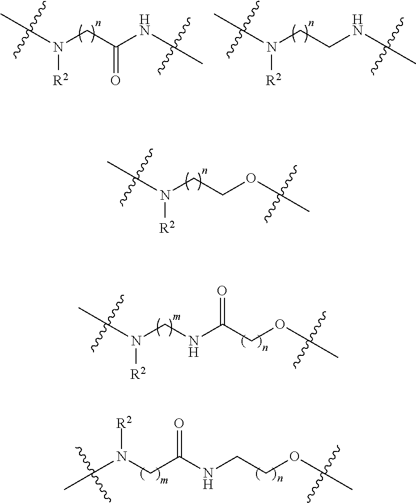

For example, the compounds may have Core 3c linked to Unit B:

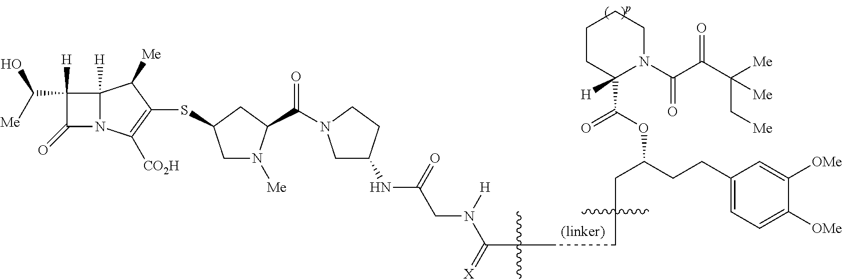

with linkers selected from

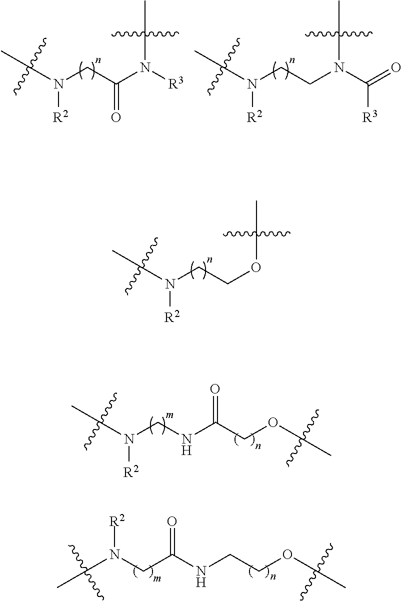

For example, the compounds may have Core 3c linked to Unit E:

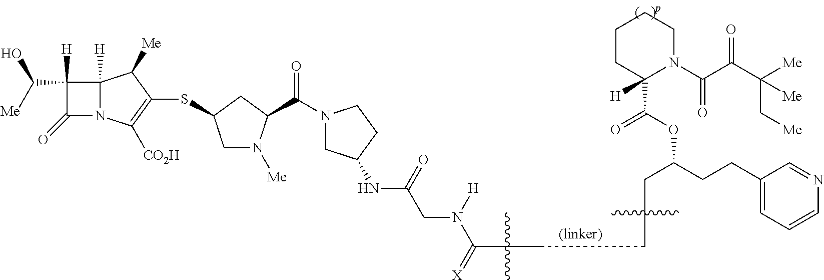

with linkers selected from

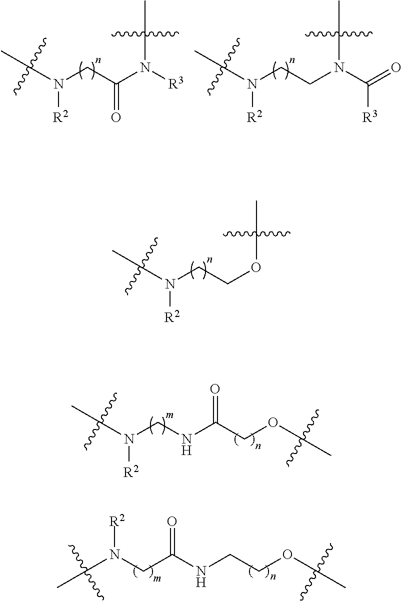

For example, the compounds may have Core 3c linked to Unit C:

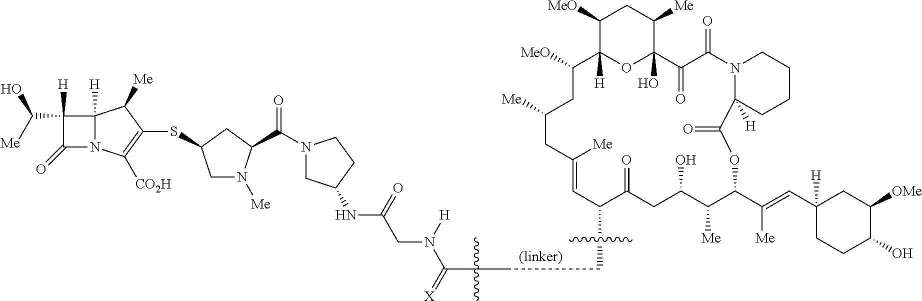

with linkers selected from

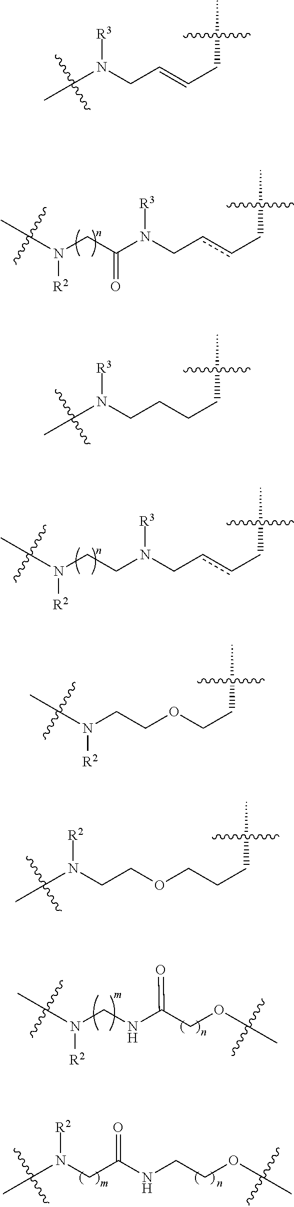

For example, the compounds may have Core 4a linked to Unit A:

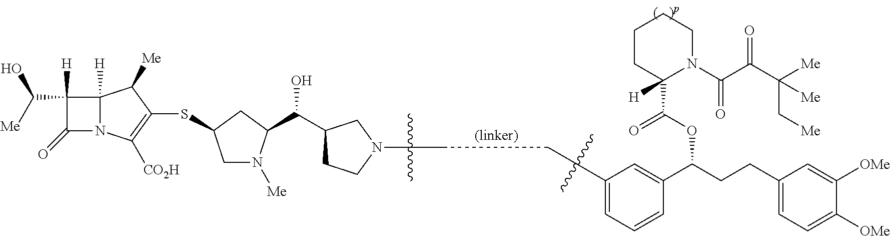

with linkers selected from

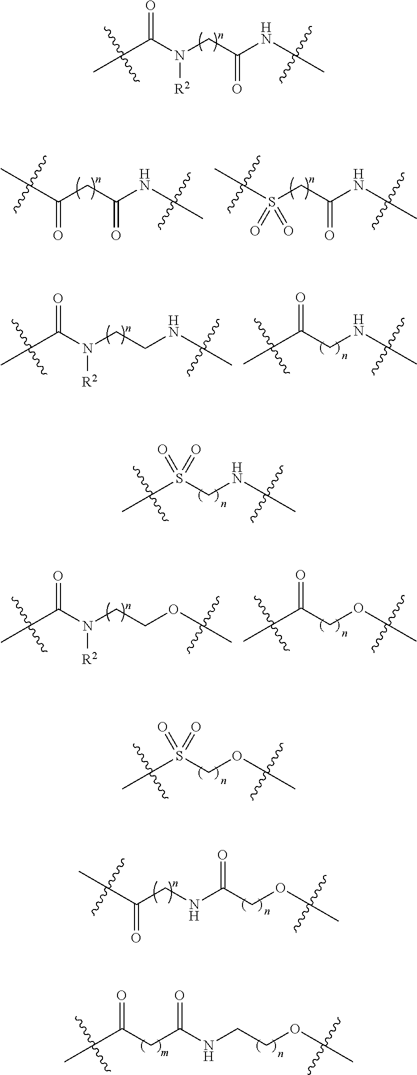

For example, the compounds may have Core 4a linked to Unit B:

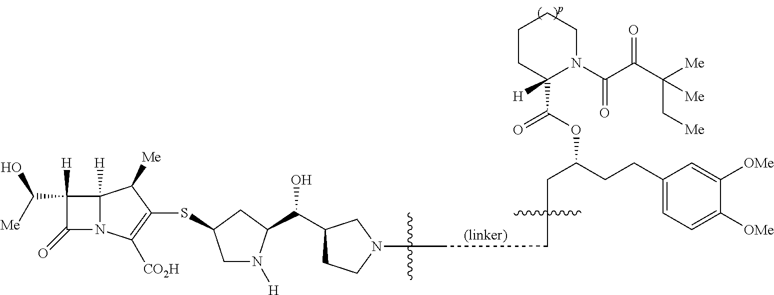

with linkers selected from

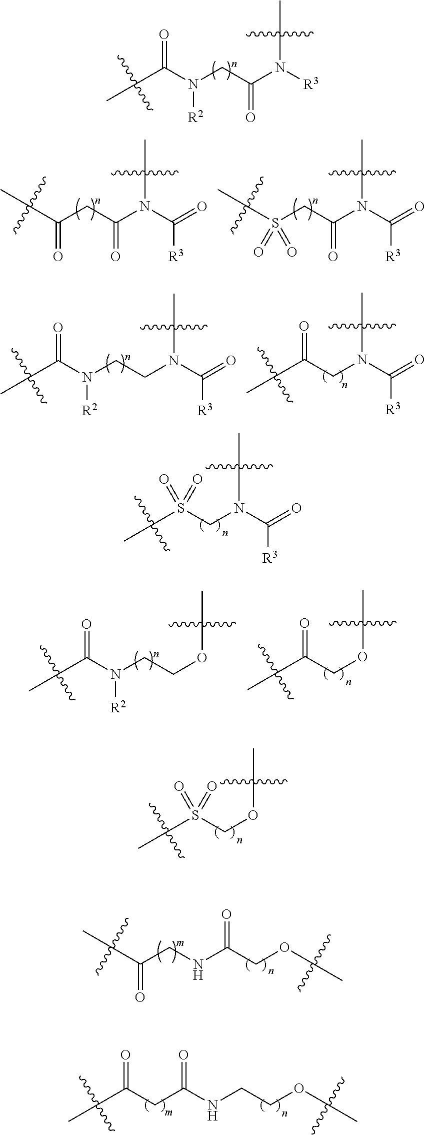

For example, the compounds may have Core 4a linked to Unit E:

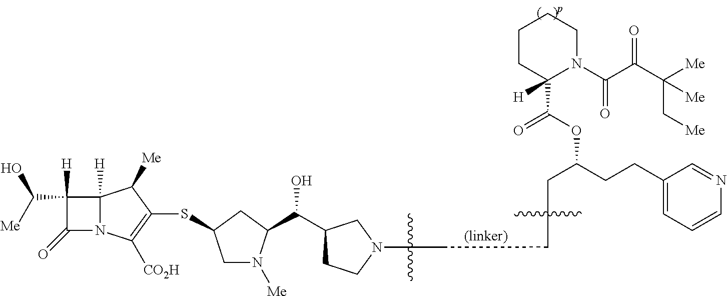

with linkers selected from

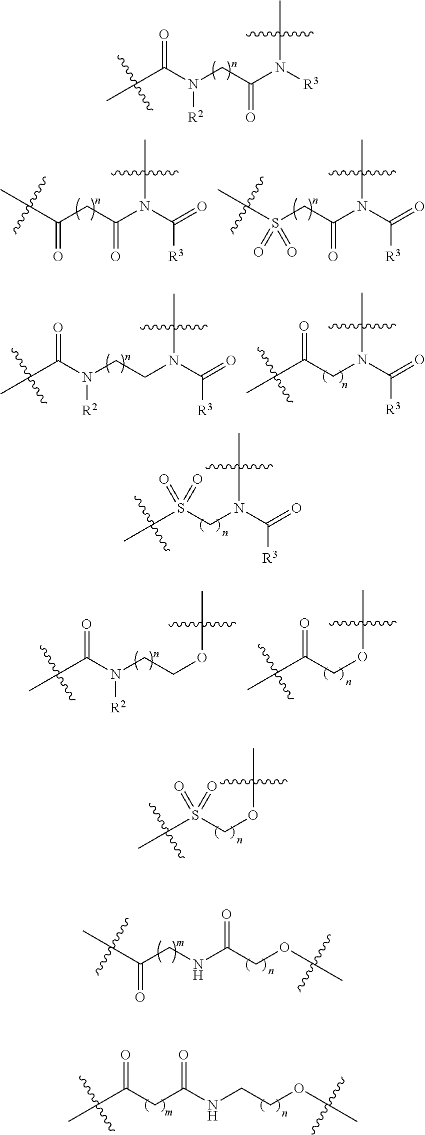

For example, the compounds may have Core 4a linked to Unit C:

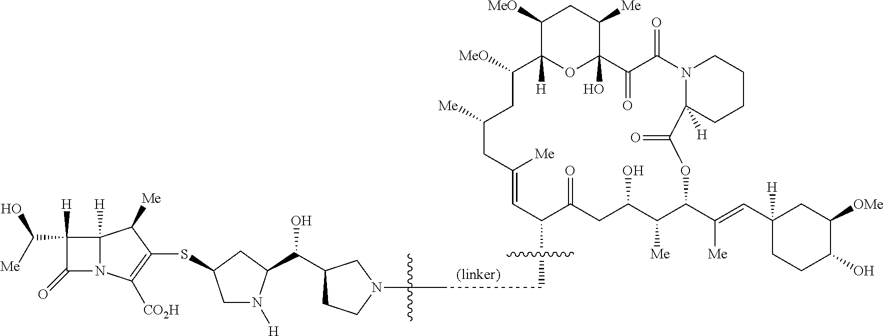

with linkers selected from

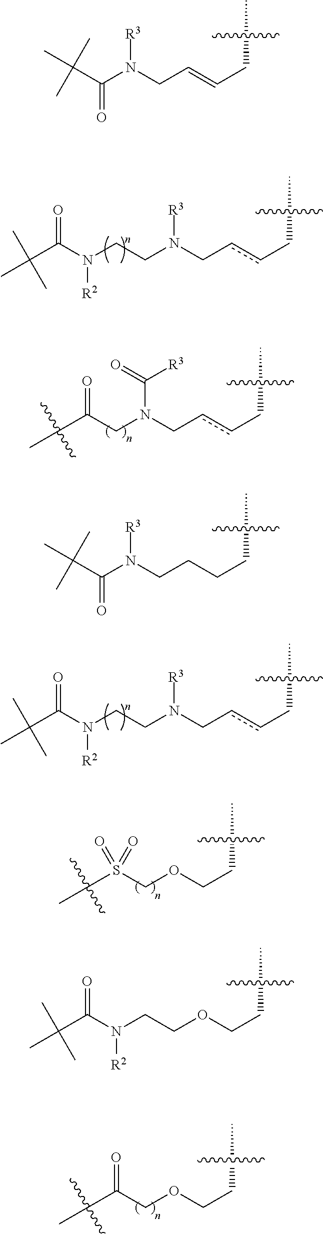

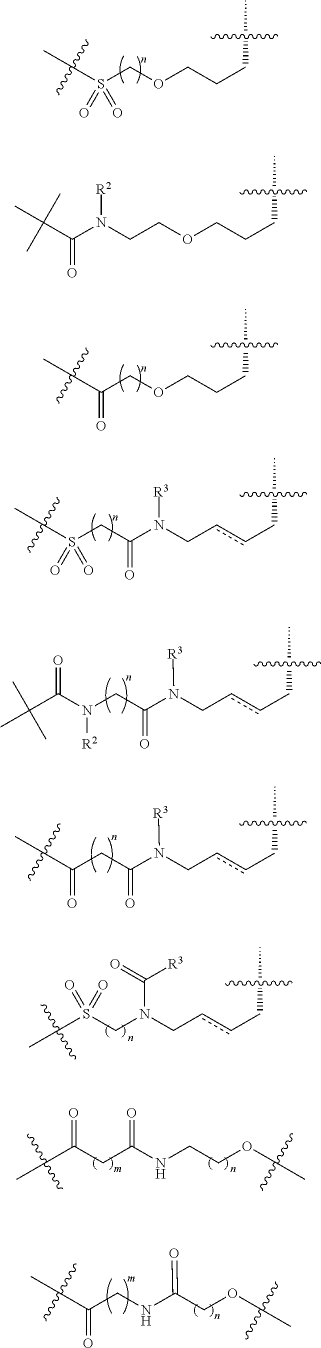

For example, the compounds may have Core 4b linked to Unit A:

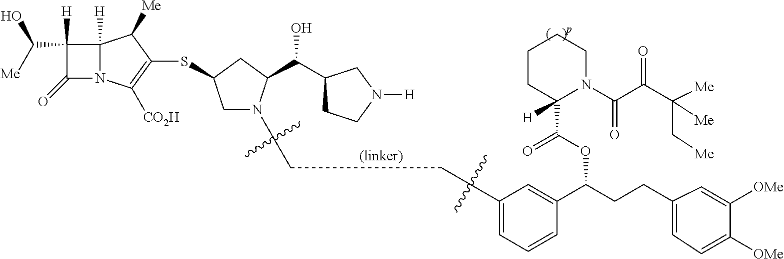

with linkers selected from

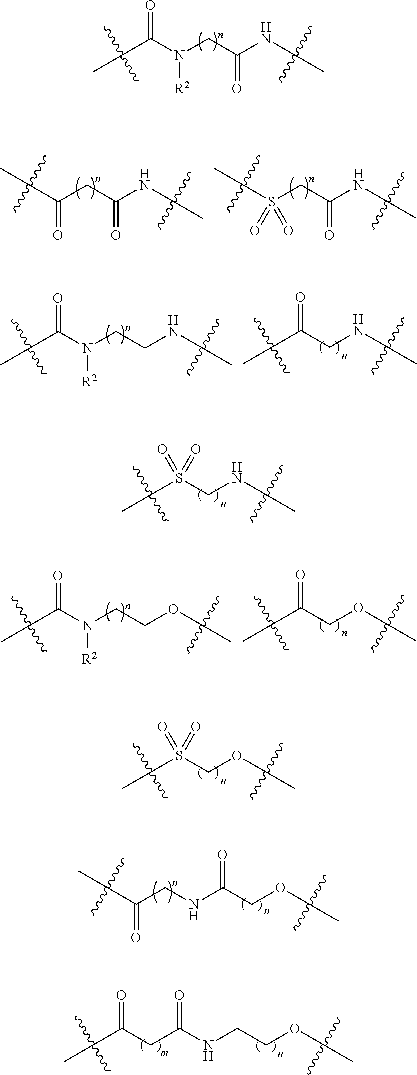

For example, the compounds may have Core 4b linked to Unit B:

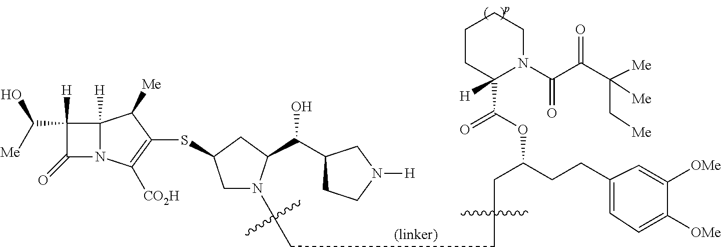

with linkers selected from

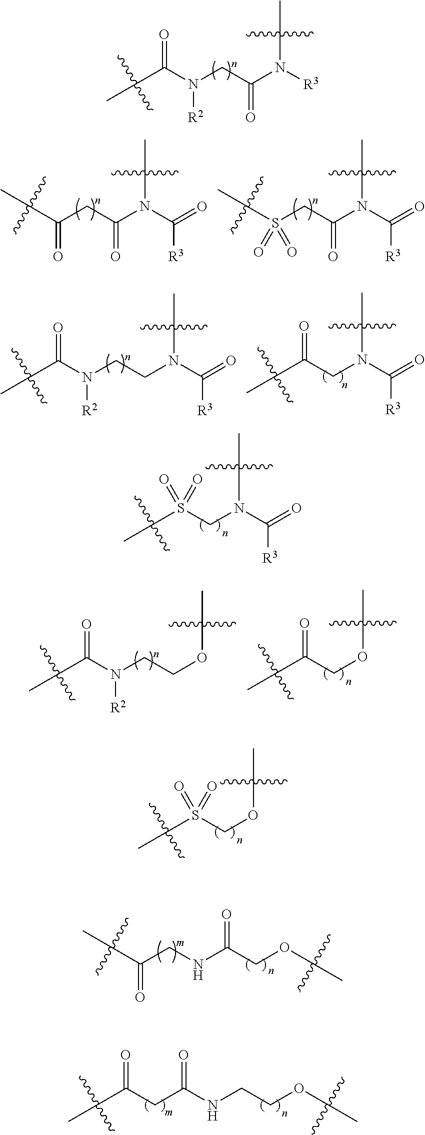

For example, the compounds may have Core 4b linked to Unit E:

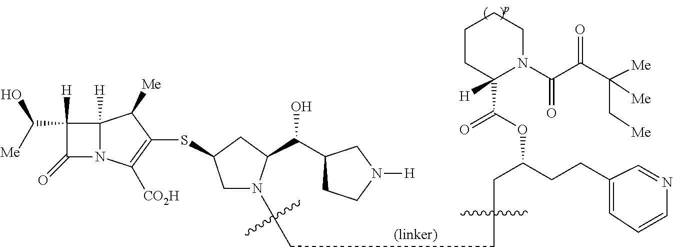

with linkers selected from

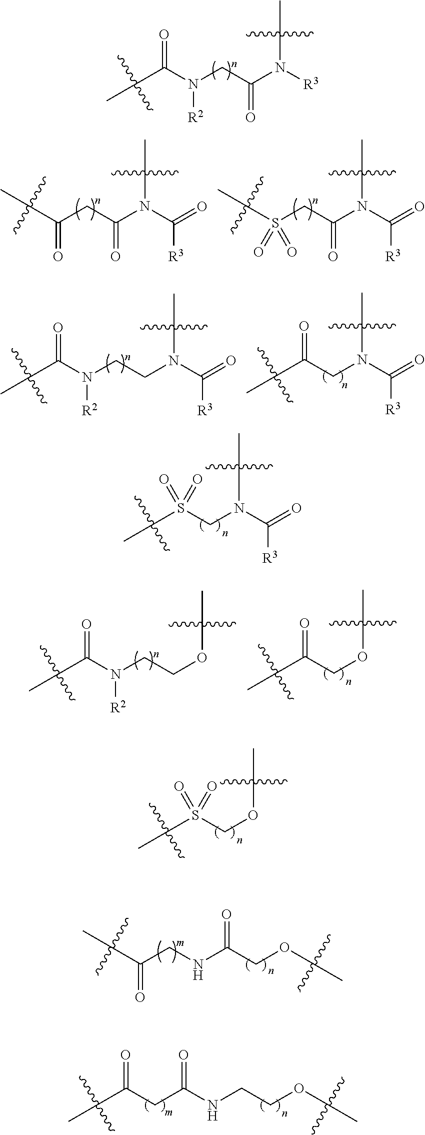

For example, the compounds may have Core 4b linked to Unit C:

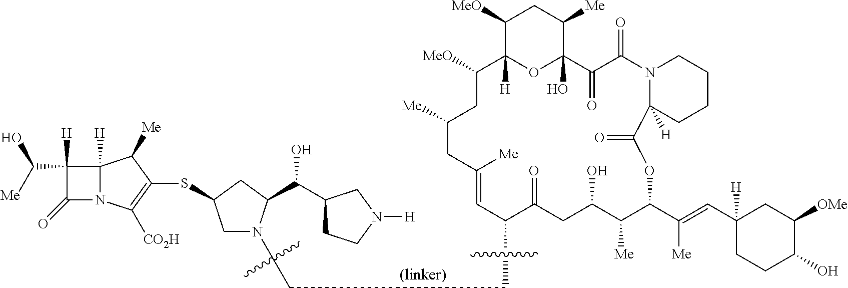

with linkers selected from

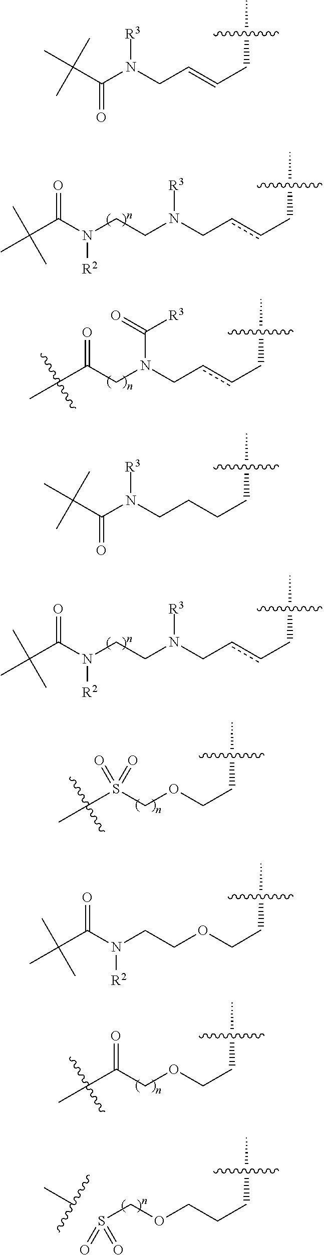

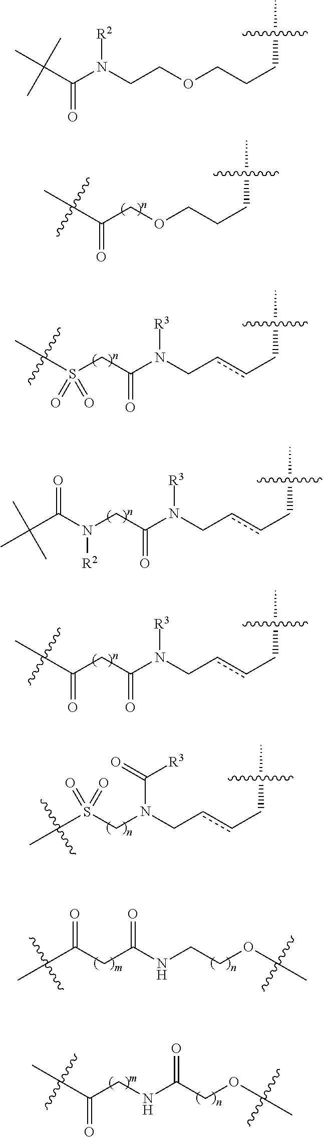

For example, the compounds may have Core 5a linked to Unit A:

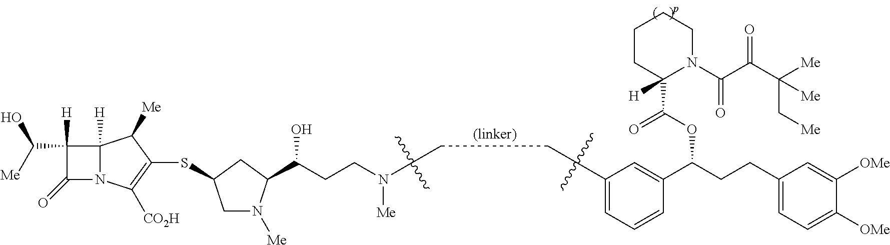

with linkers selected from

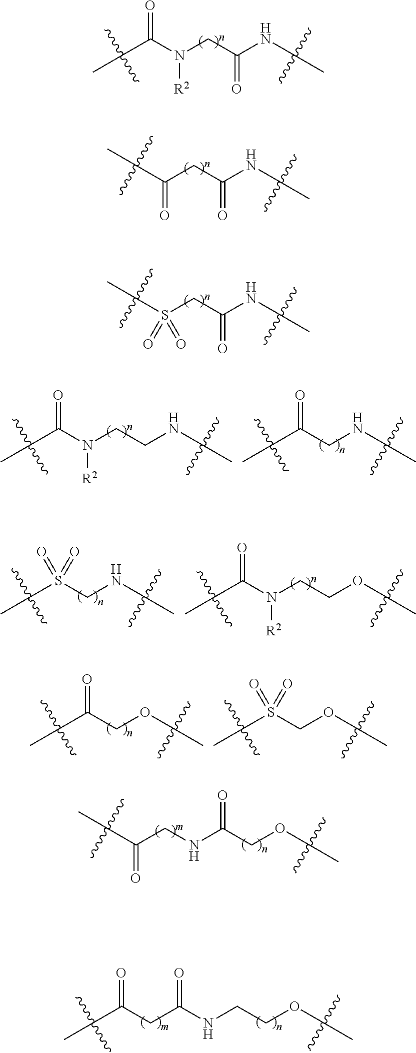

For example, the compounds may have Core 5a linked to Unit B:

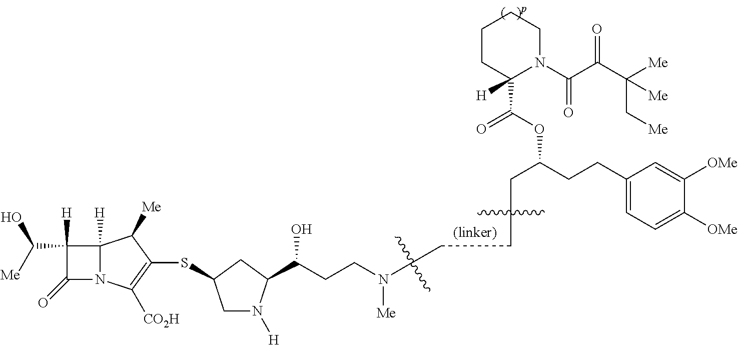

with linkers selected from

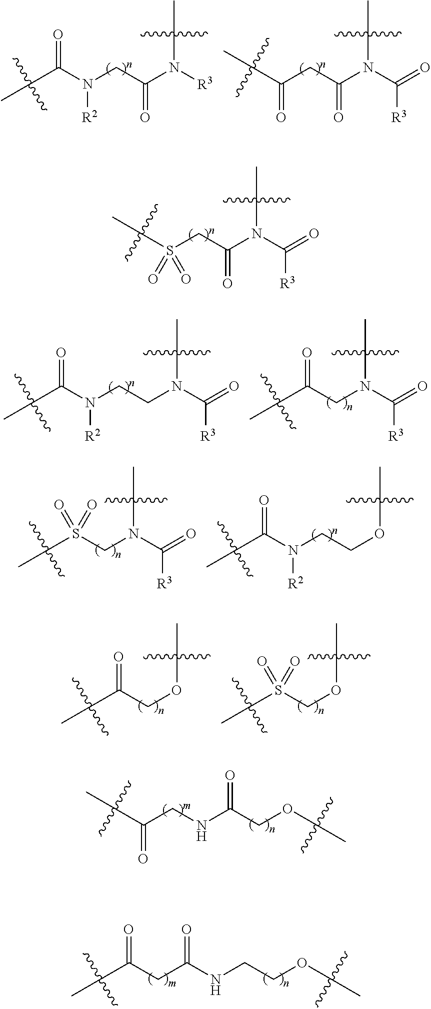

For example, the compounds may have Core 5a linked to Unit E:

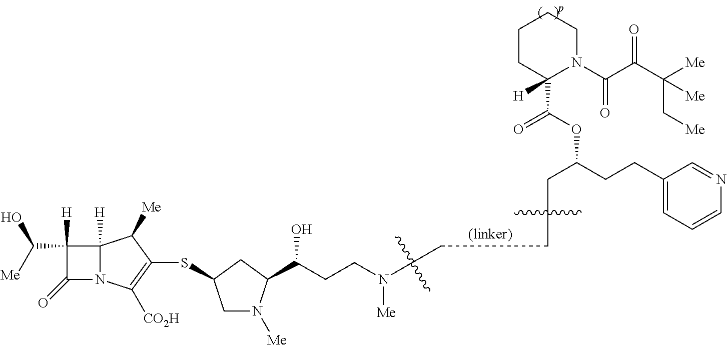

with linkers selected from

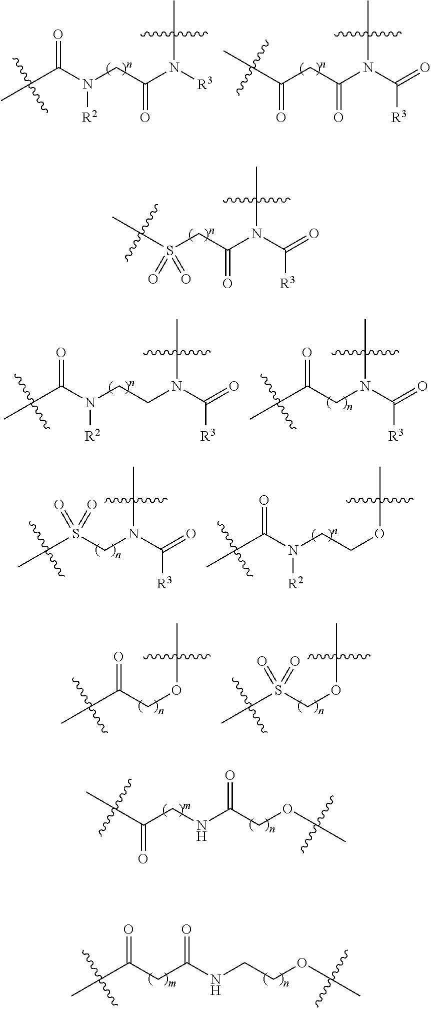

For example, the compounds may have Core 5a linked to Unit C:

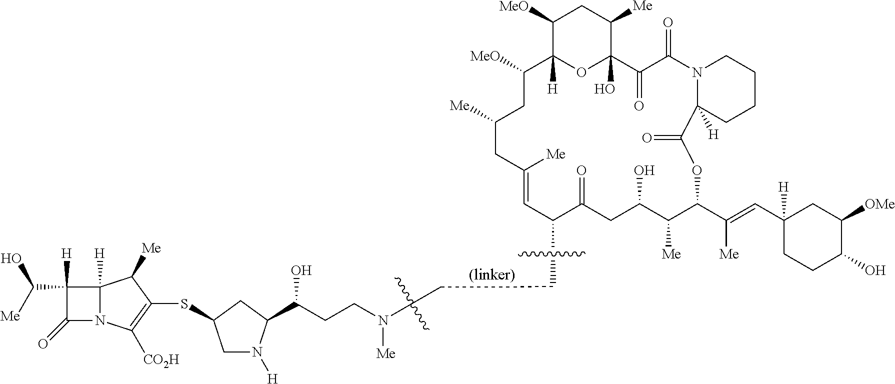

with linkers selected from

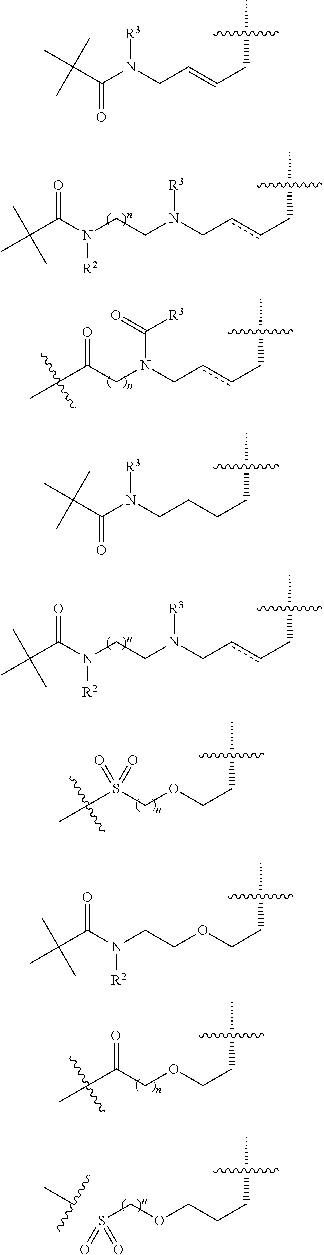

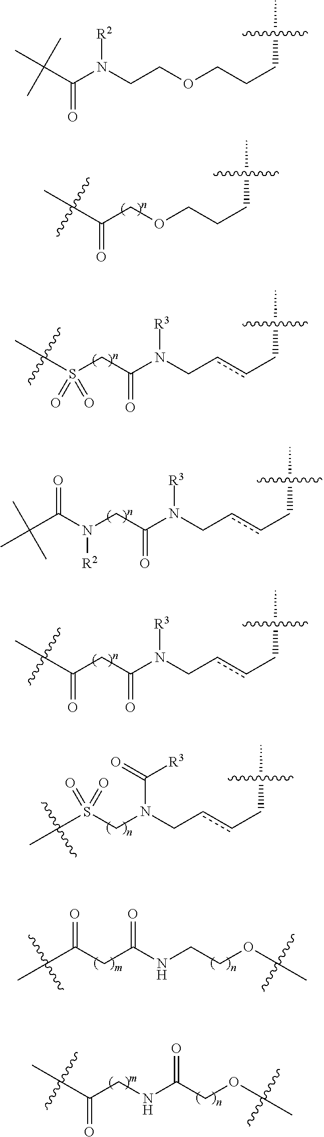

For example, the compounds may have Core 5b linked to Unit A:

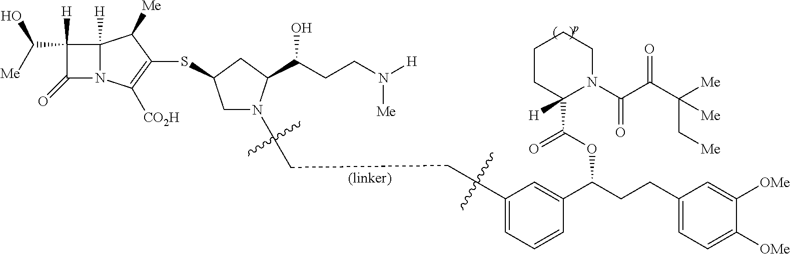

with linkers selected from

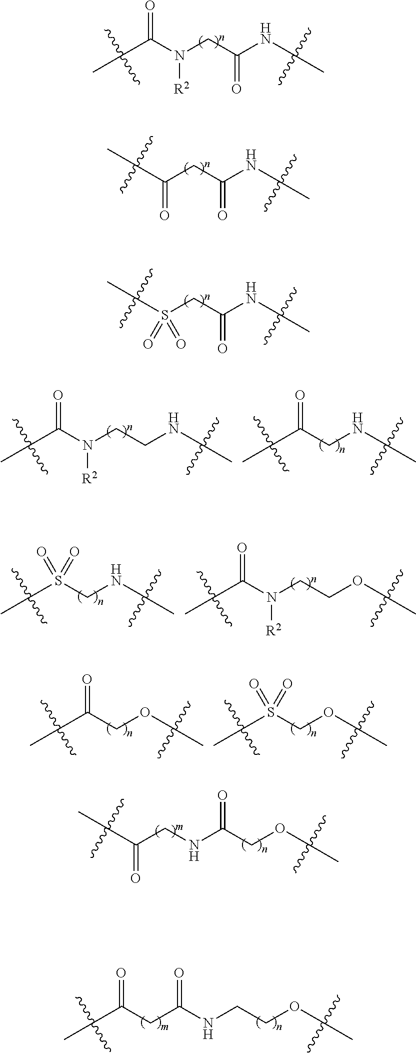

For example, the compounds may have Core 5b linked to Unit B:

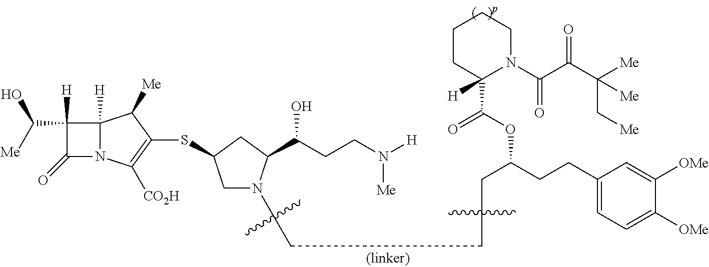

with linkers selected from

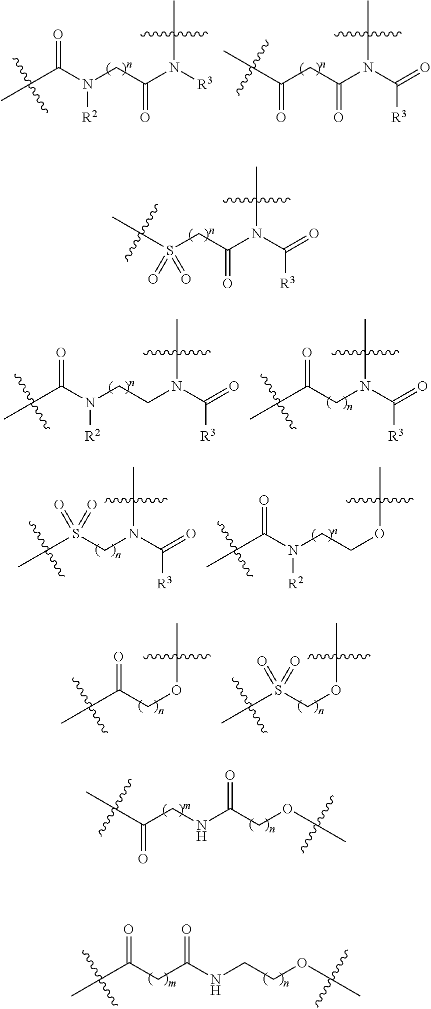

For example, the compounds may have Core 5b linked to Unit E:

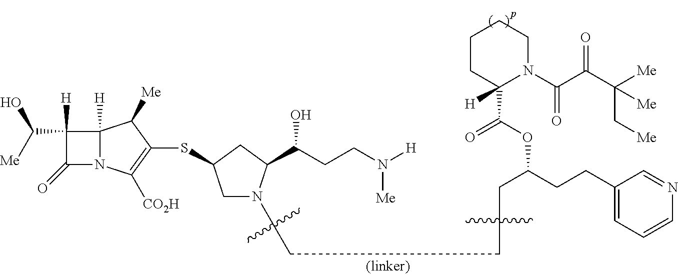

with linkers selected from

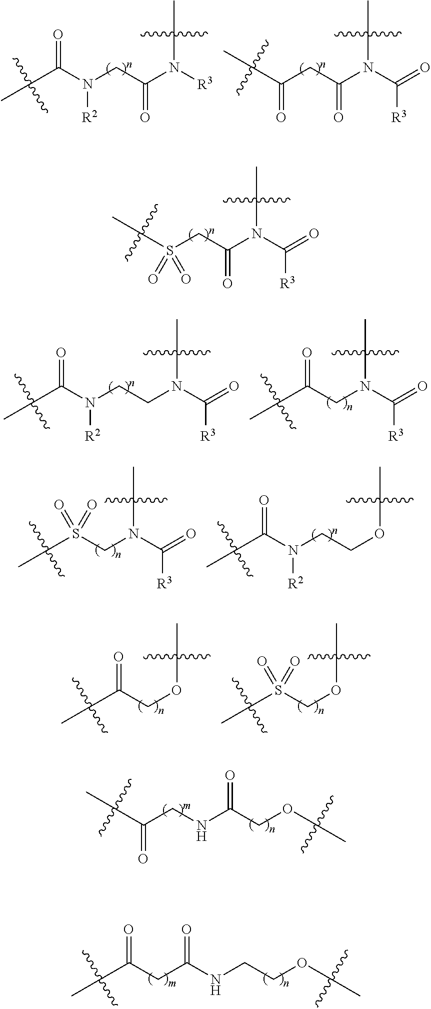

For example, the compounds may have Core 5b linked to Unit C:

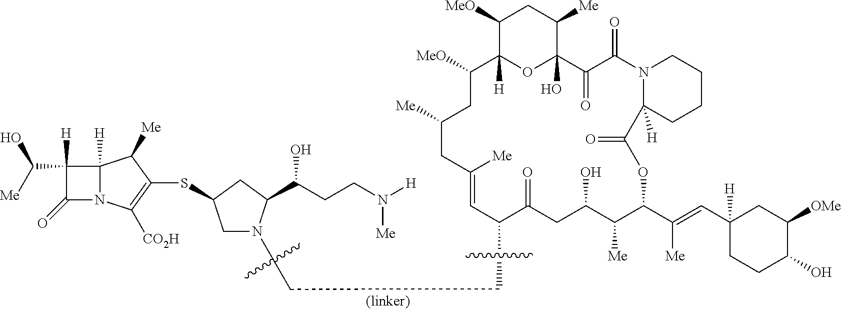

with linkers selected from

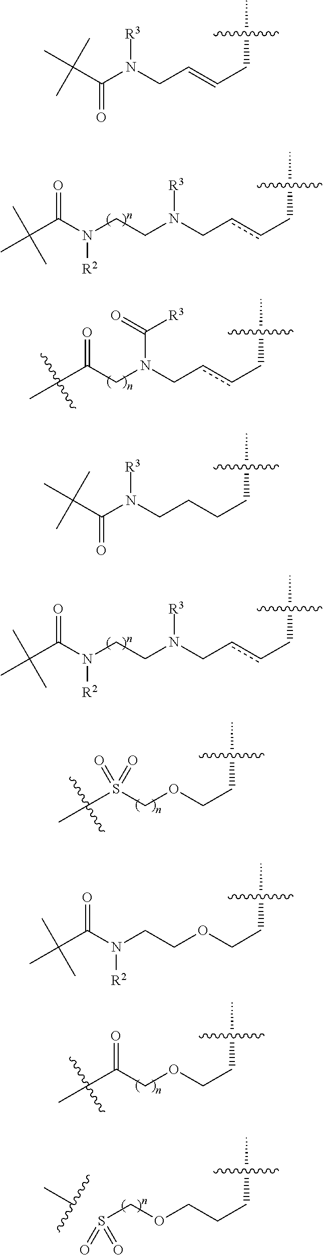

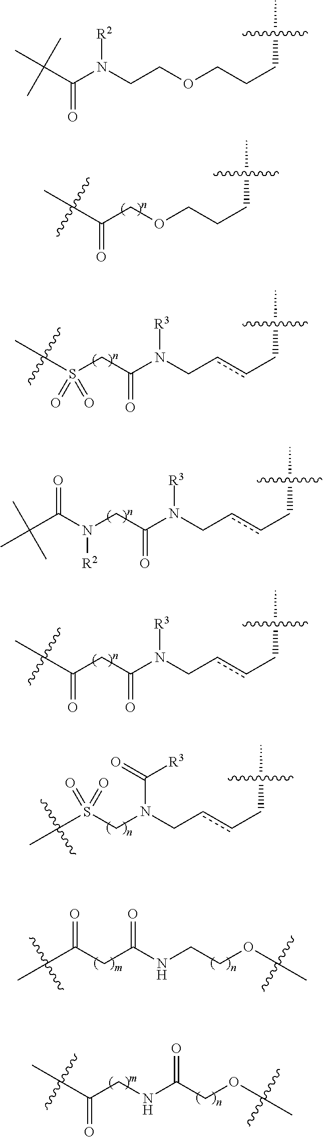

For example, the compounds may have Core 6a linked to Unit A:

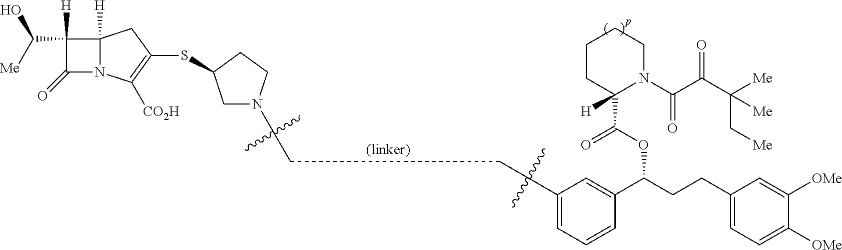

with linkers selected from

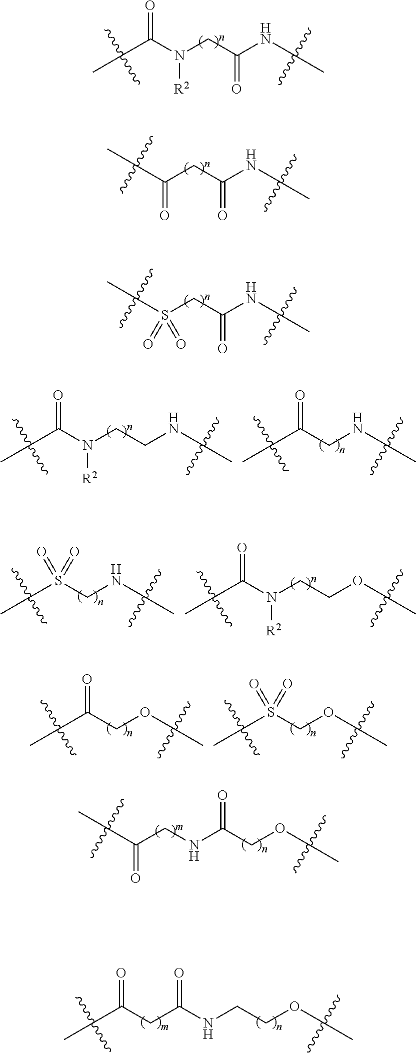

For example, the compounds may have Core 6a linked to Unit B:

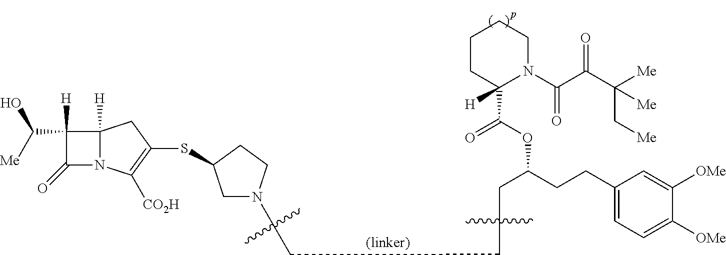

with linkers selected from

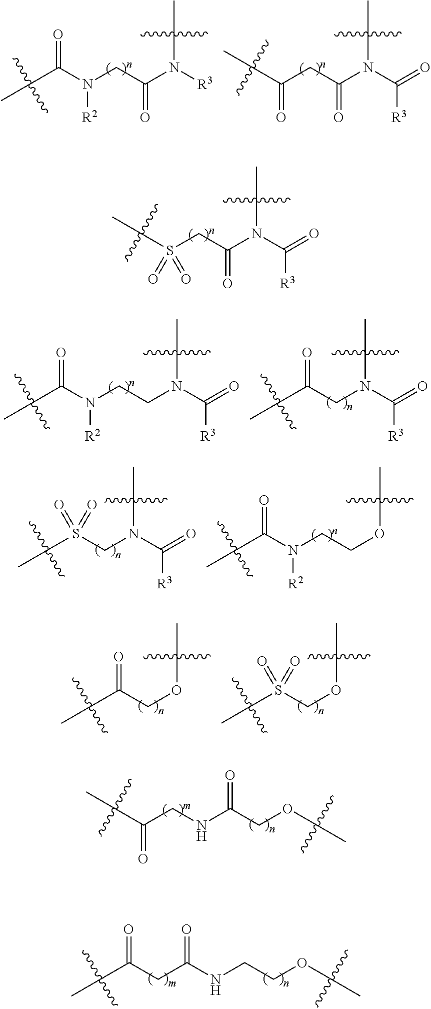

For example, the compounds may have Core 6a linked to Unit E:

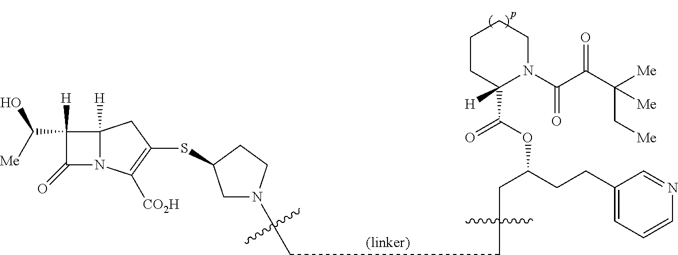

with linkers selected from

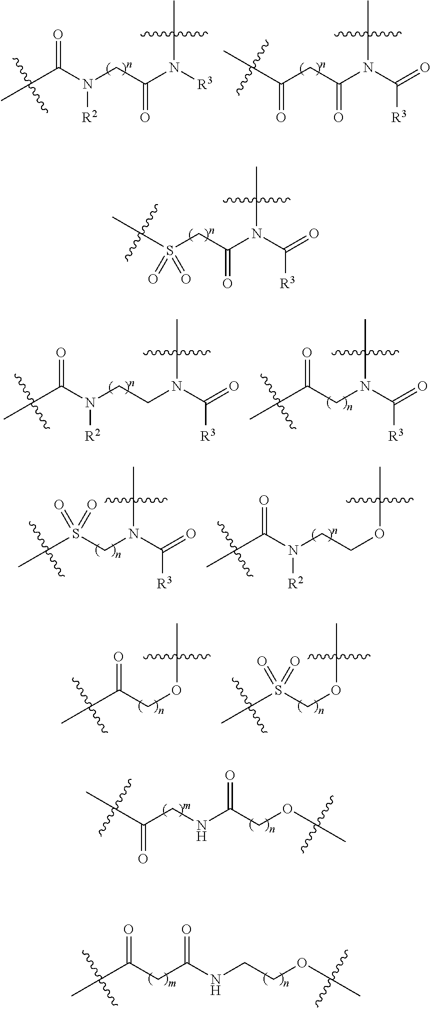

For example, the compounds may have Core 6a linked to Unit C:

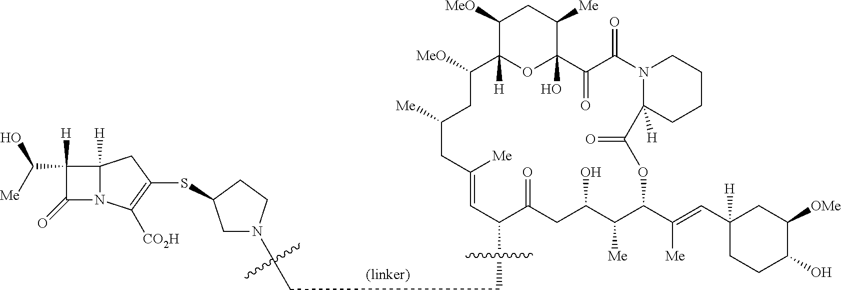

with linkers selected from

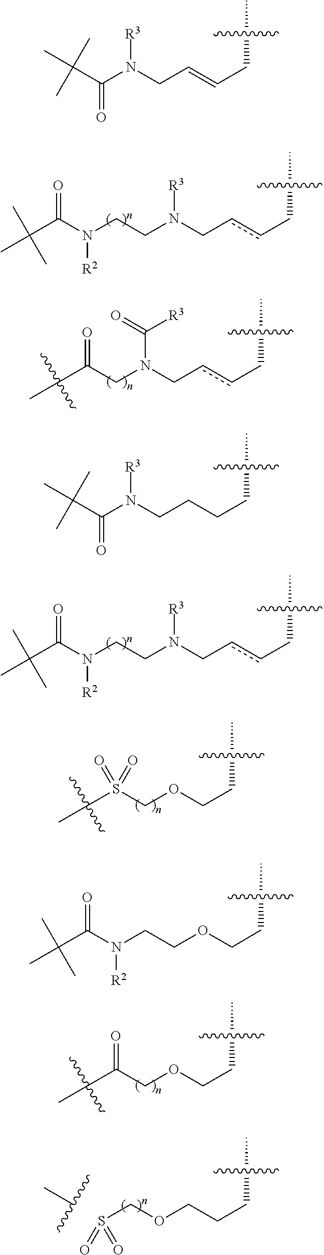

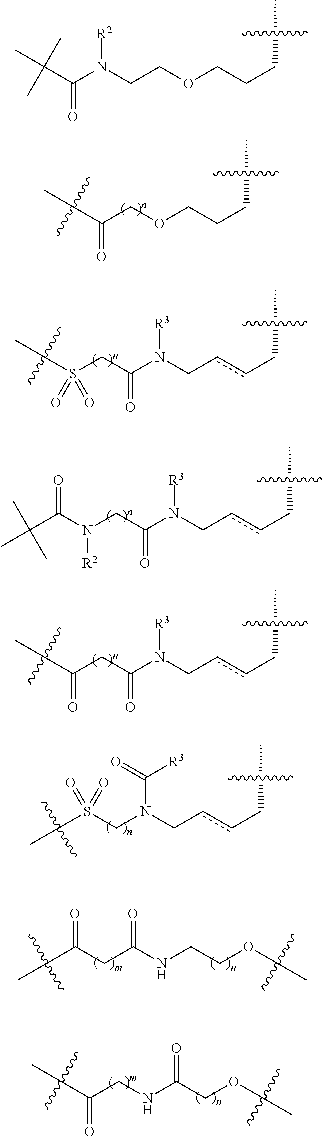

For example, the compounds may have Core 6b linked to Unit A:

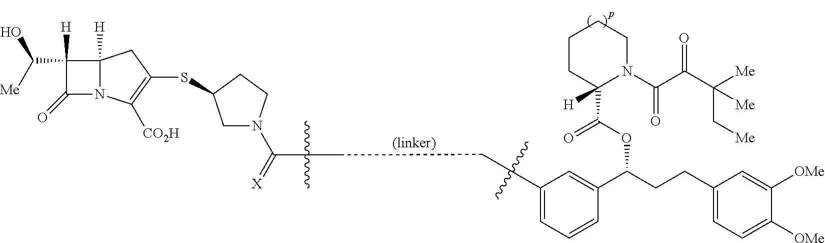

with linkers selected from

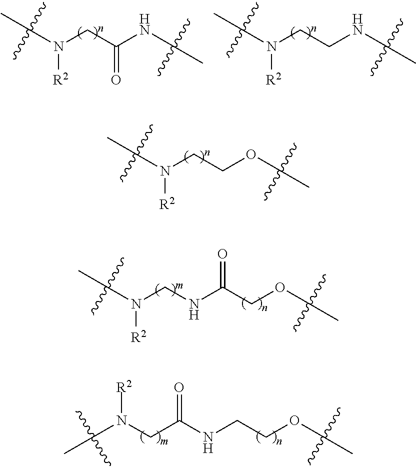

For example, the compounds may have Core 6b linked to Unit B:

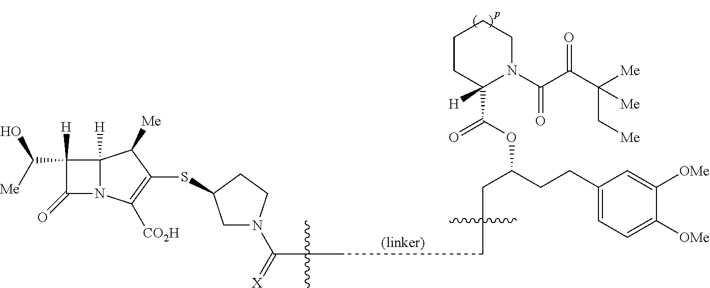

with linkers selected from

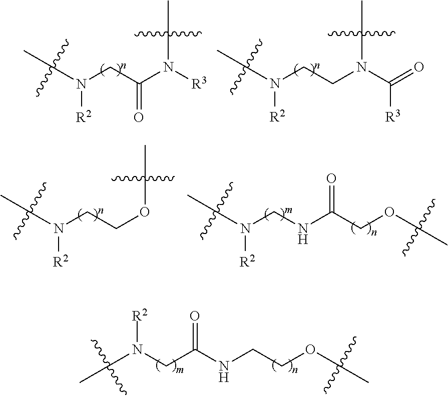

For example, the compounds may have Core 6b linked to Unit E:

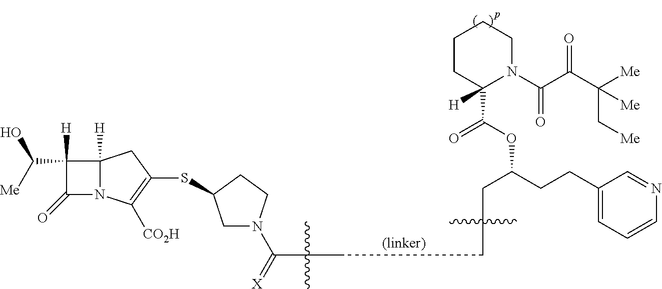

with linkers selected from

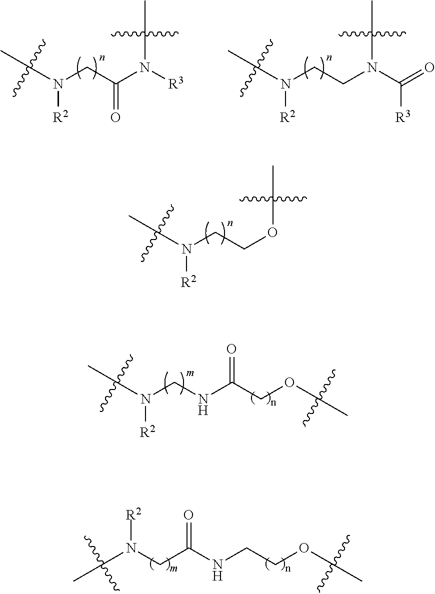

For example, the compounds may have Core 6b linked to Unit C:

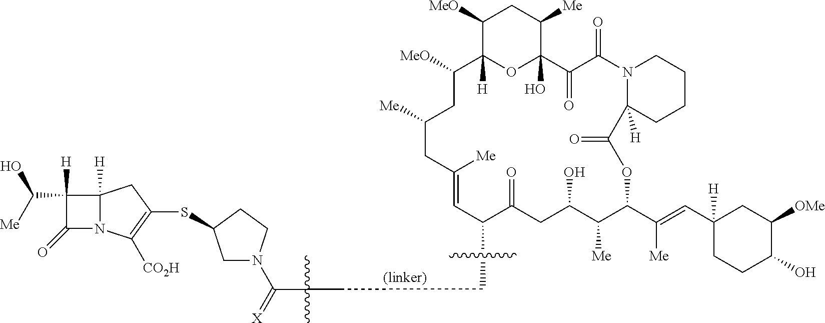

with linkers selected from

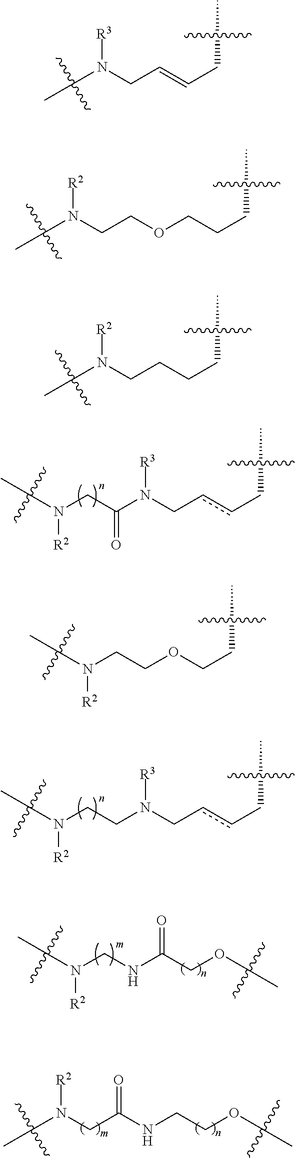

For example, the compounds may have Core 7a linked to Unit A:

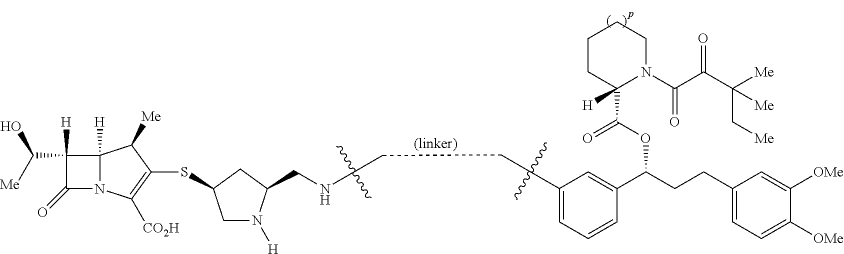

with linkers selected from

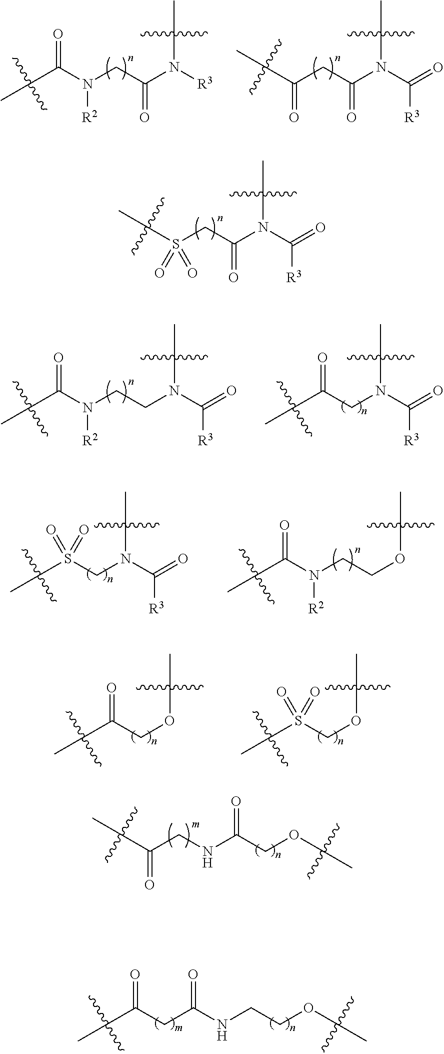

For example, the compounds may have Core 7a linked to Unit B:

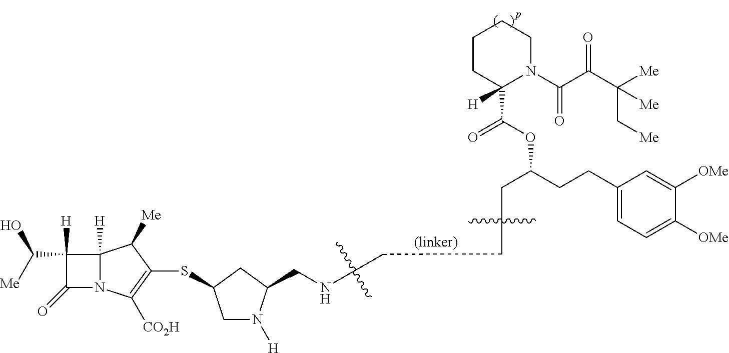

with linkers selected from

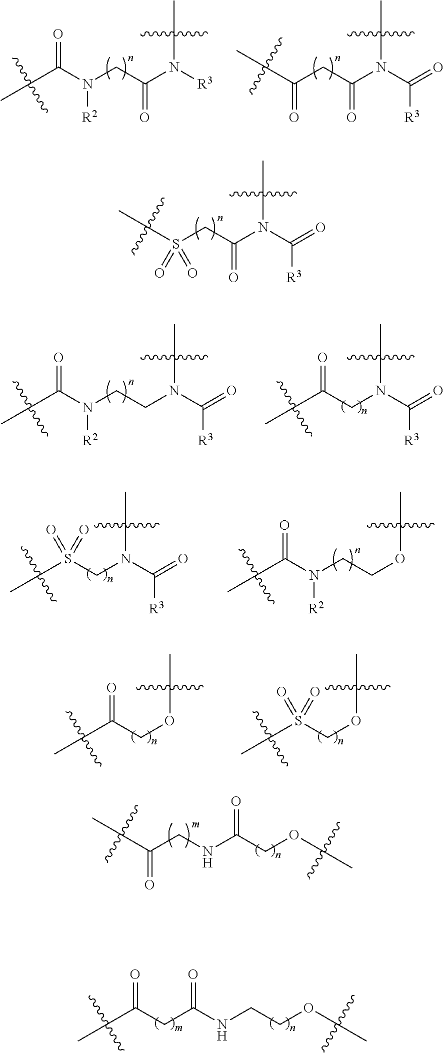

For example, the compounds may have Core 7a linked to Unit E:

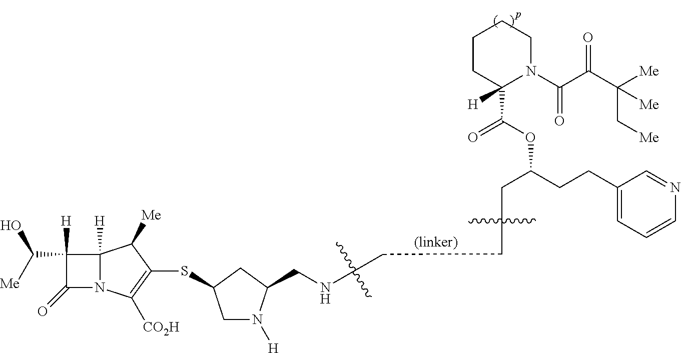

with linkers selected from

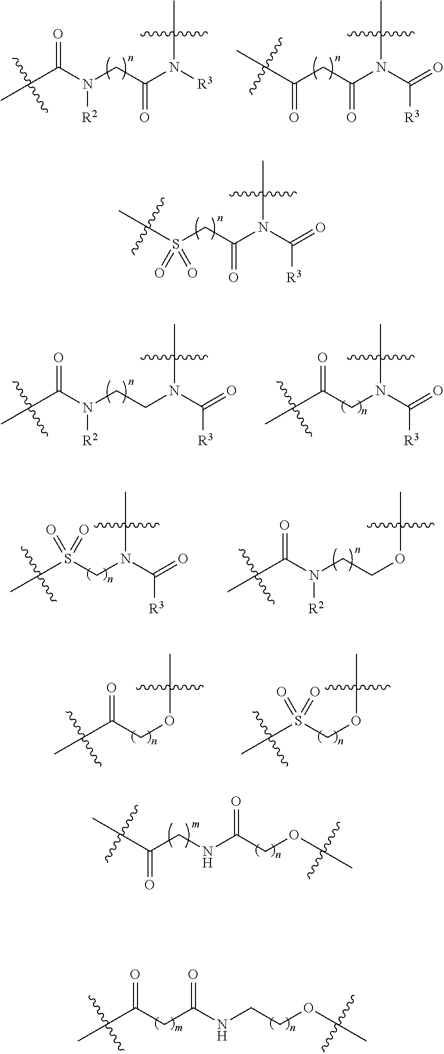

For example, the compounds may have Core 7a linked to Unit C:

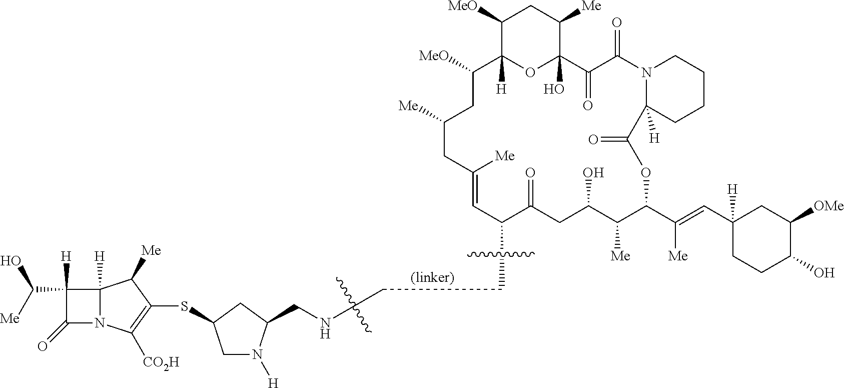

with linkers selected from

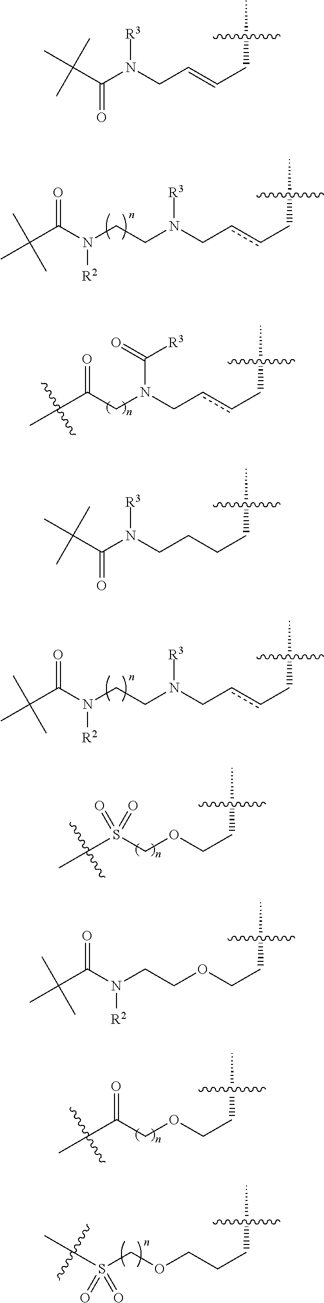

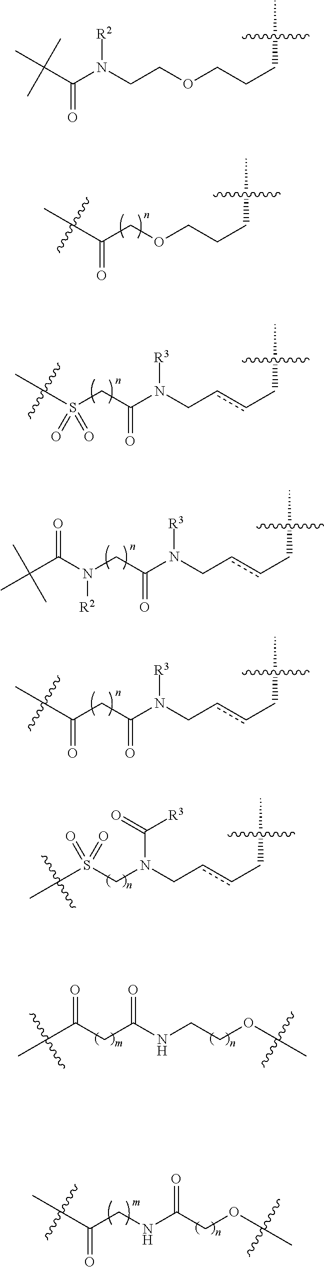

For example, the compounds may have Core 7b linked to Unit A:

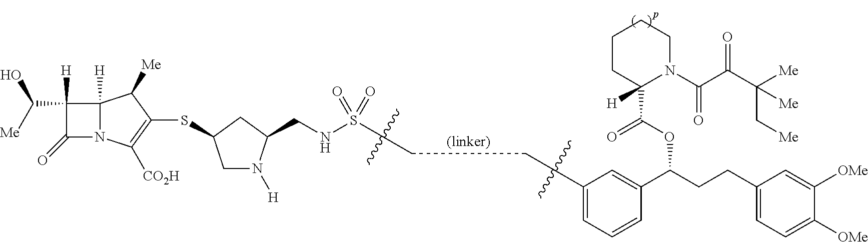

with linkers selected from

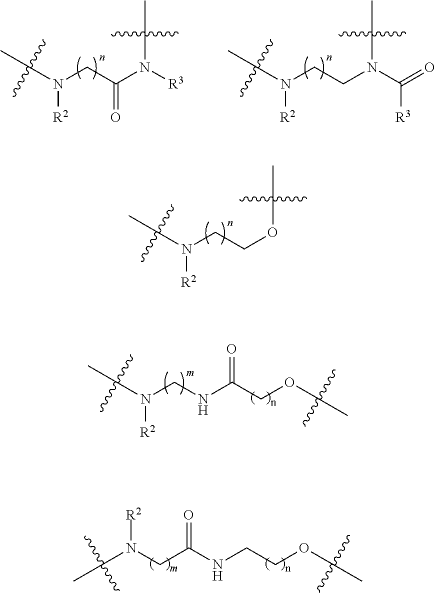

For example, the compounds may have Core 7b linked to Unit B:

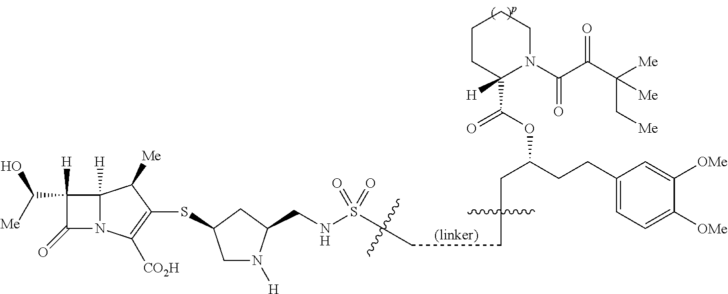

with linkers selected from

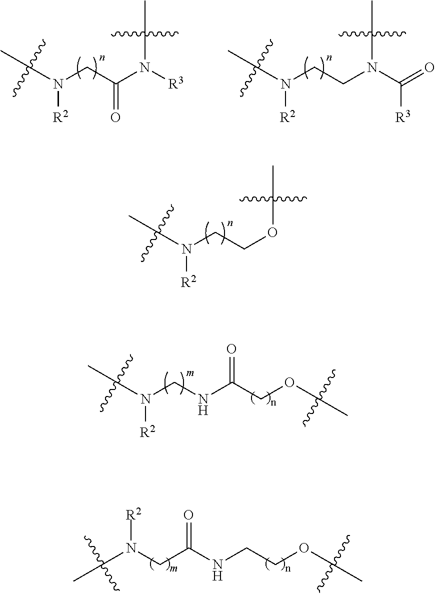

For example, the compounds may have Core 7b linked to Unit E:

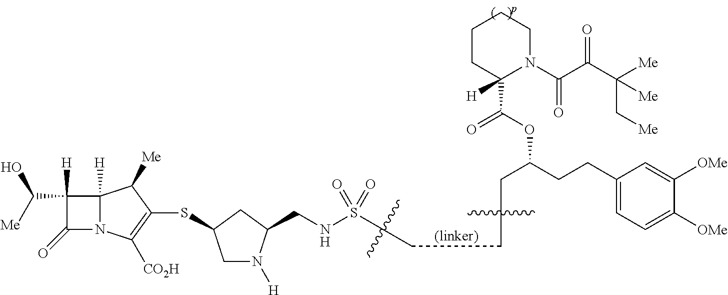

with linkers selected from

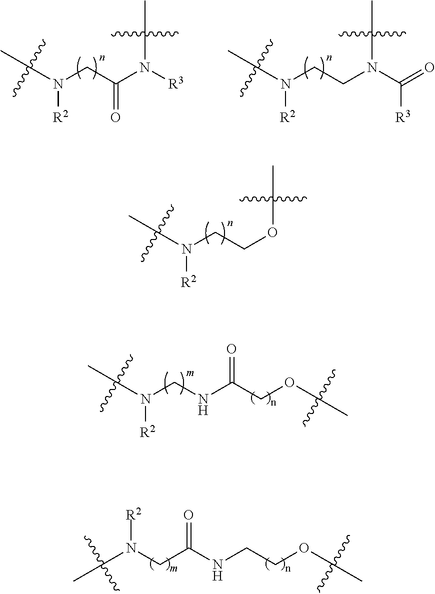

For example, the compounds may have Core 7b linked to Unit C:

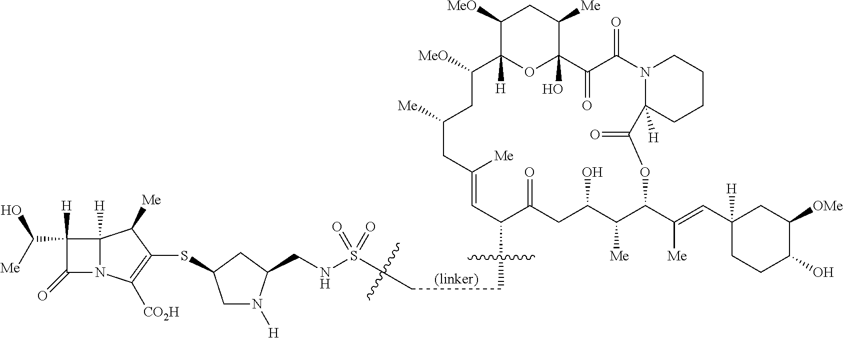

with linkers selected from

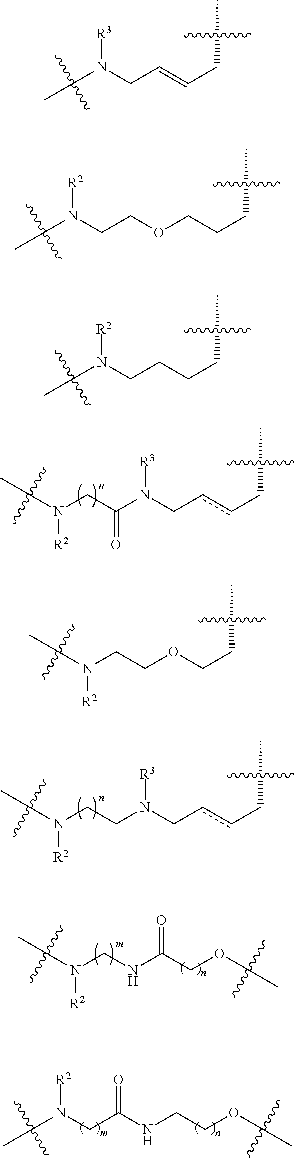

For example, the compounds may have Core 7c linked to Unit A:

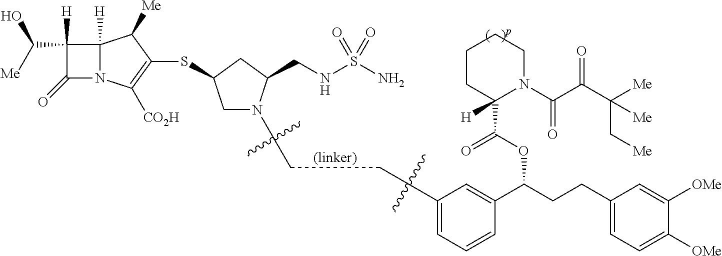

with linkers selected from

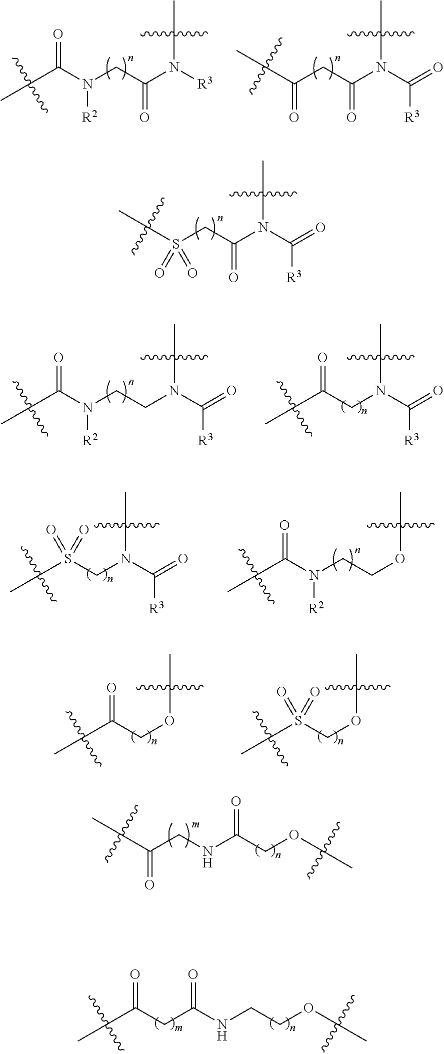

For example, the compounds may have Core 7c linked to Unit B:

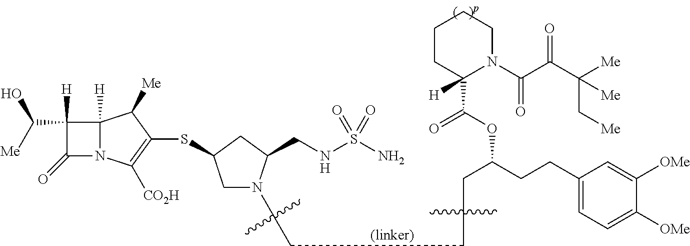

with linkers selected from

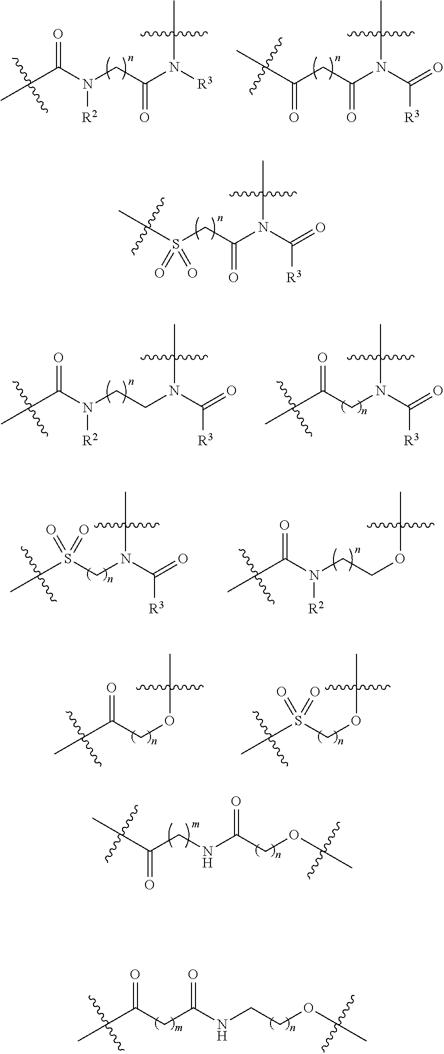

For example, the compounds may have Core 7c linked to Unit E:

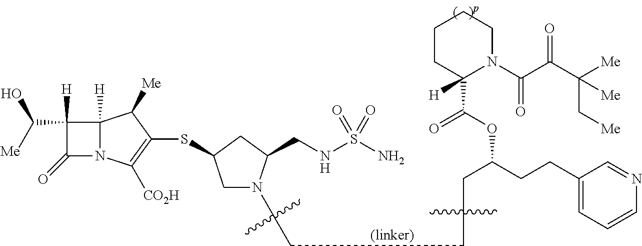

with linkers selected from

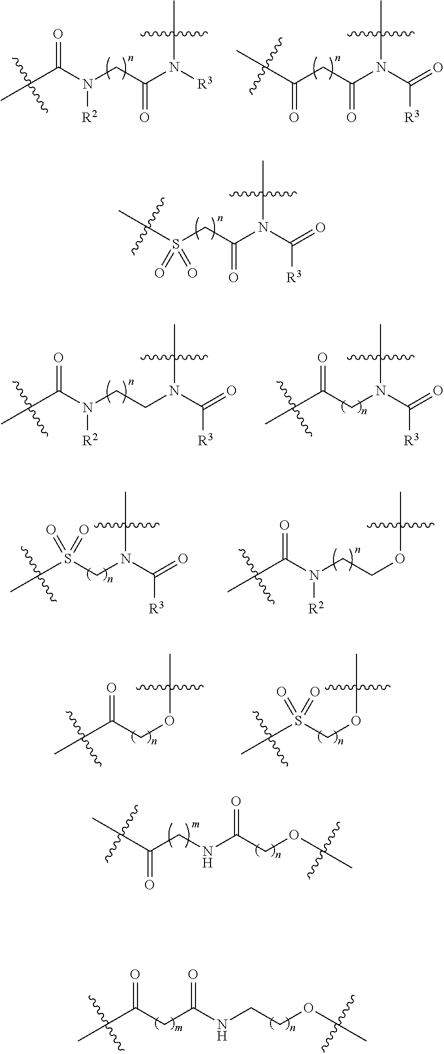

For example, the compounds may have Core 7c linked to Unit C:

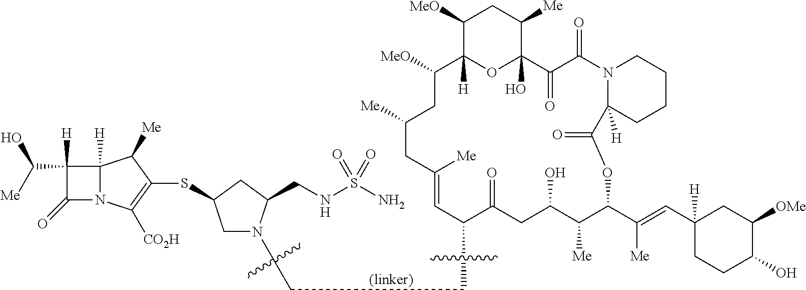

with linkers selected from

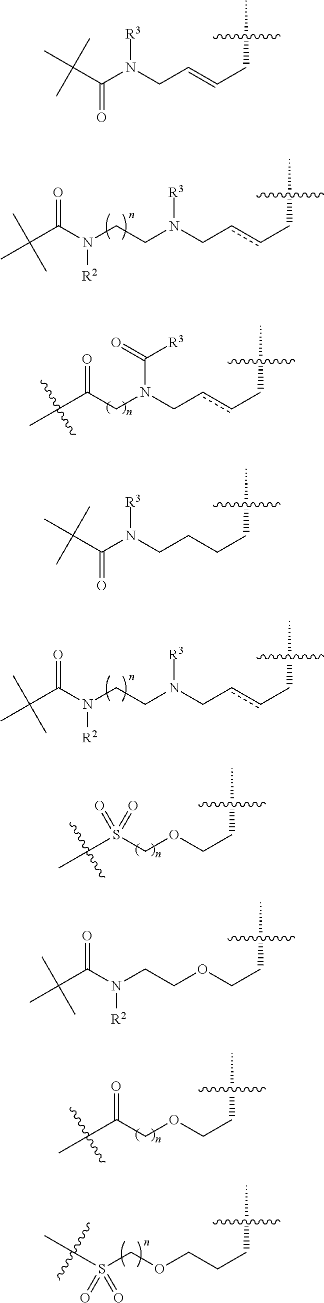

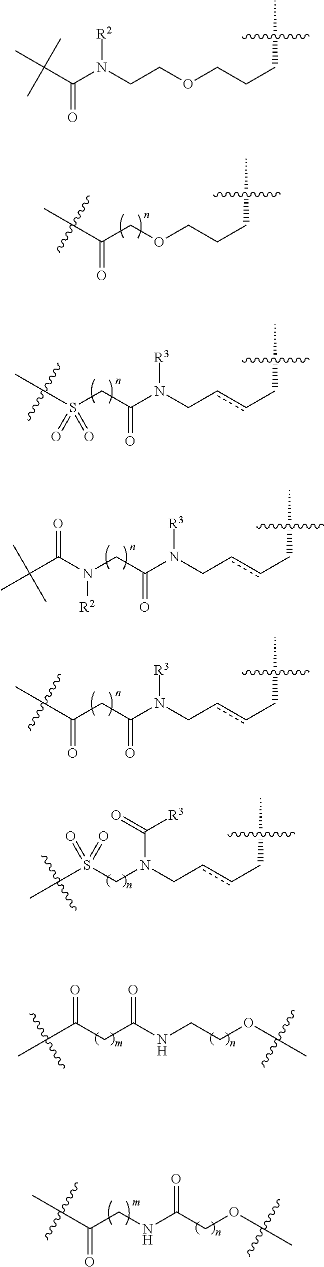

A selection of example compounds according to formula (I) include the following structures:

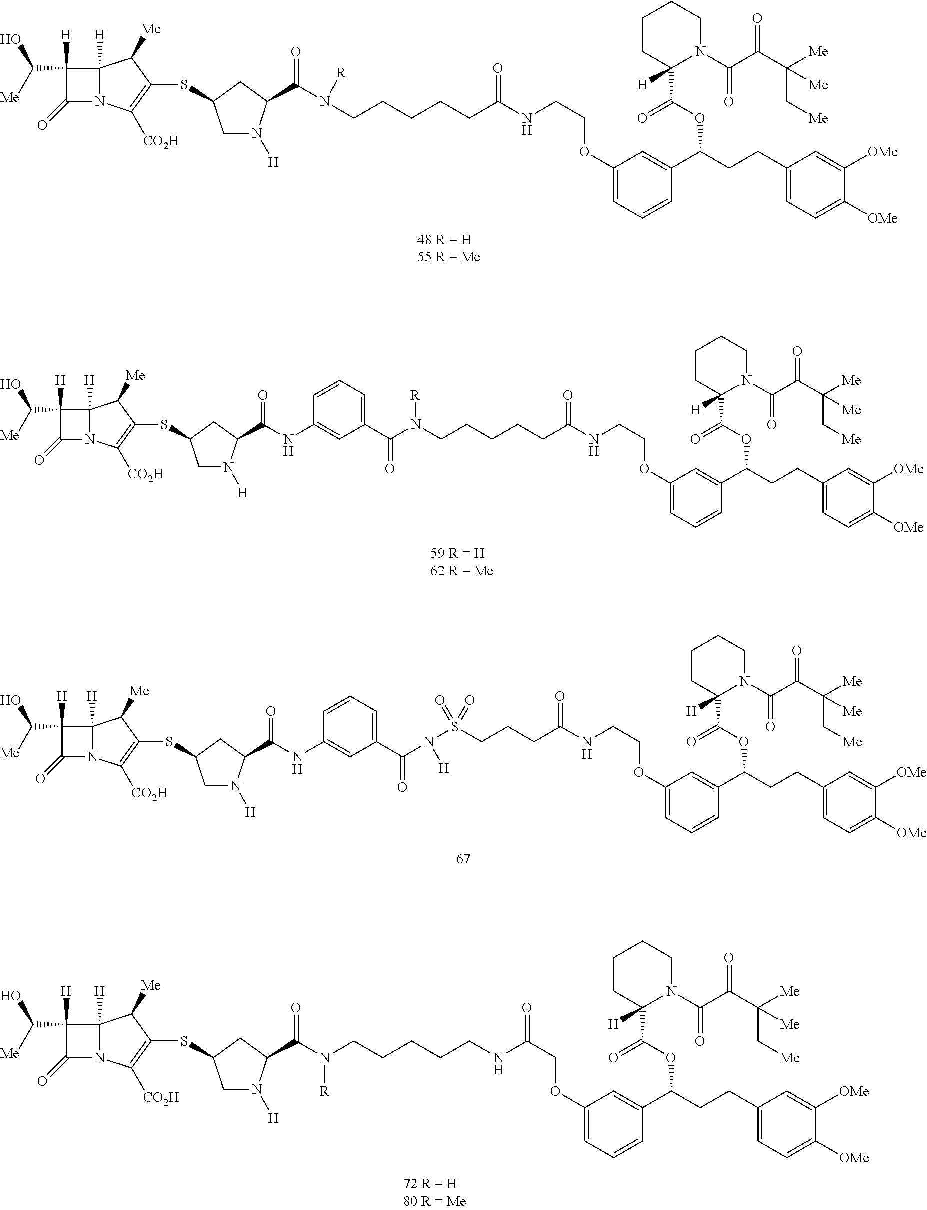

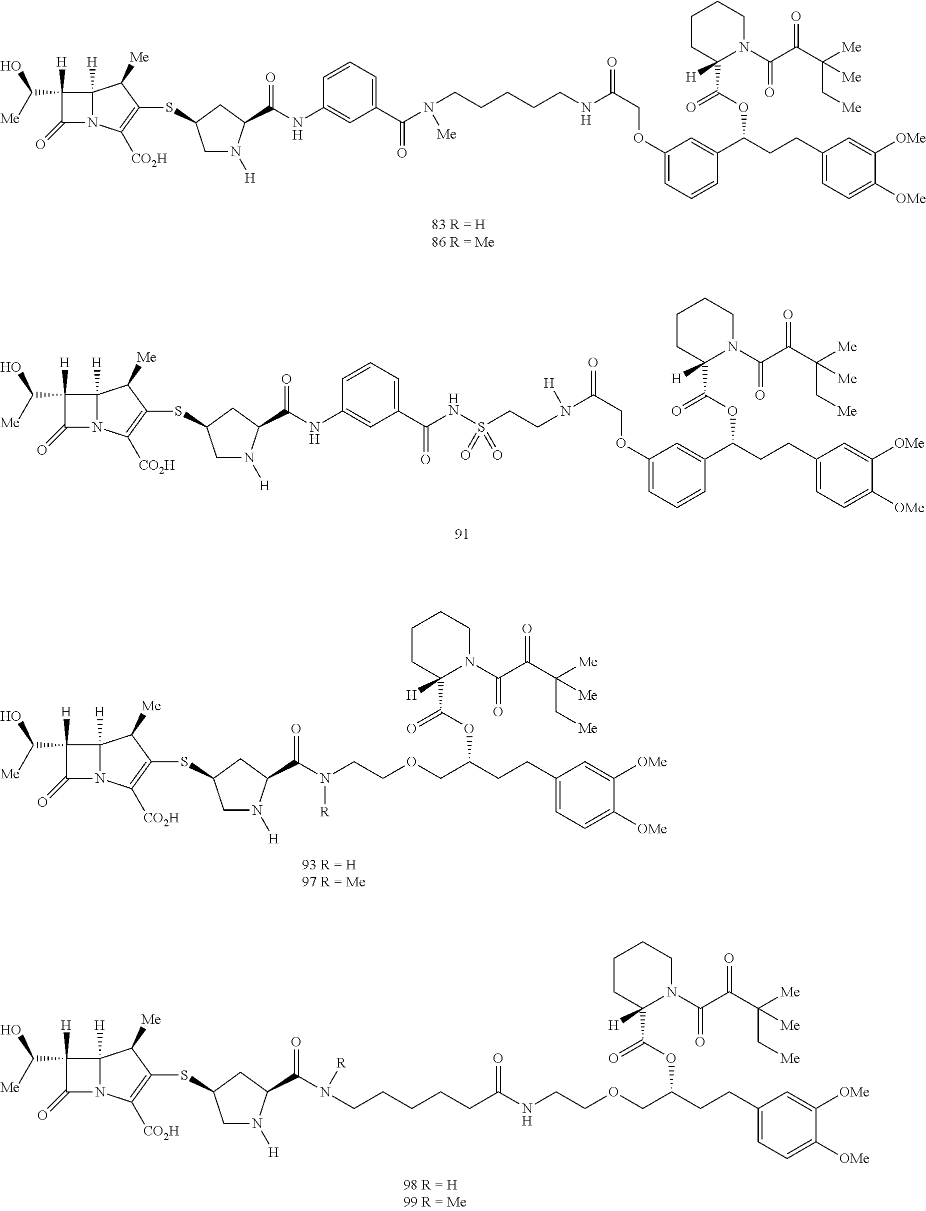

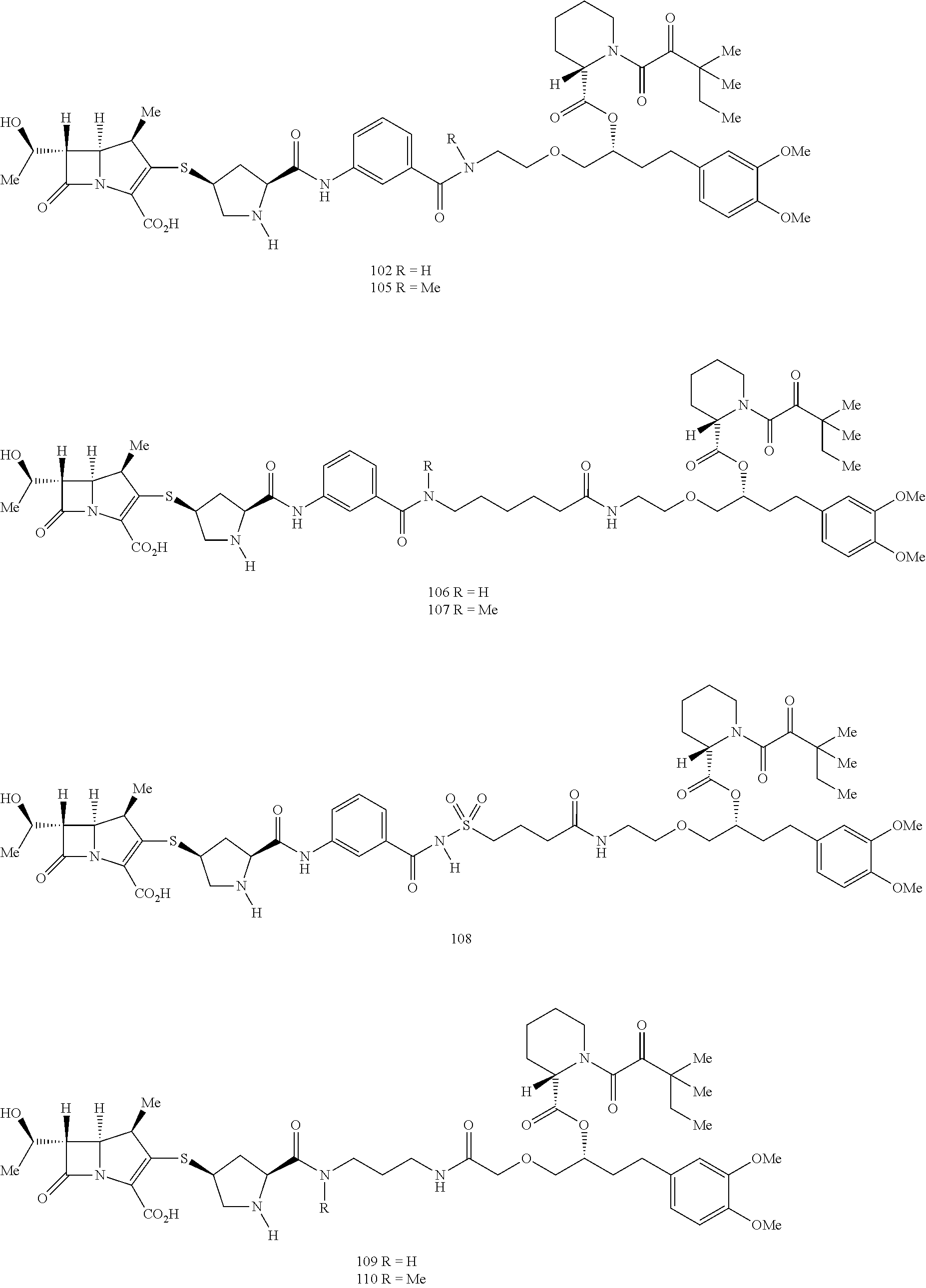

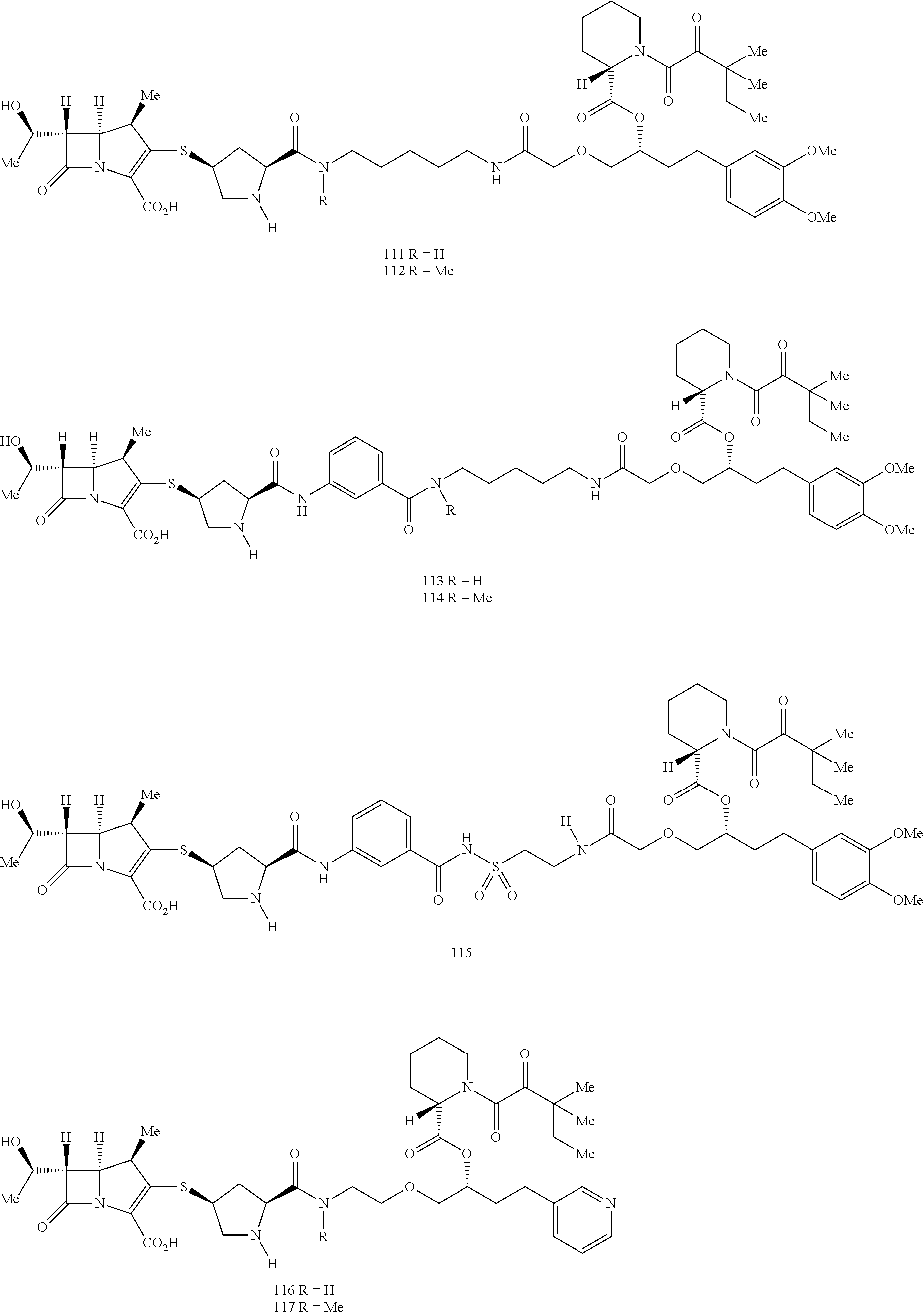

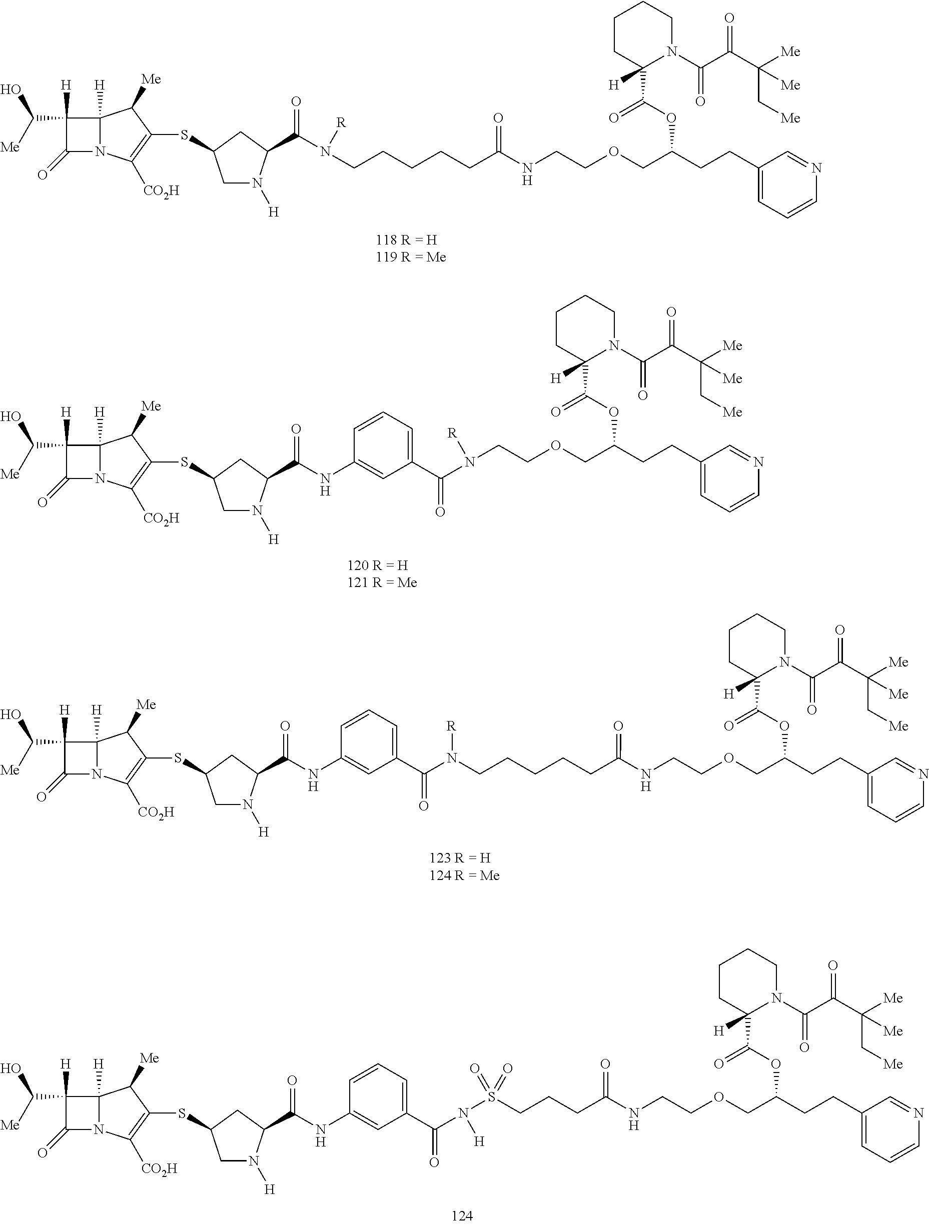

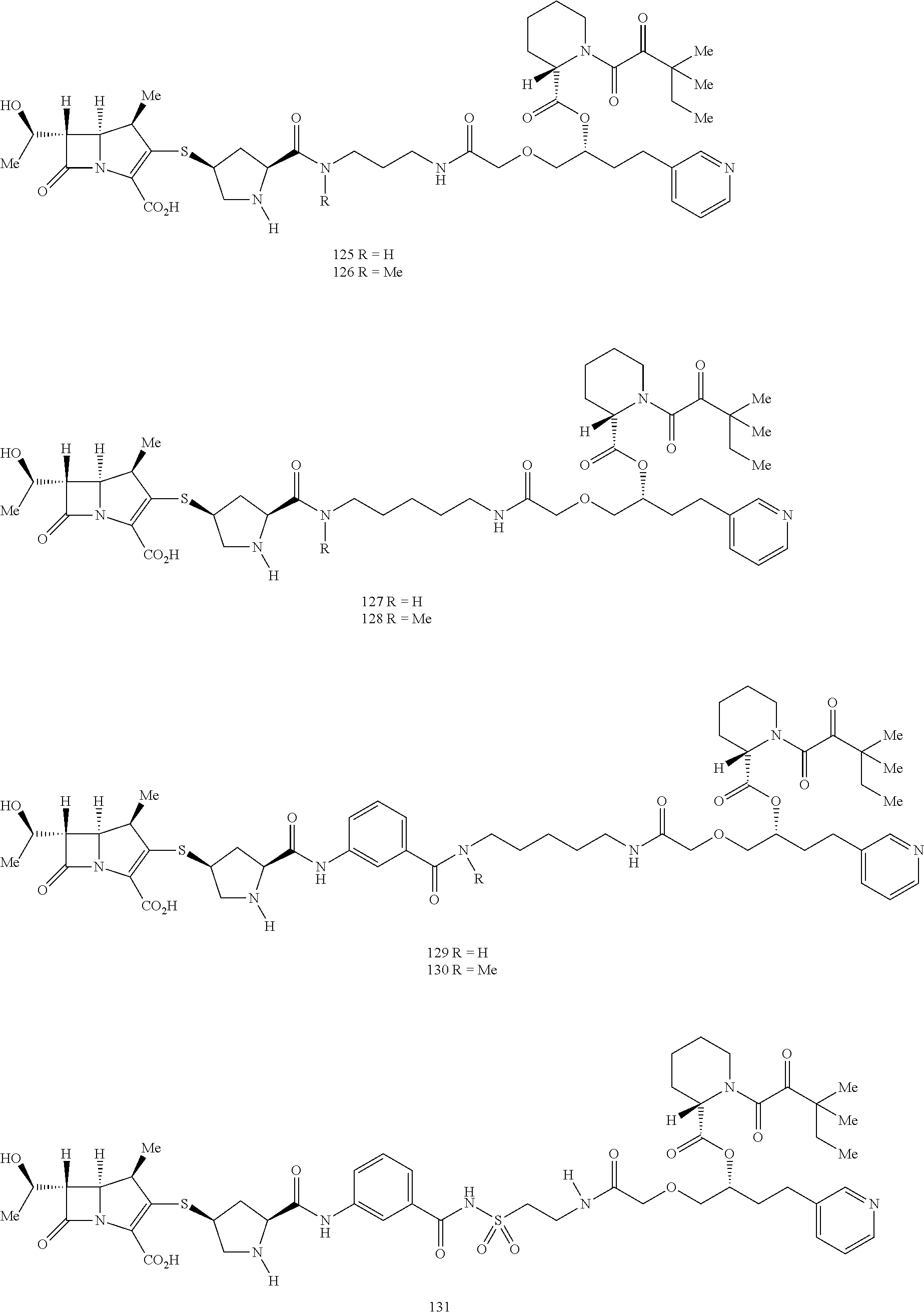

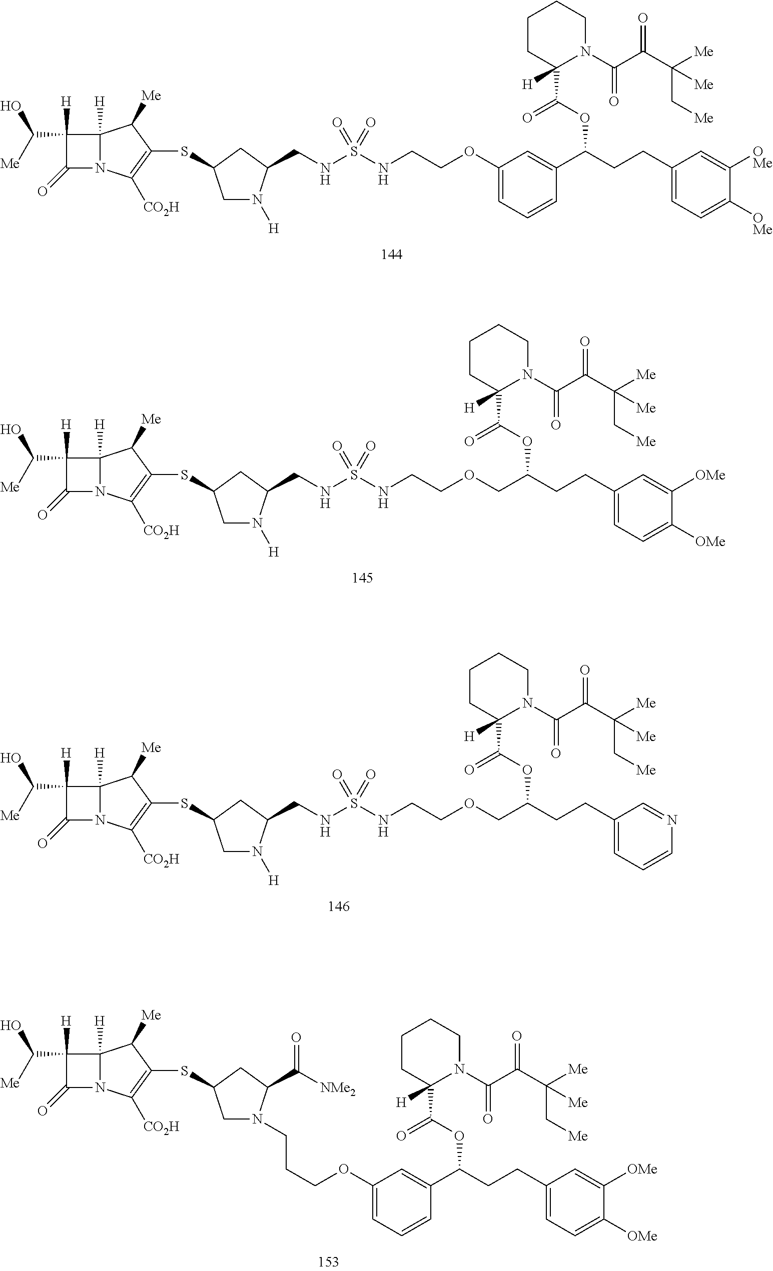

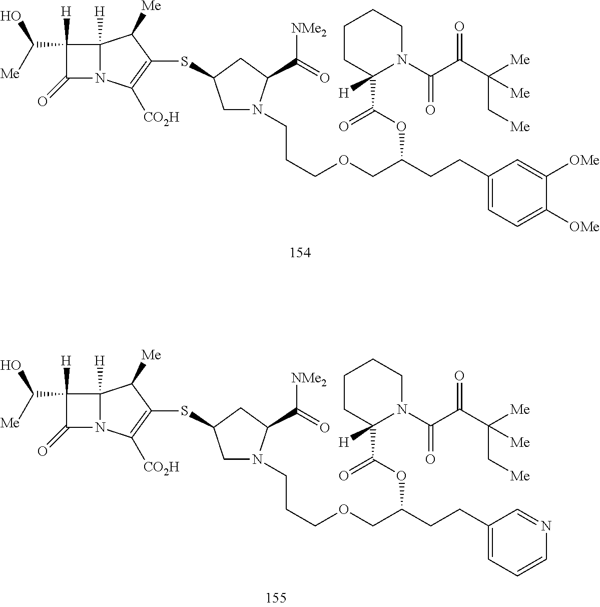

In some embodiments, the disclosure provides compounds having the core structure of formula (II)

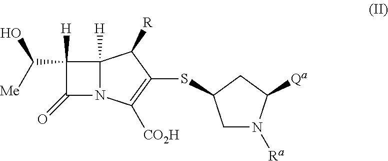

wherein:

R is selected from H and lower alkyl;

R^{a} is hydrocarbyl;

Q^{a} is selected from —(CH₂)ₘ₁—X^{a}—R^{b}, and —X^{a}—NH—Ar^{a};

m1 is selected from 0 and 1;

X^{a} is selected from a bond and —C(═O)—;

Ar^{a} is aryl or heteroaryl substituted with one or more R^{b} groups; and

R^{b} is selected from H, hydrocarbyl, and functional groups.

In some embodiments, R may be selected from H, methyl, ethyl, propyl, and butyl. In some embodiments, R is methyl.

In some embodiments, Ar^{a} is aryl and has the structure

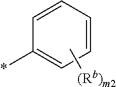

wherein m2 is selected from 2, 3, 4, and 5. Alternatively, Ar^{a} is heteroaryl containing one or more heteroatoms and substituted with 1-4 R^{b} groups.

In some embodiments, R^{b} may be selected from —NR′R″, —OR′″, —CO₂R′″, —CONR′R″, —NR′″SO₂NR′R″, alkyl (including lower alkyl), heteroalkyl, aryl, heteroaryl, aralkyl, heteroatom-containing aralkyl, halo, cyano, nitro, carboxamide, hydroxy, hydroxyalkyl, amino, aminoalkyl, aminoacyl, thiol, and thioalkyl.

R′, R″, and R′″ are independently selected from H, alkyl (including lower alkyl), heteroalkyl, aryl, heteroaryl, aralkyl, and heteroatom-containing aralkyl. For example, R′, R″, and R′″ are independently H, methyl, ethyl, propyl, phenyl, pyridyl, and benzyl.

Some examples of R^{b} groups include —NMe₂, —SO₂Me, OMe, —CO₂Me, —NMeSO₂NMe₂, —NH₂, OH, —CO₂H, —NHSO₂NH₂, and the like.

In some embodiments, R^{a} is substituted or unsubstituted alkyl, aryl, heteroaryl, aralkyl, or heteroatom-containing aralkyl.

In some embodiments, R^{a} is substituted or unsubstituted aralkyl that may contain one or more heteroatoms. Examples of R^{a} include the following structures:

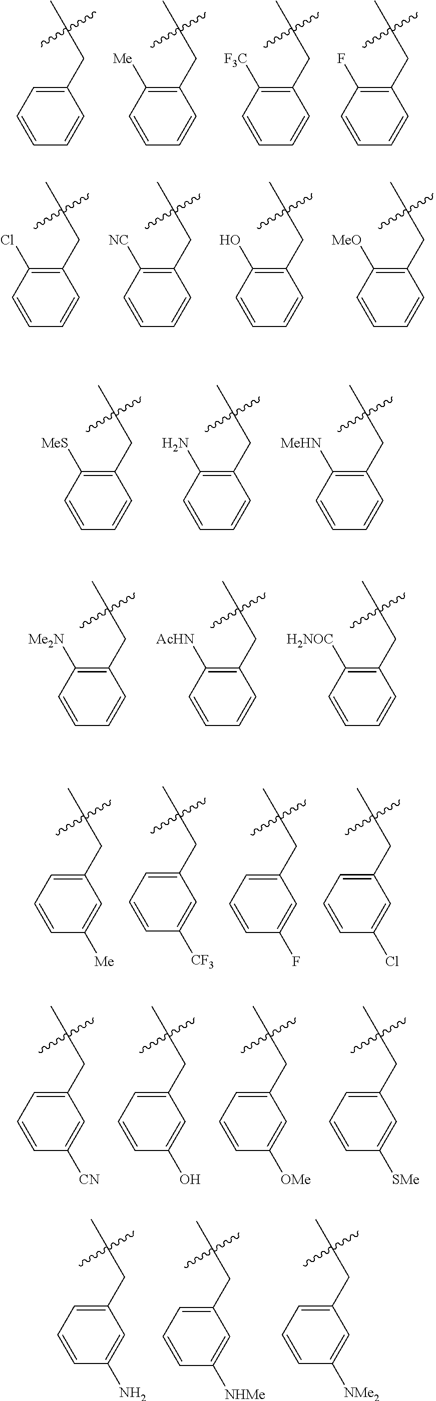

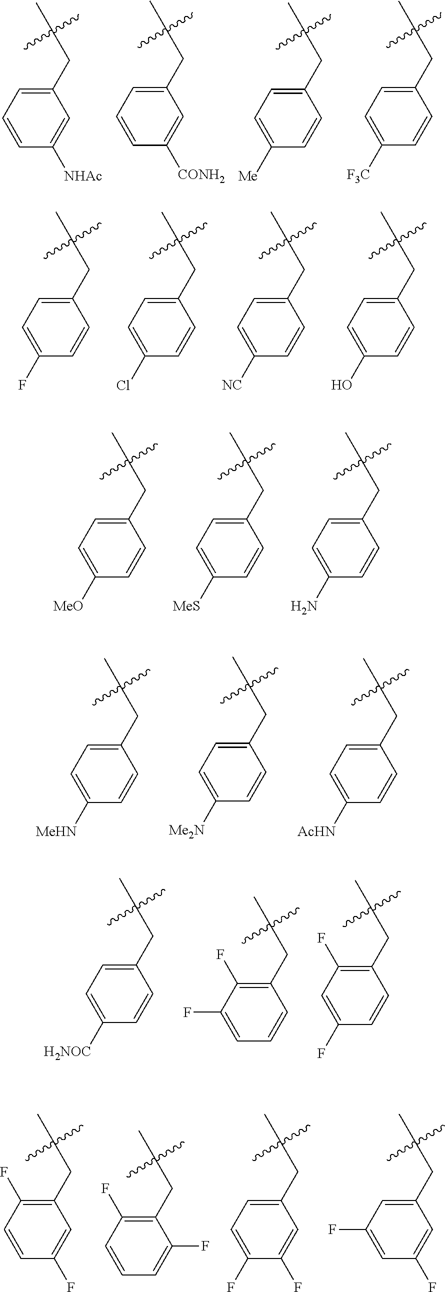

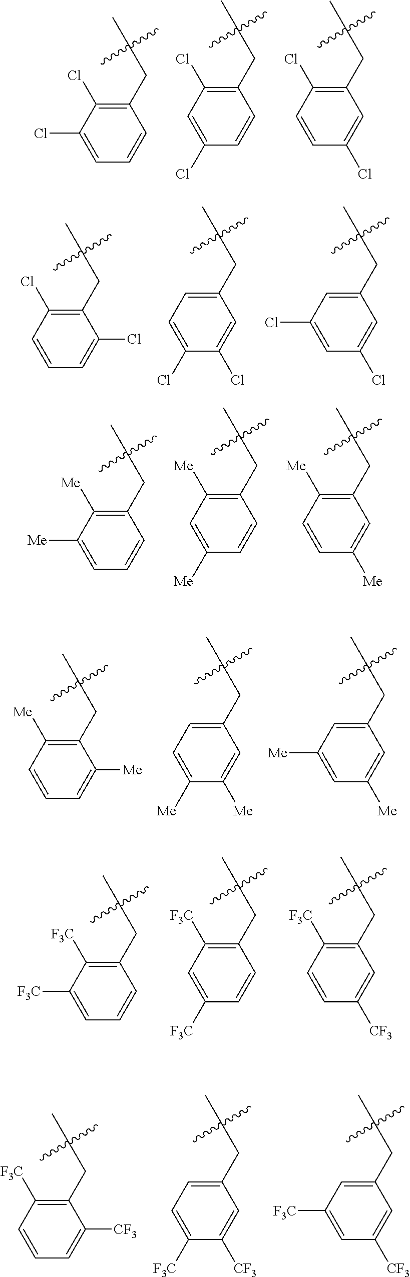

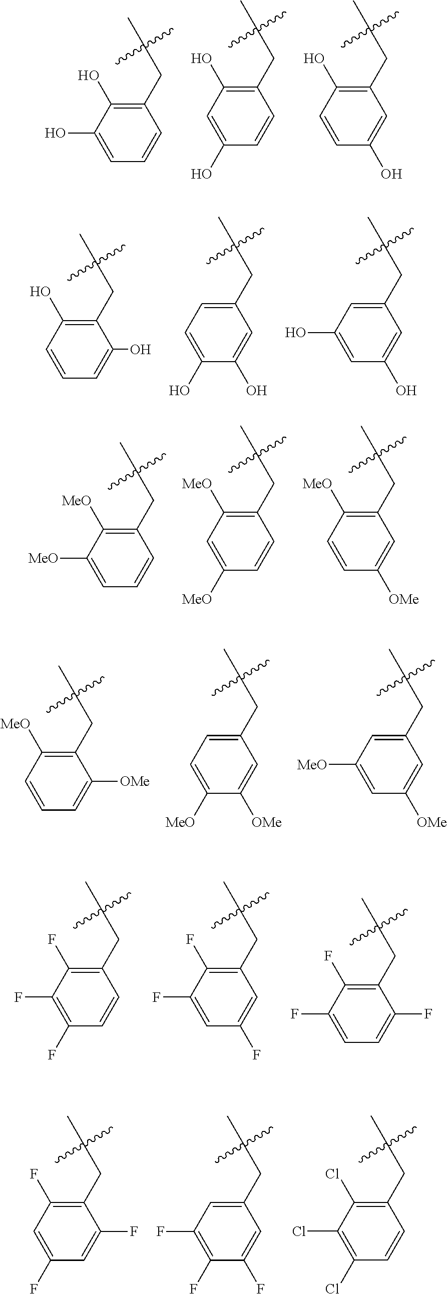

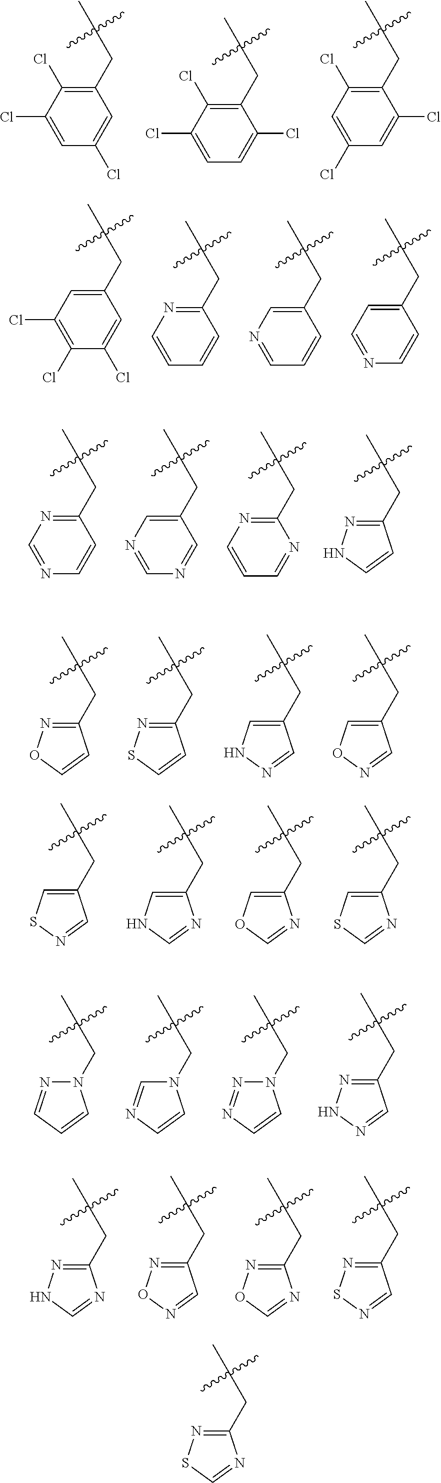

wherein the wavy line represents the attachment point to the remainder of the compound. It will be appreciated that any of the above structures may be further substituted where appropriate.
In some embodiments, R^{a} has the structure -L^{a}-U, wherein L^{a} is a linker moiety and U is as defined previously with respect to compounds of formula (I).

In some embodiments, L^{a} is as defined for L¹ with respect to compounds of formula (I).

In some embodiments, L^{a} is an aralkylene linker (including heteroatom-containing aralkylene, substituted aralkylene, and heteroatom-containing substituted aralkylene linkers) such as aralkylene versions of any of the aralkyl groups described above for R^{a}. Some examples include benzylene (i.e., —CH₂—C₆H₄—) and substituted benzylenes, heteroatom-containing benzylenes (e.g., —CH₂—C₅H₃N—), furanylmethyl (i.e., —CH₂—C₄H₂O—), and the like.

Some examples of the linker moiety L^{a} include the following:

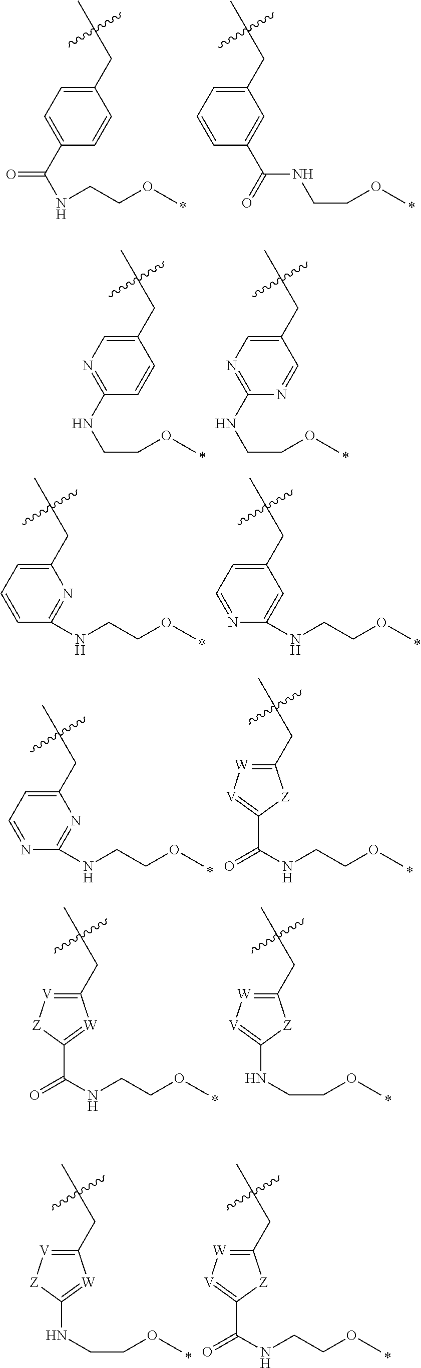

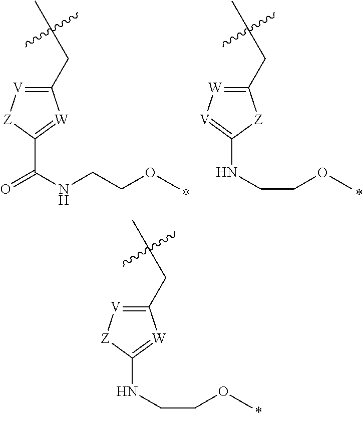

wherein:

V and W are independently selected from —CH— and —N—;

Z is selected from —O—, —NH—, —NMe-, and —S—; and

the wavy lines represent attachment points to the core portion of formula (II) and the stars represent attachment points to the additional unit U.

In other embodiments, the linker L^{a} is an alkyl linker which may be substituted or unsubstituted and is optionally heteroatom-containing. For example, the linker L^{a} may have the structure

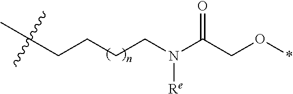

wherein n has the value of 0, 1, 2, 3, or 4, and R^{e} is selected from H and alkyl. Again, the wavy line represents the attachment point to the core of the compound, and the star represents the point of attachment to the additional unit U.

It will be appreciated that any combination of additional unit and linker moiety may be used to construct R^{a}.

In some embodiments, the compounds of the disclosure have the structure of formula (IIa)

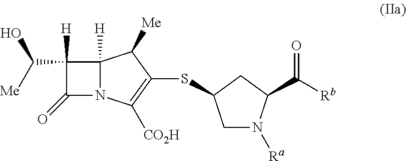

In some embodiments, the compounds of the disclosure have the structure of formula (IIb)

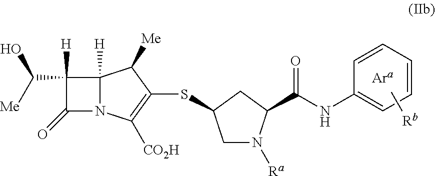

In some embodiments, the compounds of the disclosure have the structure of formula (IIc)

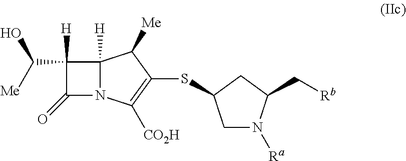

Examples of compounds of formula (IIa) include:

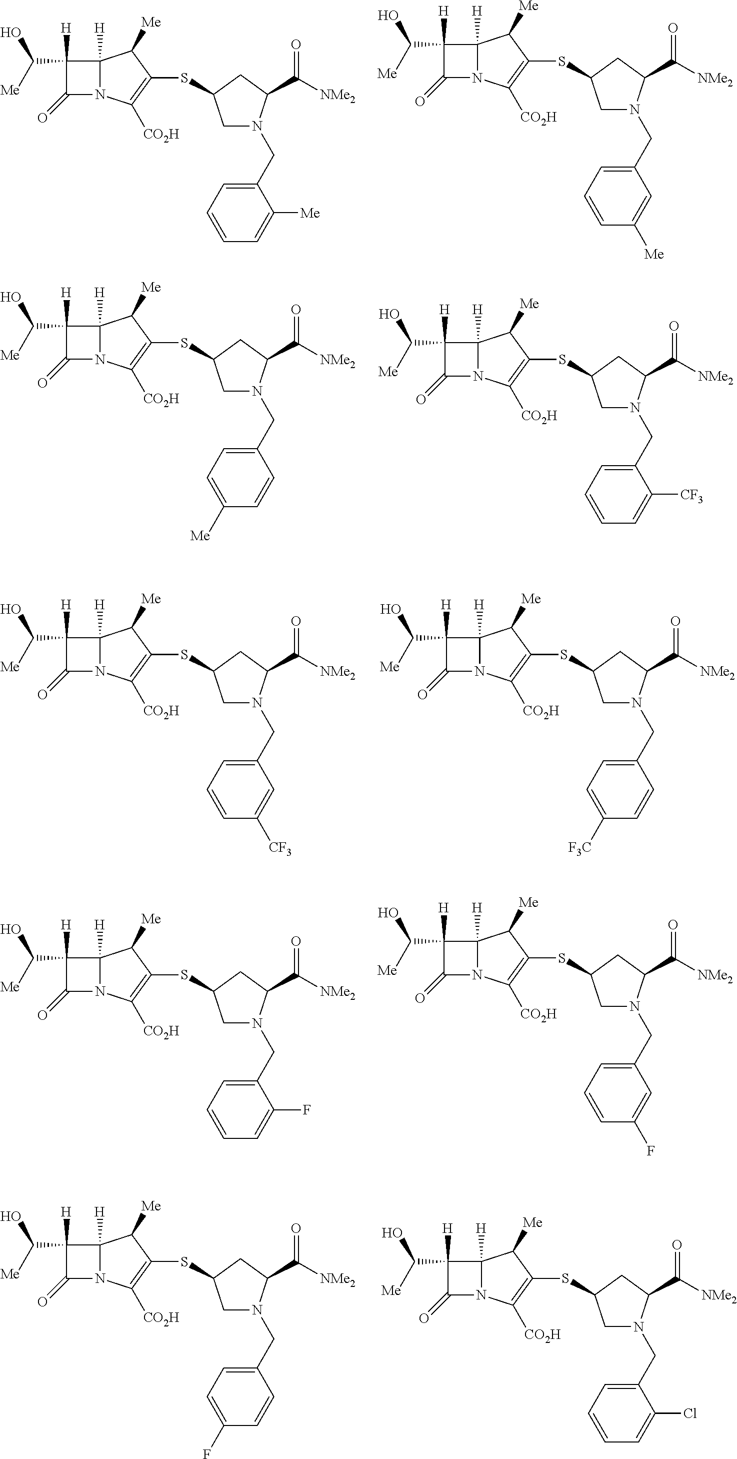

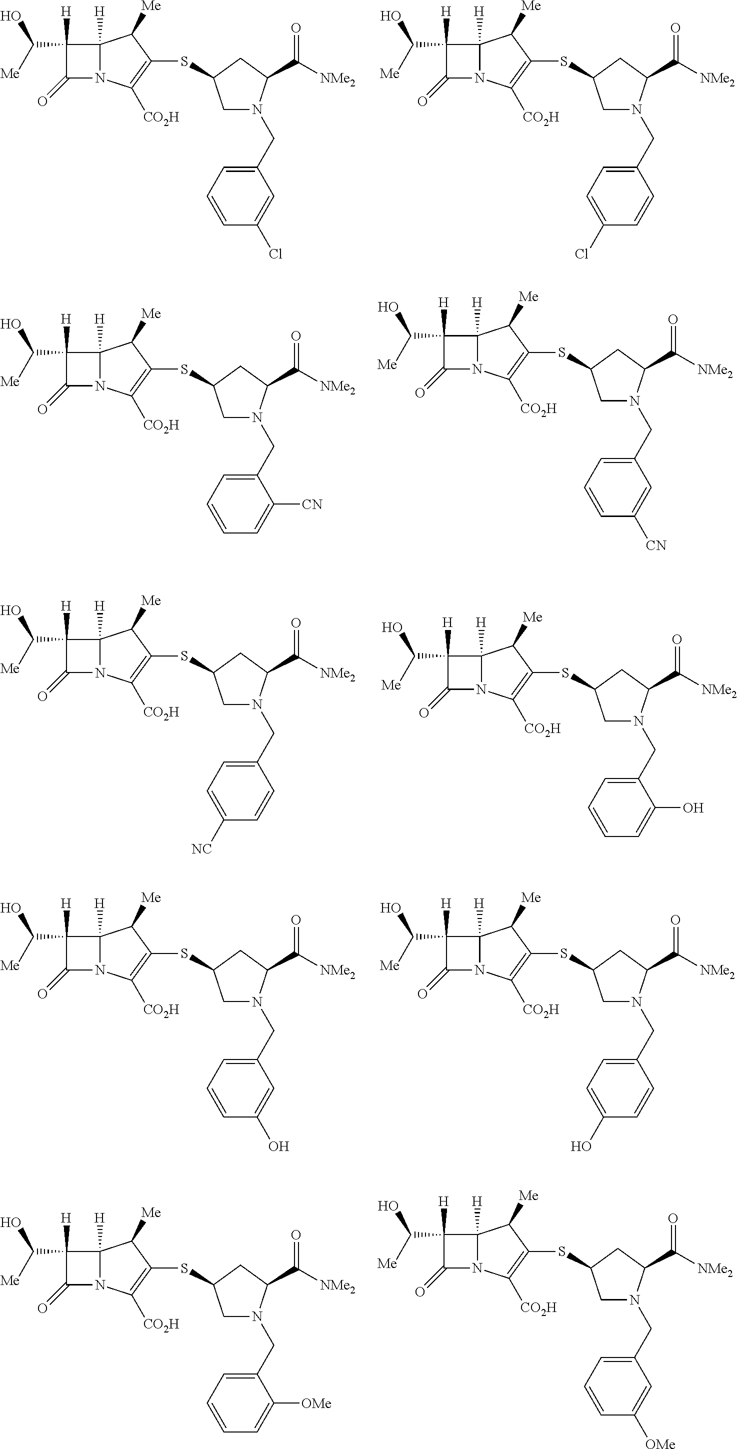

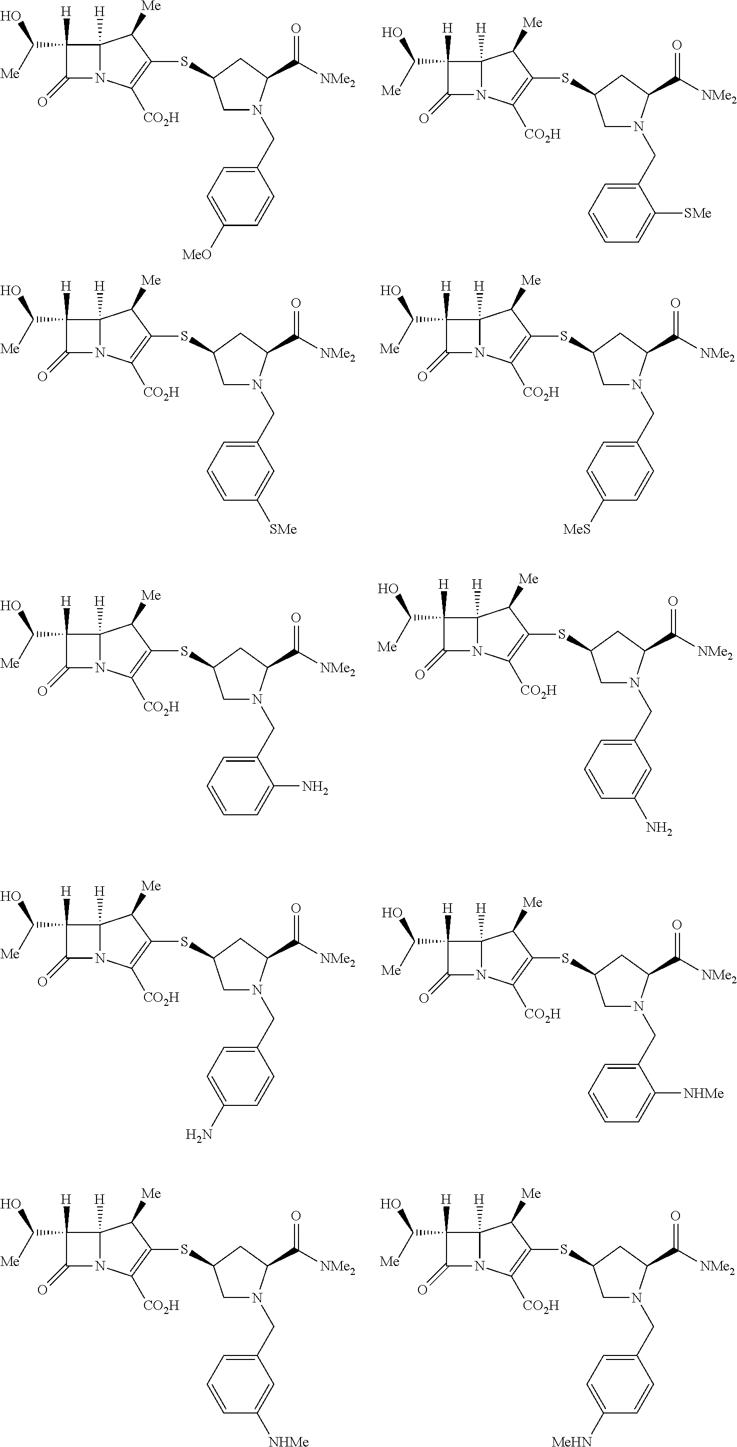

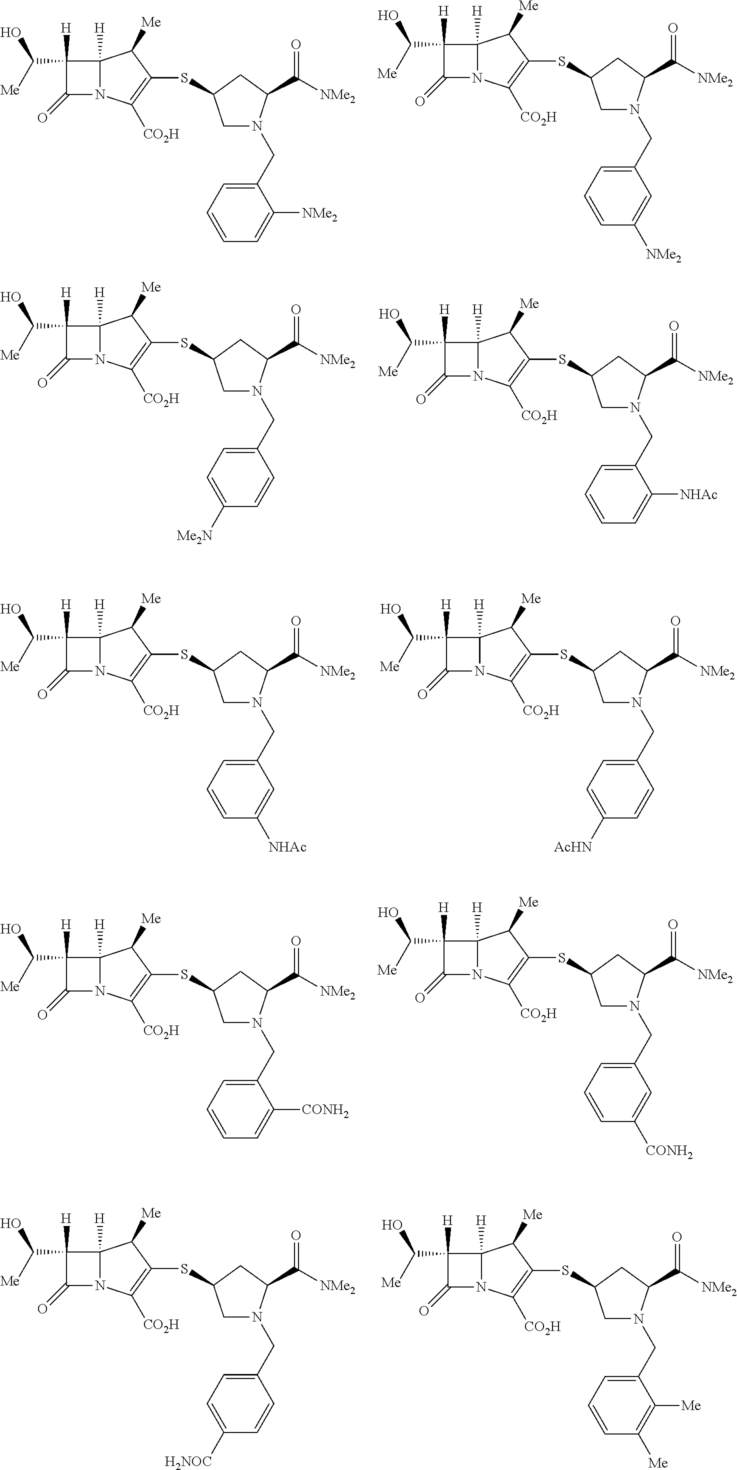

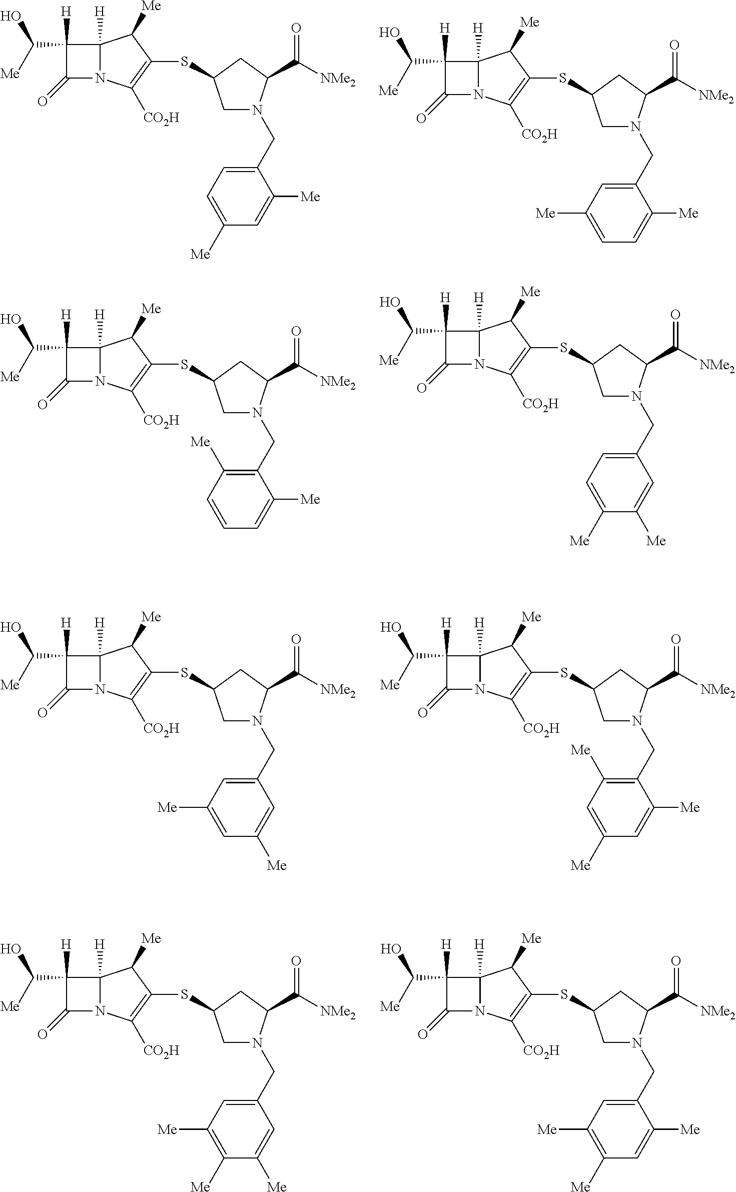

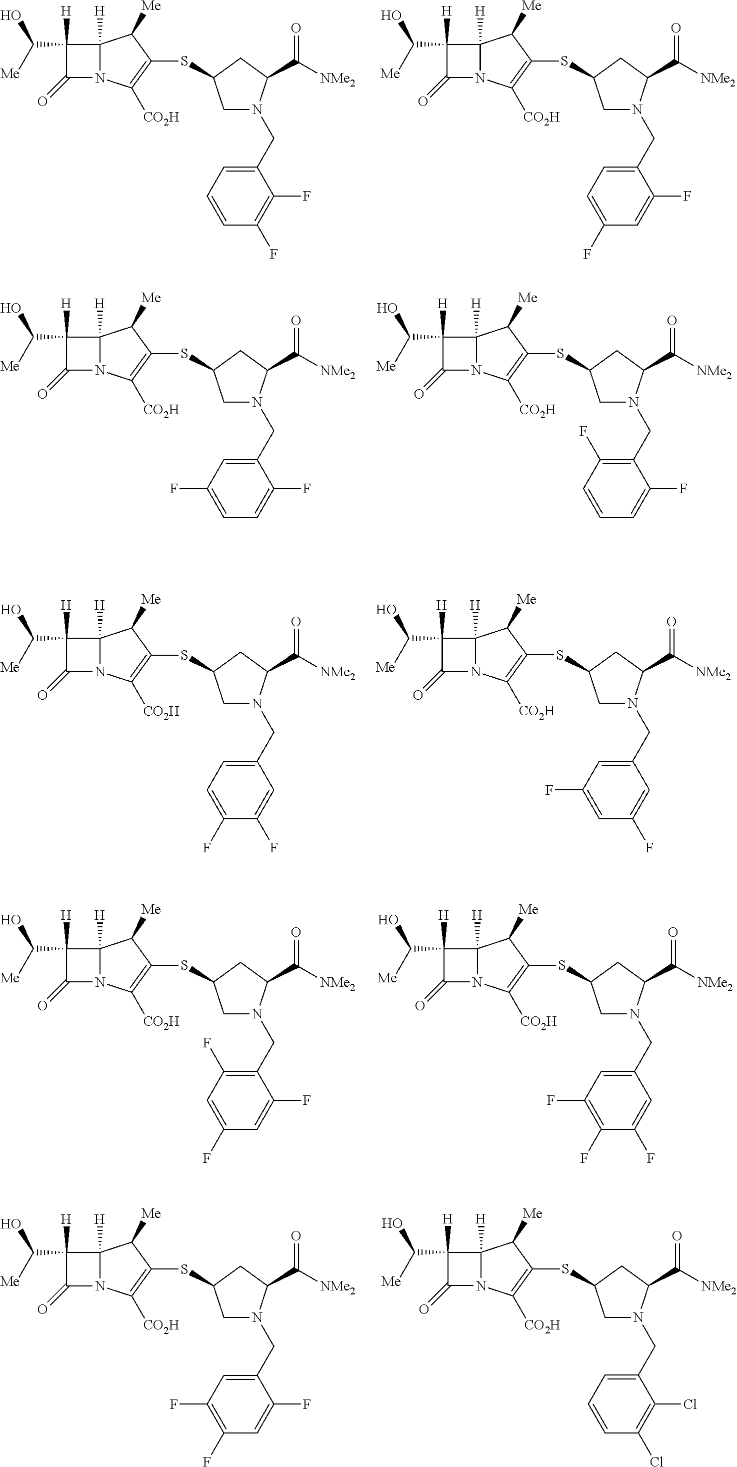

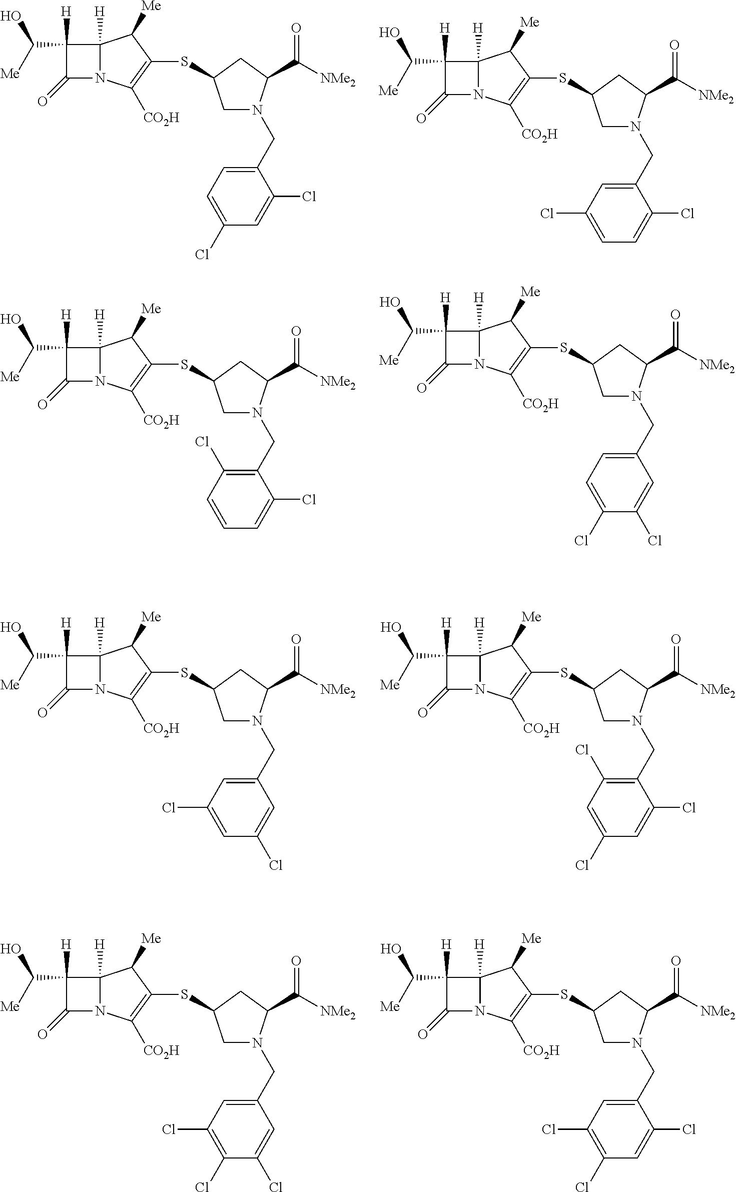

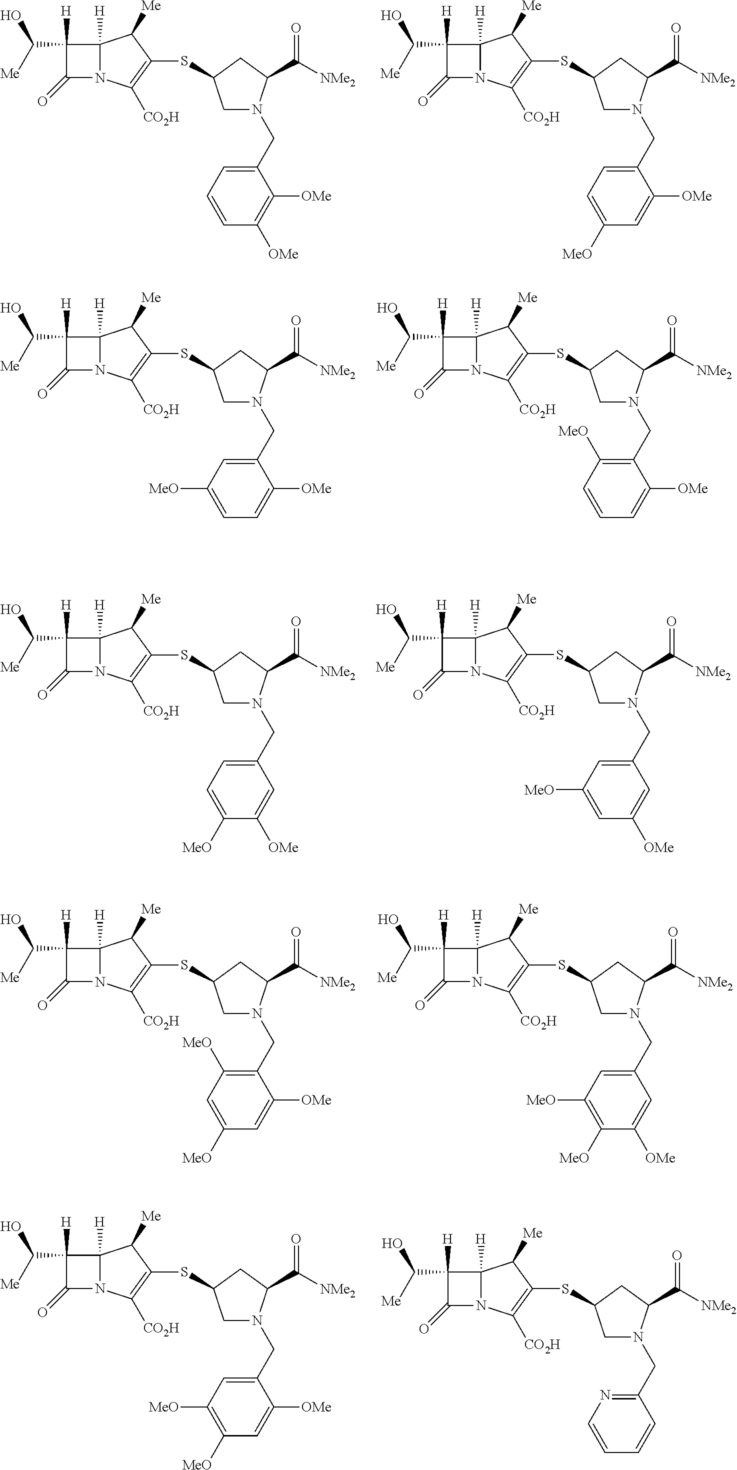

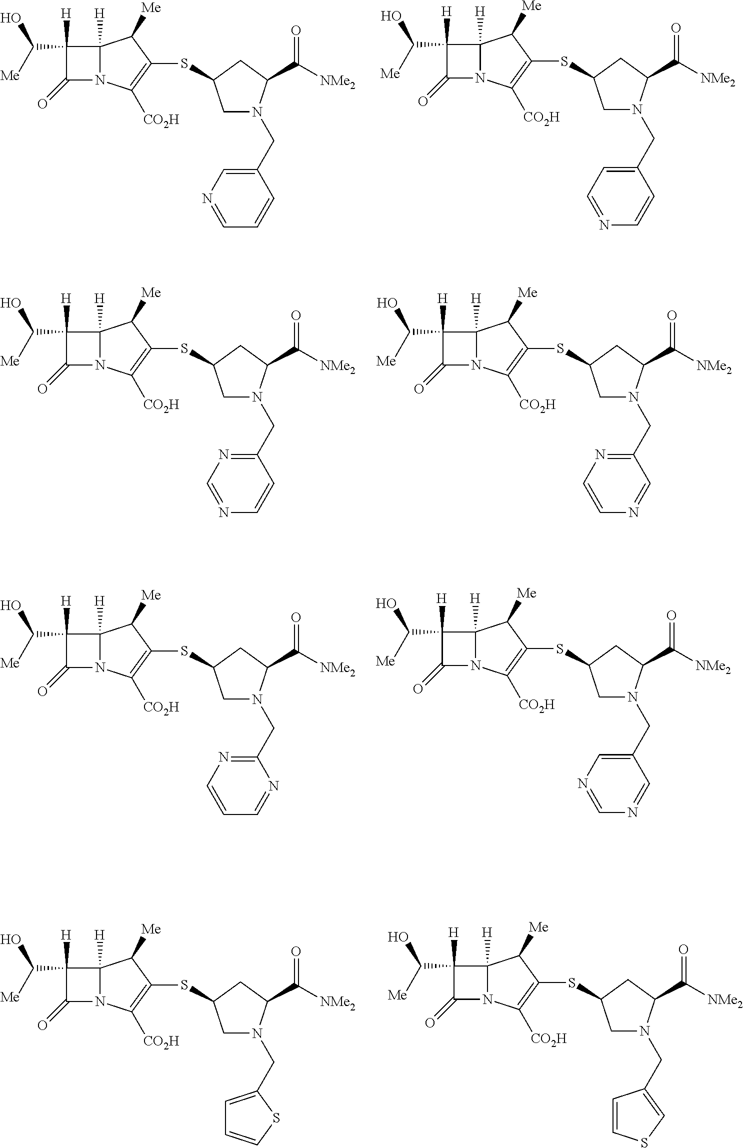

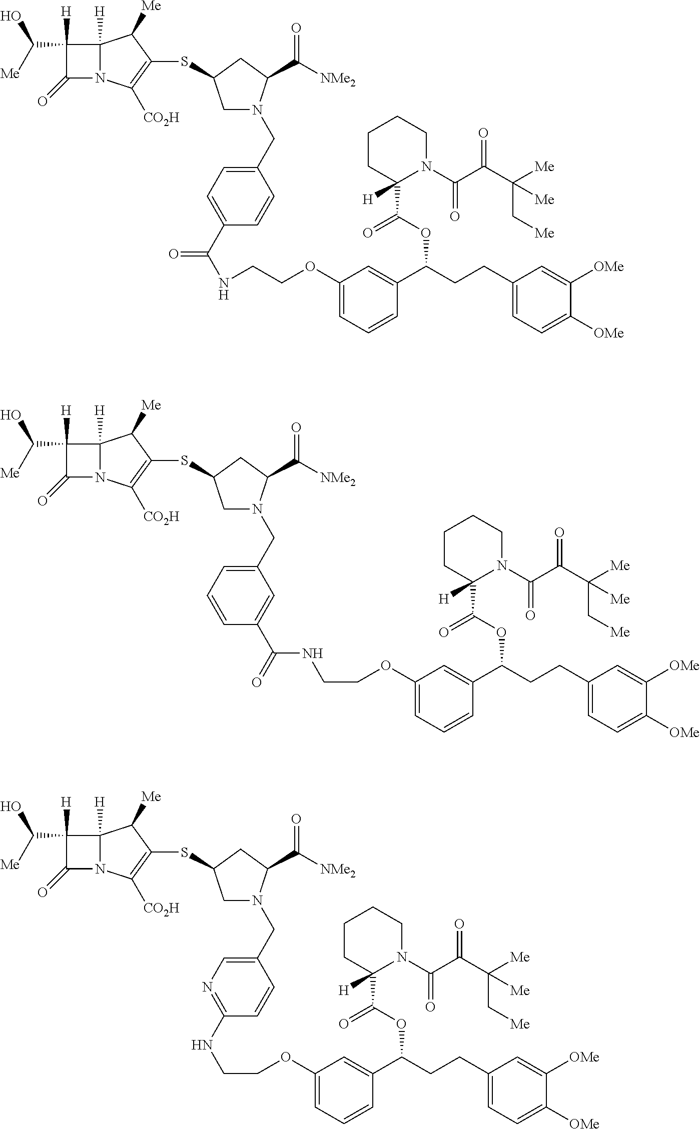

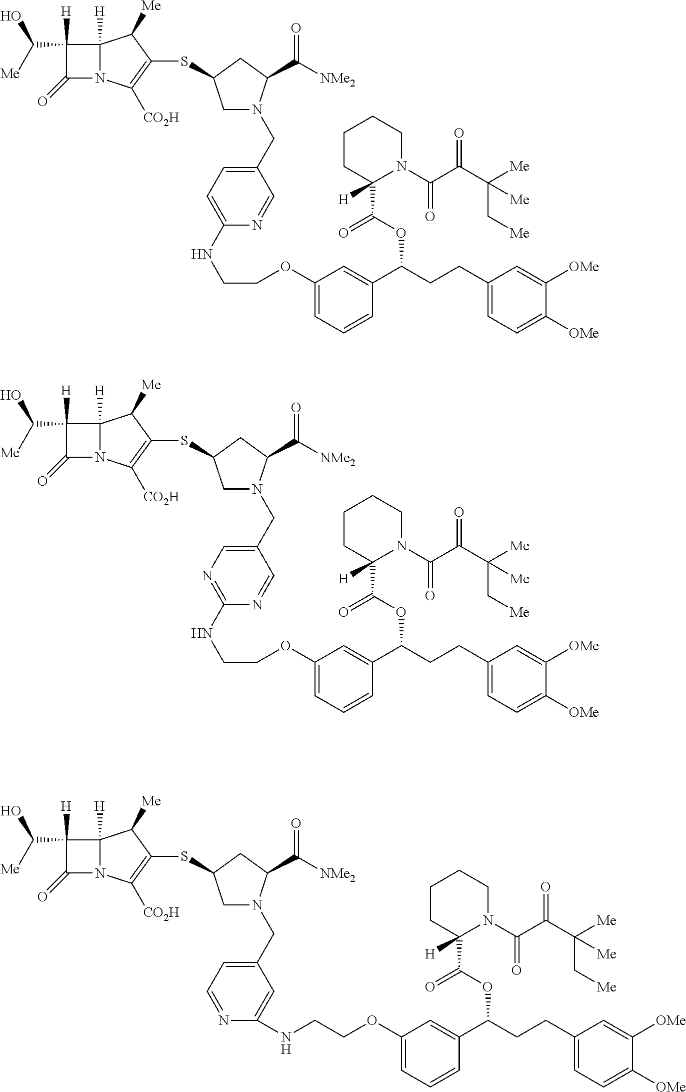

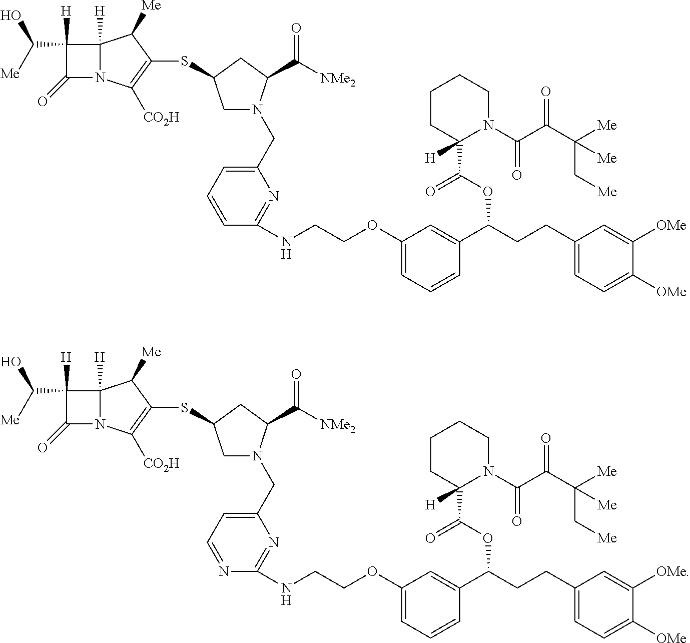

Examples of compounds of formula (IIb) include:

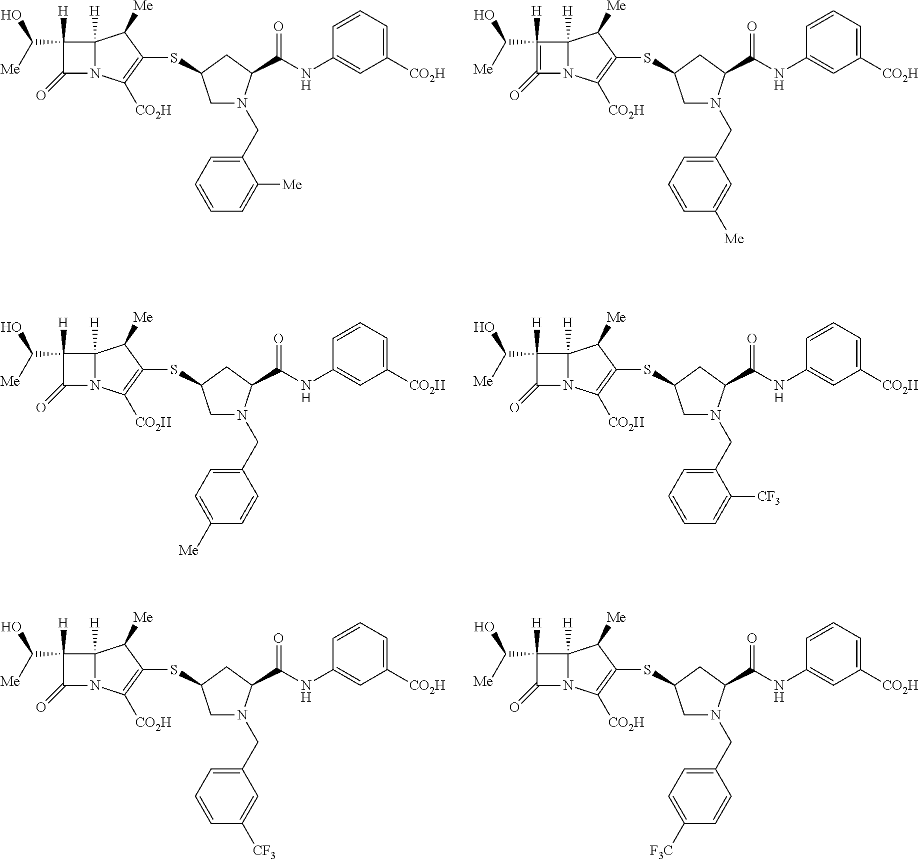

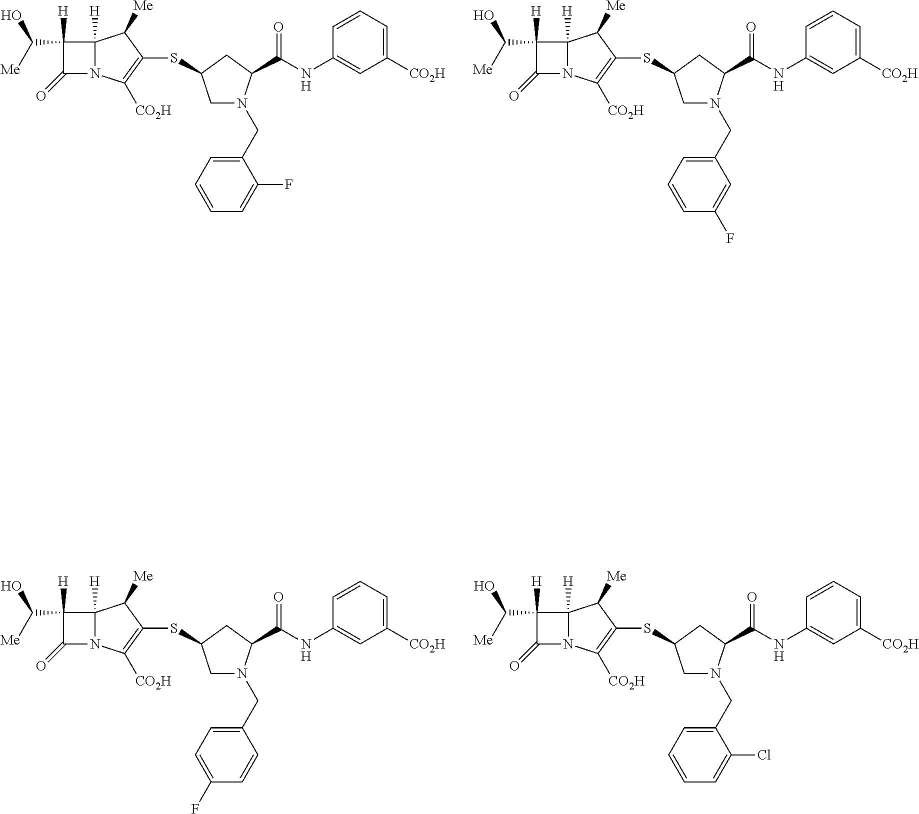

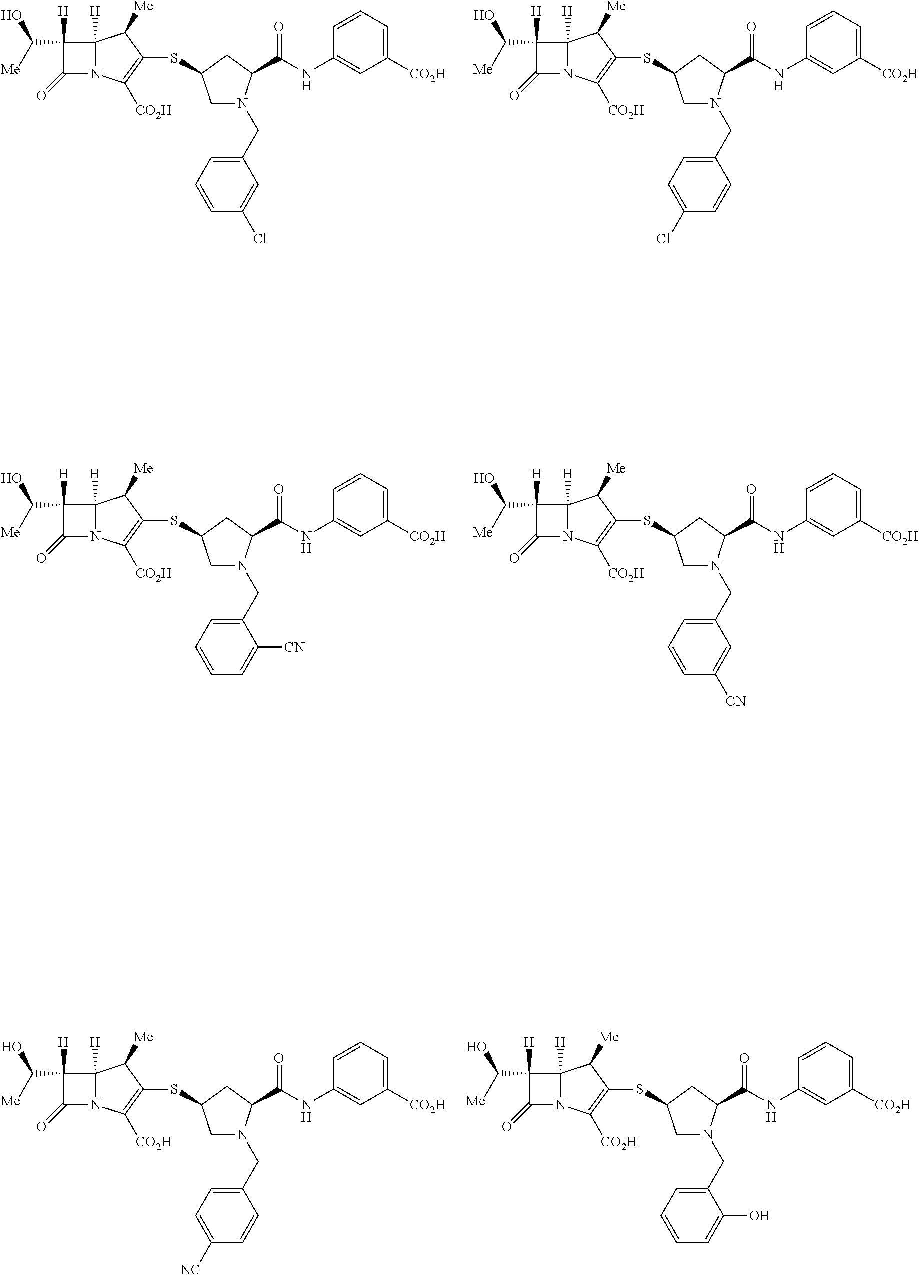

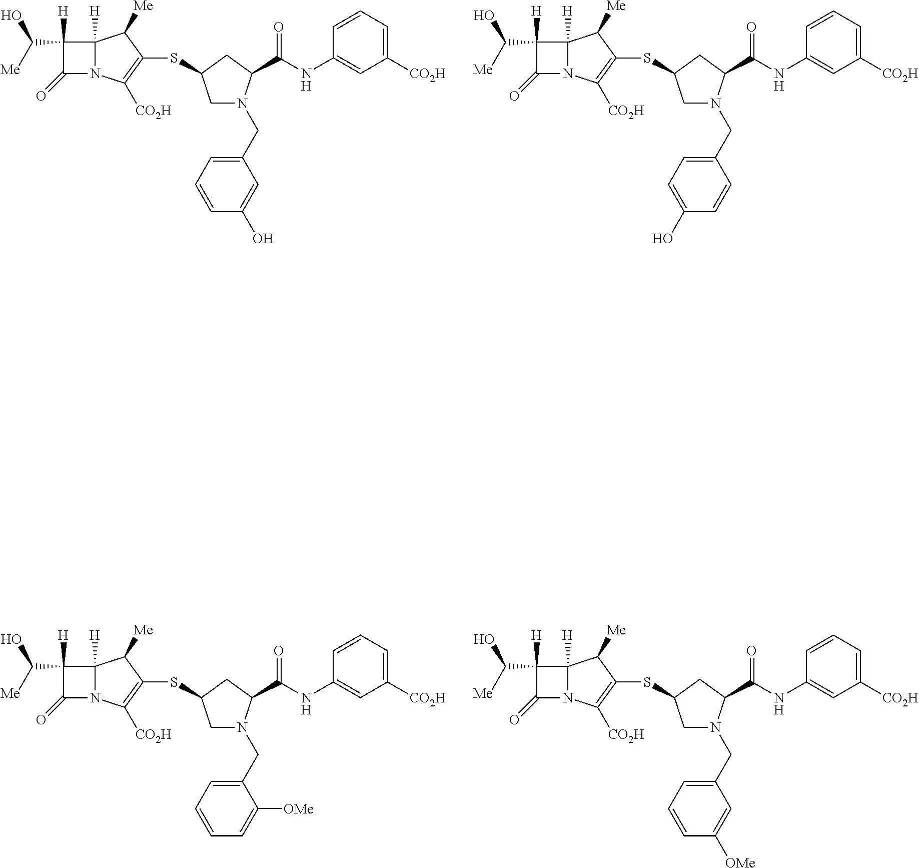

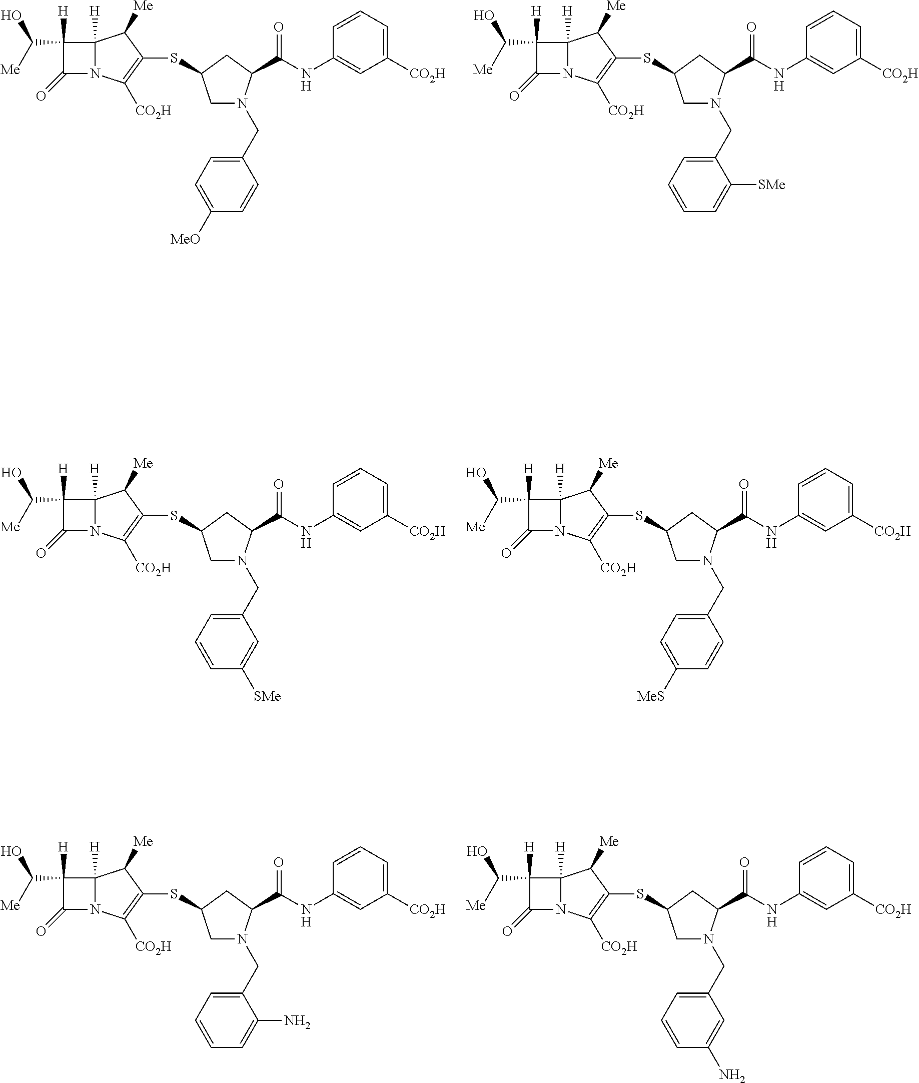

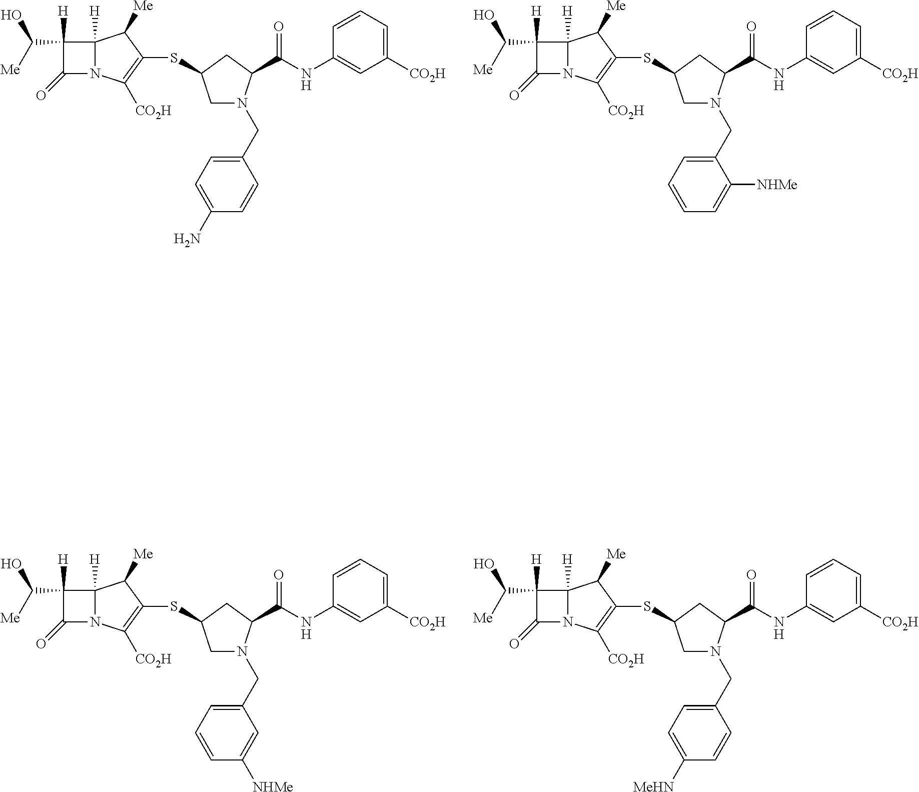

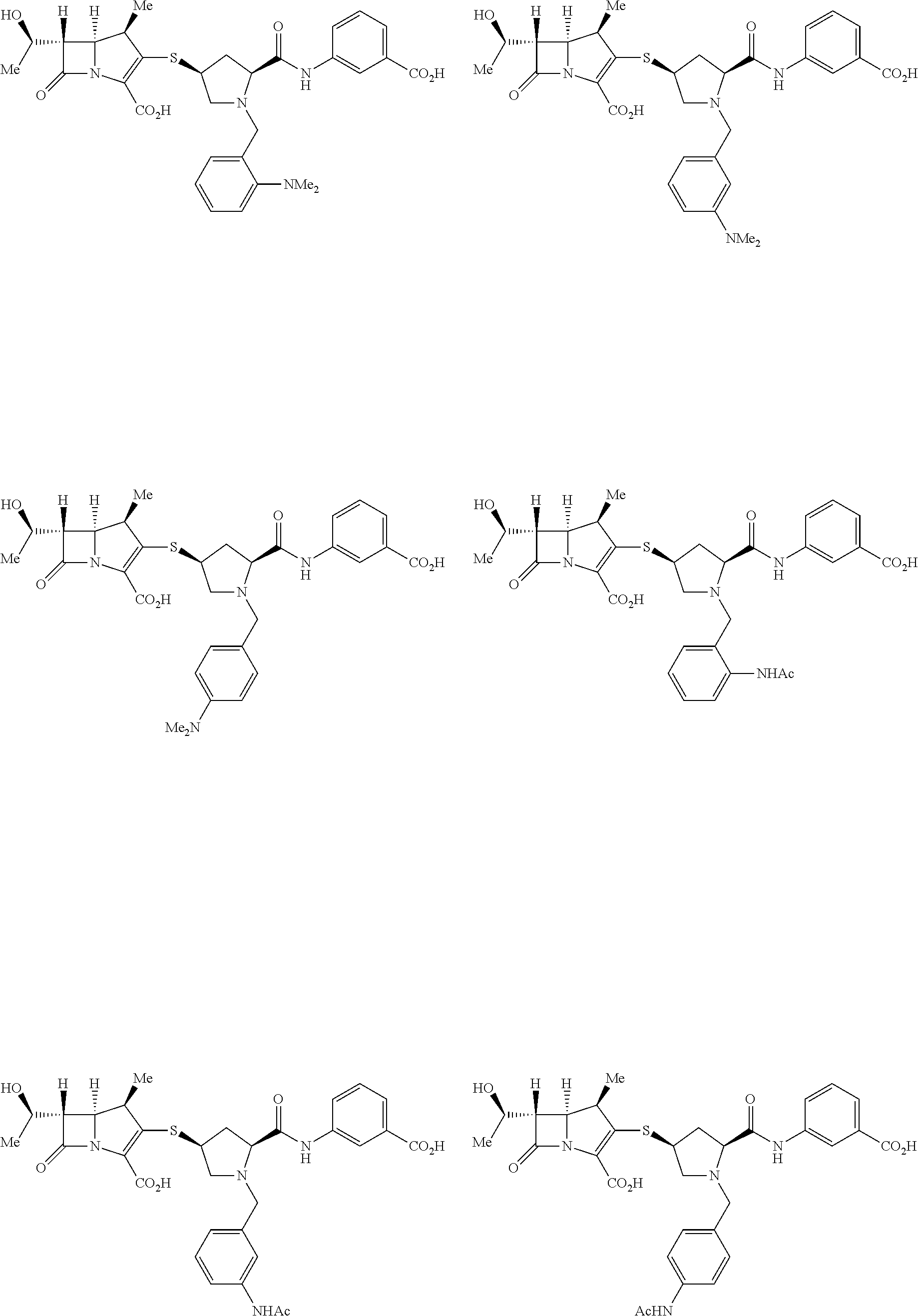

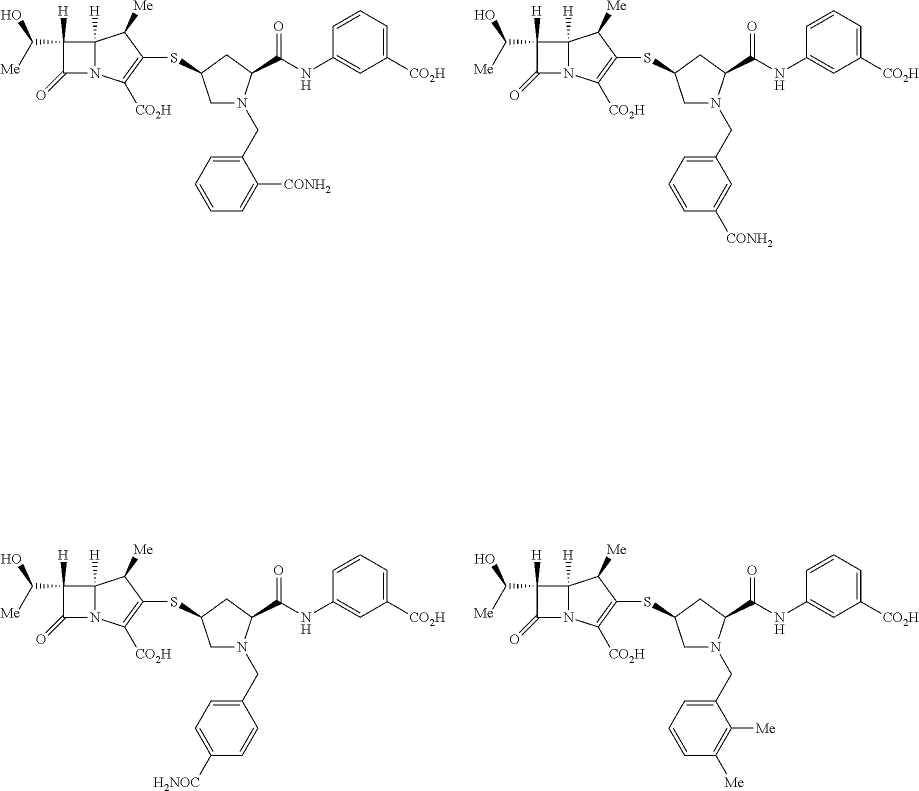

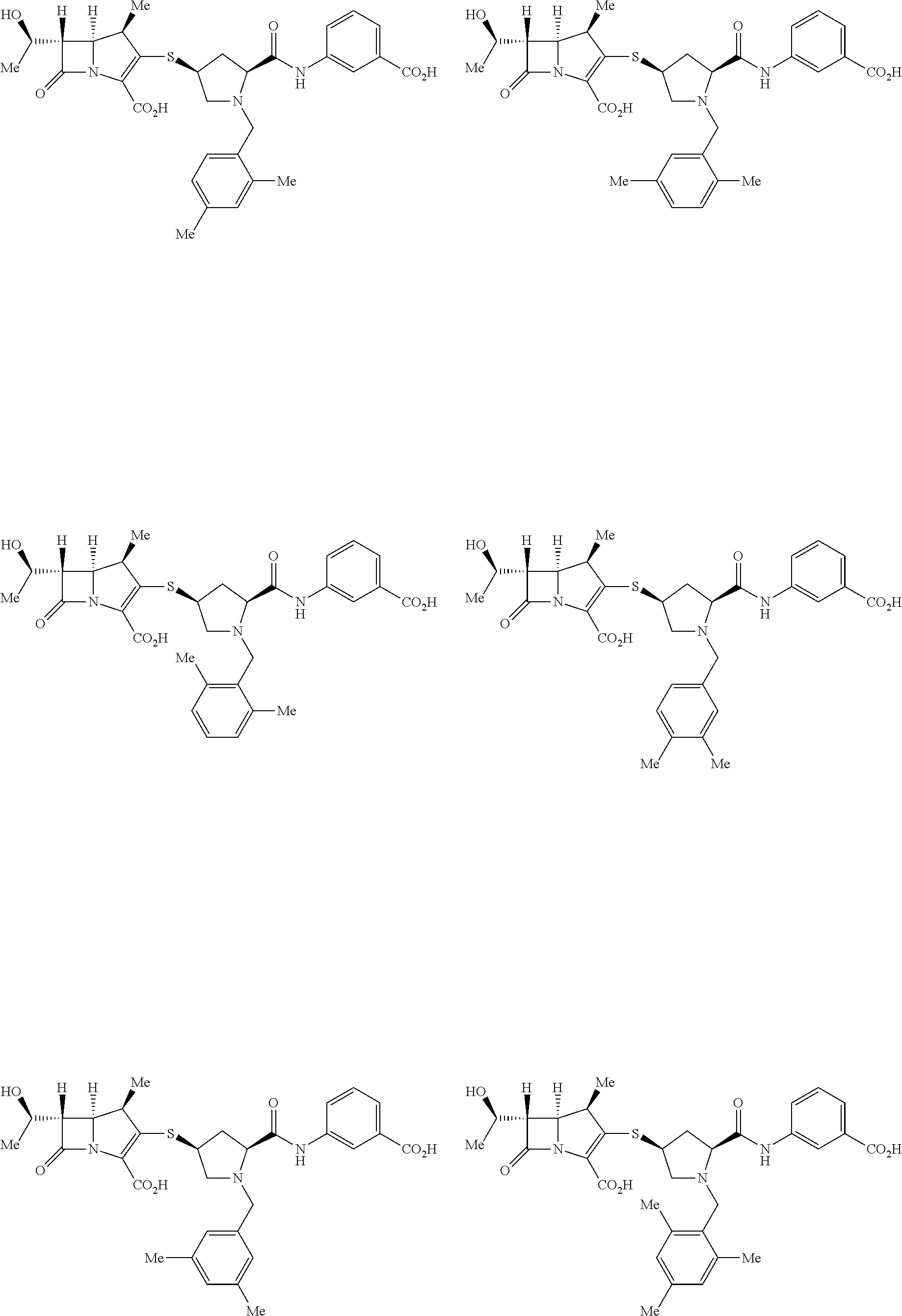

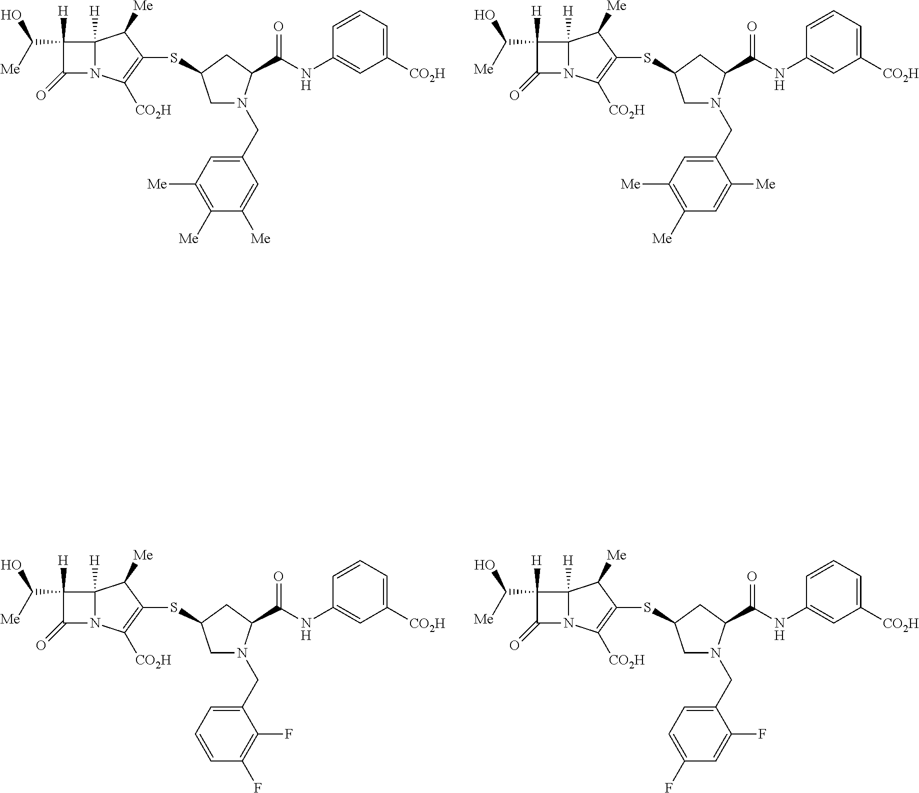

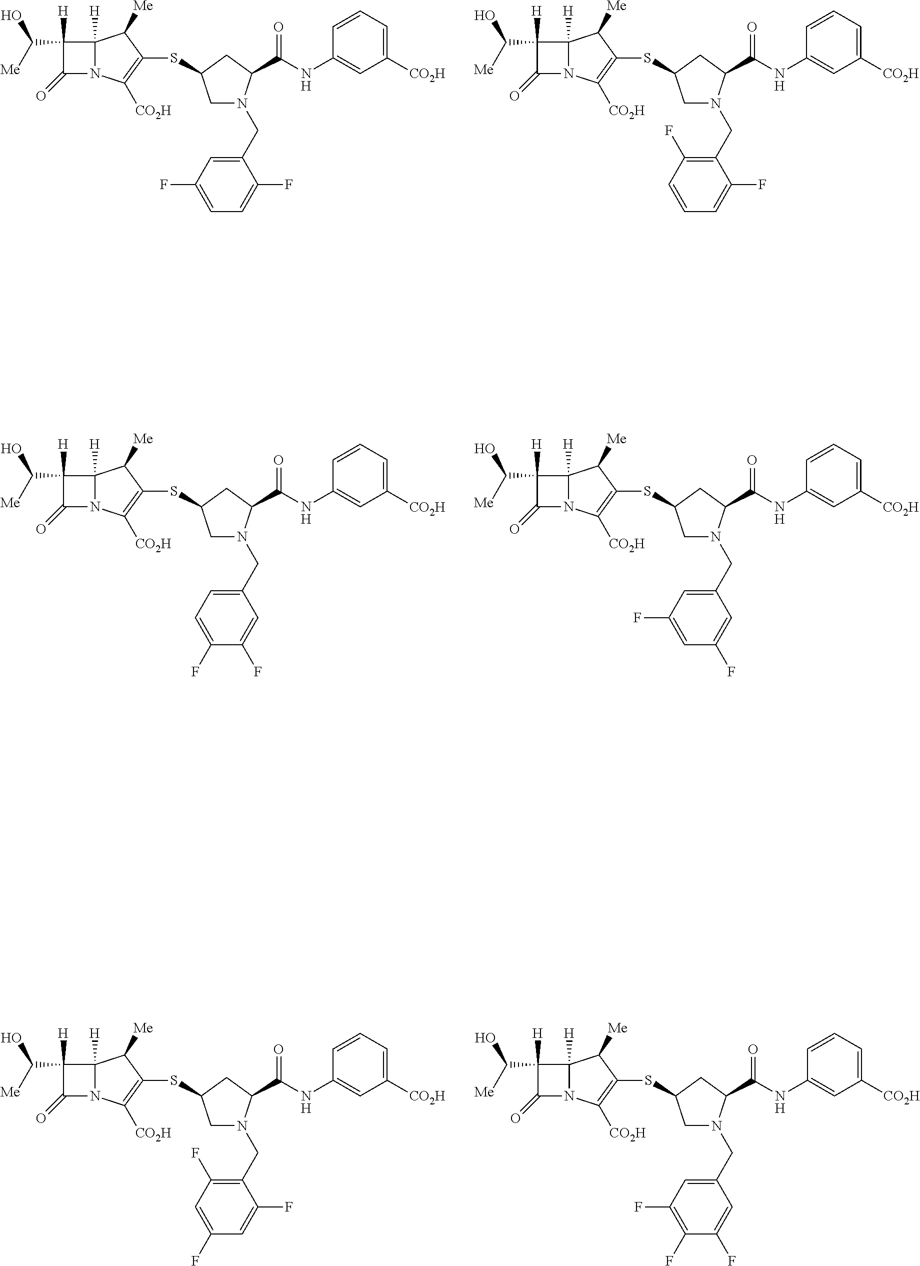

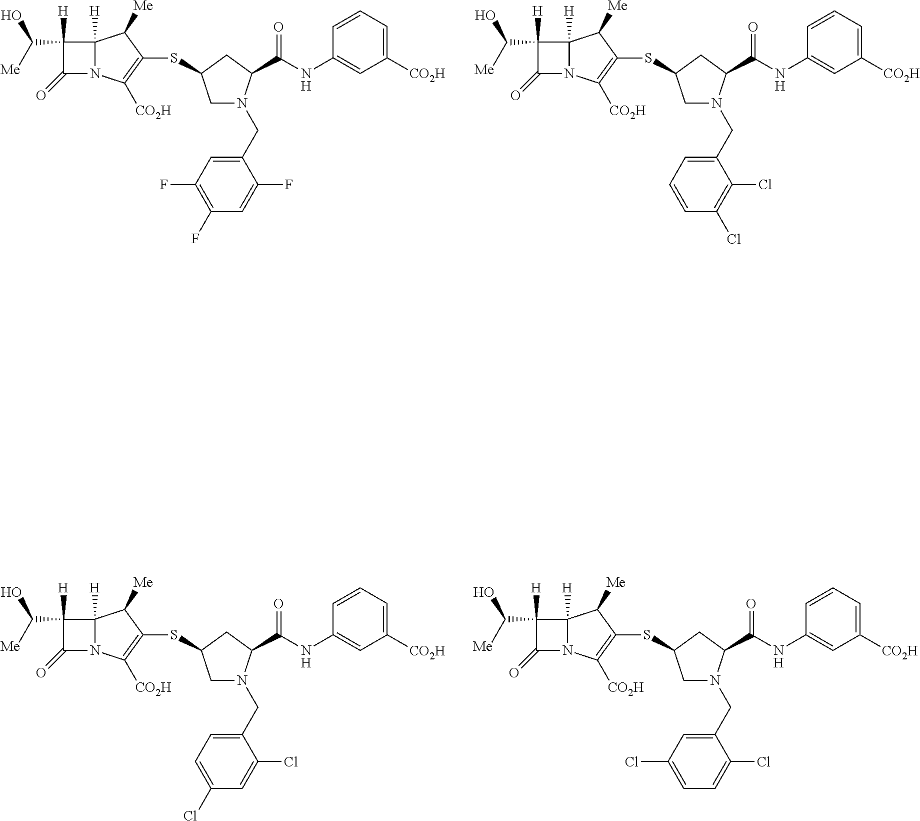

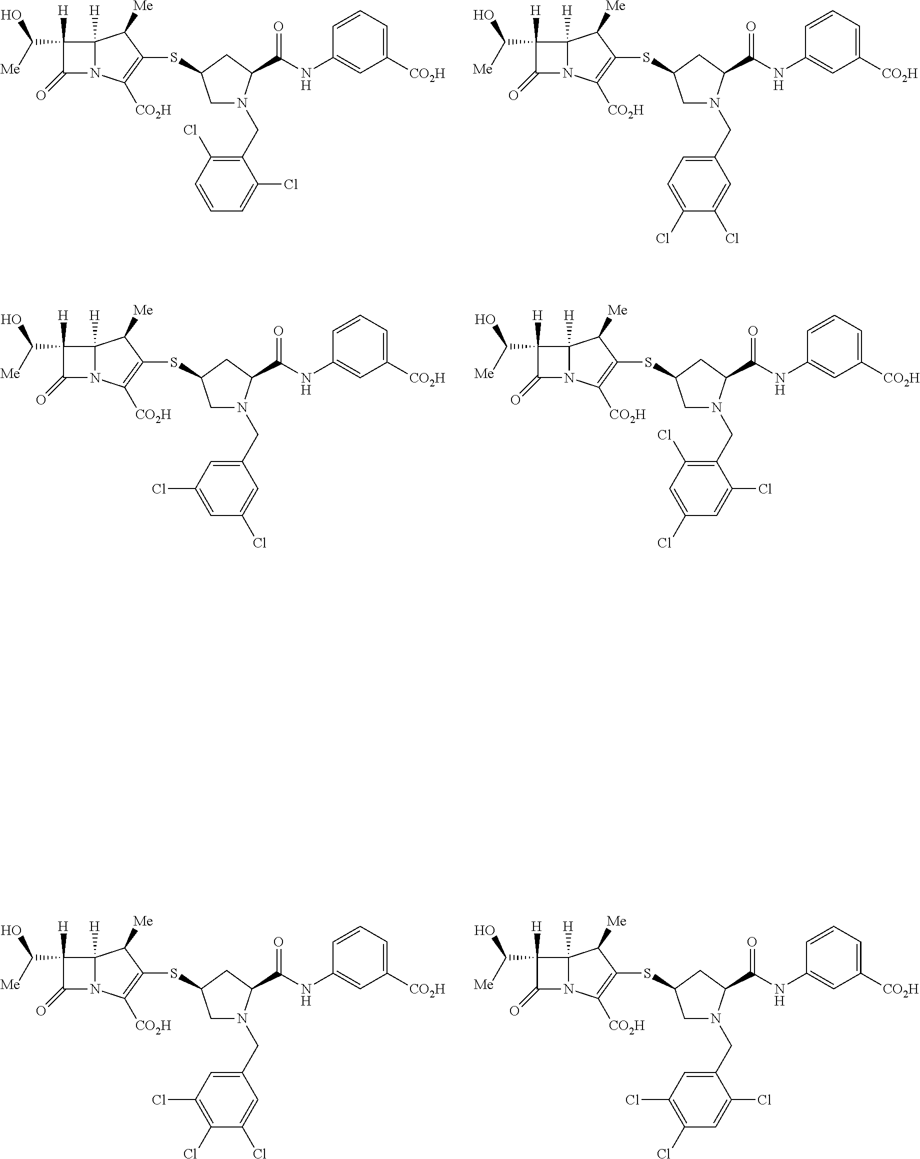

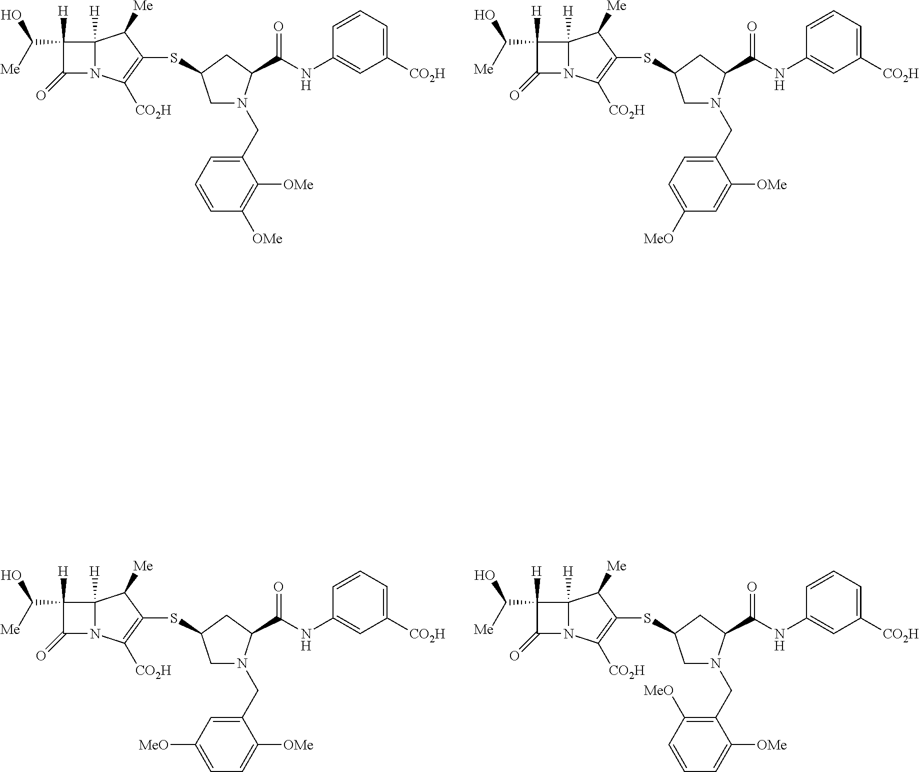

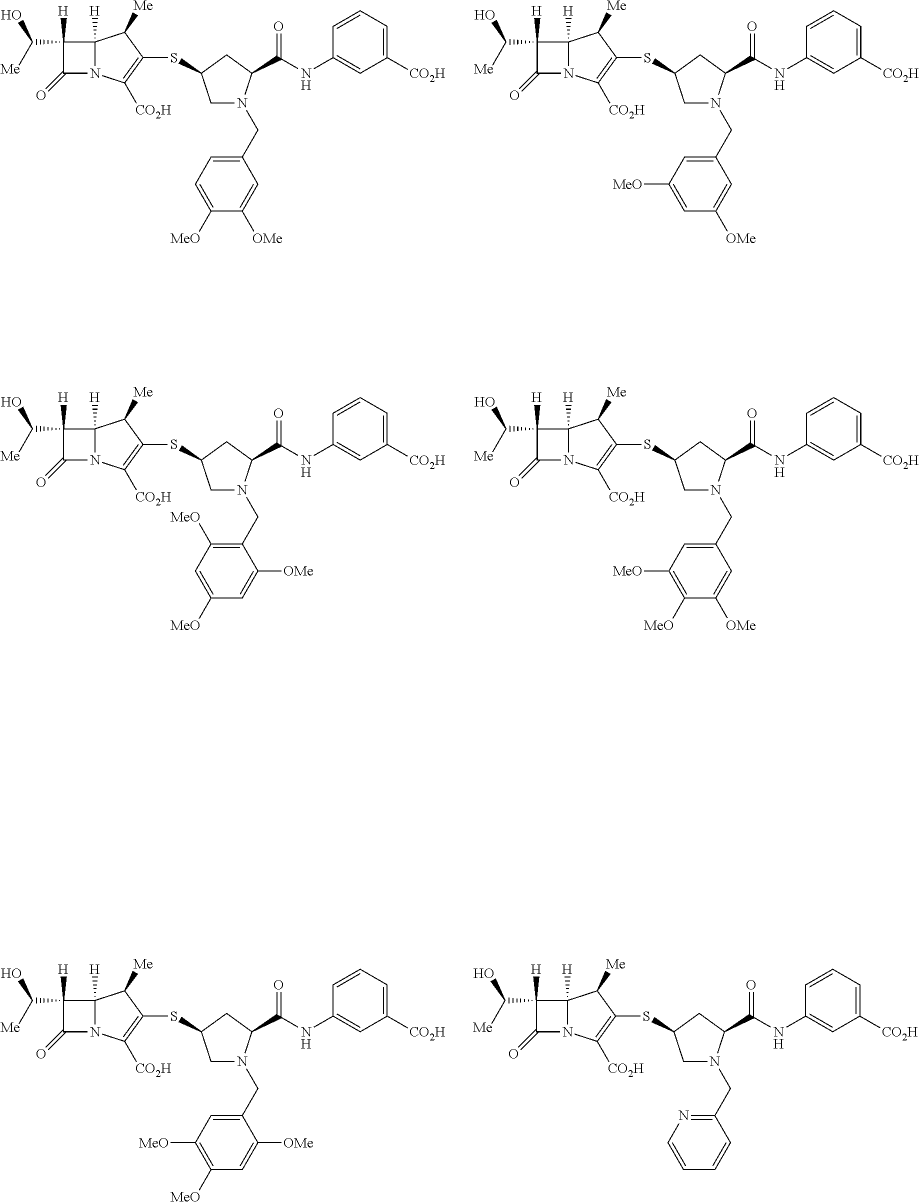

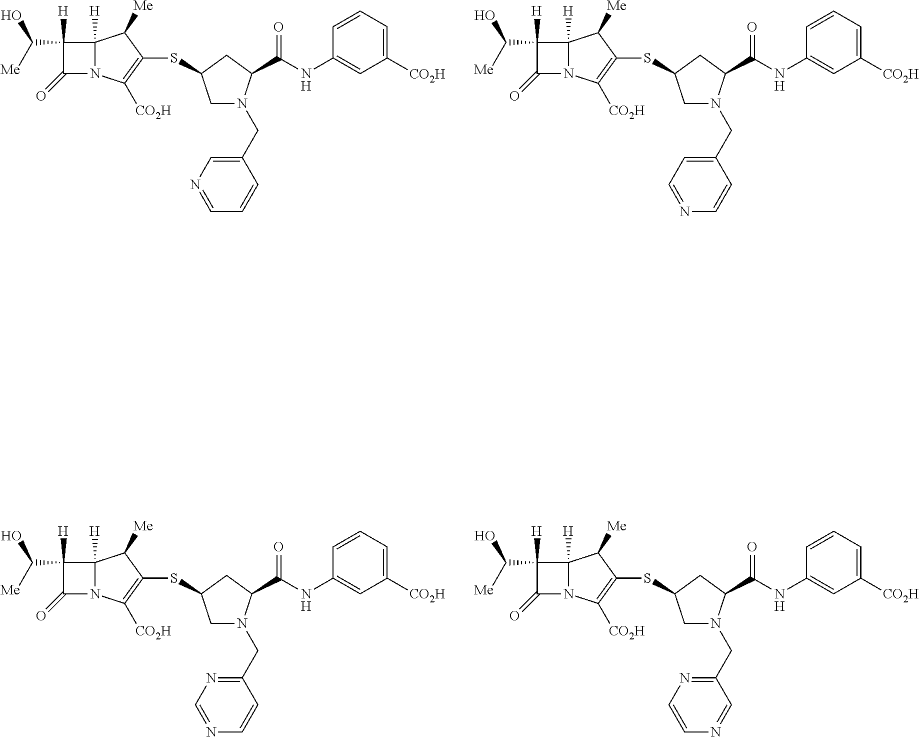

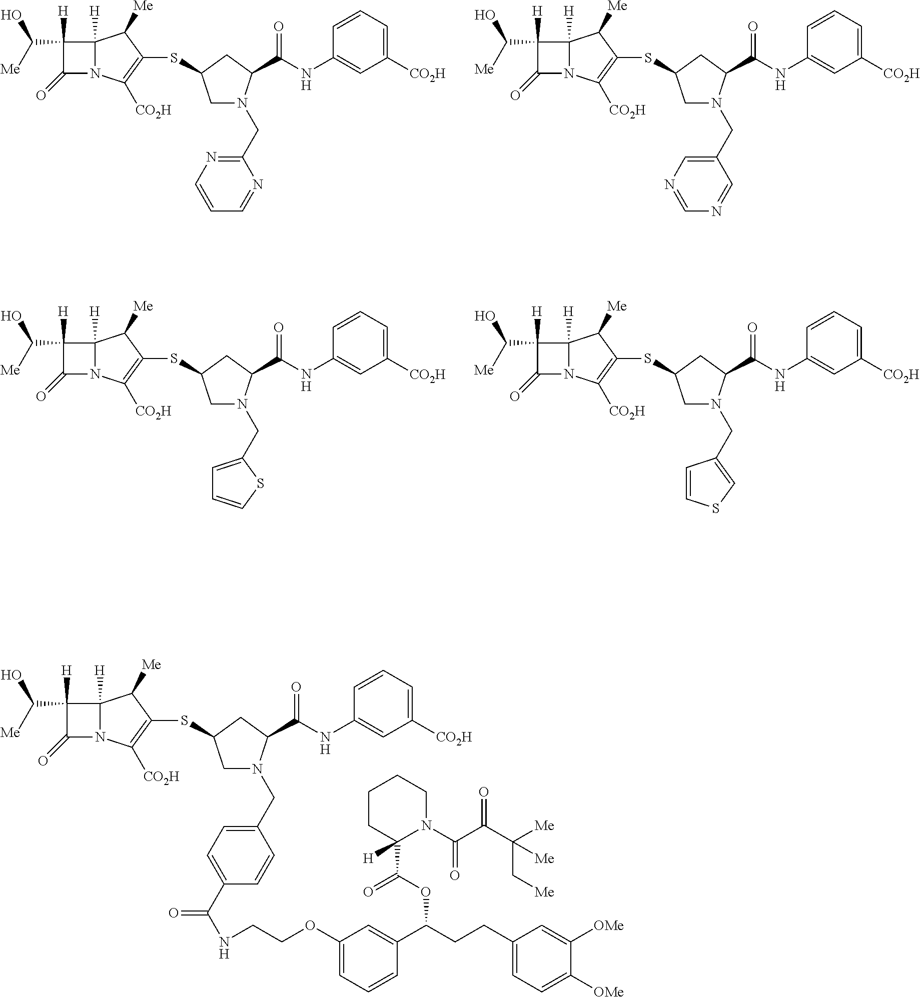

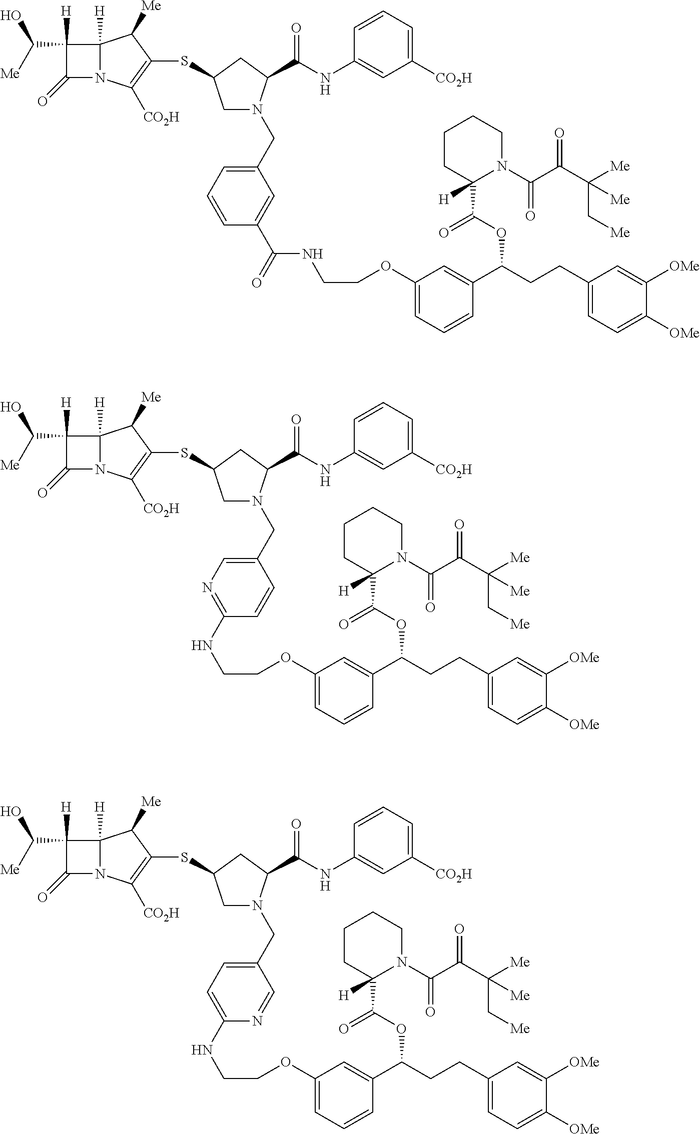

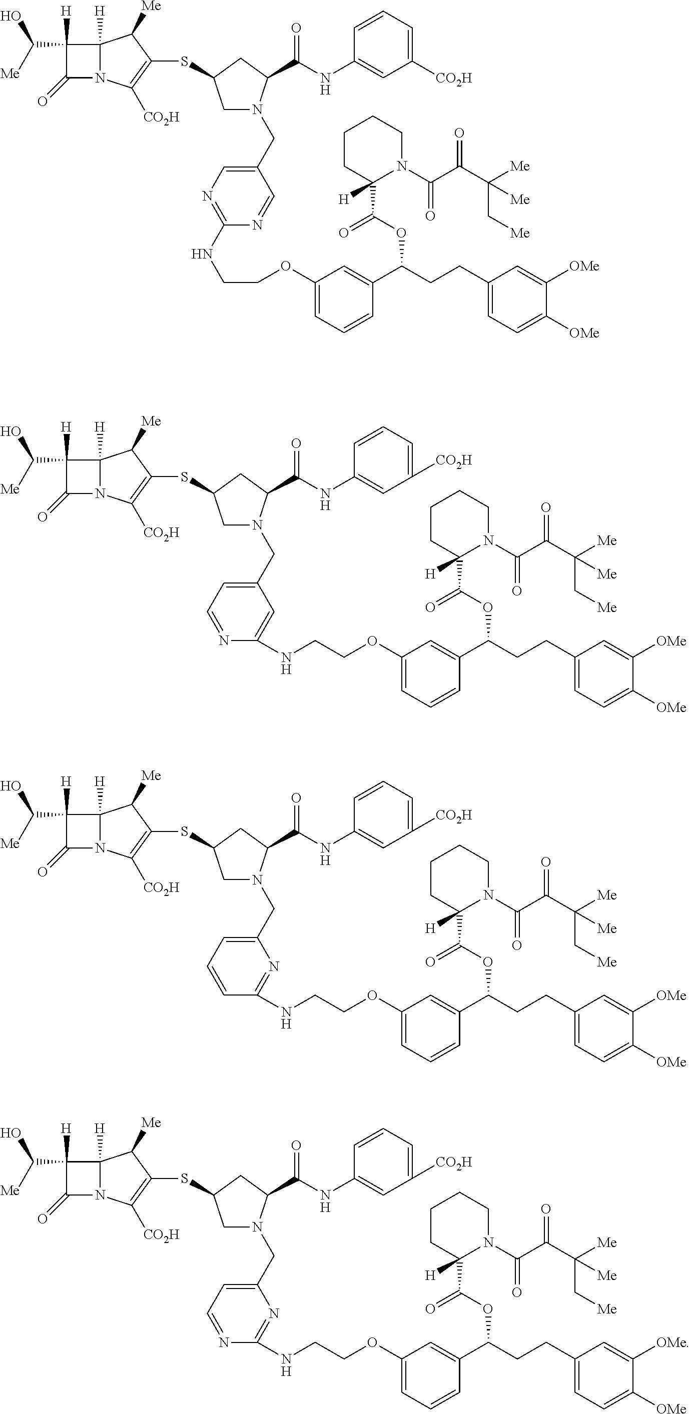

Examples of compounds of formula (IIc) include:

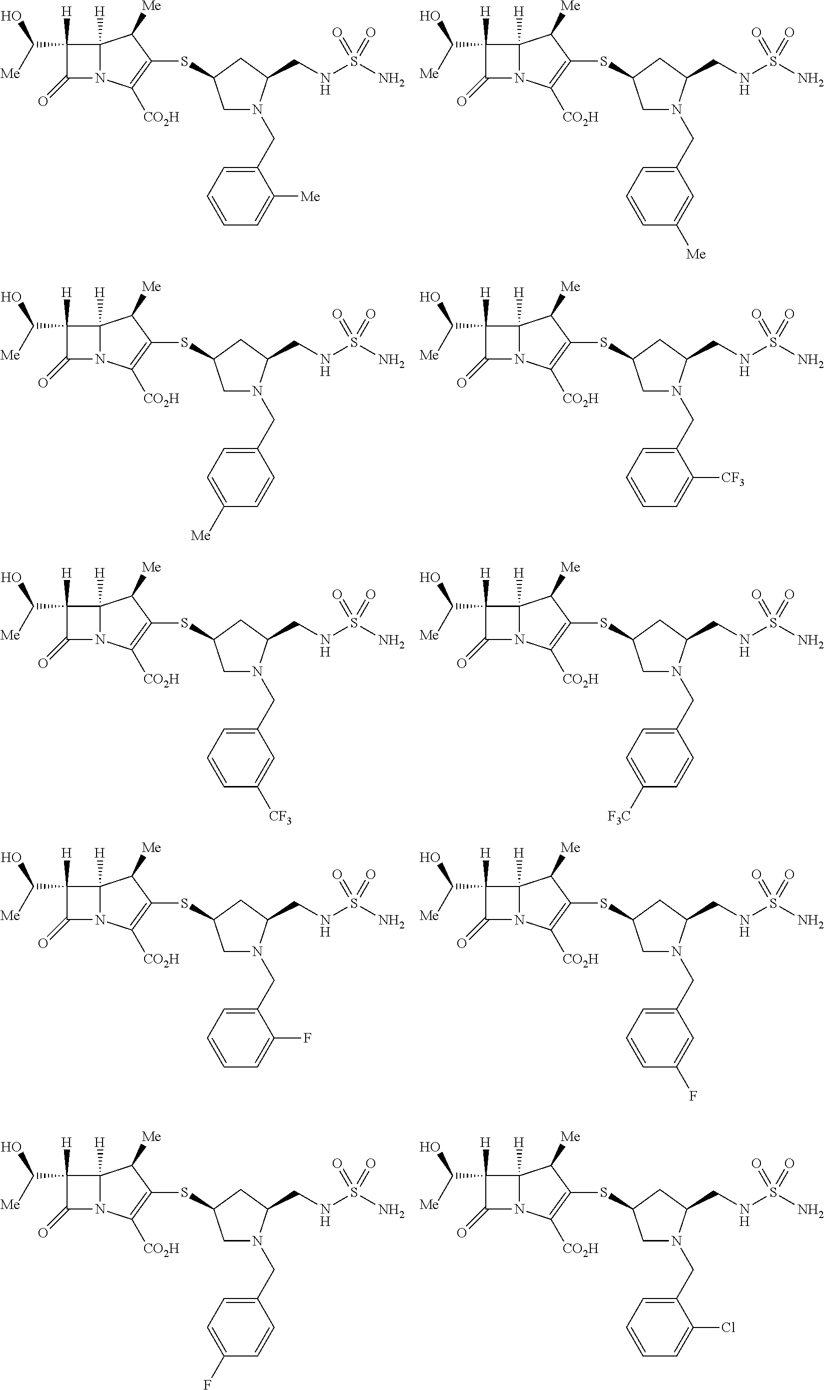

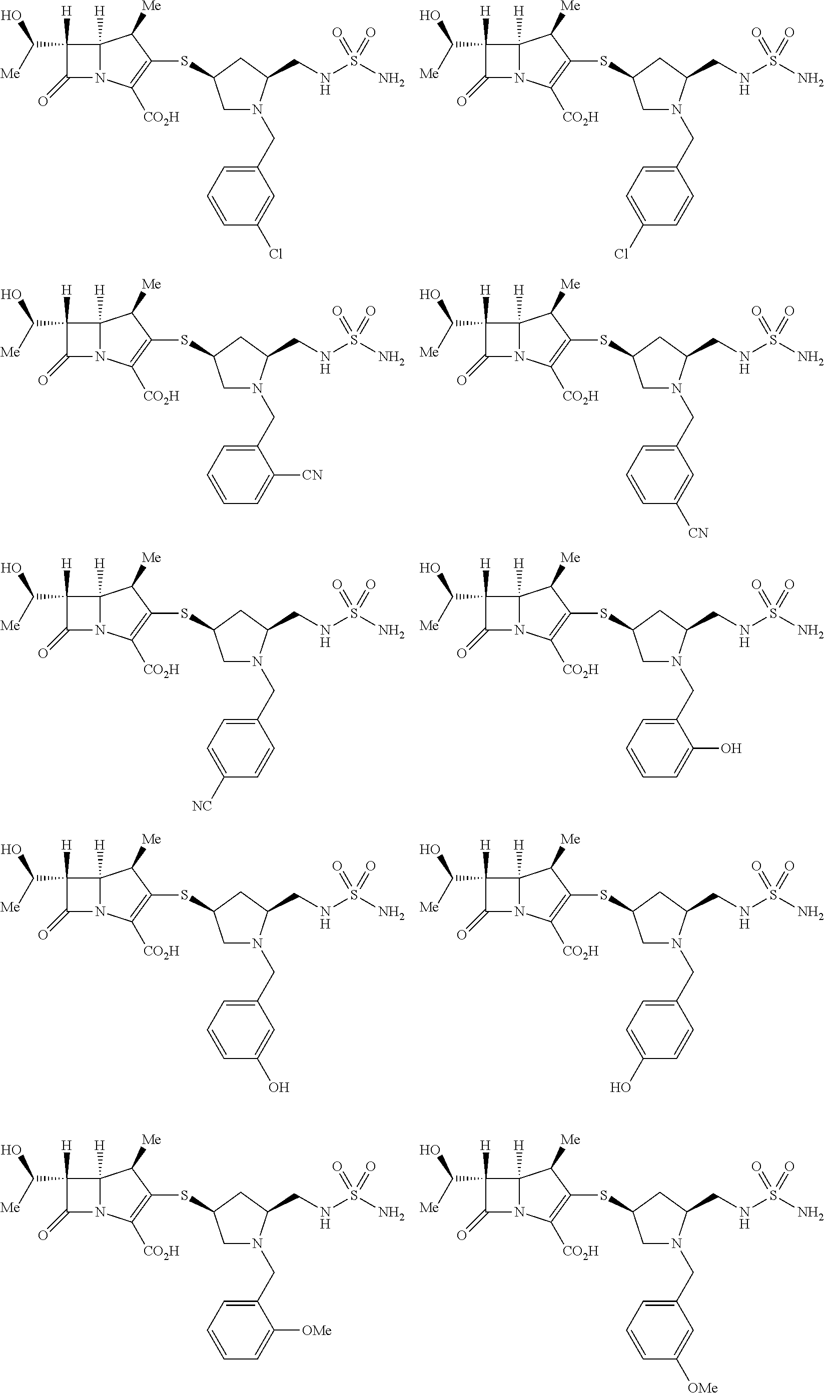

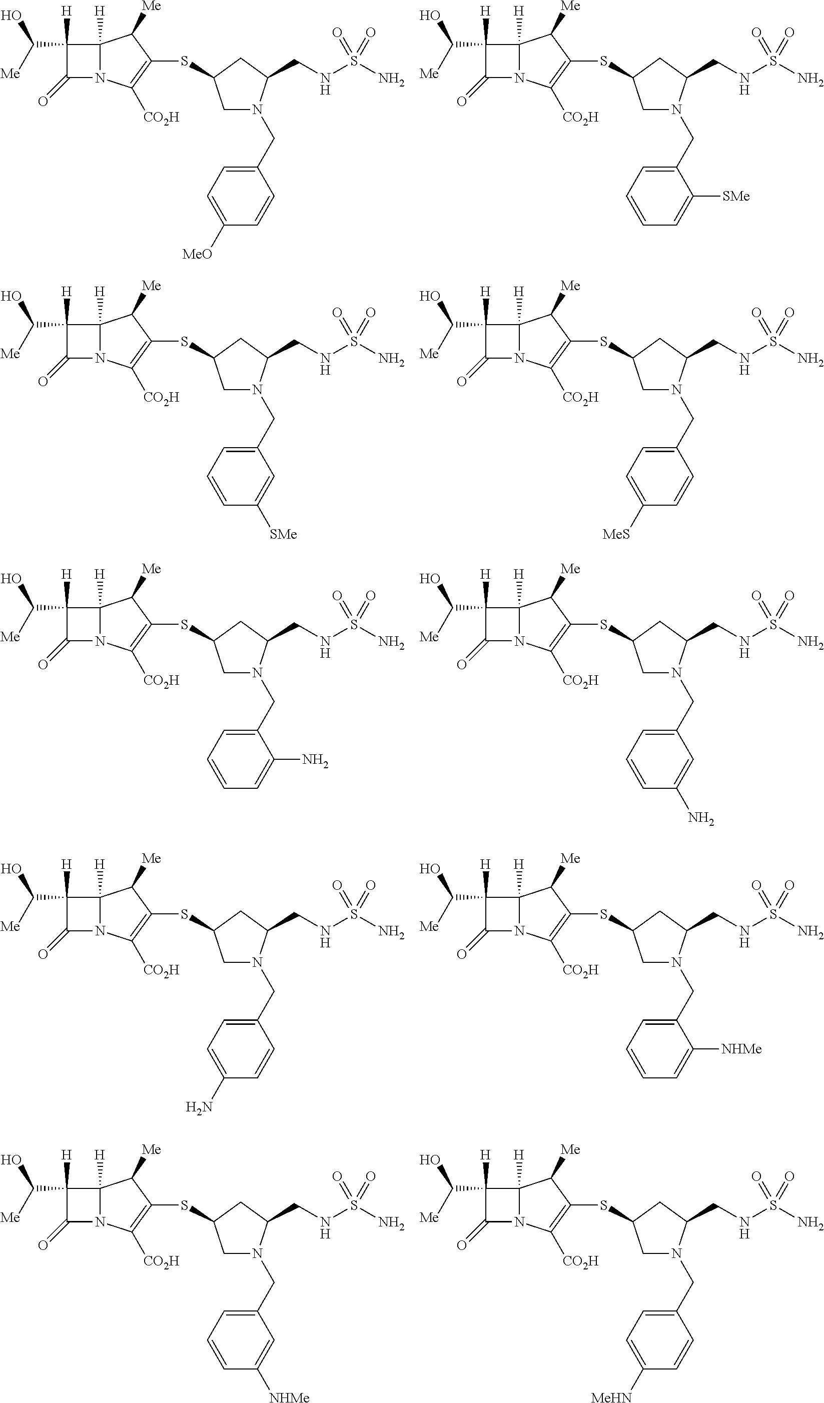

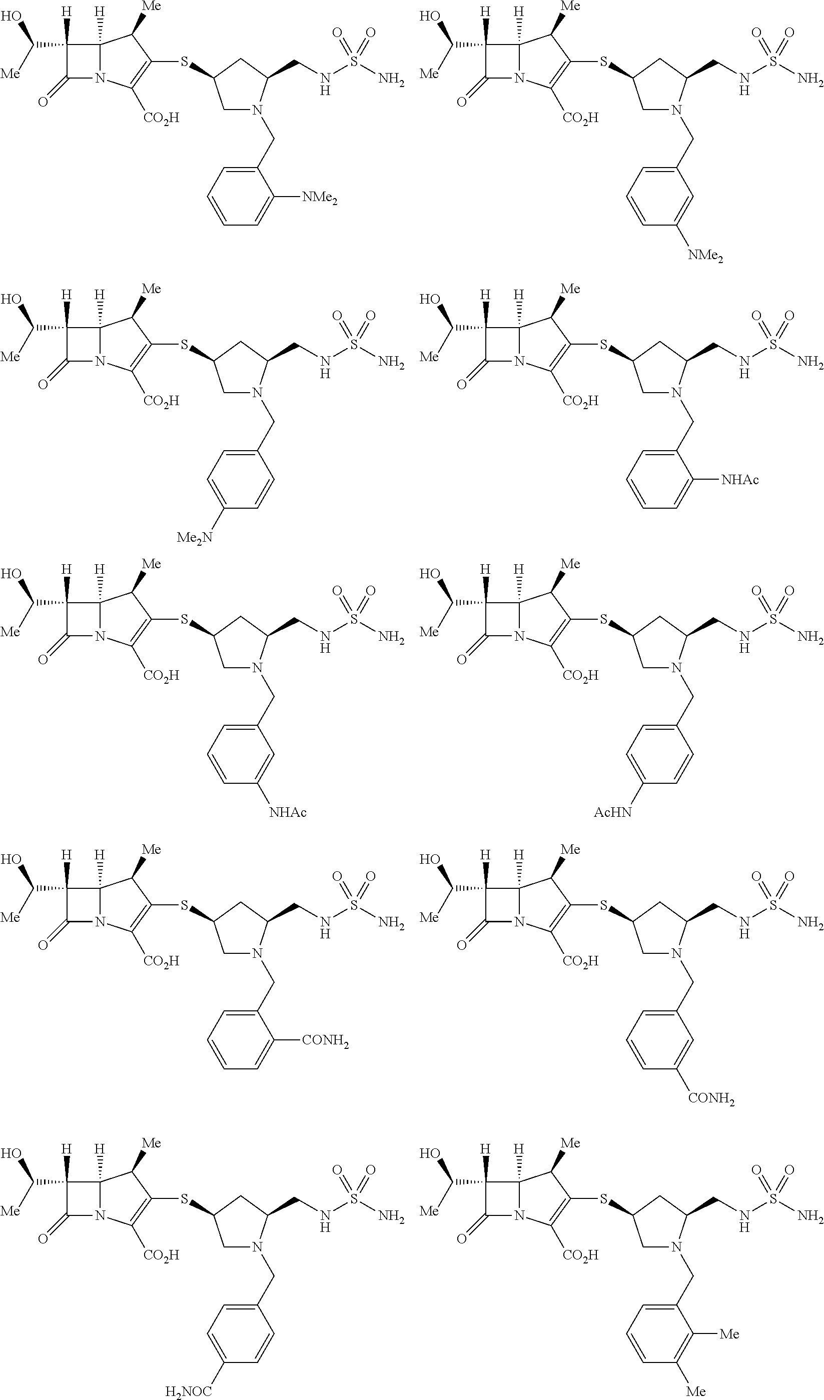

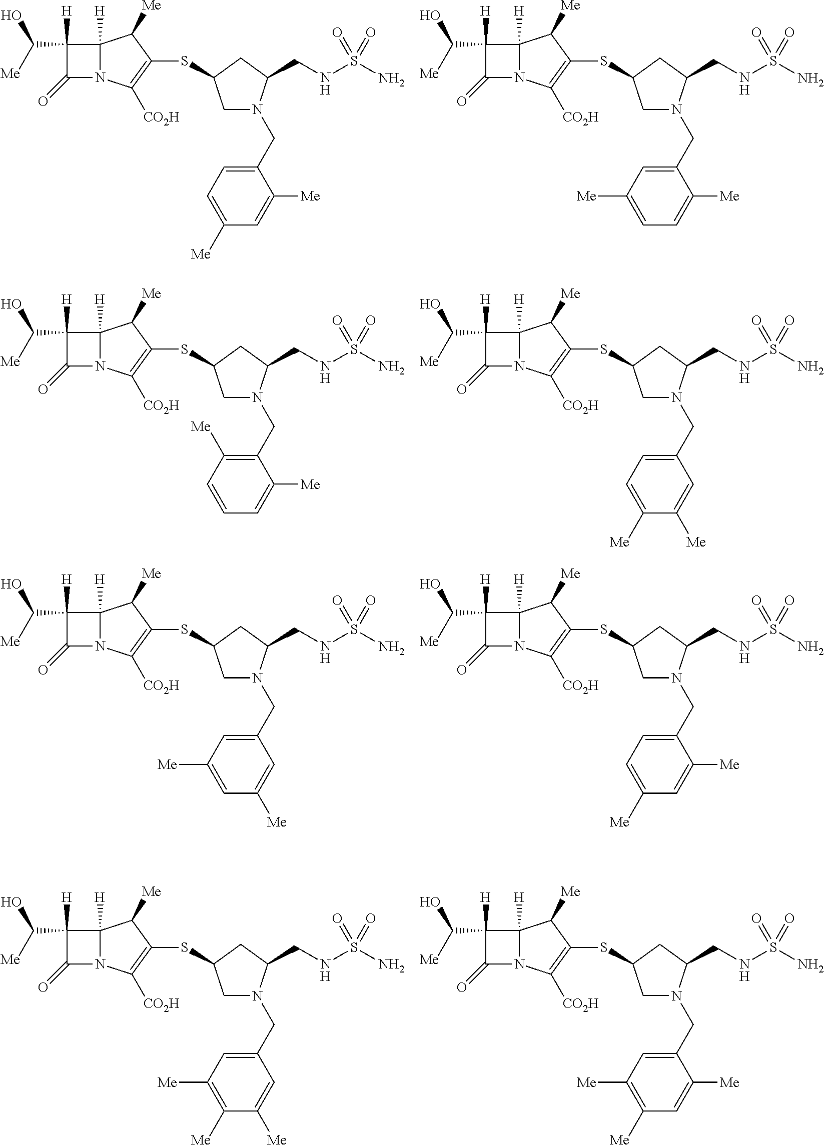

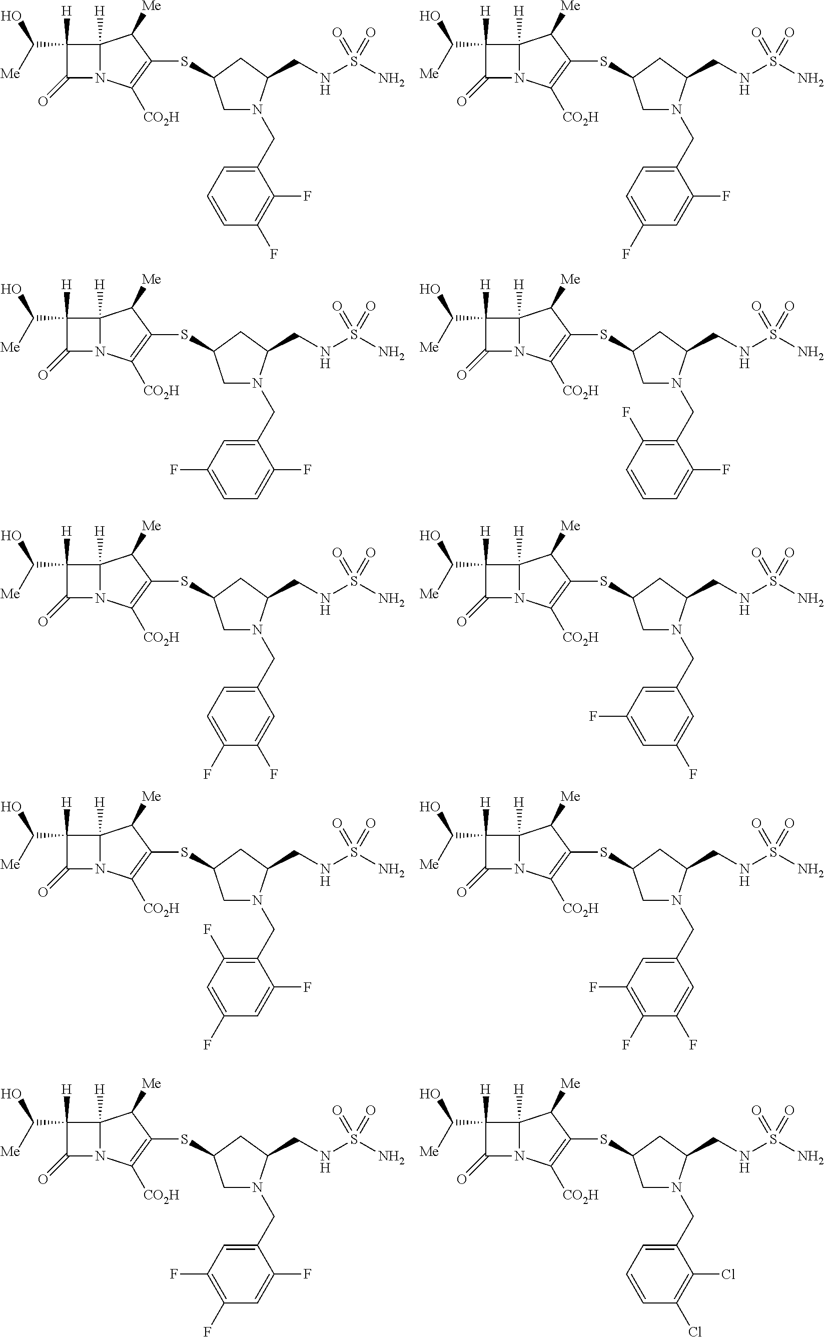

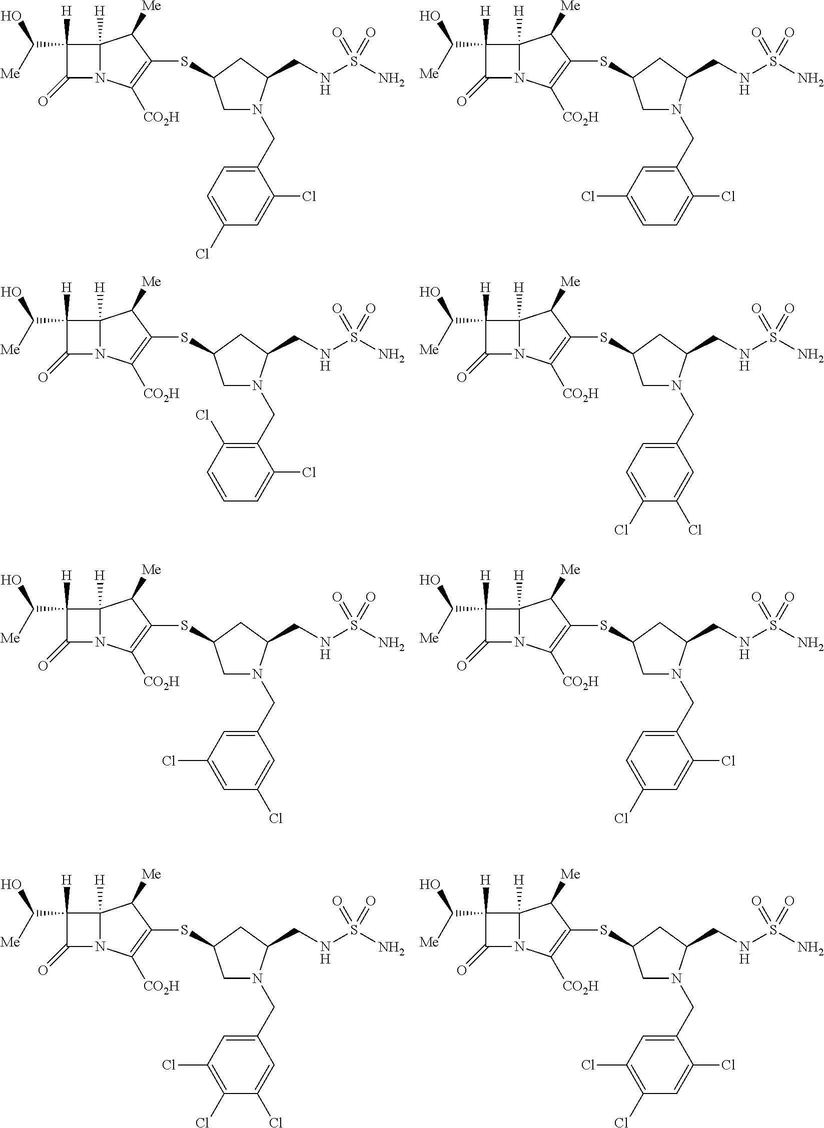

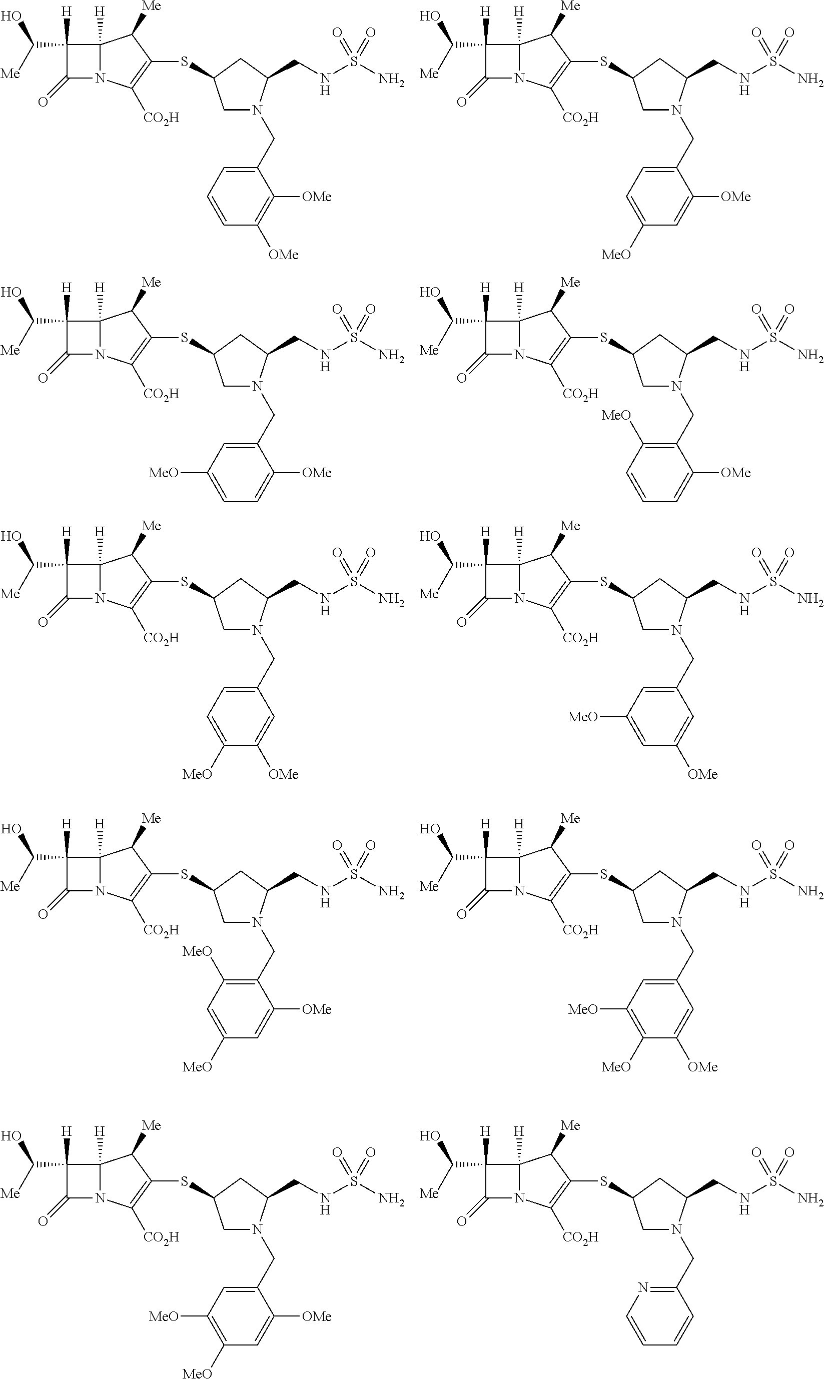

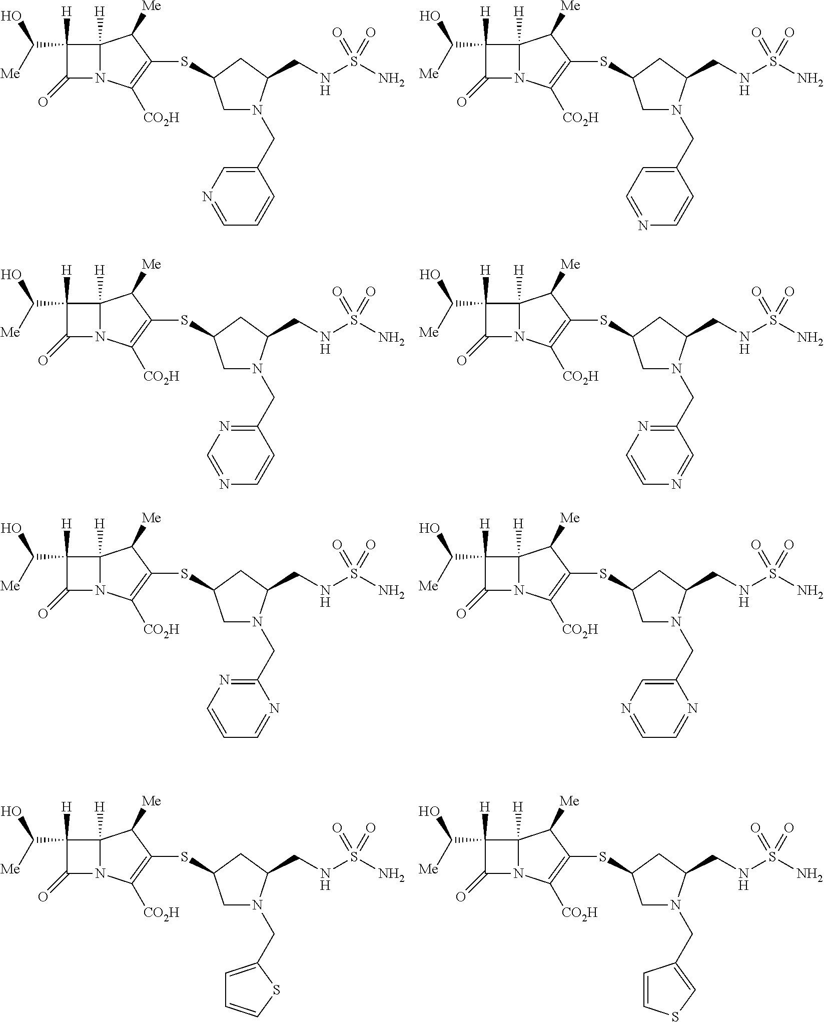

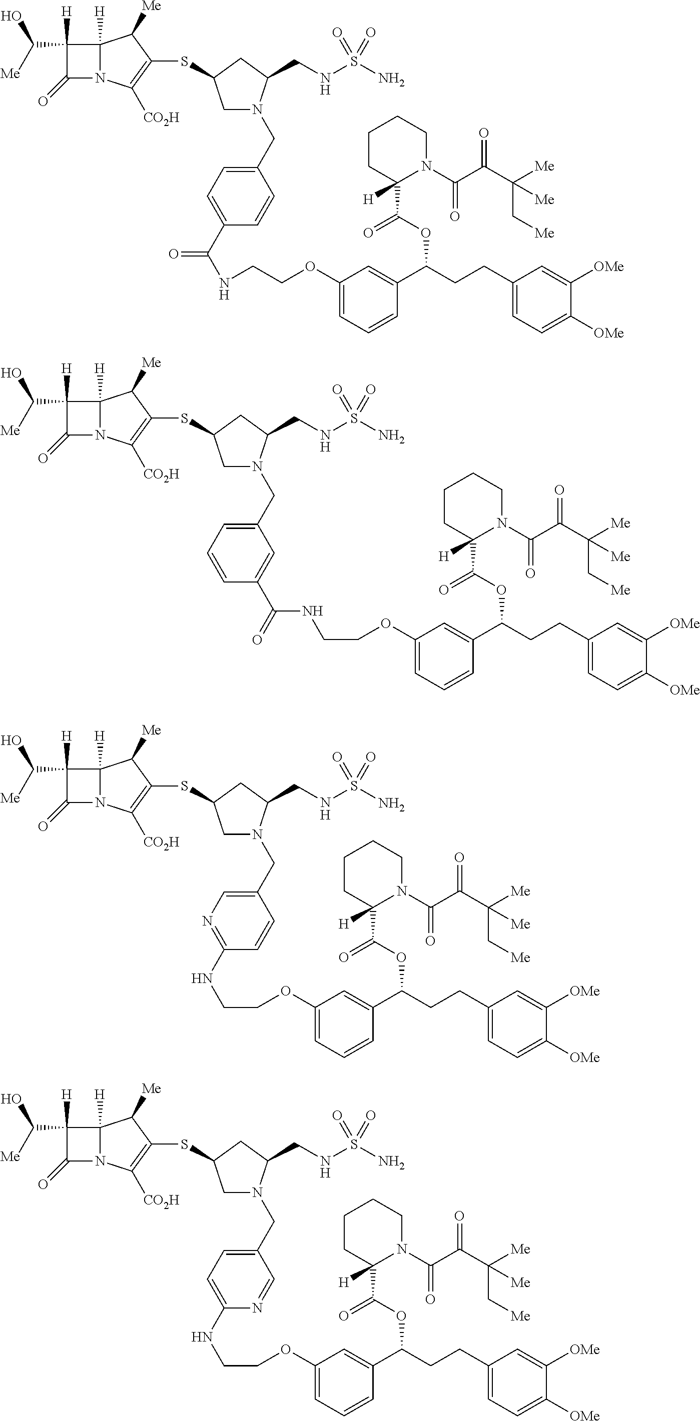

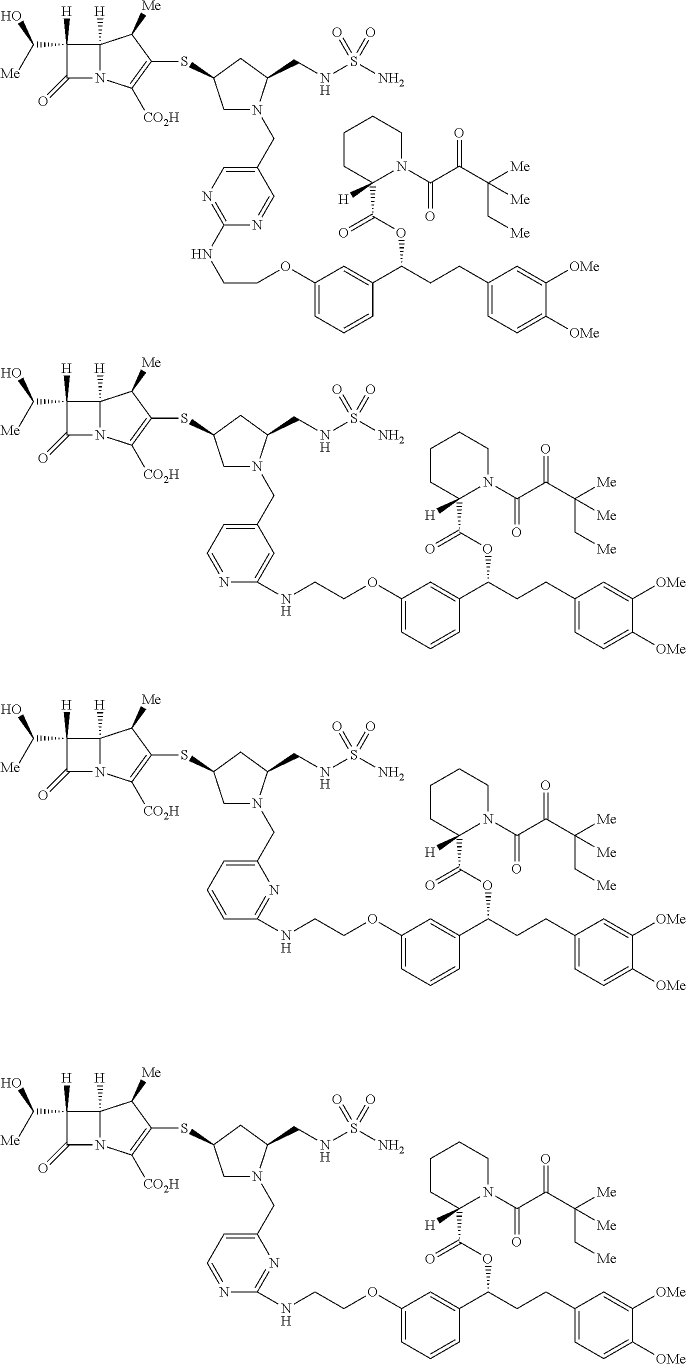

A compound of the disclosure may be administered in the form of a salt, ester, amide, prodrug, active metabolite, analog, or the like, provided that the salt, ester, amide, prodrug, active metabolite or analog is pharmaceutically acceptable and pharmacologically active in the present context. Salts, esters, amides, prodrugs, active metabolites, analogs, and other derivatives of the active agents may be prepared using standard procedures known to those skilled in the art of synthetic organic chemistry and described, for example, by J. March, Advanced Organic Chemistry: Reactions, Mechanisms and Structure, 5th Ed. (New York: Wiley-Interscience, 2001). Furthermore, where appropriate, functional groups on the compounds of the disclosure may be protected from undesired reactions during preparation or administration using protecting group chemistry. Suitable protecting groups are described, for example, in Green, Protective Groups in Organic Synthesis, 3rd Ed. (New York: Wiley-Interscience, 1999).

For example, where appropriate, any of the compounds described herein may be in the form of a pharmaceutically acceptable salt. A pharmaceutically acceptable salt may be prepared from any pharmaceutically acceptable organic acid or base, any pharmaceutically acceptable inorganic acid or base, or combinations thereof. The acid or base used to prepare the salt may be naturally occurring.

Suitable organic acids for preparing acid addition salts include, e.g., C₁-C₆ alkyl and C₆-C₁₂ aryl carboxylic acids, di-carboxylic acids, and tri-carboxylic acids such as acetic acid, propionic acid, succinic acid, maleic acid, fumaric acid, tartaric acid, glycolic acid, citric acid, pyruvic acid, oxalic acid, malic acid, malonic acid, benzoic acid, cinnamic acid, mandelic acid, salicylic acid, phthalic acid, and terephthalic acid, and aryl and alkyl sulfonic acids such as methanesulfonic acid, ethanesulfonic acid, and p-toluenesulfonic acid, and the like. Suitable inorganic acids for preparing acid addition salts include, e.g., hydrochloric acid, hydrobromic acid, hydroiodic acid, sulfuric acid, nitric acid, and phosphoric acid, and the like. An acid addition salt may be reconverted to the free base by treatment with a suitable base.

Suitable organic bases for preparing basic addition salts include, e.g., primary, secondary and tertiary amines, such as trimethylamine, triethylamine, tripropylamine, N,N-dibenzylethylenediamine, 2-dimethylaminoethanol, ethanolamine, ethylenediamine, glucamine, glucosamine, histidine, and polyamine resins, cyclic amines such as caffeine, N-ethylmorpholine, N-ethylpiperidine, and purine, and salts of amines such as betaine, choline, and procaine, and the like. Suitable inorganic bases for preparing basic addition salts include, e.g., salts derived from sodium, potassium, ammonium, calcium, ferric, ferrous, aluminum, lithium, magnesium, or zinc such as sodium hydroxide, potassium hydroxide, calcium carbonate, sodium carbonate, and potassium carbonate, and the like. A basic addition salt may be reconverted to the free acid by treatment with a suitable acid.

Preparation of esters involves transformation of a carboxylic acid group via a conventional esterification reaction involving nucleophilic attack of an RO⁻ moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

Any of the compounds of the disclosure may be the active agent in a formulation as described herein. Formulations containing the compounds of the disclosure may include 1, 2, 3 or more of the compounds described herein, and may also include one or more additional active agents such as analgesics and other antibiotics.

The amount of active agent in the formulation typically ranges from about 0.05 wt % to about 95 wt % based on the total weight of the formulation. For example, the amount of active agent may range from about 0.05 wt % to about 50 wt %, or from about 0.1 wt % to about 25 wt %. Alternatively, the amount of active agent in the formulation may be measured so as to achieve a desired dose.

Formulations containing the compounds of the disclosure may be presented in unit dose form or in multi-dose containers with an optional preservative to increase shelf life.

The compositions of the disclosure may be administered to the patient by any appropriate method. In general, both systemic and localized methods of administration are acceptable. It will be obvious to those skilled in the art that the selection of a method of administration will be influenced by a number of factors, such as the condition being treated, frequency of administration, dosage level, and the wants and needs of the patient. For example, certain methods may be better suited for rapid delivery of high doses of active agent, while other methods may be better suited for slow, steady delivery of active agent. Examples of methods of administration that are suitable for delivery of the compounds of the disclosure include parental and transmembrane absorption (including delivery via the digestive and respiratory tracts). Formulations suitable for delivery via these methods are well known in the art.

For example, formulations containing the compounds of the disclosure may be administered parenterally, such as via intravenous, subcutaneous, intraperitoneal, or intramuscular injection, using bolus injection and/or continuous infusion. Generally, parenteral administration employs liquid formulations.

The compositions may also be administered via the digestive tract, including orally and rectally. Examples of formulations that are appropriate for administration via the digestive tract include tablets, capsules, pastilles, chewing gum, aqueous solutions, and suppositories.

The formulations may also be administered via transmucosal administration. Transmucosal delivery includes delivery via the oral (including buccal and sublingual), nasal, vaginal, and rectal mucosal membranes. Formulations suitable for transmucosal deliver are well known in the art and include tablets, chewing gums, mouthwashes, lozenges, suppositories, gels, creams, liquids, and pastes.

The formulations may also be administered transdermally. Transdermal delivery may be accomplished using, for example, topically applied creams, liquids, pastes, gels and the like as well as what is often referred to as transdermal “patches.”

The formulations may also be administered via the respiratory tract. Pulmonary delivery may be accomplished via oral or nasal inhalation, using aerosols, dry powders, liquid formulations, or the like. Aerosol inhalers and imitation cigarettes are examples of pulmonary dosage forms.

Liquid formulations include solutions, suspensions, and emulsions. For example, solutions may be aqueous solutions of the active agent and may include one or more of propylene glycol, polyethylene glycol, and the like. Aqueous suspensions can be made by dispersing the finely divided active agent in water with viscous material, such as natural or synthetic gums, resins, methylcellulose, sodium carboxymethylcellulose, or other well known suspending agents. Also included are formulations of solid form which are intended to be converted, shortly before use, to liquid form.

Tablets and lozenges may comprise, for example, a flavored base such as compressed lactose, sucrose and acacia or tragacanth and an effective amount of an active agent. Pastilles generally comprise the active agent in an inert base such as gelatin and glycerine or sucrose and acacia. Mouthwashes generally comprise the active agent in a suitable liquid carrier.

For topical administration to the epidermis the chemical compound according to the disclosure may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Transdermal patches typically comprise: (1) a impermeable backing layer which may be made up of any of a wide variety of plastics or resins, e.g. aluminized polyester or polyester alone or other impermeable films; and (2) a reservoir layer comprising, for example, a compound of the disclosure in combination with mineral oil, polyisobutylene, and alcohols gelled with USP hydroxymethylcellulose. As another example, the reservoir layer may comprise acrylic-based polymer adhesives with resinous crosslinking agents which provide for diffusion of the active agent from the reservoir layer to the surface of the skin. The transdermal patch may also have a delivery rate-controlling membrane such as a microporous polypropylene disposed between the reservoir and the skin. Ethylene-vinyl acetate copolymers and other microporous membranes may also be used. Typically, an adhesive layer is provided which may comprise an adhesive formulation such as mineral oil and polyisobutylene combined with the active agent.

Other typical transdermal patches may comprise three layers: (1) an outer layer comprising a laminated polyester film; (2) a middle layer containing a rate-controlling adhesive, a structural non-woven material and the active agent; and (3) a disposable liner that must be removed prior to use. Transdermal delivery systems may also involve incorporation of highly lipid soluble carrier compounds such as dimethyl sulfoxide (DMSO), to facilitate penetration of the skin. Other carrier compounds include lanolin and glycerin.

Rectal or vaginal suppositories comprise, for example, an active agent in combination with glycerin, glycerol monopalmitate, glycerol, monostearate, hydrogenated palm kernel oil and fatty acids. Another example of a suppository formulation includes ascorbyl palmitate, silicon dioxide, white wax, and cocoa butter in combination with an effective amount of an active agent.

Nasal spray formulations may comprise a solution of active agent in physiologic saline or other pharmaceutically suitable carder liquids. Nasal spray compression pumps are also well known in the art and can be calibrated to deliver a predetermined dose of the solution.

Aerosol formulations suitable for pulmonary administration include, for example, formulations wherein the active agent is provided in a pressurized pack with a suitable propellant. Suitable propellants include chlorofluorocarbons (CFCs) such as dichlorodifluoromethane, trichlorofluoromethane, or dichlorotetrafluoroethane, carbon dioxide, or other suitable gases. The aerosol may also contain a surfactant such as lecithin. The dose of drug may be controlled by provision of a metered valve.

Dry powder suitable for pulmonary administration include, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. Unit doses for dry powder formulations may be, for example, in the form of capsules or cartridges of, e.g., gelatin, or blister packs from which the powder may be administered by means of an inhaler.

In addition to the foregoing components, it may be necessary or desirable in some cases (depending, for instance, on the particular composition or method of administration) to incorporate any of a variety of additives, e.g., components that improve drug delivery, shelf-life, patient acceptance, etc. Suitable additives include acids, antioxidants, antimicrobials, buffers, colorants, crystal growth inhibitors, defoaming agents, diluents, emollients, fillers, flavorings, gelling agents, fragrances, lubricants, propellants, thickeners, salts, solvents, surfactants, other chemical stabilizers, or mixtures thereof. Examples of these additives can be found, for example, in M. Ash and I. Ash, Handbook of Pharmaceutical Additives (Hampshire, England: Gower Publishing, 1995), the contents of which are herein incorporated by reference.

Appropriate dose and regimen schedules will be apparent based on the present disclosure and on information generally available to the skilled artisan. When the compounds of the disclosure are used in the treatment of infections, achievement of the desired effects may require weeks, months, or years of controlled, low-level administration of the formulations described herein. Other dosage regimens, including less frequent administration of high-intensity dosages, are also within the scope of the disclosure.

The amount of active agent in formulations that contain the compounds of the disclosure may be calculated to achieve a specific dose (i.e., unit weight of active agent per unit weight of patient) of active agent. Furthermore, the treatment regimen may be designed to sustain a predetermined systemic level of active agent. For example, formulations and treatment regimen may be designed to provide an amount of active agent that ranges from about 0.001 mg/kg/day to about 100 mg/kg/day for an adult. As a further example, the amount of active agent may range from about 0.1 mg/kg/day to about 50 mg/kg/day, about 0.1 mg/kg/day to about 25 mg/kg/day, or about 1 mg/kg/day to about 10 mg/kg/day. One of skill in the art will appreciate that dosages may vary depending on a variety of factors, including method and frequency of administration, and physical characteristics of the patient.

The compounds of the disclosure may be prepared using standard procedures that are known to those skilled in the art of synthetic organic chemistry and used for the preparation of analogous compounds. Appropriate synthetic procedures may be found, for example, in J. March, Advanced Organic Chemistry: Reactions, Mechanisms and Structure, 5th Edition (New York: Wiley-Interscience, 2001). Syntheses of representative compounds are detailed in the Examples.

Accordingly, the compounds find utility in treating infections by microorganisms. Accordingly, the disclosure provides a method for treating an infected patient, the method comprising administering to the patient an effective amount of any of the compounds disclosed herein. The disclosure also provides a method for preventing infection, the method comprising administering an effective amount of any of the compounds disclosed herein. The disclosure also provides a method for treating a patient suffering from an infection, the method comprising administering an effective amount of any of the compounds disclosed herein to a patient in need thereof. The disclosure also provides a method for inhibiting the spread of an infection, the method comprising contacting a cell infected with a microorganism with an effective amount of any of the compounds disclosed herein. As described in more detail herein, in any of the aforementioned methods, the compound may be administered in a composition comprising one or more active agents and one or more additives.

The compounds of the invention are useful in the prevention and treatment of many different bacterial infections. Bacterial infections that may be treated or prevented using the compounds of the invention include, without limitation, infections resulting from bacteria of the genus Listeria, Enterococcus, Pseudomonas, Staphylococcus, Escherichia, Enterobacter, Salmonella, Shigella, Aerobacter, Helicobacter, Klebsiella, Proteus, Streptococcus, Chlamydia, Mycoplasma, Pneumococcus, Neisseria, Clostridium, Bacillus, Corynebacterium, Mycobacterium, Campylobacter, Vibrio, Serratia, Providencia, Candida, Chromobacterium, Brucella, Yersinia, Haemophilus, Bordetella, Burkholderia, Acinetobacter, or Francisella. Other intracellular bacterial strains can also be treated with the compounds of the invention.

For example, the present compounds exhibit efficacy with respect to the treatment of infections of Staphylococcus aureus (including methicillin-resistant and methicillin-susceptible), Pseudomonas aeruginosa, Klebsiella pneumonia, Escherichia coli, Vancomycin-sensitive enterococci faecium (VSE), Mycobacterium tuberculosis; Mycobacterium bovis; Mycobacterium africanum; Mycobacterium canetti; Mycobacterium microti; etc.

Generally, the compounds of the invention may be effective at treating one or more of the abovementioned bacterial strains. In some embodiments, the compounds may be effective at treating one or more bacterial strain not listed herein. In some embodiments, the compounds may be effective against a broad spectrum of bacteria, and in some embodiments, the compounds may be effective against a specific bacterial strain.

Accordingly, the invention provides methods for treating a patient (typically, although not necessarily, a human patient) in need of such treatment. The methods involve administration of one or more compounds described herein. Typically, the compound is administered in the form of a composition as described herein. The methods include therapeutic treatment of a patient having a bacterial infection, as well as prophylactic treatment of a patient (i.e., a patient not having a bacterial infection). For example, the methods include treatment of a patient having Tuberculosis.

Furthermore, the invention provides methods for reducing the number of bacteria in a patient by administration of the compounds described herein. The invention further provides methods for eliminating a colony of bacteria from a patient using the compound disclosed herein. The invention further provides methods for killing and/or disrupting the growth of bacteria using the compounds disclosed herein.

Generally, in prophylactic use, the patient will have been identified as being at an elevated risk of developing a bacterial infection. Such patients include, for example, those expecting to be exposed to an environment with an increased level of bacteria present. Commonly, such patients include those undergoing surgery or other procedures in hospitals. Other examples include armed-service personnel who may be exposed to bacteria as part of routine operations, or individuals (military or civilian) who are at increased risk of exposure to bacteria as a result of an attack with biological weapons.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains.

### EXAMPLES

Experimental. Unless otherwise noted, reagents and solvents were used as received from commercial suppliers. Proton nuclear magnetic resonance spectra as well as variable temperature (VT) proton nuclear magnetic resonance spectra were obtained on a Bruker Avance II 400 spectrometer at 400 MHz. Tetramethylsilane was used as an internal standard in CDCl₃. Thin-layer chromatography (TLC) was performed using Merck silica-gel 60 F₂₅₄ plates and visualized by ultraviolet (UV) light or iodine. The HPLC-UV-MS analysis was performed on a Waters Acquity HPLC/MS system equipped with a Waters Acquity BEH C18 1.7 μm column and Waters 2489 UV/VIS detector.

HPLC-UV-MS conditions. Eluent A=95:5 Water/Acetonitrile with 20 mM HCOONH₄/NH₄OH buffer, pH 7.4. Eluent B=20:80 Water/Acetonitrile with 20 mM HCOONH₄/NH₄OH buffer, pH 7.4. Gradient elution program: adjusted according to the compound properties. Column Temperature: 50° C. Flow Rate: 0.6 mL/min. Sample Concentration: 1 mg/mL. Sample Solvent: Acetonitrile, water. Injection: 0.1-1 μL. UV detection wavelength: 220 nm. UV detection sampling rate: 20 points/sec

MS conditions. Measured Mass Range: 150-1500 Da. Scan Time: 0.2 s. Ion mode: ESI+; APCI+. Cone Voltage: typically 30 V or lower depending on the compound properties.

Chromatographic purification performed using a LaChrom HPLC system (Merck-Hitachi). Chromatographic conditions: C18 reverse phase columns: A: Purospher Star RP-18e, 5 μm HitHunter 100-10 mm (0.1 mg-5 mg/injection). B: Gemini-NX 5 μm C18 110A AXIA Packed 100×21.2 mm (5 mg-100 mg/injection). Column Temperature: room temperature. Flow Rate: up to 40 ml/min. Detection: UV detector.

Eluents: Three component systems, adjusted according to the compound properties. Eluent System 1. Eluent (A): Water. Eluent (B): Acetonitrile. Eluent (C): Acetonitrile/Isopropanol/formic acid (70/30/0.1) (column wash). Eluent System 2. Eluent (A): Acetonitrile/H₂O (5/95), 10 mM NH₄HCO₃ buffer, pH 8.0. Eluent (B): Acetonitrile/H₂O (80/20), 10 mM NH₄HCO₃ buffer, pH 8.0. Eluent (C): Acetonitrile/Isopropanol/formic acid (70/30/0.1) (column wash).

### Example 1

The syntheses of compounds 12, 16, 25, 27, 34, 36, and others are shown below.

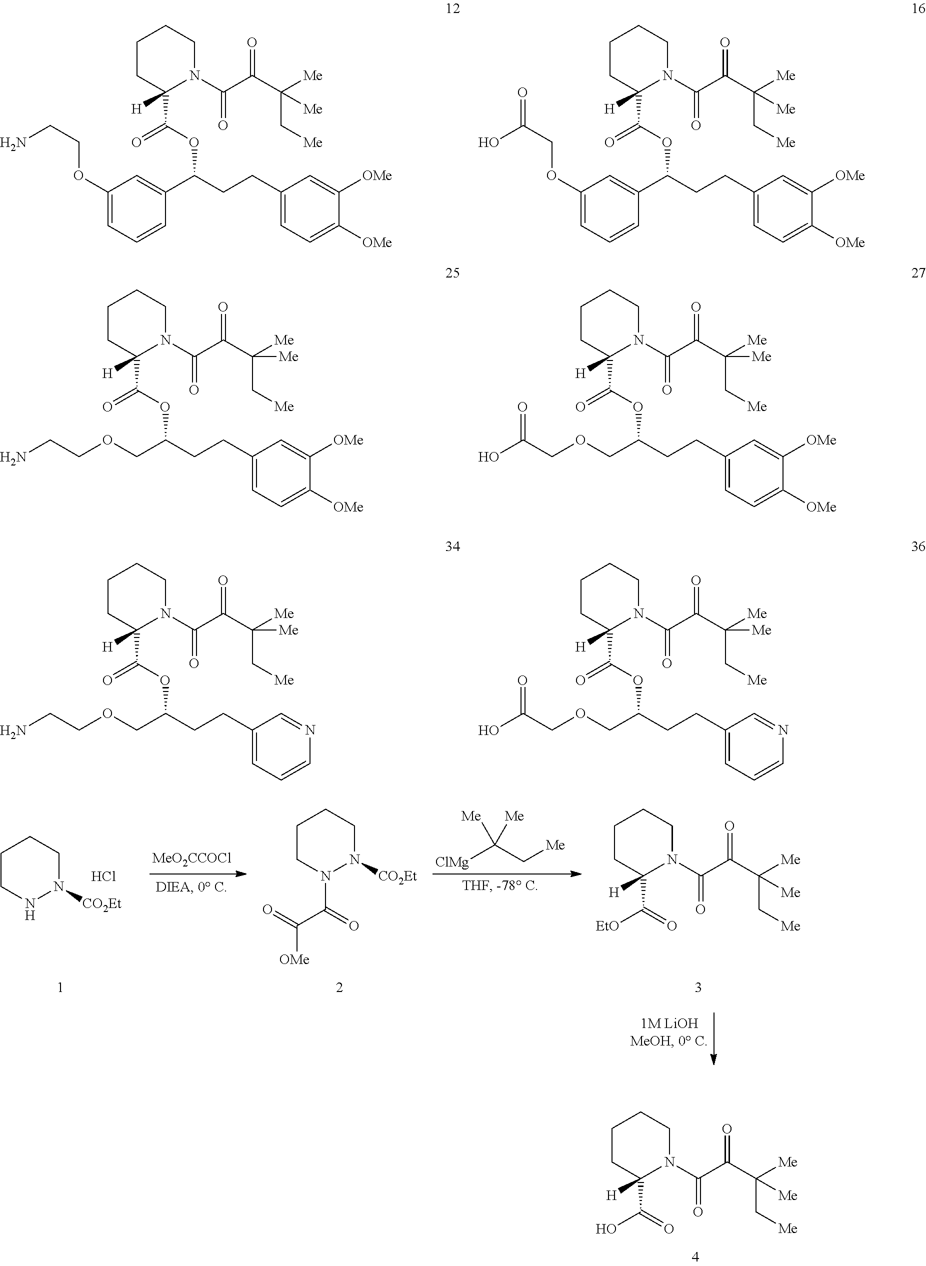

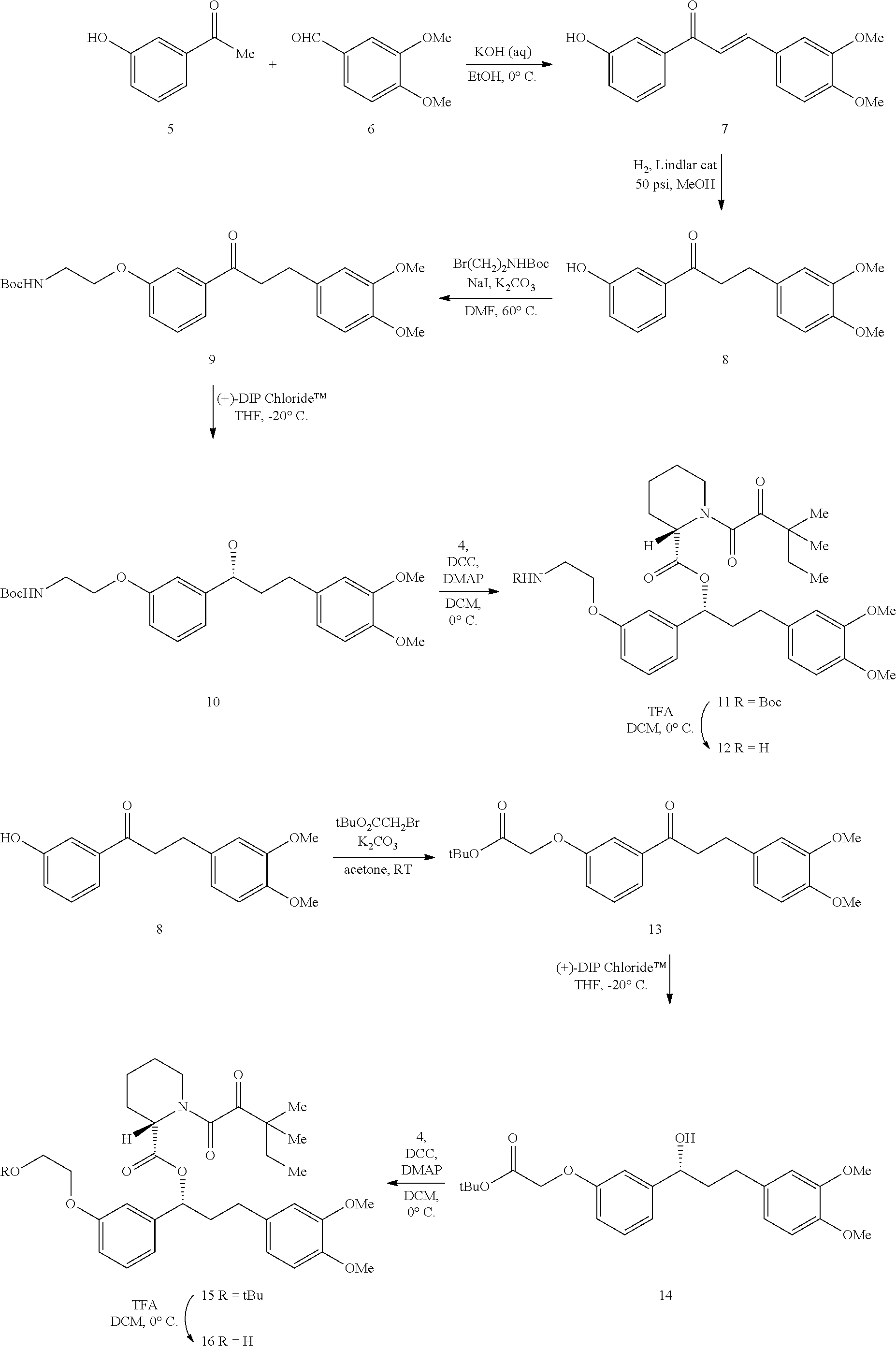

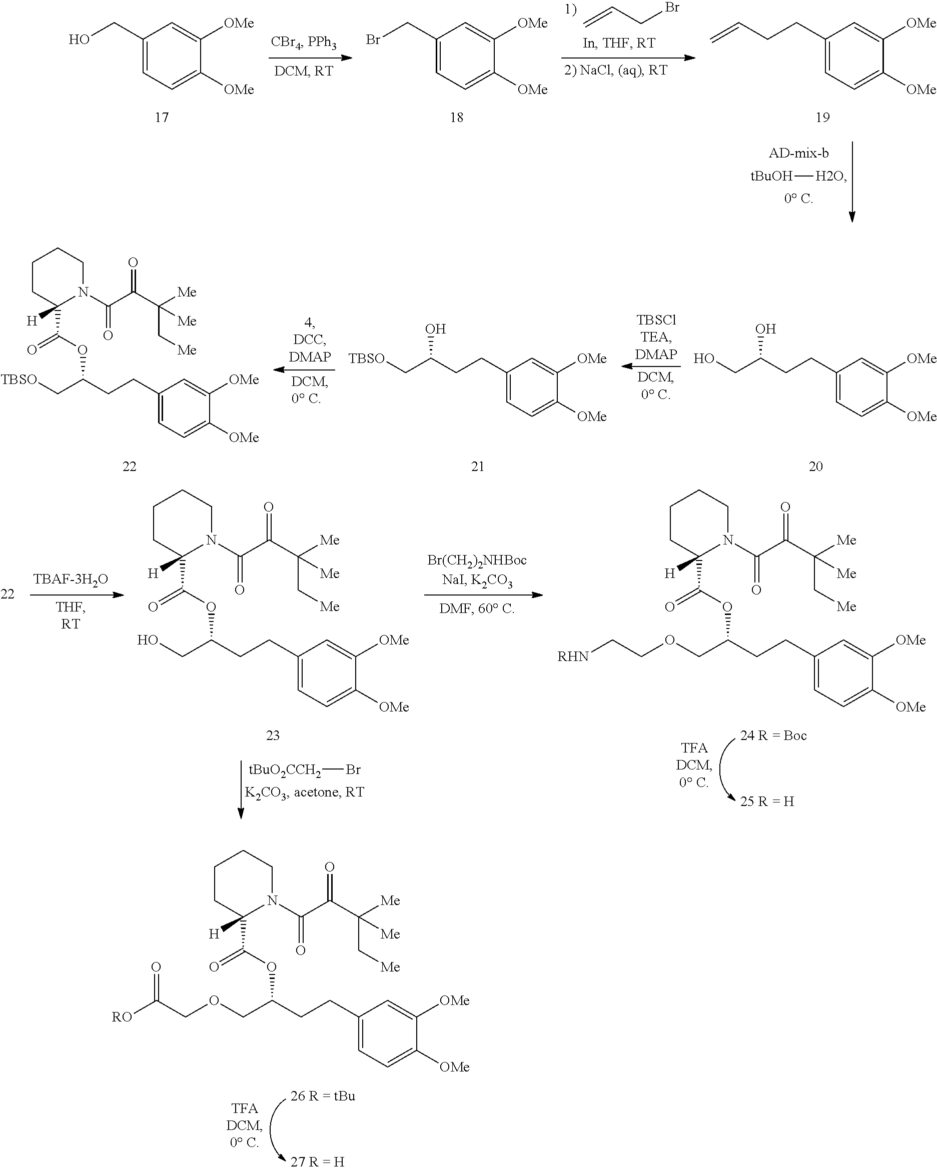

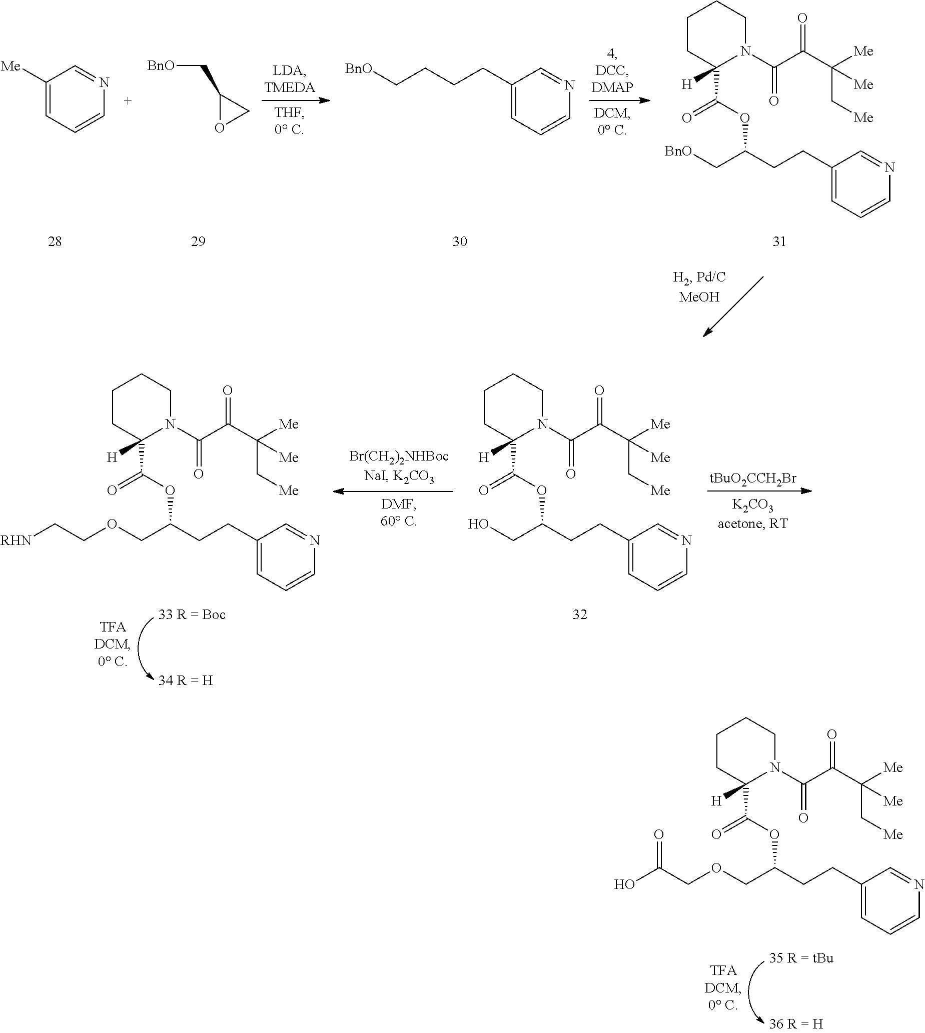

### Example 2

The synthesis of compound 38 is shown below.

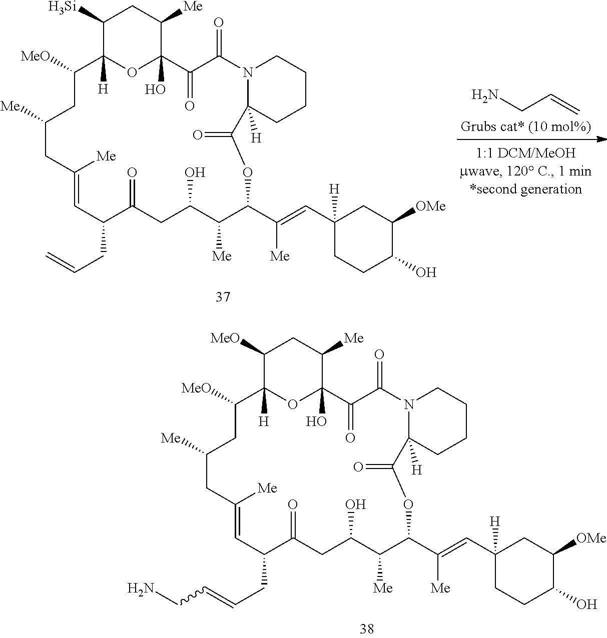

### Example 3

The syntheses of compounds 48, 55, and others are shown below.

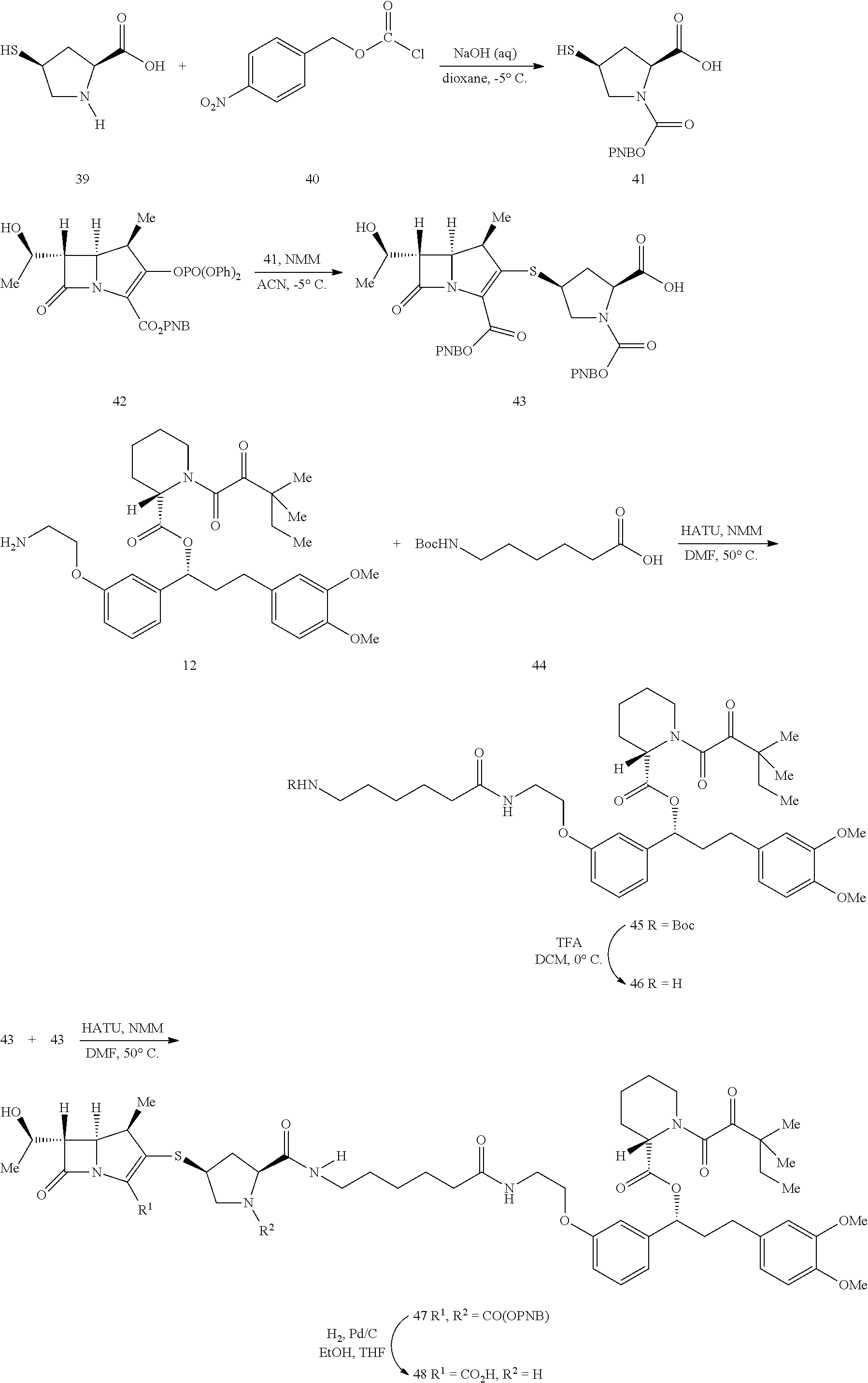

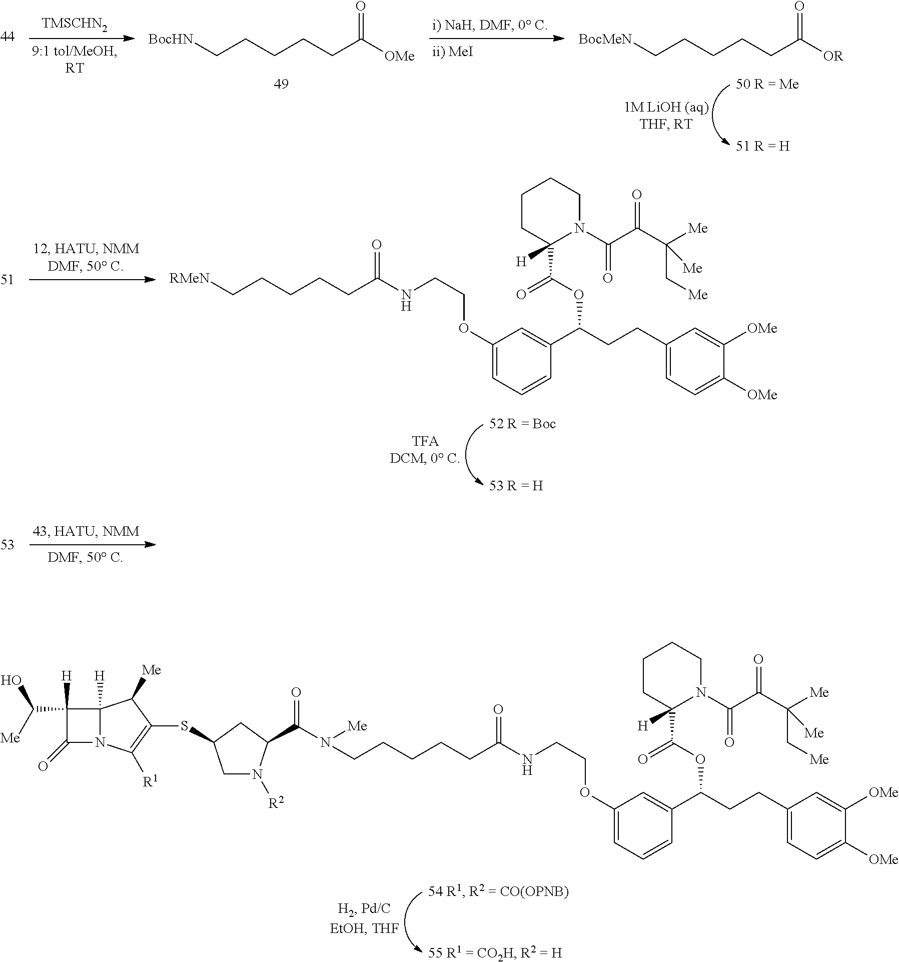

### Example 4

The syntheses of compounds 59, 62, 67, and others are shown below.

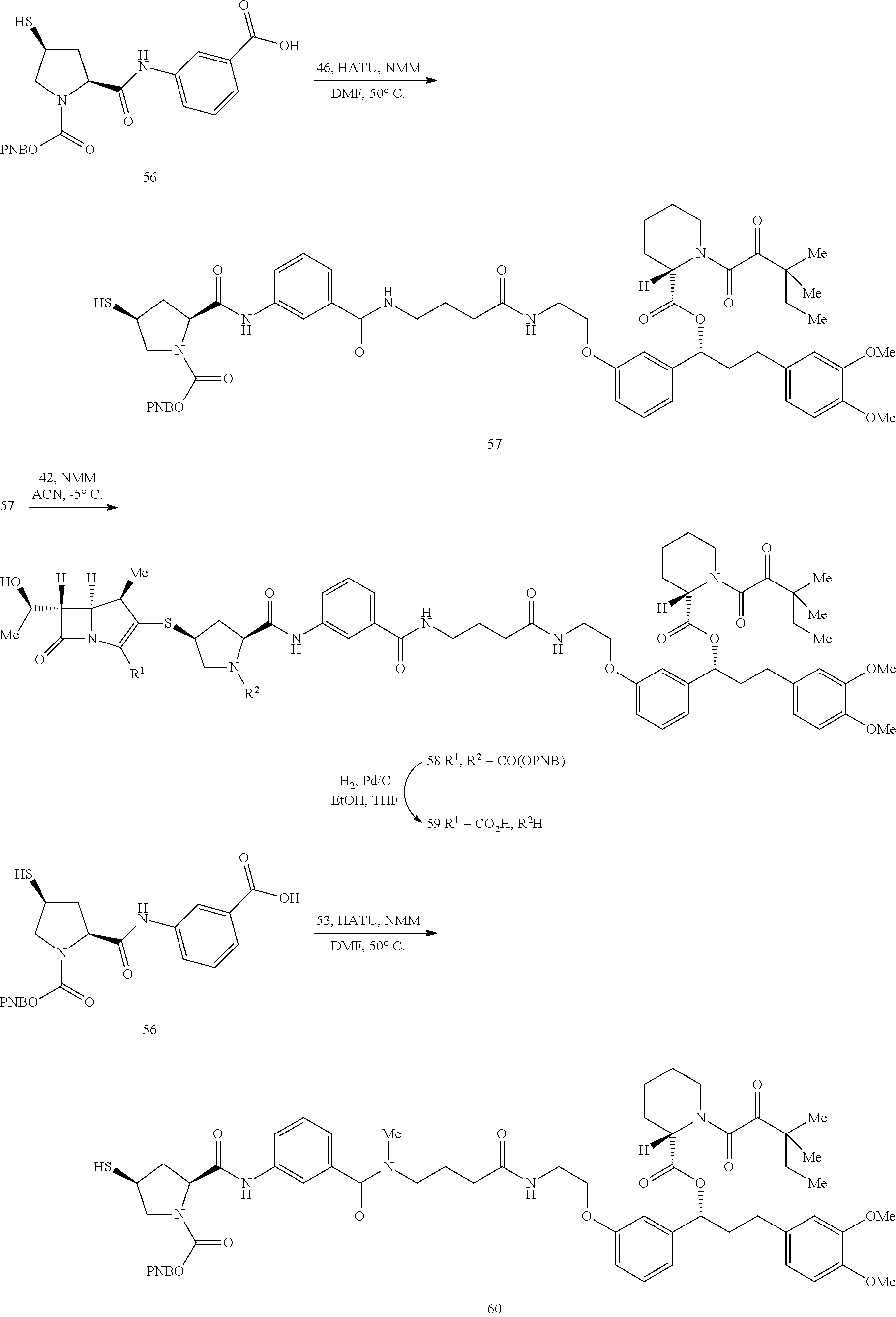

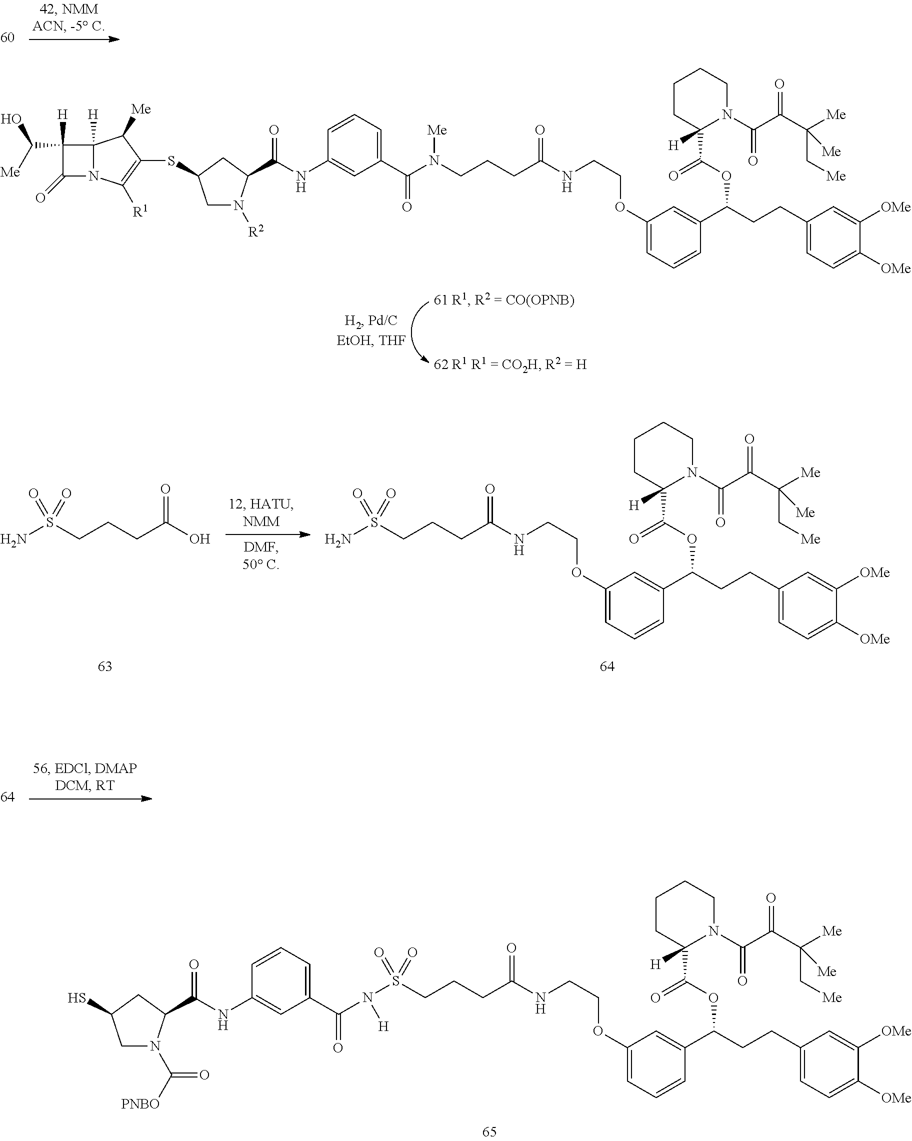

### Example 5

The synthesis of compound 72 is shown below.

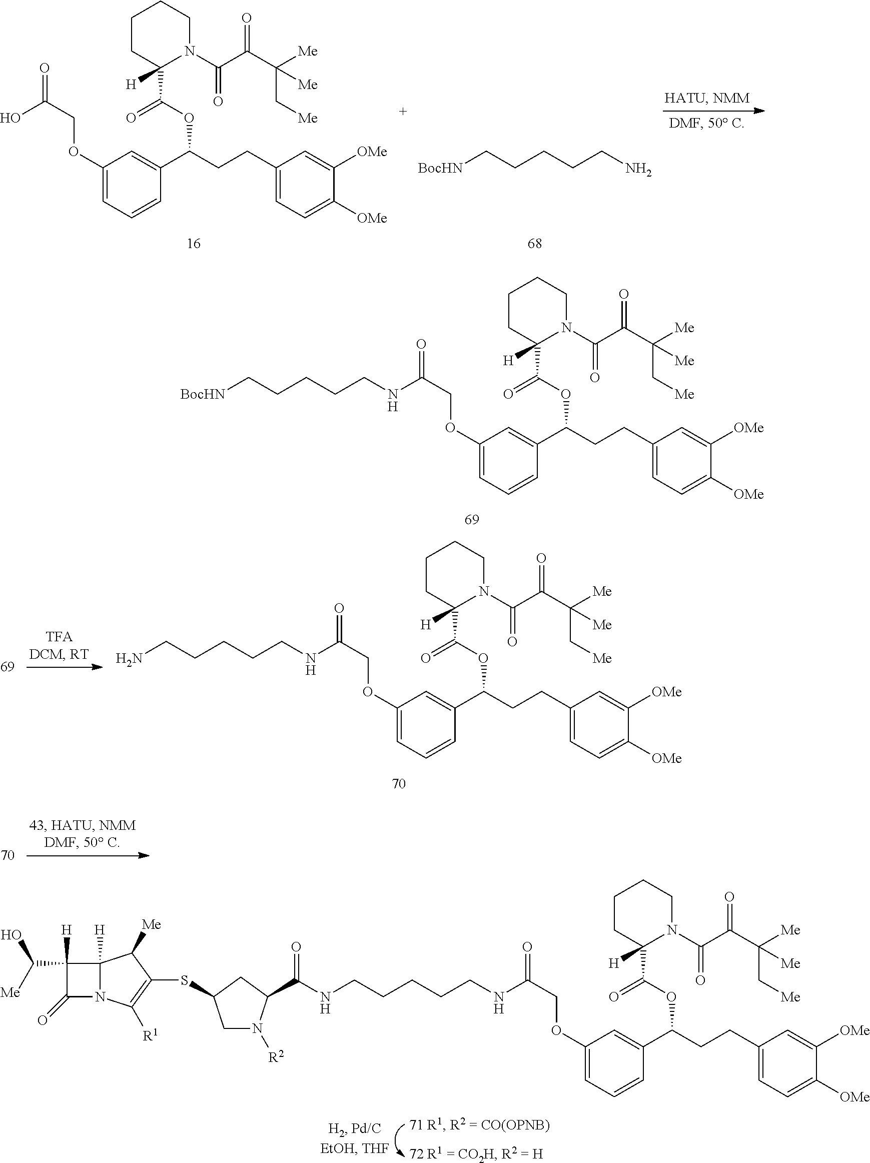

### Example 6

The syntheses of compounds 80, 83, 86, and others are shown below.

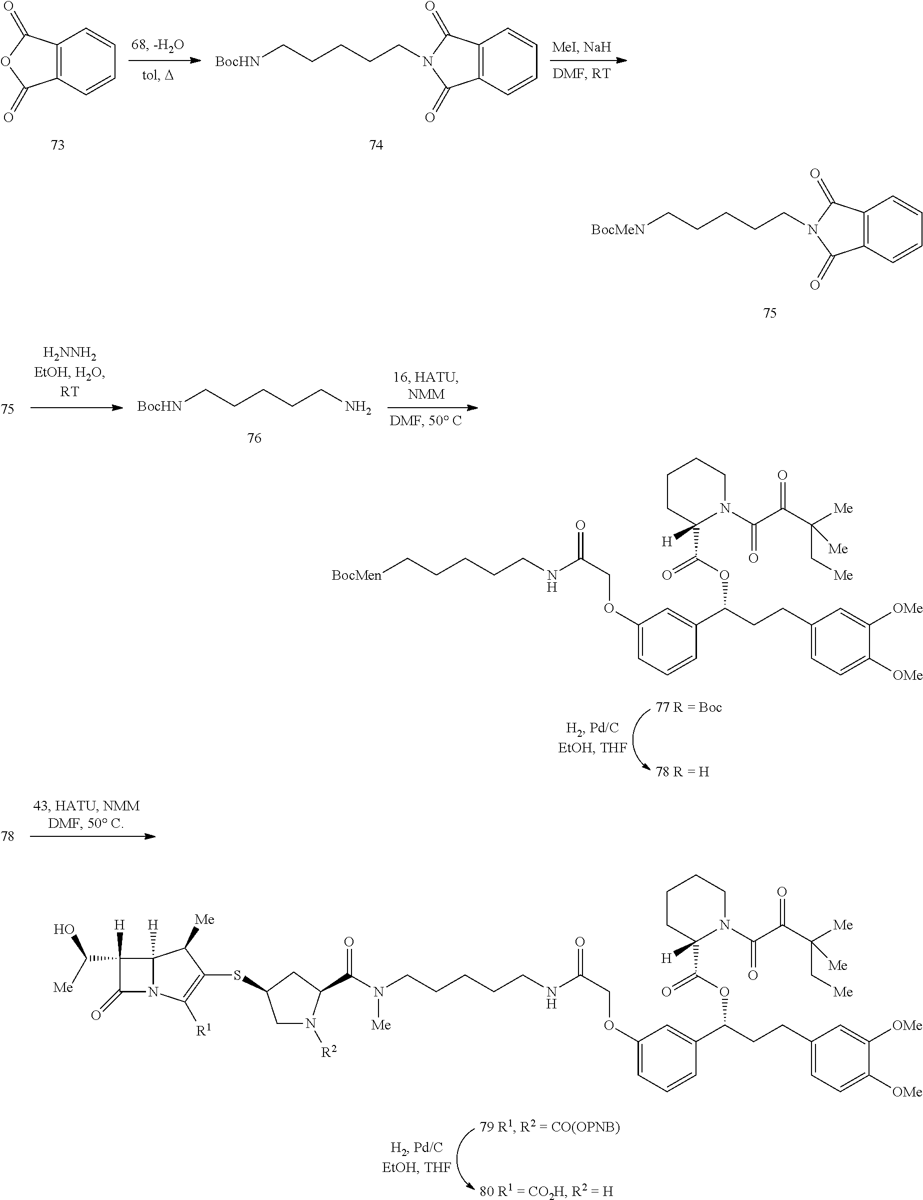

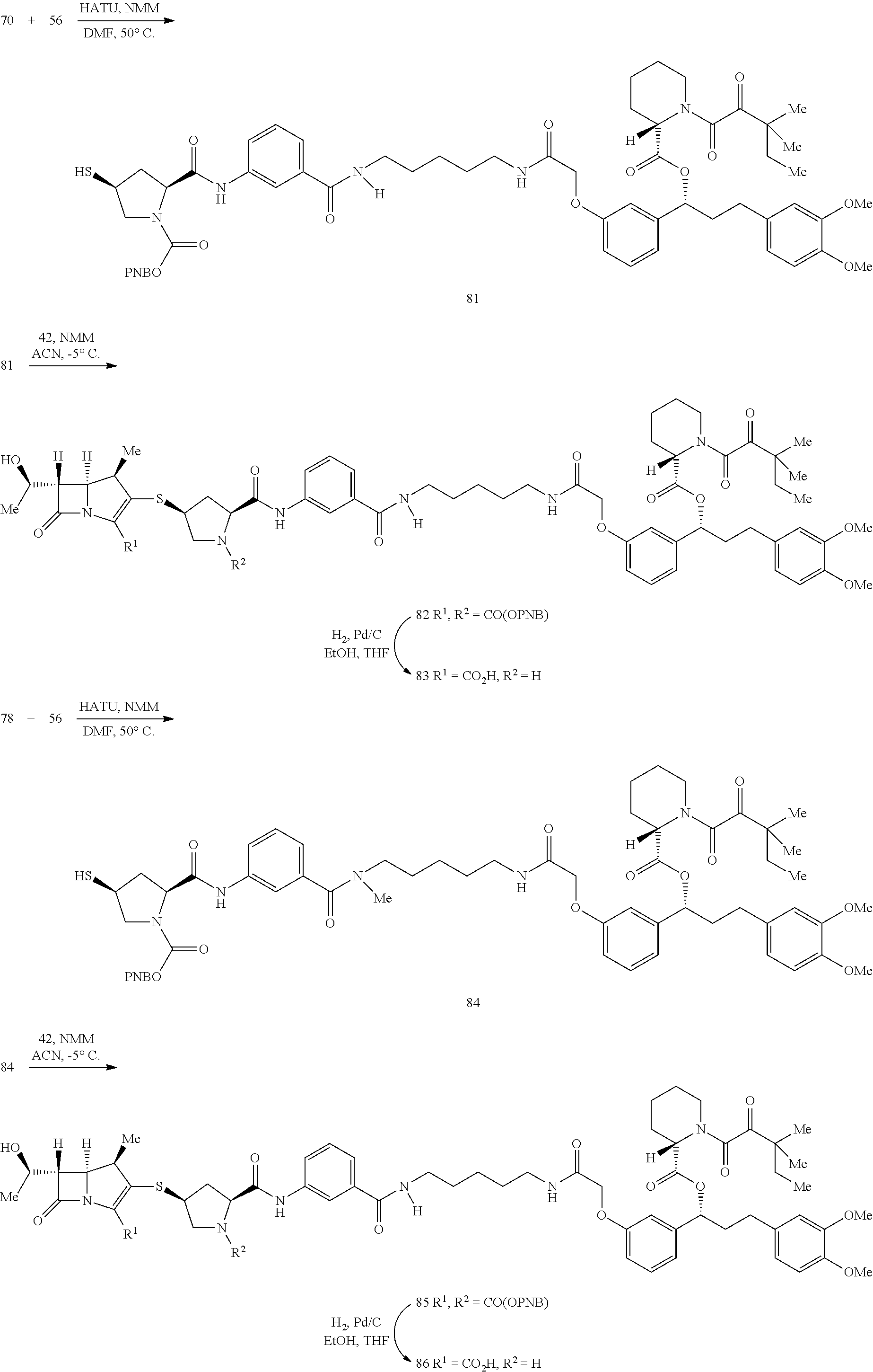

### Example 7

The syntheses of compounds 91, 93, 97, and others are shown below.

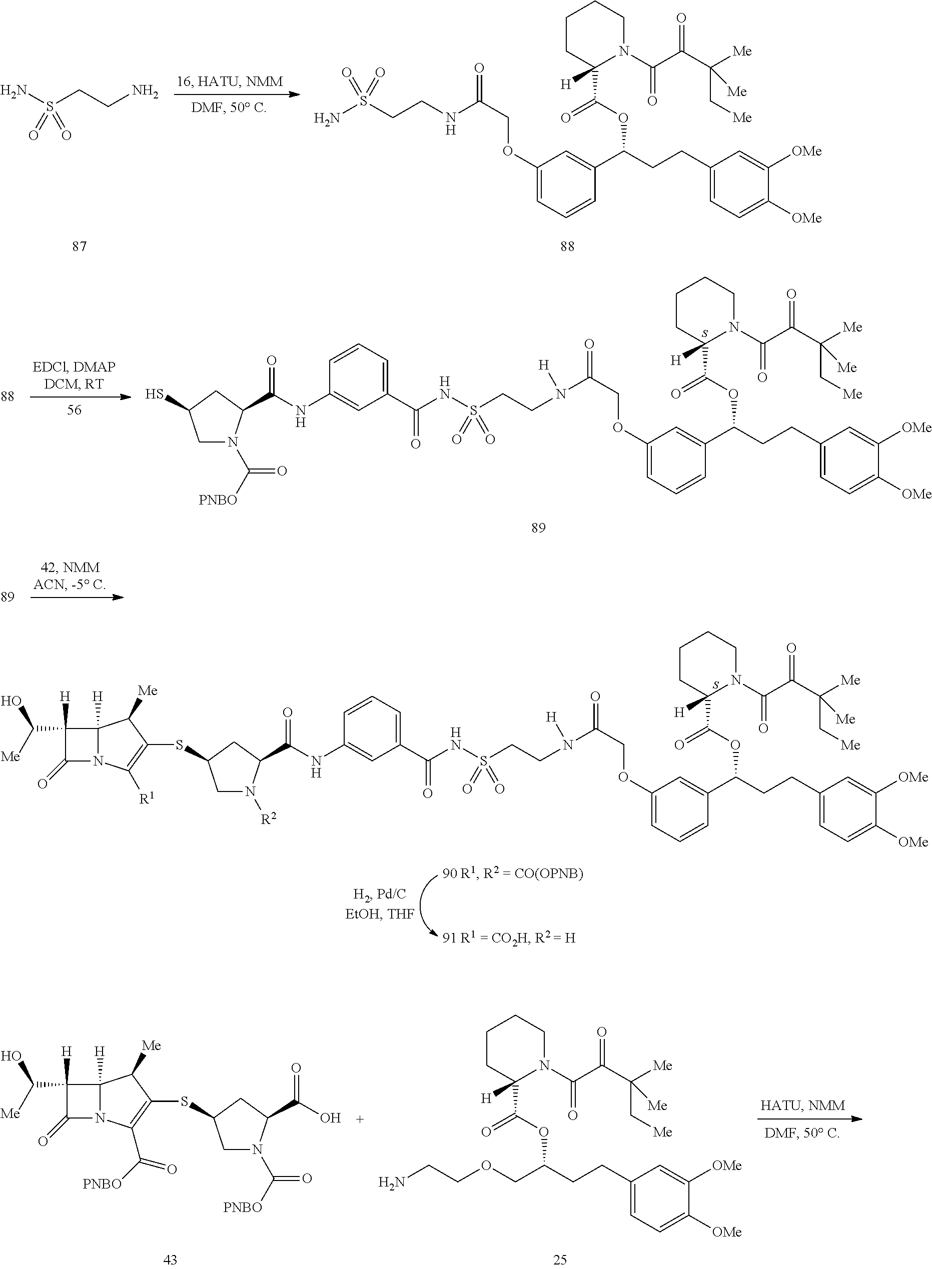

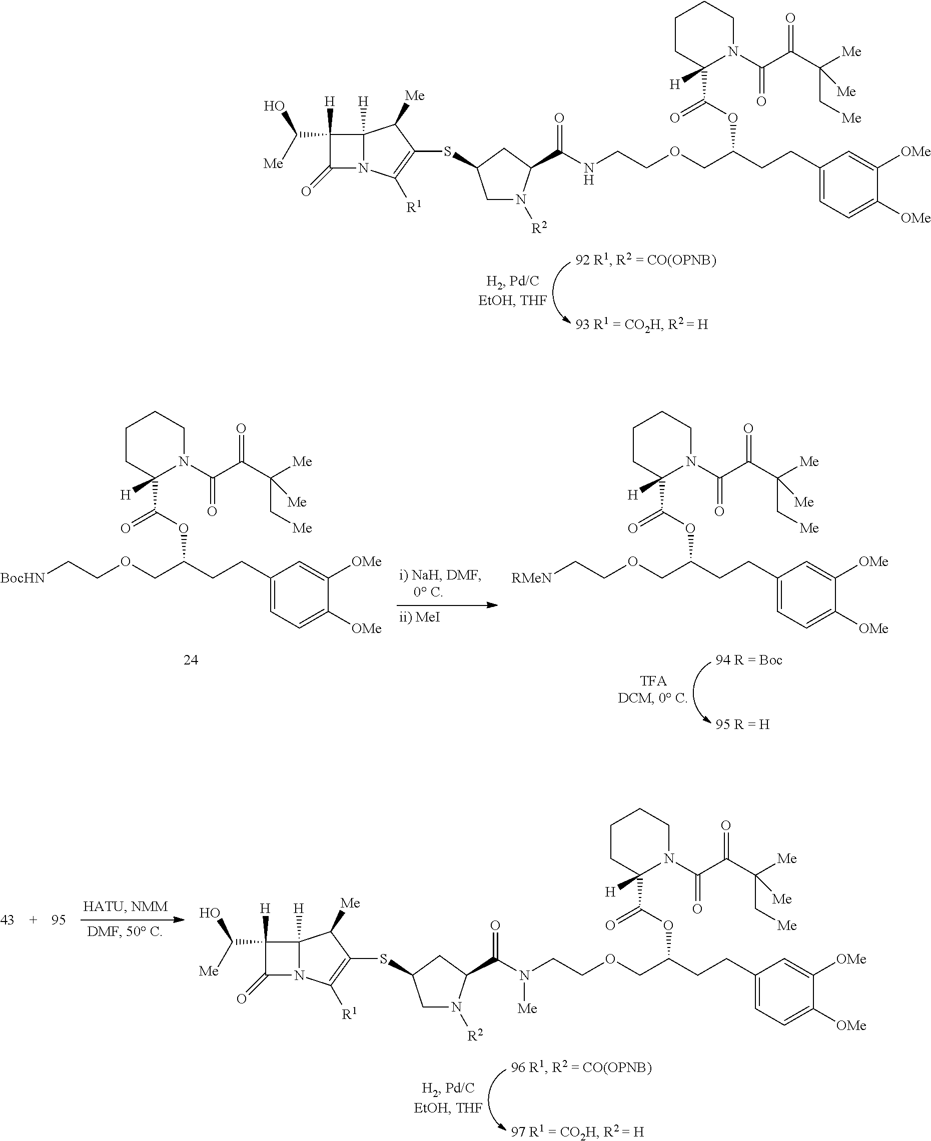

### Example 8

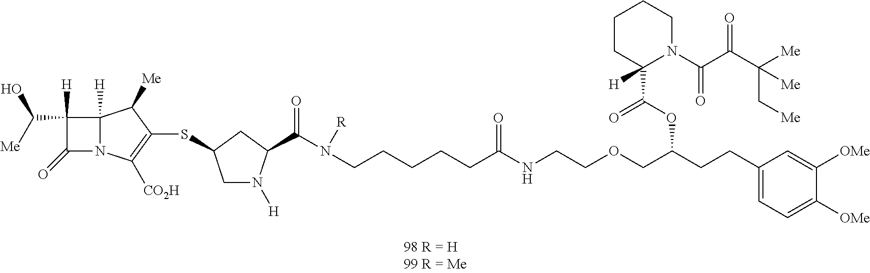

Cmpd 98 is prepared in a similar manner as outlined for cmpd 48. Cmpd 99 is prepared in a similar manner as outlined for cmpd 55.

### Example 9

The syntheses of compounds 102, 105, and others are shown below.

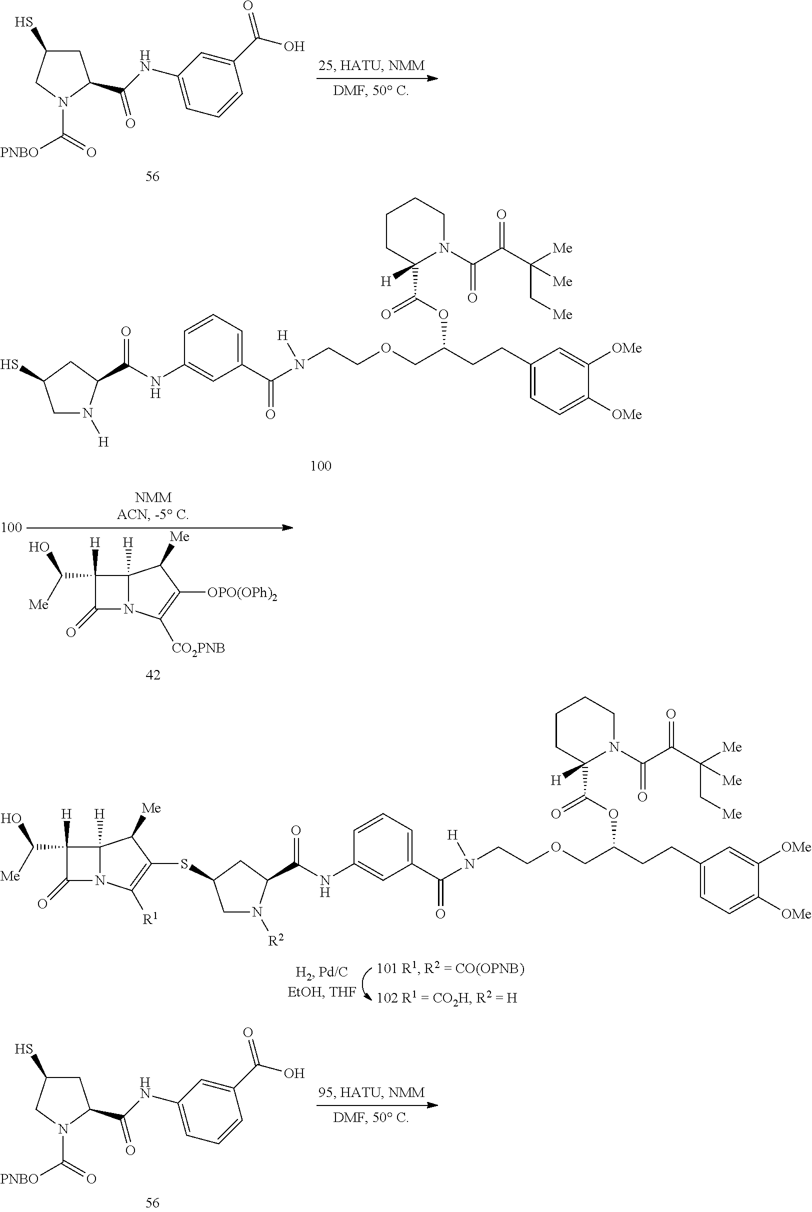

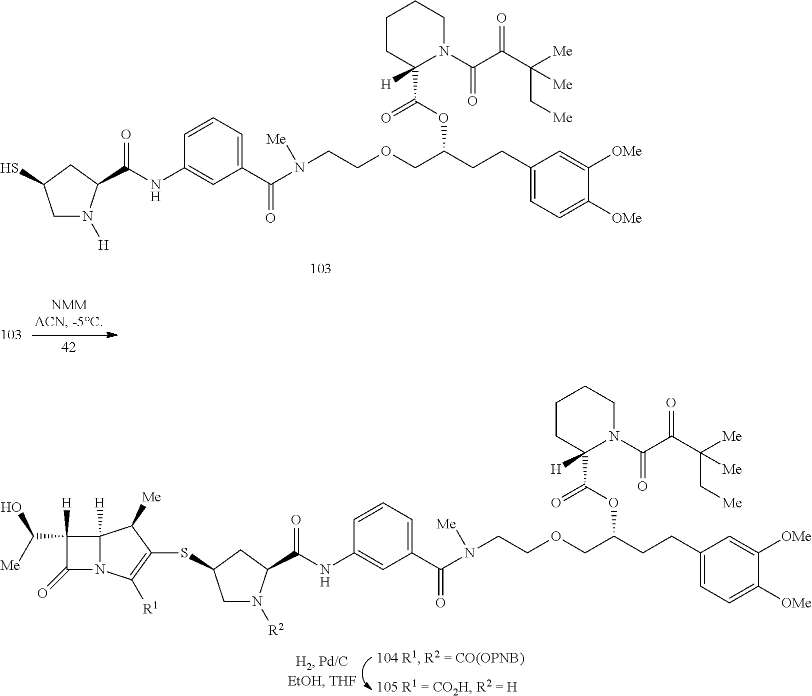

### Example 10

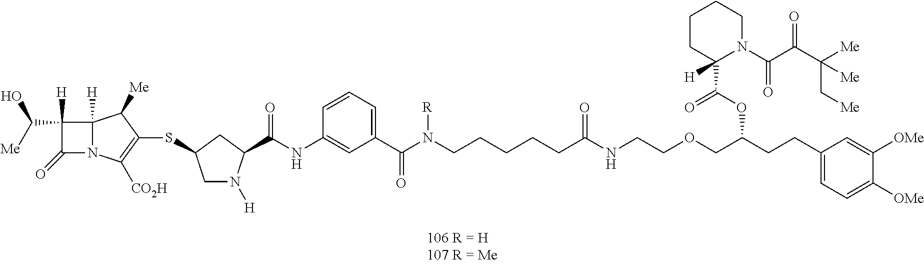

Cmpd 106 is prepared in a similar manner as outlined for cmpd 59. Cmpd 107 is prepared in a similar manner as outlined for cmpd 62.

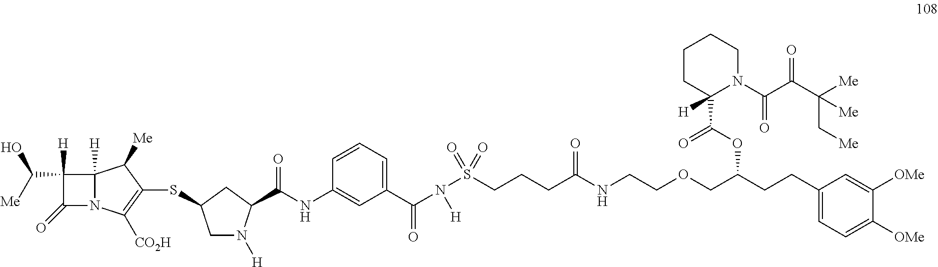

Cmpd 108 is prepared in a similar manner as outlined for cmpd 67.

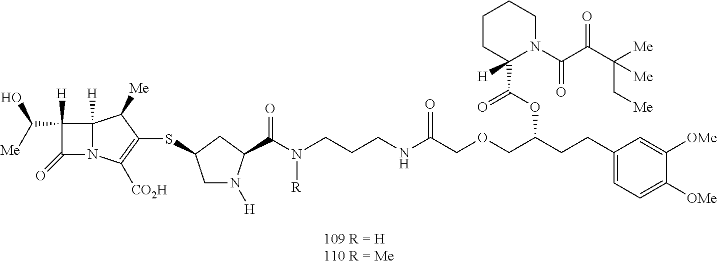

Cmpd 109 is prepared in a similar manner as outlined for cmpd 72. Cmpd 110 is prepared in a similar manner as outlined for cmpd 80.

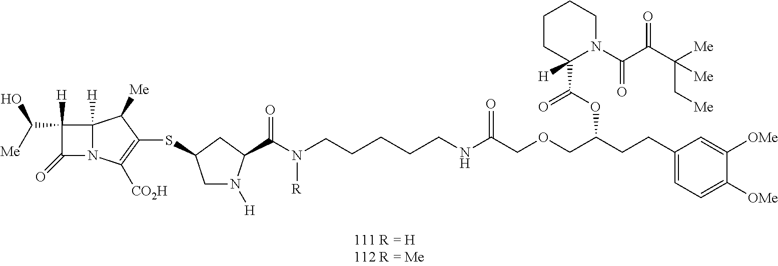

Cmpd 111 is prepared in a similar manner as outlined for cmpd 72. Cmpd 112 is prepared in a similar manner as outlined for cmpd 80.

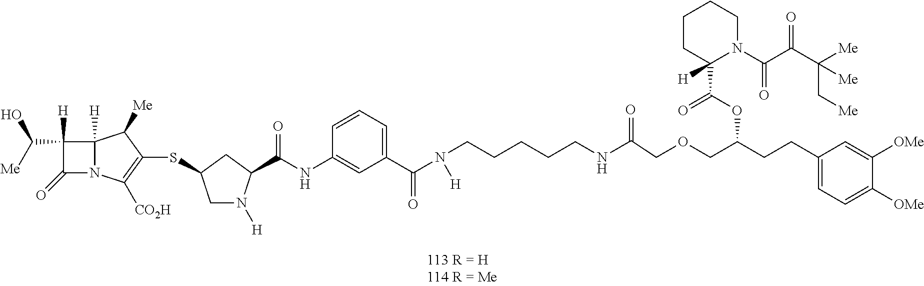

Cmpd 113 is prepared in a similar manner as outlined for cmpd 83. Cmpd 114 is prepared in a similar manner as outlined for cmpd 86.

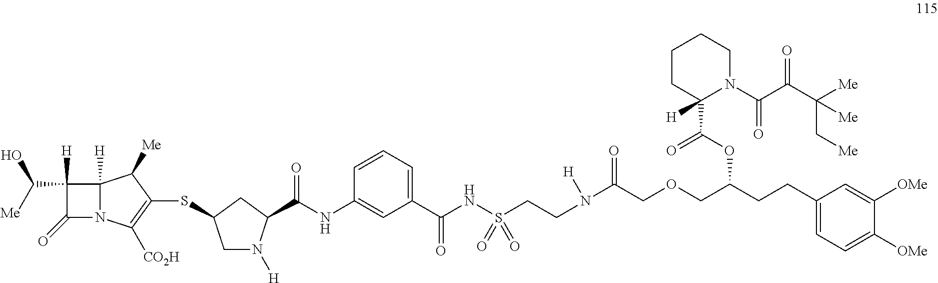

Cmpd 115 is prepared in a similar manner as outlined for cmpd 91.

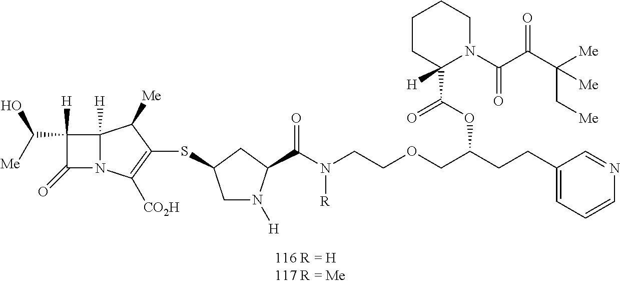

Cmpd 116 is prepared in a similar manner as outlined for cmpd 93. Cmpd 117 is prepared in a similar manner as outlined for cmpd 97.

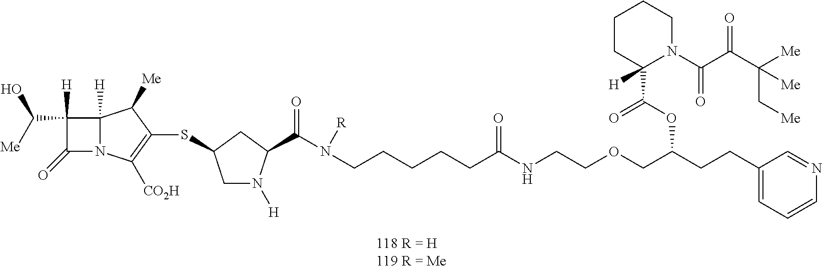

Cmpd 118 is prepared in a similar manner as outlined for cmpd 48. Cmpd 119 is prepared in a similar manner as outlined for cmpd 55.

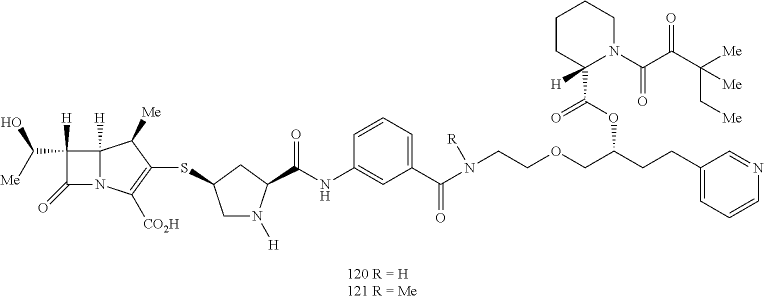

Cmpd 120 is prepared in a similar manner as outlined for cmpd 102. Cmpd 121 is prepared in a similar manner as outlined for cmpd 105.

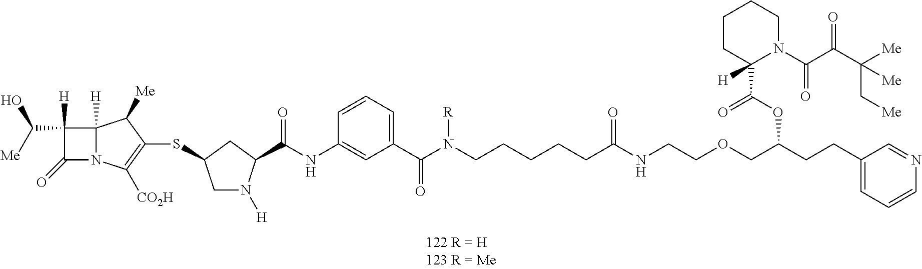

Cmpd 122 is prepared in a similar manner as outlined for cmpd 59. Cmpd 123 is prepared in a similar manner as outlined for cmpd 62.

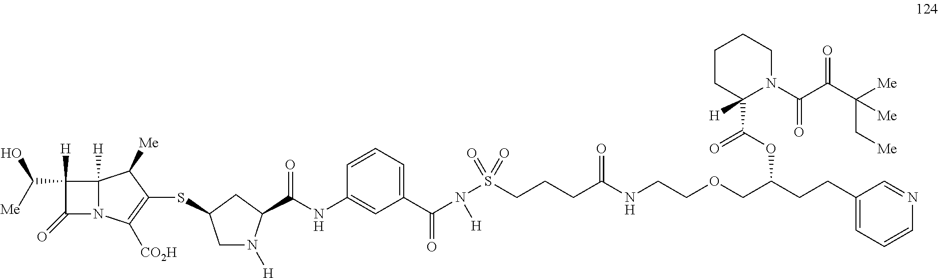

Cmpd 124 is prepared in a similar manner as outlined for cmpd 67.

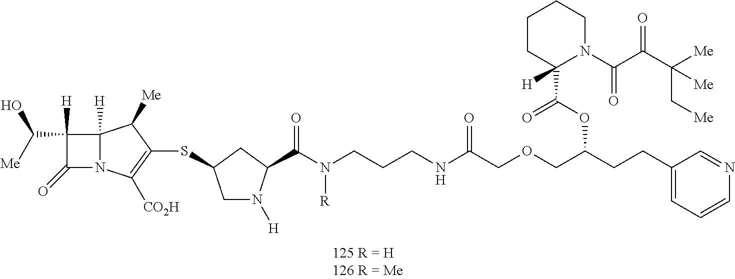

Cmpd 125 is prepared in a similar manner as outlined for cmpd 72. Cmpd 126 is prepared in a similar manner as outlined for cmpd 80.

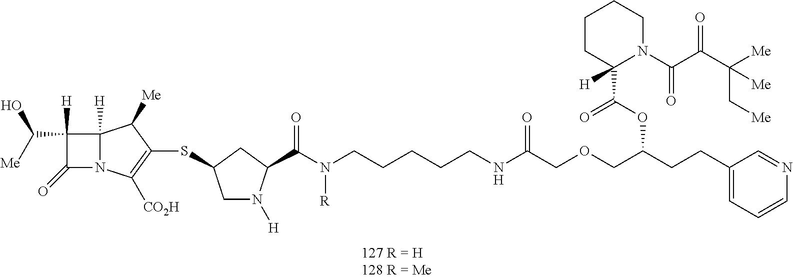

Cmpd 127 is prepared in a similar manner as outlined for cmpd 72. Cmpd 128 is prepared in a similar manner as outlined for cmpd 80.

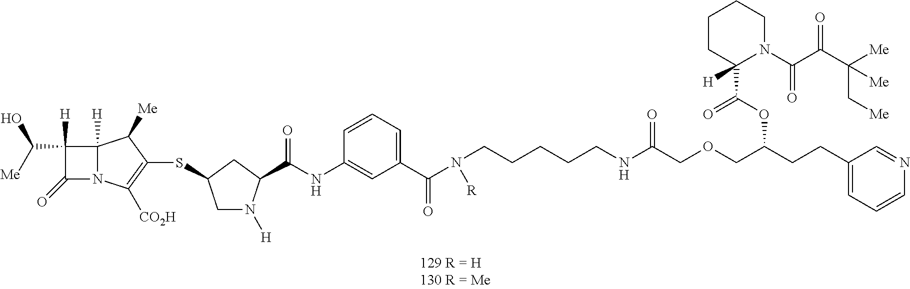

Cmpd 129 is prepared in a similar manner as outlined for cmpd 83. Cmpd 130 is prepared in a similar manner as outlined for cmpd 86.

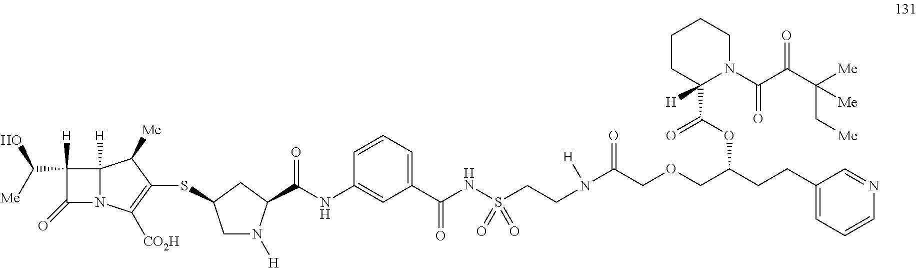

Cmpd 131 is prepared in a similar manner as outlined for cmpd 91.

### Example 11

The synthesis of compound 144 (and others) is shown below.

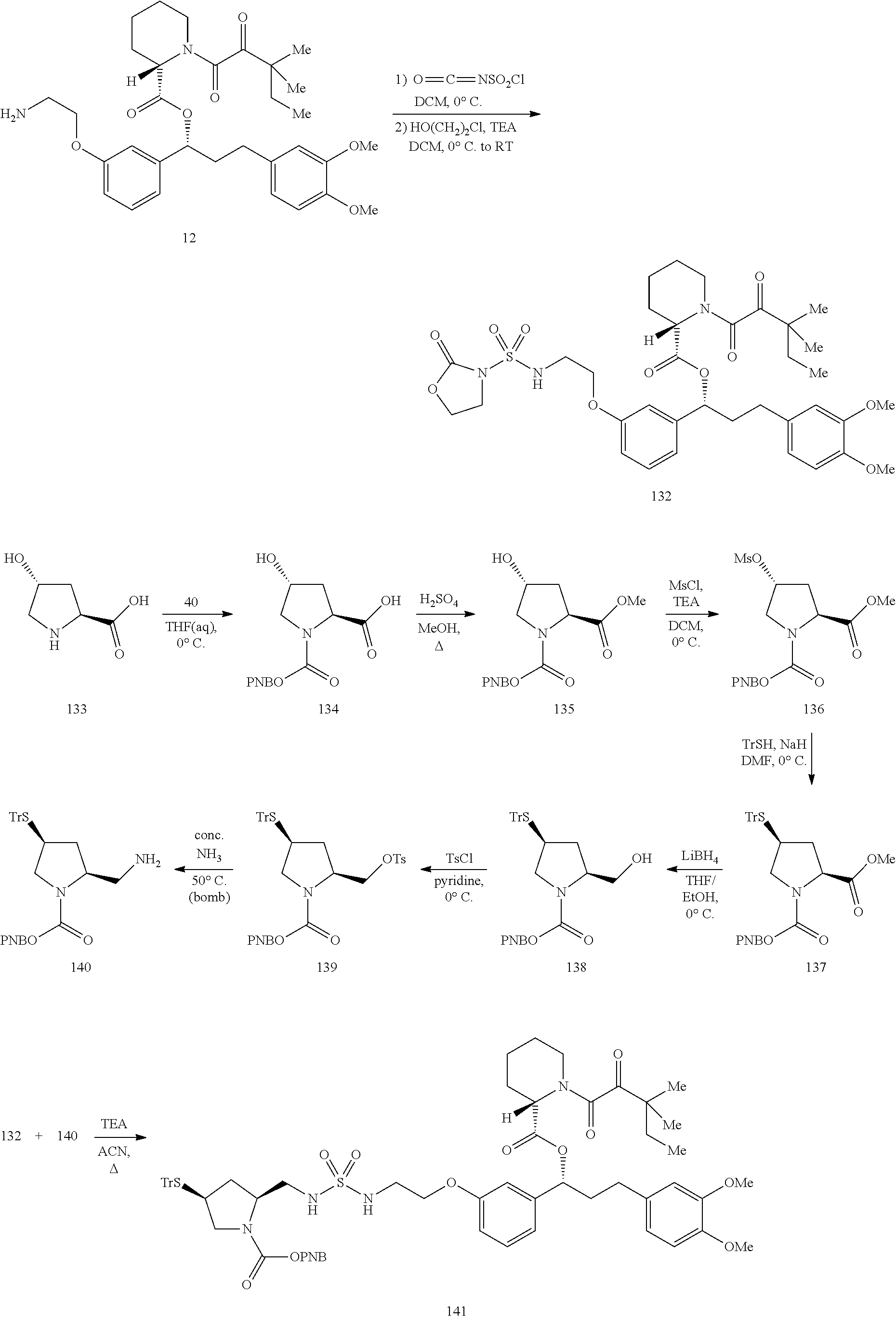

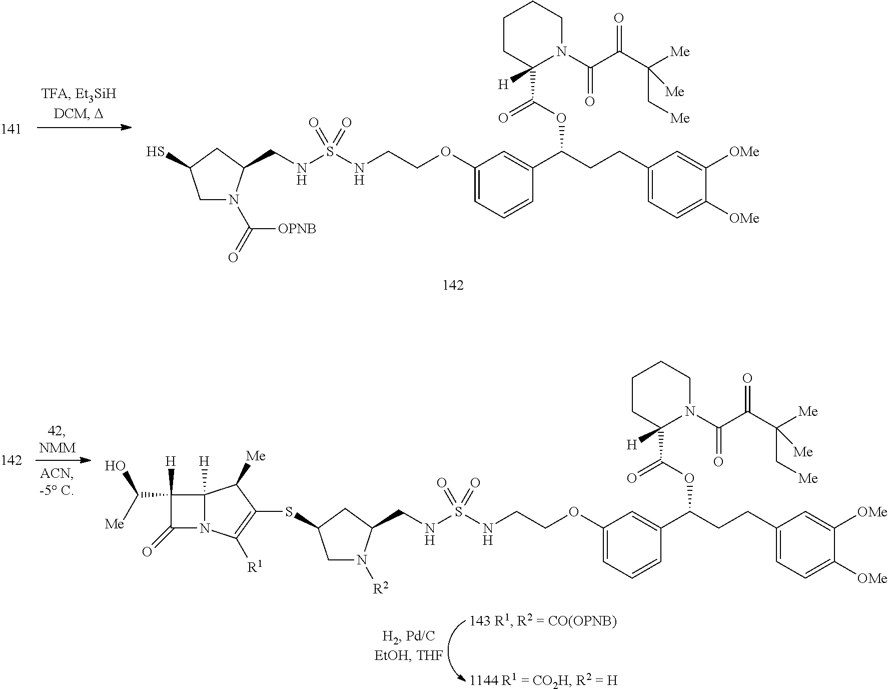

### Example 12

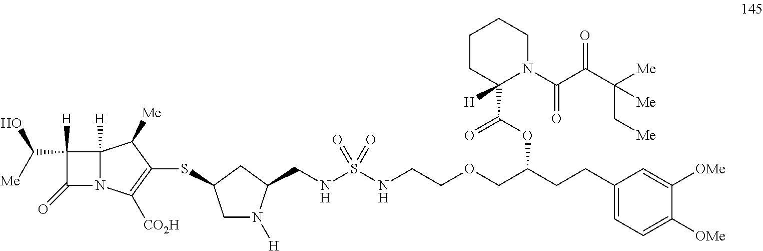

Cmpd 145 is prepared in a similar manner as outlined for cmpd 144.

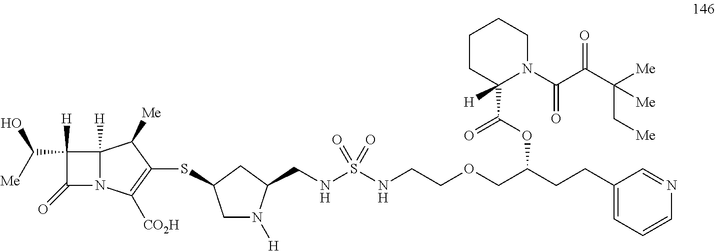

Cmpd 146 is prepared in a similar manner as outlined for cmpd 144.

### Example 13

The synthesis of compound 153 is shown below.

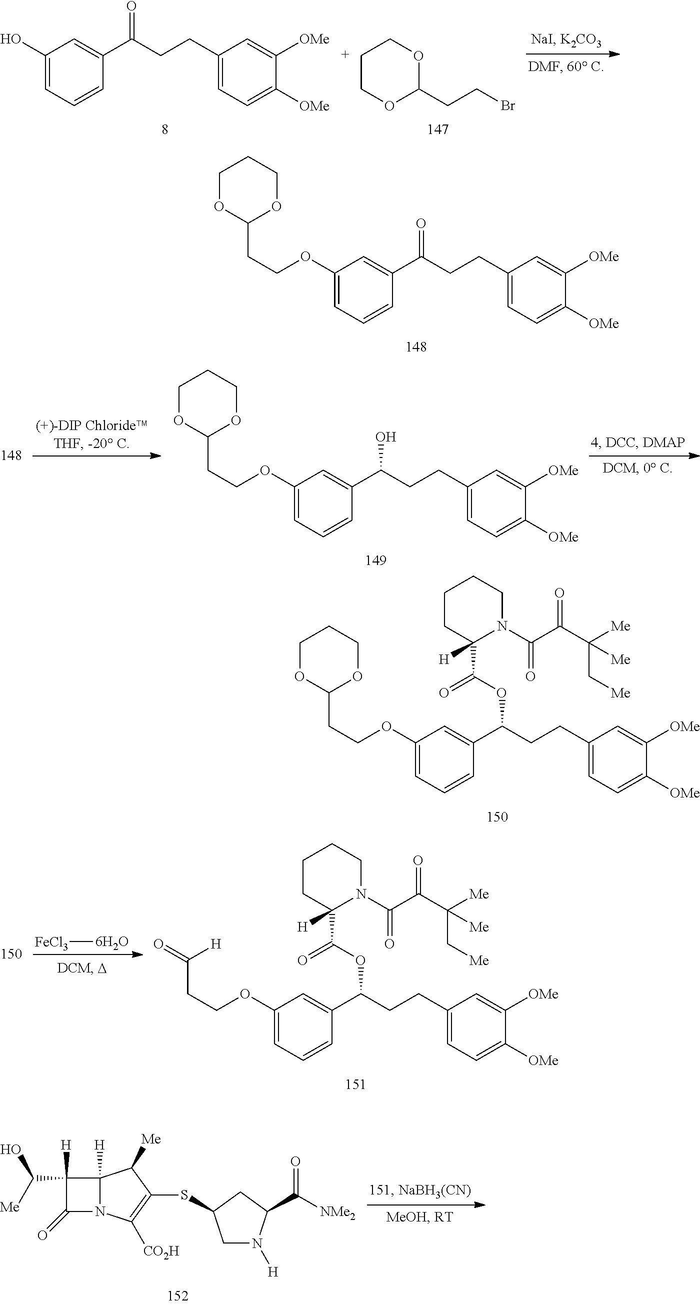

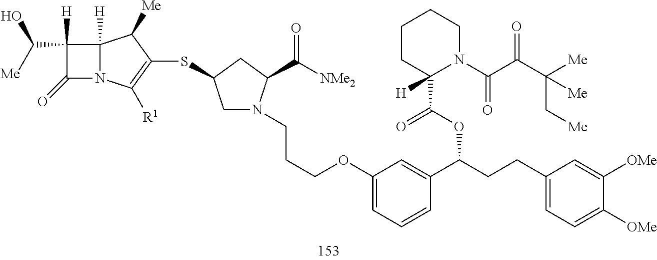

### Example 14

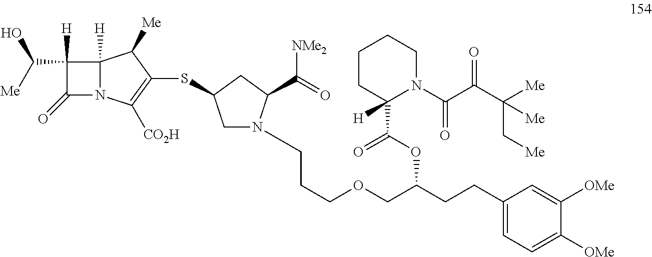

Cmpd 154 is prepared in a similar manner as outlined for cmpd 153.

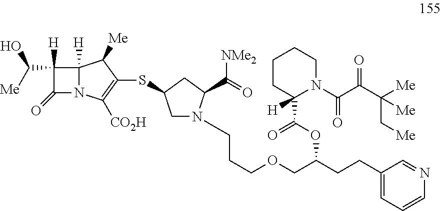

Cmpd 155 is prepared in a similar manner as outlined for cmpd 153.

### Example 15

Compound (3) was prepared according to Scheme 1.

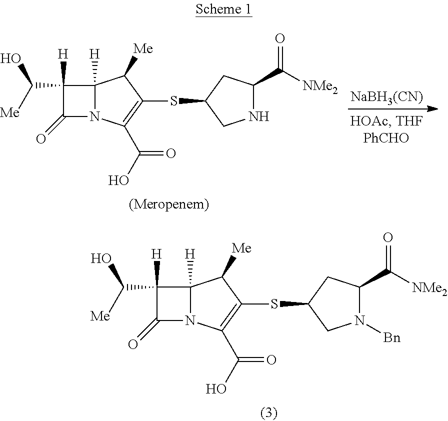

Details for the preparation of (4R,5S,6S)-3-(3S,5S)-1-benzyl-5-(dimethylcarbamoyl)pyrrolidin-3-ylthio)-6-((R)-1-hydroxyethyl)-4-methyl-7-oxo-1-azabicyclo[3.2.0]hept-2-ene-2-carboxylic acid (Compound (3)). Meropenem (0.26 mmol, 100 mg) was dissolved in dry THF (2.5 mL), acetic acid (1.2 equiv, 0.313 mmol, 18.8 mg, 18 pt) was added and the solution was cooled to 0° C. Then sodium cyanoborohydride (1.2 equiv, 0.313 mmol, 19.7 mg) was added, followed by benzaldehyde (1.2 equiv, 0.31 mmol, 33.2 mg, 32 μL). After 3 h the solvent was removed by a stream of nitrogen gas and the residue was submitted to prep. HPLC purification applying Eluent system 2, column B first and then column A to afford the title compound (15 mg, 12%).

¹H NMR (400 MHz, DMSO-d₆) δ ppm 7.20-7.36 (m, 5H), 4.92 (br. s., 1H), 3.92 (dd, J=9.2, 2.4 Hz, 1H), 3.84-3.90 (m, 1H), 3.84-3.98 (m, 2H), 3.57-3.66 (m, 1H), 3.55 (t, J=8.0 Hz, 1H), 3.41 (d, J=13.3 Hz, 1H), 3.05-3.12 (m, 1H), 3.02 (dd, J=6.9, 2.4 Hz, 1H), 2.98 (s, 3H), 2.85 (dd, J=9.8, 4.5 Hz, 1H), 2.79 (s, 3H), 2.72 (dd, J=9.5, 7.3 Hz, 1H), 2.53-2.61 (m, 1H), 1.50-1.66 (m, 1H), 1.13 (d, J=6.3 Hz, 3H), 1.02 (d, J=7.0 Hz, 3H); HPLC-UV-MS: ESI MS m/z 474.2 [C₂₄H₃₁N₃O₅S+H]⁺; 93.4% (AUC) at 220 nm.

### Example 16

Compound (2) was prepared according to Scheme 2.

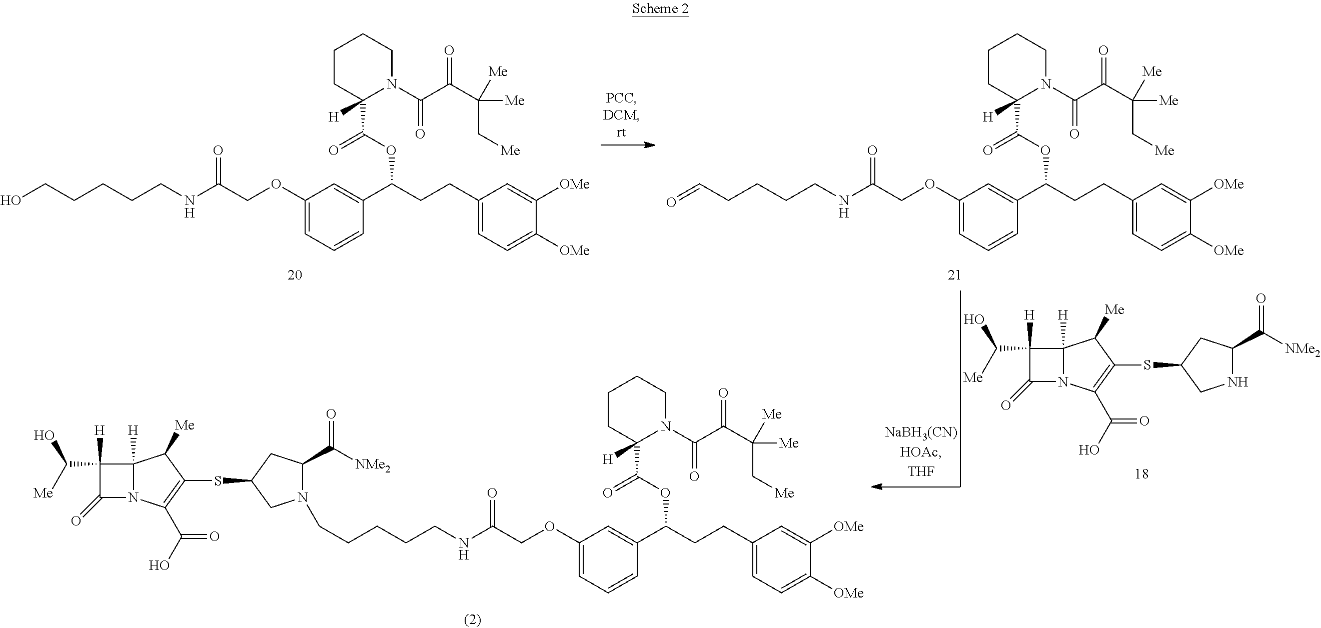

Preparation of (S)—((R)-3-(3,4-dimethoxyphenyl)-1-(3-(2-oxo-2-(5-oxopentylamino)ethoxy)phenyl)propyl) 1-(3,3-dimethyl-2-oxopentanoyl)piperidine-2-carboxylate (compound 21). Alcohol 20 (0.21 mmol, 140 mg) was dissolved in dry DCM (5 mL), and pyridinium chlorochromate (PCC) (1 equiv, 0.21 mmol, 45.3 mg) was added in one portion at rt. After 5 h the solution was treated with cc. NaHCO₃, the organic phase was dried over MgSO₄, and then concentrated. The residue was column chromatographed (isocratic hexane/EtOAc 1:2) to afford compound 21 (46 mg, 33%). ¹H-NMR (400 MHz, CDCl₃) δ ( ) HPLC-UV-MS: ESI MS m/z 667.3 [C₃₇H₅₀N₂O₉+H]⁺; 78.6% (AUC) at 220 nm.

Preparation of (4R,5S,6S)-3-((3S,5S)-1-(5-(2-(3-((R)-3-(3,4-dimethoxyphenyl)-1-((S)-1-(3,3-dimethyl-2-oxopentanoyl)piperidine-2-carbonyloxy)propyl)phenoxy)acetamido)pentyl)-5-(dimethylcarbamoyl)pyrrolidin-3-ylthio)-6-((R)-1-hydroxyethyl)-4-methyl-7-oxo-1-azabicyclo[3.2.0]hept-2-ene-2-carboxylic acid (compound (2)). Meropenem (18, 0.06 mmol, 23 mg) was dissolved in dry THF (2 mL) and the solution was cooled to 0° C. Aldehyde 21 (1 equiv, 0.06 mmol, 40 mg) was added followed by acetic acid (2 equiv, 0.12 mmol, 7.2 mg, 7 μL) and sodium cyanoborohidride (2 equiv, 0.12 mmol, 7.5 mg). The mixture was allowed to warm up to rt, and after 5 h the solvent was removed by a stream of nitrogen gas and the residue was submitted to prep. HPLC purification applying Eluent system 2, column B first and then column A to afford compound (2) (5 mg, 8%). ¹H NMR (400 MHz, DMSO-d₆) δ ppm 8.74 (br. s., 1H), 8.31 (br. s., 1H), 7.30 (t, J=8.2 Hz, 1H), 6.87-6.99 (m, 3H), 6.84 (d, J=8.3 Hz, 1H), 6.73-6.78 (m, 1H), 6.67 (dd, J=8.2, 2.1 Hz, 1H), 5.69 (dd, J=8.2, 4.9 Hz, 1H), 5.15 (d, J=5.3 Hz, 1H), 4.95 (br. s., 1H), 4.48 (s, 2H), 4.27 (br. s., 1H), 4.05 (br. s., 1H), 3.92 (br. s., 1H), 3.72 (s, 3H), 3.71 (s, 3H), 3.60 (br. s., 1H), 3.04-3.14 (m, 6H), 3.00 (dd, J=9.8, 4.0 Hz, 1H), 2.80 (s, 3H), 2.57 (br. s., 2H), 1.94-2.29 (m, 6H), 1.47-1.80 (m, 7H), 1.18-1.47 (m, 10H), 1.02-1.18 (m, 12H), 0.80 (t, J=7.5 Hz, 3H); HPLC-UV-MS: APCI+ m/z 1034.4 [C₅₄H₇₅N₅O₁₃S+H]⁺; 91.4% (AUC) at 220 nm.

### Example 17

Compound (1) was prepared according to Scheme 3.

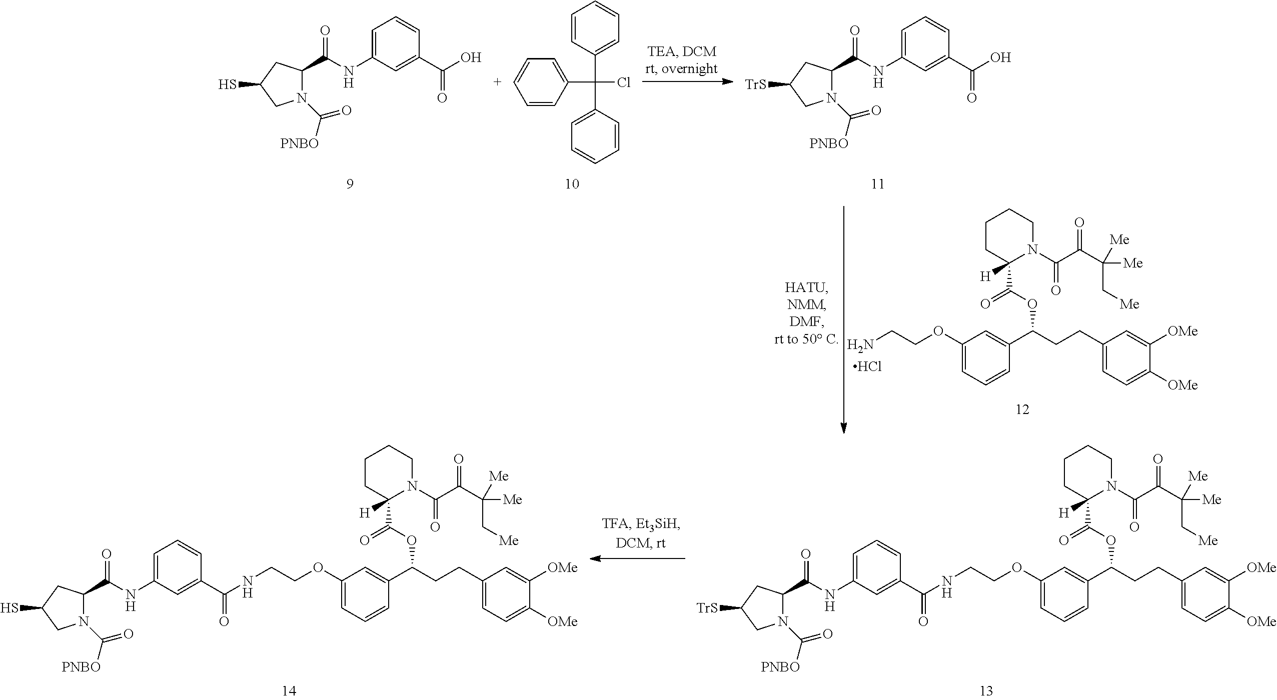

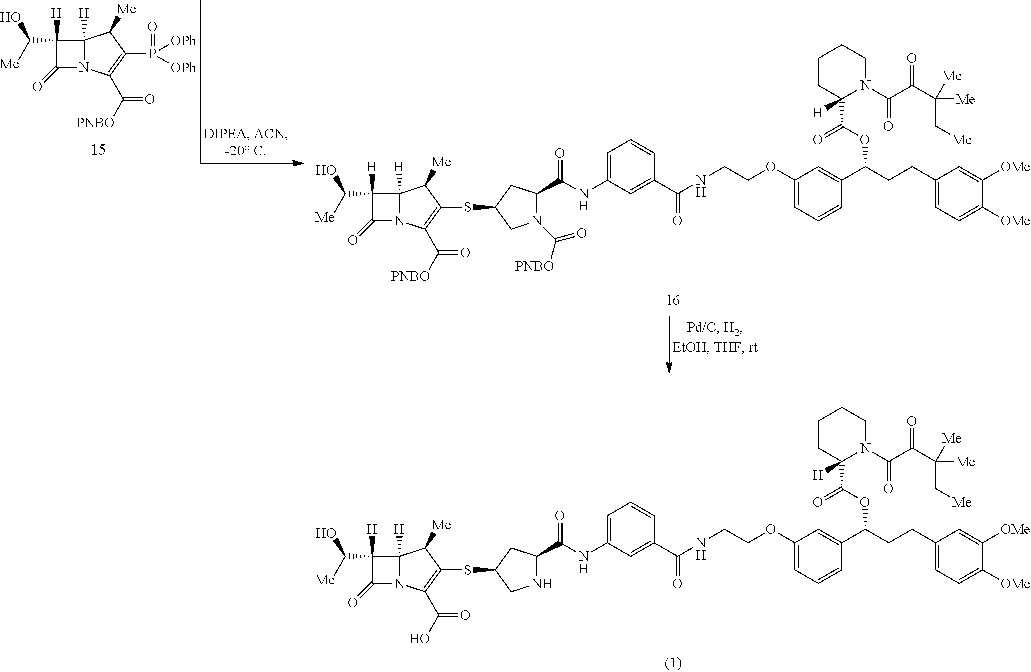

Preparation of 3-((2S,4S)-1-(4-nitrobenzyloxy)carbonyl)-4-(tritylthio)pyrrolidine-2-carboxamido)benzoic acid (compound 11). Thiol 9 (1336 mg, 3.00 mmol) was dissolved in dry DCM (25 mL), TEA (3 equiv, 9 mmol, 910.7 mg, 1248 μL) was added, followed by the addition of Tr-Cl (10, 1.1 equiv., 920 mg, 3.3 mmol) at rt and stirring was maintained overnight. The reaction mixture was washed with water, dried over Na₂SO₄, and then concentrated. The residue was column chromatographed (gradient elution with DCM/MeOH 100:2 to 100:5) to afford compound 11 (500 mg, 24%). ¹H NMR (400 MHz) δ ( ); HPLC-UV-MS: APCI+ and ESI+: can not be ionized in ion source; the main peak in UV 88.9% (AUC) at 220 nm.

Preparation of (S)—((R)-3-(3,4-dimethoxyphenyl)-1-(3-(2-(3-((2S,4S)-1-((4-nitrobenzyloxy)carbonyl)-4-(tritylthio)pyrrolidine-2-carboxamido)benzamido)ethoxy)phenyl)propyl) 1-(3,3-dimethyl-2-oxopentanoyl)piperidine-2-carboxylate (compound 13). Acid 11 (0.5 mmol, 343.9 mg) was dissolved in dry DMF (3.5 mL), HATU (1 equiv, 0.5 mmol, 190.1 mg) and N-methylmorpholine (3 equiv, 1.5 mmol, 151.7 mg, 165 μL) were added at rt. After 30 min 12 (1 equiv, 0.5 mmol, 284.4 mg) was added, and after 60 min stirring the temperature was raised to 50° C. and maintained there overnight. The volatiles were removed in vacuo, and the residue was partitioned between DCM and 5% aq. NaHCO₃ solution. The organic phase was dried over Na₂SO₄, and then concentrated. The residue was column chromatographed (isocratic hexane/EtOAc 1:2) to afford 13 (325 mg, 53%). ¹H-NMR (400 MHz) δ ( ); HPLC-UV-MS: APCI+ m/z 1238.8 [C₇₁H₇₅N₅O₁₃S+H]⁺; 91.0% (AUC) at 220 nm.

Preparation of (S)—((R)-3-(3,4-dimethoxyphenyl)-1-(3-(2-(3-((2S,4S)-4-mercapto-1-((4-nitrobenzyloxy)carbonyl)pyrrolidine-2-carboxamido)benzamido)ethoxy)phenyl)propyl) 1-(3,3-dimethyl-2-oxopentanoyl)piperidine-2-carboxylate (compound 14). Protected thiol 13 (0.26 mmol, 320 mg) was dissolved in dry DCM (10 mL), TFA (2 equiv, 0.517 mmol, 58.9 mg, 40 μl) and triethyl silane (1.3 equiv, 25 μL) were added at rt. The progress of the reaction was monitored by TLC with an eluent mixture of chloroform/MeOH 20:1 after treating of aliquots with cc. NaHCO₃. Additional portions of TFA (2×50 μL) and Et₃SiH (2×25 μL) were added until the reaction was judged to be completed by TLC. The mixture was treated with cc. NaHCO₃, dried over Na₂SO₄, and then concentrated. The residue was column chromatographed (isocratic DCM/ⁱPrOH 40:1) to afford compound 14 (234 mg, 91%). ¹H-NMR (400 MHz) δ ( ); HPLC-UV-MS: APCI+ m/z 996.2 [C₅₂H₆₁N₅O₁₃S+H]⁺; 89.9% (AUC) at 220 nm.

Preparation of (4R,5S,6S)-4-nitrobenzyl 3-((3S,5S)-5-(3-(2-(3-((R)-3-(3,4-dimethoxyphenyl)-1-((S)-1-(3,3-dimethyl-2-oxopentanoyl)piperidine-2-carbonyloxy)propyl)phenoxy)ethylcarbamoyl)phenylcarbamoyl)-1-((4-nitrobenzyloxy)carbonyl)pyrrolidin-3-ylthio)-6-((R)-1-hydroxyethyl)-4-methyl-7-oxo-1-azabicyclo[3.2.0]hept-2-ene-2-carboxylate (compound 16). Thiol 14 (0.22 mmol, 224 mg) was dissolved in dry MeCN (5 mL) and the solution was cooled to −20° C. DIPEA (3 equiv, 0.675 mmol, 87.2 mg, 118 μL) was added, followed by CP-1.04 (15, 1 equiv, 0.22 mmol, 133.7 mg) and stirring was maintained overnight. Then volatiles were removed in vacuo and residue was column chromatographed (gradient elution with DCM/ⁱPrOH 40:0 to 40:1) to afford compound 16 (236 mg, 78%). ¹H-NMR (400 MHz) δ ( ); HPLC-UV-MS: APCI+ m/z 1340.2 [C₆₉H₇₇N₇O₁₉S+H]⁺; 87.2% (AUC) at 220 nm.

Preparation of (4R,5S,6S)-3-((3S,5S)-5-(3-(2-(3-((R)-3-(3,4-dimethoxyphenyl)-1-((S)-1-(3,3-dimethyl-2-oxopentanoyl)piperidine-2-carbonyloxy)propyl)phenoxy)ethylcarbamoyl)phenylcarbamoyl)pyrrolidin-3-ylthio)-6-((R)-1-hydroxyethyl)-4-methyl-7-oxo-1-azabicyclo[3.2.0]hept-2-ene-2-carboxylic acid (compound (1)). PNBO-derivative 16 (0.07 mmol, 100 mg) was dissolved in a mixture of dry THF (5 mL) and ⁱPrOH (7.5 mL) followed by the addition of Pd/C (10 wt. %, 10 mg), and the mixture was placed under Hydrogen atmosphere (5 bar) for 18 h in a stainless steel autoclave. The catalyst was filtered off, the filtrate was concentrated in vacuo, and the residue was submitted to prep. HPLC purification applying Eluent system 2, column B first and then column A to afford compound (1) (6 mg, 8%). ¹H NMR (400 MHz, DMSO-d₆) δ ppm 10.02 (s, 1H), 8.67 (br. s., 1H), 8.08 (s, 1H), 7.84 (d, J=8.0 Hz, 1H), 7.54 (d, J=7.8 Hz, 1H), 7.38 (t, J=7.9 Hz, 1H), 7.24-7.33 (m, 1H), 6.86-6.99 (m, 3H), 6.83 (d, J=8.3 Hz, 1H), 6.75 (s, 1H), 6.67 (d, J=7.3 Hz, 1H), 5.69 (dd, J=8.3, 5.5 Hz, 1H), 5.15 (d, J=5.0 Hz, 1H), 5.00 (d, J=5.0 Hz, 1H), 4.02-4.20 (m, 3H), 3.95 (q, J=6.0 Hz, 1H), 3.83 (t, J=8.3 Hz, 1H), 3.68-3.74 (m, 6H), 3.53-3.67 (m, 3H), 3.41 (br. s., 2H), 3.03-3.21 (m, 2H), 2.57-2.84 (m, 3H), 1.96-2.37 (m, 4H), 1.44-1.76 (m, 6H), 1.21-1.42 (m, 3H), 1.13 (d, J=8.5 Hz, 13H), 0.79 (t, J=7.4 Hz, 3H); HPLC-UV-MS: ESI MS m/z 1026.5 [C₅₄H₆₇N₅O₁₃S+H]⁺; 85.5% (AUC) at 220 nm.

### Example 18

Compounds (1), (2), and (3) were tested to determine MIC₉₀ values. Data are presented in Table 1.

Rules for determining MIC values: Each extract has a starting concentration which is diluted 7 times for a total of 8 dilutions. The MIC is the lowest dilution without growth. Each serial dilution is repeated three times for three identical dilutions. For reporting the results, the following rules were used. Inconsistent values were thrown out. For instance, the MIC value reported for an extract with MIC readings of 4, 4, and 2 would be four (the 2 is thrown out). If the readings are all different and in series like 2, 4, and 8, the median MIC value of 4 would be reported. If the three values are all different and not in series, an error is reported. Besides the three identical dilutions at the same starting concentration, each extract has dilutions starting at three different concentrations: 64, 4, and 0.25 μg/mL. Each extract therefore has 3 identical dilution series at 3 different starting concentrations—a total of 24 dilutions. The dilutions overlap which can result in a disagreement in MIC values for two different starting dilutions. Since a small amount of error is introduced with each dilution, the MIC readings with the fewest dilutions are selected as the correct MIC values.

Abbreviations: MRSA=methicillin-resistant Staphylococcus aureus; SA=Staphylococcus aureus; PA=Pseudomonas aeruginosa; Kleb=Klebsiella pneumonia; EC=Escherichia coli; VSE=Vancomycin-sensitive enterococci faecium.

TABLE 1
MIC₉₀ values for various compounds.

| | | Max | MIC₉₀ (μg/mL) | | | | | | |
|---|---|---|---|---|---|---|---|---|---|
| | | Conc. | MRSA | SA | PA | Kleb | EC | VSE | VSE |
| Exp | Compound | (μg/mL) | 43300 | 29213 | 27853 | 4352 | 25922 | 49452 | 6569 |
| 1 | (3) | 64 | 4 | | 16 | | | 4 | 8 |
| 2 | (3) | 2 | | 0.25 | | | 1 | | |
| 3 | (3) | 0.12500 | | | | 0.06 | | | |
| 4 | (2) | 64 | 8 | | >64 | >64 | >64 | 16 | 32 |
| 5 | (2) | 2 | | 2 | | | | | |
| 6 | (2) | 0.12500 | | | | | | | |
| 7 | (1) | 64 | 8 | | >64 | >64 | >64 | 16 | 64 |
| 8 | (1) | 2 | | 2 | | | | | |
| 9 | (1) | 0.12500 | | | | | | | |
| 10 | Meropenem | 32 | 4 | | | | | 16 | 32 |
| 11 | Meropenem | 1 | | 0.25 | 1 | 0.12 | | | |
| 12 | Meropenem | 0.06000 | | | | | 0.06 | | |
| 13 | Chloramphenicol | 64 | 4 | 8 | >64 | ≦2 | 8 | 4 | ≦2 |
| 14 | Nitrofurantoin | 32 | 8 | 16 | >32 | 16 | 8 | 8 | 32 |
| 15 | Erythromycin | 64 | >64 | ≦2 | >64 | 8 | 64 | ≦2 | 8 |
| 16 | Amikacin | 8 | | | 1 | | | | |
| 17 | Doxycycline | 64 | | | 32 | | | | |

### Example 19

Compounds (1), (2), and (3) were tested to determine IC₅₀ values. IC₅₀ was determined using XlFit add-in using dose response model 200. The average of 3 replicates is presented for each compound in Table 2.

TABLE 2
IC₅₀values for various compounds.

| | | IC₅₀ (mg/mL) | | | | | | |
|---|---|---|---|---|---|---|---|---|
| | | MRSA | SA | PA | Kleb | EC | VSE | VSE |
| Exp | Compound | 43300 | 29213 | 27853 | 4352 | 25922 | 49452 | 6569 |
| 1 | (3) | 0.030 | 0.081 | 7.808 | 0.025 | 0.499 | 0.403 | 1.801 |
| 2 | (2) | 0.170 | 0.871 | >64 | >64 | >64 | 1.600 | 7.227 |
| 3 | (1) | 0.265 | 0.942 | >64 | >64 | >64 | 6.905 | 15.207 |
| 4 | Meropenem | 0.037 | 0.126 | 0.945 | 0.121 | 0.122 | 6.737 | 11.821 |
| 5 | Chloramphenicol | <2 | >64 | >64 | >64 | <2 | <2 | <2 |
| 6 | Nitrofurantoin | 1.826 | 7.706 | >32 | 3.677 | 3.267 | 3.566 | 2.873 |
| 7 | Erythromycin | >64 | <2 | >64 | 2.206 | 20.725 | <2 | 2.755 |
| 8 | Amikacin | | | 0.274 | | | | |
| 9 | Doxycycline | | | 6.302 | | | | |

### Example 20

Compounds (1), (2), and (3) were tested to determine IC₉₀ values against common bacteria. Data are presented for each compound in Table 3.

TABLE 3
IC₉₀ values for various compounds.

| | Max | MIC90 | | | | | | |
|---|---|---|---|---|---|---|---|---|
| | Conc. | MRSA | SA | PA | Kleb | EC | VRE | VSE |
| Compound | ug/mL | 43300 | 29213 | 27853 | 4352 | 25922 | 49452 | 6569 |
| (3) | 64 | 4 | | 16 | | | 4 | 8 |
| (3) | 2 | | 0.25 | | | 1 | | |
| (3) | 0.125 | | | | 0.06 | | | |
| (2) | 64 | 8 | | >64 | >64 | >64 | 16 | 32 |
| (2) | 2 | | 2 | | | | | |
| (2) | 0.125 | | | | | | | |
| (1) | 64 | 8 | | >64 | >64 | >64 | 16 | 64 |
| (1) | 2 | | 2 | | | | | |
| (1) | 0.125 | | | | | | | |
| Meropenem | 64 | 8 | | | | | 32 | 64 |
| Meropenem | 2 | | 0.5 | 2 | 0.25 | | | |
| Meropenem | 0.125 | | | | | 0.12 | | |

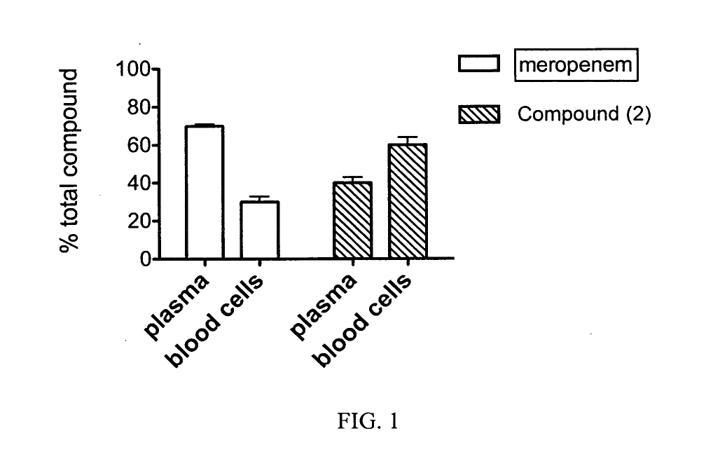

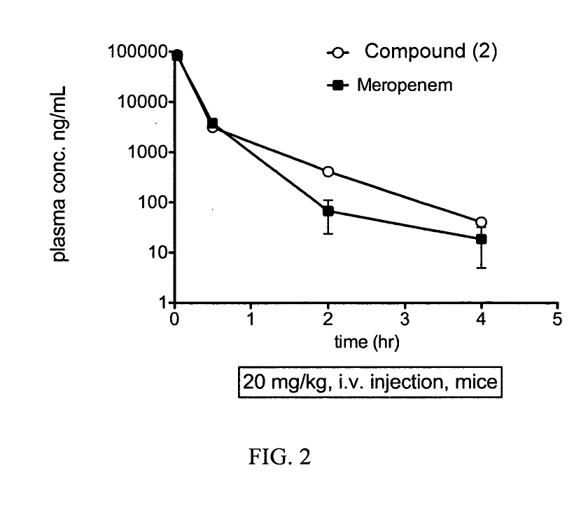

Whole mouse blood was incubated at 37° C. for 1 hour with compound (2), the samples centrifuged to separate cells from plasma, and the resulting amounts were assessed by LC-MS spectroscopy. Sample data are provided in FIGS. 1 and 2.

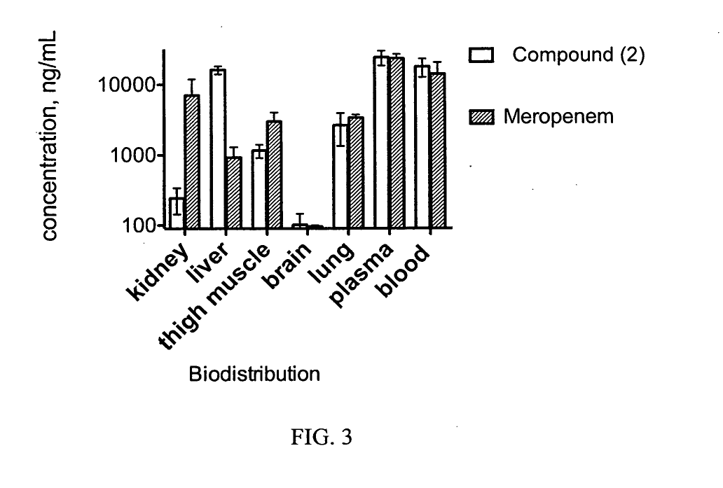

The biodistribution was determined by looking at concentration vs. time and determining the area under the curve for each compartment using LC-MS-MS spectroscopy. Data are provided in FIG. 3.

1. A compound having the structure of formula (I) 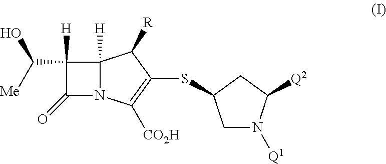
wherein:
R is selected from H and lower alkyl;
one of Q¹ and Q² is -L¹-U, and the other is selected from H, hydrocarbyl, heteroatom-containing hydrocarbyl, substituted hydrocarbyl, heteroatom-containing substituted hydrocarbyl, and functional groups;
L¹ is a linking moiety selected from hydrocarbylene, heteroatom-containing hydrocarbylene, substituted hydrocarbylene, heteroatom-containing substituted hydrocarbylene and functional groups;
U is a group selected from Units A, B, C, and E: 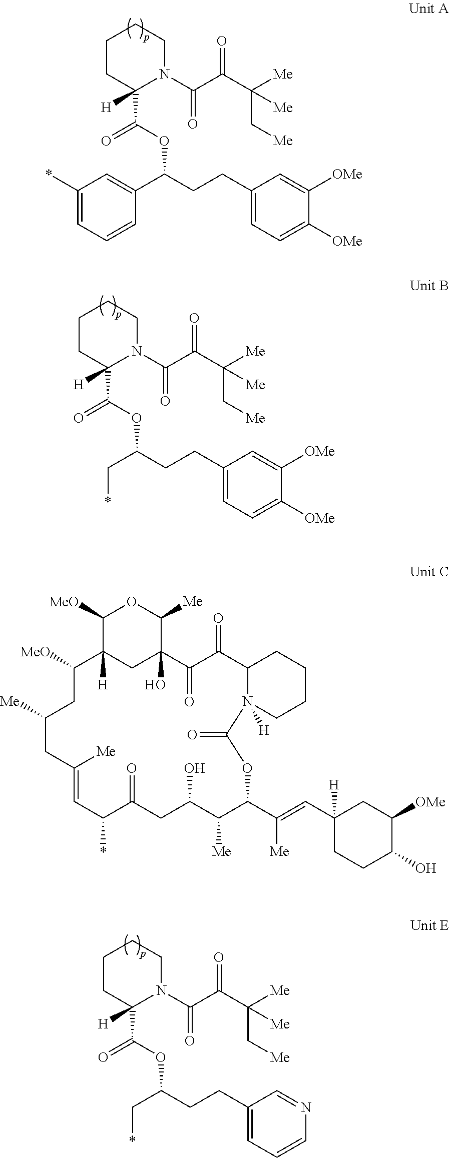
wherein p represents an integer from 0 to 2 and the stars represent the point of connection to L¹, as well as pharmaceutically acceptable salts, prodrugs, and metabolites thereof.

2. The compound of claim 1, wherein Q¹ is -L¹-U.

3. The compound of claim 2, wherein:
Q² is selected from H, —(CH₂)ₙ₂—X³—R¹, and —X³—NH—Ar¹;
n2 is an integer in the range of 0 to 5;
X³ is selected from a bond and —C(═O)—;
Ar¹ is aryl or heteroaryl substituted with one or more R¹ groups; and
R¹ is selected from H, hydrocarbyl, heteroatom-containing hydrocarbyl, substituted hydrocarbyl, heteroatom-containing substituted hydrocarbyl, and functional groups.

4. The compound of claim 3, wherein Ar¹ is selected from —C₆H₅ and —C₆H₅₋ₘR¹ₘ wherein m is an integer from 1 to 5.

5. The compound of claim 3, wherein each R¹ group is a hydrocarbyl moiety independently selected from C₁-C₂₄ alkyl, C₂-C₂₄ alkenyl, C₂-C₂₄ alkynyl, C₅-C₃₀ aryl, and C₆-C₃₀ aralkyl or is a functional group independently selected from halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-substituted C₁-C₂₄ alkylcarbamoyl, di-substituted alkylcarbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₅-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, and phosphino, mono- and di-(C₁-C₂₄ alkyl)-substituted phosphino, and mono- and di-(C₅-C₂₀ aryl)-substituted phosphino.

6. The compound of claim 1, wherein Q² is -L¹-U.

7. The compound of claim 6, wherein Q¹ is selected from H and lower alkyl.

8. The compound of claim 1, wherein L¹ is selected from alkylenes, alkenylenes, arylenes, alkarylenes, and aralkylenes, any of which may contain one or more heteroatoms and one or more substituents.

9. The compound of claim 8, wherein L¹ has the formula —Y-L-, wherein:
L is a linker selected from alkylenes, alkenylenes, amides, ureas, sulfoxides, sulfonamides, ethers, amines, carbonyls, and combinations thereof; and
Y is a linker selected from a bond, —C(═O)—, —C(═NH)—, —CH(OH)—(CH₂)ₙ₃—NR⁴—, and —(CH₂)ₙ₃—NH—(SO₂)ₙ₄—;
n3 is an integer in the range of 1 to 3;
n4 is 0 or 1; and
R⁴ is selected from H and lower alkyl.

10. The compound of claim 1, wherein:
Q¹ is selected from H, hydrocarbyl, substituted hydrocarbyl, heteroatom-containing hydrocarbyl, substituted heteroatom-containing hydrocarbyl, and functional groups;
Q²-Q is -L¹-U;
L¹ is —C(X¹)(X²)-Q³-;
X¹ and X² are independently selected from H and OH, or may be taken together to form ═O;
Q³ is selected from —(CH₂)ₙ₅—N(R⁵)(Q⁴) and -L-, or Q³ has a structure selected from 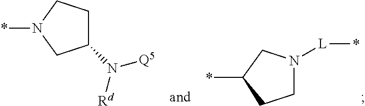
n5 is in the range of 0 to 5;
R⁵ is selected from H and lower alkyl;
Q⁴ is selected from -L-, —SO₂-L-, and aryl substituted with —C(═O)-L-;
Q⁵ is selected from -L- and —C(═O)—CH₂—NH-Q⁶;
Q⁶ is selected from -L-, —C(═NH)-L, and —C(═O)-L; and
L is a linker moiety selected from alkylenes, alkenylenes, amides, ureas, sulfoxides, sulfonamides, ethers, amines, carbonyls, and combinations thereof.

11. The compound of claim 1, wherein:
Q¹ is -L¹-U;
Q² is selected from H and —C(X¹)(X²)-Q³-;
X¹ and X² are independently selected from H and OH, or may be taken together to form ═O;
Q³ is —(CH₂)ₙ₅—N(R⁵)(Q⁴), or has the structure 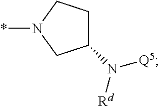
n5 is in the range of 0 to 5;
R⁵ is selected from H and lower alkyl;
Q⁴ is selected from lower alkyl, —SO₂NH₂, and aryl substituted with —COOH;
Q⁵ is —C(═O)—CH₂—NH—C(═O)—NH₂ or —C(═O)—CH₂—NH—C(═NH)—NH₂; and
L is a linker moiety selected from alkylenes, alkenylenes, amides, ureas, sulfoxides, sulfonamides, ethers, amines, carbonyls, and combinations thereof.

12. The compound of claim 9, wherein L is selected from 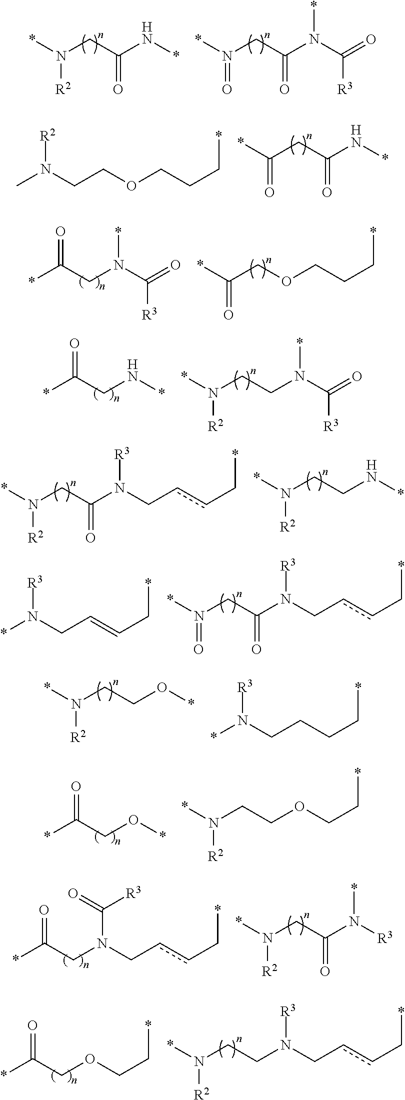 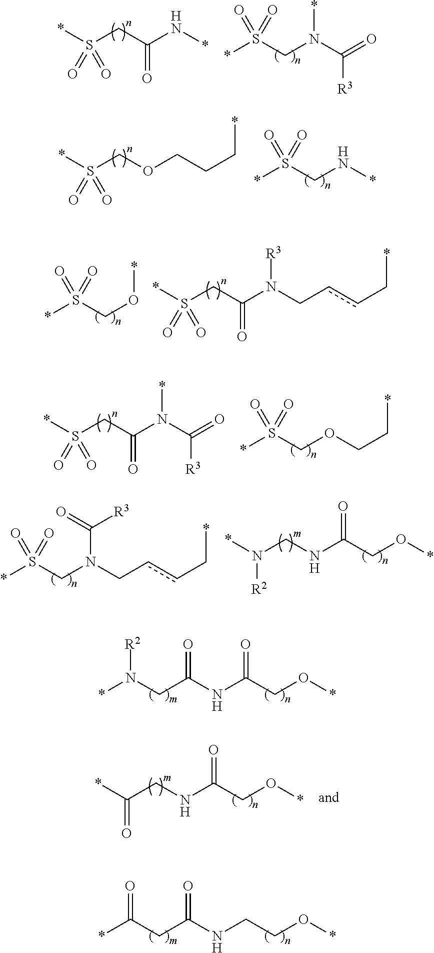
wherein:
R2 and R3 are independently selected from H, hydrocarbyl, and functional groups;
the stars represent attachment points to Y and to U; and
m and n are independently selected from 0, 1, and 2.

13. A compound having the structure of formula (II) 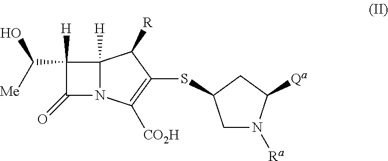
wherein:
R is selected from H and lower alkyl;
R^{a} is selected from H, hydrocarbyl, heteroatom containing hydrocarbyl, substituted hydrocarbyl, and substituted heteroatom-containing hydrocarbyl;
Q^{a} is selected from —(CH₂)ₘ₁—X^{a}—R^{b}, and —X^{a}—NH—Ar^{a};
m1 is selected from 0 and 1;
X^{a} is selected from a bond and —C(═O)—;
Ar^{a} is aryl or heteroaryl substituted with one or more R^{b} groups; and
R^{b} is selected from H, hydrocarbyl, and functional groups,
as well as pharmaceutically acceptable salts, prodrugs, and metabolites thereof.

14. The compound of claim 13, wherein R^{a} is aralkyl which may be substitute or unsubstituted, and which may contain one or more heteroatoms.

15. The compound of claim 13, wherein R^{a} has the formula -L^{a}-U, wherein:
L^{a} is a linking moiety; and
U is a group selected from Units A, B, C, and E: 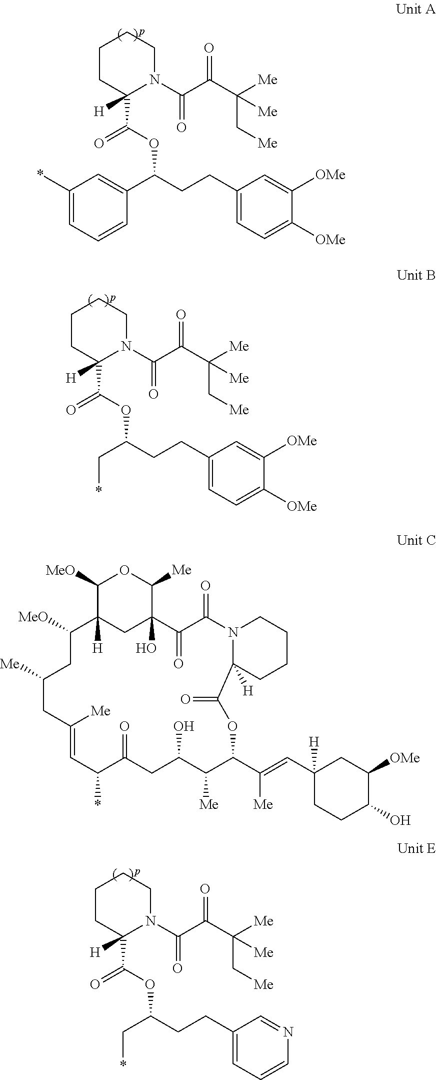
wherein p represents an integer from 0 to 2 and the stars represent the point of connection to L^{a}.

16. The compound of claim 15, wherein L^{a} is a linking moiety selected from alkyl, and aralkylene.

17. A pharmaceutical formulation comprising a compound according to claim 1.

18. The pharmaceutical formulation of claim 17, further comprising a pharmaceutically acceptable carrier.

19. A method of treating a microbial infection in a patient comprising administering to the patient a pharmaceutical formulation according to claim 17.

20. A method of use of a compound according to claim 1 in the preparation of a medicament for treating a microbial infection.
